SEQUENCE LISTING (156 sequences)

SEQ ID NO: 1 - DNA, 48 bp, Mus musculus
  CDS (1)..(48)
agatctagtcagagcattgtacatagtaatggaaacacctatttacaa

SEQ ID NO: 2 - protein, 16 aa, Mus musculus
RSSQSIVHSNGNTYLQ

SEQ ID NO: 3 - DNA, 21 bp, Mus musculus
  CDS (1)..(21)
aaagtttccaaccgactttat

SEQ ID NO: 4 - protein, 7 aa, Mus musculus
KVSNRLY

SEQ ID NO: 5 - DNA, 27 bp, Mus musculus
  CDS (1)..(27)
tttcaaggttcacatgttccgtggacg

SEQ ID NO: 6 - protein, 9 aa, Mus musculus
FQGSHVPWT

SEQ ID NO: 7 - DNA, 18 bp, Mus musculus
  CDS (1)..(18)
ggtggttatttatggaac

SEQ ID NO: 8 - protein, 6 aa, Mus musculus
GGYLWN

SEQ ID NO: 9 - DNA, 48 bp, Mus musculus
  CDS (1)..(48)
tacataagctacgacggtaccaataactacaaaccatctctcaaagat

SEQ ID NO: 10 - protein, 16 aa, Mus musculus
YISYDGTNNYKPSLKD

SEQ ID NO: 11 - DNA, 24 bp, Mus musculus
  CDS (1)..(24)
tacggtagggtcttctttgactac

SEQ ID NO: 12 - protein, 8 aa, Mus musculus
YGRVFFDY

SEQ ID NO: 13 - DNA, 26 bp, Mus musculus
atgaaatgcagctgggtcatsttctt

SEQ ID NO: 14 - DNA, 26 bp, Mus musculus
atgggatggagctrtatcatsytctt

SEQ ID NO: 15 - DNA, 26 bp, Mus musculus
atgaagwtgtggttaaactgggtttt

SEQ ID NO: 16 - DNA, 23 bp, Mus musculus
atgractttgggytcagcttgrt

SEQ ID NO: 17 - DNA, 26 bp, Mus musculus
atggactccaggctcaatttagtttt

SEQ ID NO: 18 - DNA, 26 bp, Mus musculus
atggctgtcytrgsgctrctcttctg

SEQ ID NO: 19 - DNA, 26 bp, Mus musculus
atggratggagckggrtctttmtctt

SEQ ID NO: 20 - DNA, 23 bp, Mus musculus
atgagagtgctgattcttttgtg

SEQ ID NO: 21 - DNA, 26 bp, Mus musculus
atggmttgggtgtggamcttgctatt

SEQ ID NO: 22 - DNA, 26 bp, Mus musculus
atgggcagacttacattctcattcct

SEQ ID NO: 23 - DNA, 28 bp, Mus musculus
atggattttgggctgattttttttattg

SEQ ID NO: 24 - DNA, 26 bp, Mus musculus
atgatggtgttaagtcttctgtacct

SEQ ID NO: 25 - DNA, 29 bp, Mus musculus
atgaagttgcctgttaggctgttggtgct

SEQ ID NO: 26 - DNA, 29 bp, Mus musculus
atggagwcagacacactcctgytatgggt

SEQ ID NO: 27 - DNA, 23 bp, Mus musculus
atgagtgtgctcactcaggtcct

SEQ ID NO: 28 - DNA, 26 bp, Mus musculus
atgaggrcccctgctcagwttyttgg

SEQ ID NO: 29 - DNA, 29 bp, Mus musculus
atggatttwcaggtgcagattwtcagctt

SEQ ID NO: 30 - DNA, 29 bp, Mus musculus
atggatttwcargtgcagattwtcagctt

SEQ ID NO: 31 - DNA, 26 bp, Mus musculus
atgaggtkcyytgytsagytyctgrg

SEQ ID NO: 32 - DNA, 23 bp, Mus musculus
atgggcwtcaagatggagtcaca

SEQ ID NO: 33 - DNA, 29 bp, Mus musculus
atgtggggayctktttycmmtttttcaat

SEQ ID NO: 34 - DNA, 24 bp, Mus musculus
atggtrtccwcasctcagttcctt

SEQ ID NO: 35 - DNA, 26 bp, Mus musculus
atgtatatatgtttgttgtctatttc

SEQ ID NO: 36 - DNA, 26 bp, Mus musculus
atggaagccccagctcagcttctctt

SEQ ID NO: 37 - DNA, 26 bp, Mus musculus
atgragtywcagacccaggtcttyrt

SEQ ID NO: 38 - DNA, 26 bp, Mus musculus
atggagacacattctcaggtctttgt

SEQ ID NO: 39 - DNA, 26 bp, Mus musculus
atggattcacaggcccaggttcttat

SEQ ID NO: 40 - DNA, 20 bp, Mus musculus
actggatggtgggaagatgg

SEQ ID NO: 41 - DNA, 42 bp, Mus musculus
  CDS (1)..(42)
gctgatgctgcaccaactgtatccatcttcccaccatccagt

SEQ ID NO: 42 - protein, 14 aa, Mus musculus
ADAAPTVSIFPPSS

SEQ ID NO: 43 - DNA, 20 bp, Mus musculus
ccatcttcccaccatccagt

SEQ ID NO: 44 - DNA, 18 bp, Mus musculus
ccagtggatagacagatg

SEQ ID NO: 45 - DNA, 33 bp, Mus musculus
  CDS (1)..(33)
gccaaaacgacacccccatctgtctatccactg

SEQ ID NO: 46 - protein, 11 aa, Mus musculus
AKTTPPSVYPL

SEQ ID NO: 47 - DNA, 21 bp, Mus musculus
cccccatctgtctatccactg

SEQ ID NO: 48 - DNA, 438 bp, Mus musculus
  CDS (28)..(393)
atgaagttgcctgttaggctgttggtgctgatgttctggattcctgcttccagaagtgatgttttgatgacccaaattccactctccctgcctgtcagtcttggagatcaagcctccatctcttgcagatctagtcagagcattgtacatagtaatggaaacacctatttacaatggtacctgcagaaaccaggtcagtctccaaagctcctgatctacaaagtttccaaccgactttatggggtcccagacaggttcagtggcagtggatcagggacagatttcacactcaagatcagcagcgtggaggctgaggatctgggagtttattactgctttcaaggttcacatgttccgtggacgttcggtggaggcaccaagctggaaatcaaacgggctgatgctgcaccaactgtatccatcttcccaccatccagt

SEQ ID NO: 49 - protein, 122 aa, Mus musculus
LMFWIPASRSDVLMTQIPLSLPVSLGDQASISCRSSQSIVHSNGNTYLQWYLQKPGQSPKLLIYKVSNRLYGVPDRFSGSGSGTDFTLKISSVEAEDLGVYYCFQGSHVPWTFGGGTKLEIK

SEQ ID NO: 50 - DNA, 438 bp, Mus musculus
tacttcaacggacaatccgacaaccacgactacaagacctaaggacgaaggtcttcactacaaaactactgggtttaaggtgagagggacggacagtcagaacctctagttcggaggtagagaacgtctagatcagtctcgtaacatgtatcattacctttgtggataaatgttaccatggacgtctttggtccagtcagaggtttcgaggactagatgtttcaaaggttggctgaaataccccagggtctgtccaagtcaccgtcacctagtccctgtctaaagtgtgagttctagtcgtcgcacctccgactcctagaccctcaaataatgacgaaagttccaagtgtacaaggcacctgcaagccacctccgtggttcgacctttagtttgcccgactacgacgtggttgacataggtagaagggtggtaggtca

SEQ ID NO: 51 - DNA, 438 bp, Mus musculus
  CDS (25)..(405)
atgatggtgttaagtcttctgtacctcttgacagccattcctggtatcctgtctgatgtacagcttcaggagtcaggacctggcctcgtgaaaccttctcagtctctgtctctcacctgctctgtcaccggctactccatcaccggtggttatttatggaactggatccggcagtttccaggaaacaaactggagtggatgggctacataagctacgacggtaccaataactacaaaccatctctcaaagatcgaatctccatcactcgtgacacatctaagaaccagtttttcctgaagttgaattctgtgactaatgaagacacagctacatattactgtgcaagatacggtagggtcttctttgactactggggccaaggcaccactctcacagtctcctcagccaaaacgacacccccatctgtctatccactg

SEQ ID NO: 52 - protein, 127 aa, Mus musculus
LLTAIPGILSDVQLQESGPGLVKPSQSLSLTCSVTGYSITGGYLWNWIRQFPGNKLEWMGYISYDGTNNYKPSLKDRISITRDTSKNQFFLKLNSVTNEDTATYYCARYGRVFFDYWGQGTTLTVSS

SEQ ID NO: 53 - DNA, 438 bp, Mus musculus
tactaccacaattcagaagacatggacaactgtcggtaaggaccataggacagactacatgtcgaagtcctcagtcctggaccggagcactttggaagagtcagagacagagagtggacgagacagtggccgatgaggtagtggccaccaataaataccttgacctaggccgtcaaaggtcctttgtttgacctcacctacccgatgtattcgatgctgccatggttattgatgtttggtagagagtttctagcttagaggtagtgagcactgtgtagattcttggtcaaaaaggacttcaacttaagacactgattacttctgtgtcgatgtataatgacacgttctatgccatcccagaagaaactgatgaccccggttccgtggtgagagtgtcagaggagtcggttttgctgtgggggtagacagataggtgac

SEQ ID NO: 54 - protein, 112 aa, Mus musculus
DVLMTQIPLSLPVSLGDQASISCRSSQSIVHSNGNTYLQWYLQKPGQSPKLLIYKVSNRLYGVPDRFSGSGSGTDFTLKISSVEAEDLGVYYCFQGSHVPWTFGGGTKLEIK

SEQ ID NO: 55 - protein, 112 aa, Mus musculus
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPFTFGSGTKLDIK

SEQ ID NO: 56 - protein, 112 aa, Mus musculus
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPWTFGGGTKLEIK

SEQ ID NO: 57 - protein, 112 aa, Mus musculus
DVVMTQTPLSLPVSLGDQASISCRSSQSLVHSNGNTYLEWYLQKPGQSPKLLIYKVSNRFSGVPDRFSGSGSGTDFTLKISRVEAEDLGVYYCFQGTHVPYTFGGGTKLEIK

SEQ ID NO: 58 - protein, 112 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPQTFGQGTKVEIK

SEQ ID NO: 59 - protein, 100 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTP

SEQ ID NO: 60 - protein, 112 aa, Homo sapiens
  VARIANT 35, 36, 39, 99: Xaa = Any Amino Acid
DIVMTQSPLSLPVTPGEPASISCRSSQSLLHSDGXXYLXWYLQKPGQSPQLLIYLVSNRASGVPDRFSGSGSGTDFTLKISRVEAEDVGVYYCMQALQXPRTFGQGTKVEIK

SEQ ID NO: 61 - protein, 112 aa, Homo sapiens
DVVMTQSPLSLPVTPGEPASISCRSSQSIVHSNGNTYLQWYLQKPGQSPQLLIYKVSNRLYGVPDRFSGSGSGTDFTLKISRVEAEDVGVYYCFQGSHVPWTFGQGTKVEIK

SEQ ID NO: 62 - DNA, 433 bp, Homo sapiens
  CDS (22)..(414)
gtcagaacgcgtgccgccaccatgaagttgcctgttaggctgttggtgctgatgttctggtttcctgcttccagcagtgatgttgtgatgactcagtctccactctccctgcccgtcacccctggagagccggcctccatctcctgcaggtctagtcagagcattgtacatagtaatggaaacacctatttgcaatggtacctgcagaagccagggcagtctccacagctcctgatctataaagtttctaatcggctttatggggtccctgacaggttcagtggcagtggatcaggcacagattttacactgaaaatcagcagagtggaggctgaggatgttggggtttattactgctttcaaggttcacatgttccgtggacgttcggccaagggaccaaggtggaaatcaaacgtgagtggatcctctgcg

SEQ ID NO: 63 - protein, 131 aa, Homo sapiens
MKLPVRLLVLMFWFPASSSDVVMTQSPLSLPVTPGEPASISCRSSQSIVHSNGNTYLQWYLQKPGQSPQLLIYKVSNRLYGVPDRFSGSGSGTDFTLKISRVEAEDVGVYYCFQGSHVPWTFGQGTKVEIK

SEQ ID NO: 64 - DNA, 433 bp, Homo sapiens
cagtcttgcgcacggcggtggtacttcaacggacaatccgacaaccacgactacaagaccaaaggacgaaggtcgtcactacaacactactgagtcagaggtgagagggacgggcagtggggacctctcggccggaggtagaggacgtccagatcagtctcgtaacatgtatcattacctttgtggataaacgttaccatggacgtcttcggtcccgtcagaggtgtcgaggactagatatttcaaagattagccgaaataccccagggactgtccaagtcaccgtcacctagtccgtgtctaaaatgtgacttttagtcgtctcacctccgactcctacaaccccaaataatgacgaaagttccaagtgtacaaggcacctgcaagccggttccctggttccacctttagtttgcactcacctaggagacgc

SEQ ID NO: 65 - protein, 112 aa, Homo sapiens [sequence omitted]

SEQ ID NO: 66 - DNA, 433 bp, Homo sapiens
  CDS (22)..(414)
gtcagaacgcgtgccgccaccatgaagttgcctgttaggctgttggtgctgatgttctggtttcctgcttccagcagtgatattgtgatgactcagtctccactctccctgcccgtcacccctggagagccggcctccatctcctgcaggtctagtcagagcattgtacatagtaatggaaacacctatttgcaatggtacctgcagaagccagggcagtctccacagctcctgatctataaagtttctaatcggctttatggggtccctgacaggttcagtggcagtggatcaggcacagattttacactgaaaatcagcagagtggaggctgaggatgttggggtttattactgctttcaaggttcacatgttccgtggacgttcggccaagggaccaaggtggaaatcaaacgtgagtggatcctctgcg

SEQ ID NO: 67 - protein, 131 aa, Homo sapiens
MKLPVRLLVLMFWFPASSSDIVMTQSPLSLPVTPGEPASISCRSSQSIVHSNGNTYLQWYLQKPGQSPQLLIYKVSNRLYGVPDRFSGSGSGTDFTLKISRVEAEDVGVYYCFQGSHVPWTFGQGTKVEIK

SEQ ID NO: 68 - DNA, 433 bp, Homo sapiens; identical to SEQ ID NO: 64

SEQ ID NO: 69 - protein, 117 aa, Mus musculus
DVQLQESGPGLVKPSQSLSLTCSVTGYSITGGYLWNWIRQFPGNKLEWMGYISYDGTNNYKPSLKDRISITRDTSKNQFFLKLNSVTNEDTATYYCARYGRVFFDYWGQGTTLTVSS

SEQ ID NO: 70 - protein, 118 aa, Mus musculus
DVQLQESGPGLVKPSQSLSLTCSVTGYSITSGYYWNWIRQFPGNKLEWMGYINYDGNNNYNPSLKNRISITRDTSKNQFFLKLNSVTTEDTATYYCAREGYGYFFDYWGQGTTLTVSS

SEQ ID NO: 71 - protein, 118 aa, Mus musculus
EVQLQESGPSLVKPSQTLSLTCSVTGDSITSGYWNNWIRQFPGNKLEWMGYISYSGSTYYNPSLKSRISITRDTSKNQYFLQLNSVTTEDTATYYCARGGYGYGFDYWGQGTTVTVSS

SEQ ID NO: 72 - protein, 117 aa, Homo sapiens
  VARIANT 59: Xaa = Any Amino Acid
QVQLQESGPGLVKPSQTLSLTCTVSGGSVSSYWSWNWIRQPPGKGLEWIGRIYYSGSTXYNPSLKSRVTISVDTSKNQFSLKLSSVTAADTAVYYCARELPGGYDVWGQGTLVTVSS

SEQ ID NO: 73 - protein, 123 aa, Homo sapiens
QVQLQESGPGLVKPSETLSLTCTVSGYSISSGYYWSWIRQPPGKGLEWIGSMFHSGSSYYNPSLKSRVTISVDTSKNQFSLQLRSVTAADTAVYYCARGRYCSSTSCNWFDPWGQGTLVTVSS

SEQ ID NO: 74 - protein, 98 aa, Homo sapiens
QVQLQESGPGLVKPSETLSLTCTVSGYSISSGYYWSWIRQPPGKGLEWIGSIYHSGSTYYNPSLKSRVTISVDTSKNQFSLKLSSVTAADTAVYYCAR

SEQ ID NO: 75 - protein, 117 aa, Homo sapiens
QVQLQESGPGLVKPSETLSLTCTVSGYSITGGYLWNWIRQPPGKGLEWMGYISYDGTNNYKPSLKDRITISRDTSKNQFSLKLSSVTAADTAVYYCARYGRVFFDYWGQGTLVTVSS

SEQ ID NO: 76 - DNA, 445 bp, Homo sapiens
  CDS (22)...(426)
gtcagaacgcgtgccgccaccatgaaagtgttgagtctgttgtacctcttgacagccattcctggtatcctgtctcaggtgcagcttcaggagtcgggcccaggactggtgaagccttcggagaccctgtccctcacctgcactgtctctggttactccatcaccggtggttatttatggaactggatacggcagcccccagggaagggactggagtggatggggtatatcagctacgacggtaccaataactacaaaccctccctcaaggatcgaatcaccatatcacgtgacacgtccaagaaccagttctccctgaagctgagctctgtgaccgctgcggacactgcagtgtattactgtgcgagatacggtagggtcttctttgactactggggccagggaaccctggtcaccgtctcctcaggtgagtggatcctctgcg

SEQ ID NO: 77 - protein, 135 aa, Homo sapiens
MKVLSLLYLLTAIPGILSQVQLQESGPGLVKPSETLSLTCTVSGYSITGGYLWNWIRQPPGKGLEWMGYISYDGTNNYKPSLKDRITISRDTSKNQFSLKLSSVTAADTAVYYCARYGRVFFDYWGQGTLVTVSS

SEQ ID NO: 78 - DNA, 445 bp, Homo sapiens
cagtcttgcgcacggcggtggtactttcacaactcagacaacatggagaactgtcggtaaggaccataggacagagtccacgtcgaagtcctcagcccgggtcctgaccacttcggaagcctctgggacagggagtggacgtgacagagaccaatgaggtagtggccaccaataaataccttgacctatgccgtcgggggtcccttccctgacctcacctaccccatatagtcgatgctgccatggttattgatgtttgggagggagttcctagcttagtggtatagtgcactgtgcaggttcttggtcaagagggacttcgactcgagacactggcgacgcctgtgacgtcacataatgacacgctctatgccatcccagaagaaactgatgaccccggtcccttgggaccagtggcagaggagtccactcacctaggagacgc

SEQ ID NO: 79 - protein, 117 aa, Homo sapiens [sequence omitted]

SEQ ID NO: 80 - DNA, 445 bp, Homo sapiens
  CDS (22)...(426)
gtcagaacgcgtgccgccaccatgaaagtgttgagtctgttgtacctcttgacagccattcctggtatcctgtctcaggtgcagcttcaggagtcgggcccaggactggtgaagccttcggagaccctgtccctcacctgcactgtctctggttactccatcaccggtggttatttatggaactggatacggcagcccccagggaagggactggagtggatcgggtatatcagctacgacggtaccaataactacaaaccctccctcaaggatcgagtcaccatatcacgtgacacgtccaagaaccagttctccctgaagctgagctctgtgaccgctgcggacactgcagtgtattactgtgcgagatacggtagggtcttctttgactactggggccagggaaccctggtcaccgtctcctcaggtgagtggatcctctgcg

SEQ ID NO: 81 - protein, 135 aa, Homo sapiens
MKVLSLLYLLTAIPGILSQVQLQESGPGLVKPSETLSLTCTVSGYSITGGYLWNWIRQPPGKGLEWIGYISYDGTNNYKPSLKDRVTISRDTSKNQFSLKLSSVTAADTAVYYCARYGRVFFDYWGQGTLVTVSS

SEQ ID NO: 82 - DNA, 445 bp, Homo sapiens
cagtcttgcgcacggcggtggtactttcacaactcagacaacatggagaactgtcggtaaggaccataggacagagtccacgtcgaagtcctcagcccgggtcctgaccacttcggaagcctctgggacagggagtggacgtgacagagaccaatgaggtagtcgccaccaataaataccttgacctatgccgtcgggggtcccttccctgacctcacctagcccatatagtcgatgctgccatggttattgatgtttgggagggagttcctagctcagtggtatagtgcactgtgcaggttcttggtcaagagggacttcgactcgagacactggcgacgcctgtgacgtcacataatgacacgctctatgccatcccagaagaaactgatgaccccggtcccttgggaccagtggcagaggagtccactcacctaggagacgc

SEQ ID NO: 83 - protein, 117 aa, Homo sapiens
QVQLQESGPGLVKPSETLSLTCTVSGYSISGGYLWNWIRQPPGKGLEWIGYISYDGTNNYKPSLKDRVTISVDTSKNQFSLKLSSVTAADTAVYYCARYGRVFFDYWGQGTLVTVSS

SEQ ID NO: 84 - DNA, 445 bp, Homo sapiens
  CDS (22)...(426)
gtcagaacgcgtgccgccaccatgaaagtgttgagtctgttgtacctcttgacagccattcctggtatcctgtctcaggtgcagcttcaggagtcgggcccaggactggtgaagccttcggagaccctgtccctcacctgcactgtctctggttactccatcagcggtggttatttatggaactggatacggcagcccccagggaagggactggagtggatcgggtatatcagctacgacggtaccaataactacaaaccctccctcaaggatcgagtcaccatatcagtggacacgtccaagaaccagttctccctgaagctgagctctgtgaccgctgcggacactgcagtgtattactgtgcgagatacggtagggtcttctttgactactggggccagggaaccctggtcaccgtctcctcaggtgagtggatcctctgcg

SEQ ID NO: 85 - protein, 135 aa, Homo sapiens
MKVLSLLYLLTAIPGILSQVQLQESGPGLVKPSETLSLTCTVSGYSISGGYLWNWIRQPPGKGLEWIGYISYDGTNNYKPSLKDRVTISVDTSKNQFSLKLSSVTAADTAVYYCARYGRVFFDYWGQGTLVTVSS

SEQ ID NO: 86 - DNA, 445 bp, Homo sapiens
cagtcttgcgcacggcggtggtactttcacaactcagacaacatggagaactgtcggtaaggaccataggacagagtccacgtcgaagtcctcagcccgggtcctgaccacttcggaagcctctgggacagggagtggacgtgacagagaccaatgaggtagtcgccaccaataaataccttgacctatgccgtcgggggtcccttccctgacctcacctagcccatatagtcgatgctgccatggttattgatgtttgggagggagttcctagctcagtggtatagtcacctgtgcaggttcttggtcaagagggacttcgactcgagacactggcgacgcctgtgacgtcacataatgacacgctctatgccatcccagaagaaactgatgaccccggtcccttgggaccagtggcagaggagtccactcacctaggagacgc

SEQ ID NO: 87 - DNA, 18 bp, Artificial sequence
  Description de la Artificial sequence Oligonucleotide
gtcagaacgcgtgccgcc

SEQ ID NO: 88 - DNA, 32 bp, Artificial sequence
  Description of artificial sequence Oligonucleotide
accatgaagttgcctgttaggctgttggtgct

SEQ ID NO: 89 - DNA, 32 bp, Artificial sequence
  Description of artificial sequence Oligonucleotide
gatgttctggtttcctgcttccagcagtgatg

SEQ ID NO: 90 - DNA, 32 bp, Artificial sequence
  Description of artificial sequence Oligonucleotide
ttgtgatgactcagtctccactctccctgccc

SEQ ID NO: 91 - DNA, 32 bp, Artificial sequence
  Description of artificial sequence Oligonucleotide
gtcacccctggagagccggcctccatctcctg

SEQ ID NO: 92 - DNA, 32 bp, Artificial sequence
  Description of artificial sequence Oligonucleotide
caggtctagtcagaccattatacatagtaatg

SEQ ID NO: 93 - DNA, 30 bp, Artificial sequence
  Description of artificial sequence Oligonucleotide
gaaacacctatttggaatggtacctgcaga

SEQ ID NO: 94 - DNA, 32 bp, Artificial sequence
  Description of artificial sequence Oligonucleotide
ggcaacttcatggtggcggcacgcgttctgac

SEQ ID NO: 95 - DNA, 32 bp, Artificial sequence
  Description of artificial sequence Oligonucleotide
gaaaccagaacatcagcaccaacagcctaaca

SEQ ID NO: 96 - DNA, 32 bp, Artificial sequence
  Description of artificial sequence Oligonucleotide
ctgagtcatcacaacatcactgctggaagcag

SEQ ID NO: 97 - DNA, 32 bp, Artificial sequence
  Description of artificial sequence Oligonucleotide
tctccaggggtgacgggcagggagagtggaga

SEQ ID NO: 98 - DNA, 32 bp, Artificial sequence
  Description of artificial sequence Oligonucleotide
tctgactagacctgcaggagatggaggccggc

SEQ ID NO: 99 - DNA, 31 bp, Artificial sequence
  Description of artificial sequence Oligonucleotide
aaataggtgtttccattactatgtacaatgc

SEQ ID NO: 100 - DNA, 32 bp, Artificial sequence
  Description of artificial sequence Oligonucleotide
cagggcagtctccacagctcctgatctataaa

SEQ ID NO: 101 - DNA, 32 bp, Artificial sequence
  Description of artificial sequence Oligonucleotide
gtttctaatcggctttatggggtccctgacag

SEQ ID NO: 102 - DNA, 32 bp, Artificial sequence
  Description of artificial sequence Oligonucleotide
gttcagtggcagtggatcaggcacagatttta

SEQ ID NO: 103 - DNA, 32 bp, Artificial sequence
  Description of artificial sequence Oligonucleotide
cactgaaaatcagcagagtggaggctgaggat

SEQ ID NO: 104 - DNA, 32 bp, Artificial sequence
  Description of artificial sequence Oligonucleotide
gttggggtttattactgctttcaaggttcaca

SEQ ID NO: 105 - DNA, 32 bp, Artificial sequence
  Description of artificial sequence Oligonucleotide
tgttccgtggacgttcggccaagggaccaagg

SEQ ID NO: 106 - DNA, 30 bp, Artificial sequence
  Description of artificial sequence Oligonucleotide
tggaaatcaaacgtgagtggatcctctgcg

SEQ ID NO: 107 - DNA, 17 bp, Artificial sequence
  Description of artificial sequence Oligonucleotide
tctgcaggtaccattgc

SEQ ID NO: 108 - DNA, 21 bp, Artificial sequence
  Description of artificial sequence Oligonucleotide
tgcaatggtacctgcagaagc

SEQ ID NO: 109 - DNA, 32 bp, Artificial sequence
  Description of artificial sequence Oligonucleotide
agactgccctggcttctgcaggtaccattgca

SEQ ID NO: 110 - DNA, 32 bp, Artificial sequence
  Description of artificial sequence Oligonucleotide
cgattagaaactttatagatcaggagctgtgg

SEQ ID NO: 111 - DNA, 32 bp, Artificial sequence
  Description of artificial sequence Oligonucleotide
tgccactgaacctgtcagggaccccataaagc

SEQ ID NO: 112 - DNA, 32 bp, Artificial sequence
  Description of artificial sequence Oligonucleotide
gattttcagtgtaaaatctgtgcctgatccac

SEQ ID NO: 113 - DNA, 32 bp, Artificial sequence
  Description of artificial sequence Oligonucleotide
taaaccccaacatcctcagcctccactctgct

SEQ ID NO: 114 - DNA, 32 bp, Artificial sequence
  Description of artificial sequence Oligonucleotide
tccacggaacatgtgaaccttgaaagcagtaa

SEQ ID NO: 115 - DNA, 31 bp, Artificial sequence
  Description of artificial sequence Oligonucleotide
tttgatttccaccttggtcccttggccgaac

SEQ ID NO: 116 - DNA, 19 bp, Artificial sequence
  Description of artificial sequence Oligonucleotide
cgcagaggatccactcacg

SEQ ID NO: 117 - DNA, 18 bp, Artificial sequence
  Description of artificial sequence Oligonucleotide
gtcagaacgcgtgccgcc

SEQ ID NO: 118 - DNA, 34 bp, Artificial sequence
  Description of artificial sequence Oligonucleotide
accatgaaagtgttgagtctgttgtacctcttga

SEQ ID NO: 119 - DNA, 34 bp, Artificial sequence
  Description of artificial sequence Oligonucleotide
cagccattcctggtatcctgtctcaggtgcagct

SEQ ID NO: 120 - DNA, 34 bp, Artificial sequence
  Description of artificial sequence Oligonucleotide
tcaggagtcgggcccaggactggtgaagccttcg

SEQ ID NO: 121 - DNA, 33 bp, Artificial sequence
  Description of artificial sequence Oligonucleotide
gagaccctgtccctcacctgcactgtctctggt

SEQ ID NO: 122 - DNA, 33 bp, Artificial sequence
  Description of artificial sequence Oligonucleotide
tactccatcaccggtggttatttatggaactgg

SEQ ID NO: 123 - DNA, 33 bp, Artificial sequence
  Description of artificial sequence Oligonucleotide
atacggcagcccccagggaagggactggagtgg

SEQ ID NO: 124 - DNA, 33 bp, Artificial sequence
  Description of artificial sequence Oligonucleotide
atggggtatatcagctacgacggtaccaataac

SEQ ID NO: 125 - DNA, 34 bp, Artificial sequence
  Description of artificial sequence Oligonucleotide
tcaacactttcatggtggcggcacgcgttctgac

SEQ ID NO: 126 - DNA, 34 bp, Artificial sequence
  Description of artificial sequence Oligonucleotide
ataccaggaatggctgtcaagaggtacaacagac

SEQ ID NO: 127 - DNA, 34 bp, Artificial sequence
  Description of artificial sequence Oligonucleotide
tgggcccgactcctgaagctgcacctgagacagg

SEQ ID NO: 128 - DNA, 34 bp, Artificial sequence
  Description of artificial sequence Oligonucleotide
tgagggacagggtctccgaaggcttcaccagtcc

SEQ ID NO: 129 - DNA, 34 bp, Artificial sequence
  Description of artificial sequence Oligonucleotide
ccaccggtgatggagtaaccagagacagtgcagg

SEQ ID NO: 130 - DNA, 34 bp, Artificial sequence
  Description of artificial sequence Oligonucleotide
ccctgggggctgccgtatccagttccataaataa

SEQ ID NO: 131 - DNA, 32 bp, Artificial sequence
  Description of artificial sequence Oligonucleotide
tagctgatataccccatccactccagtccctt

SEQ ID NO: 132 - DNA, 16 bp, Artificial sequence
  Description of artificial sequence Oligonucleotide
gttattggtaccgtcg

SEQ ID NO: 133 - DNA, 21 bp, Artificial sequence
  Description of artificial sequence Oligonucleotide
tacgacggtaccaataactac

SEQ ID NO: 134 - DNA, 32 bp, Artificial sequence
  Description of artificial sequence Oligonucleotide
aaaccctccctcaaggatcgaatcaccatatc

SEQ ID NO: 135 - DNA, 32 bp, Artificial sequence
  Description of artificial sequence Oligonucleotide
acgtgacacgtccaagaaccagttctccctga

SEQ ID NO: 136 - DNA, 32 bp, Artificial sequence
  Description of artificial sequence Oligonucleotide
agctgagctctgtgaccgctgcggacactgca

SEQ ID NO: 137 - DNA, 32 bp, Artificial sequence
  Description of artificial sequence Oligonucleotide
gtgtattactgtgcgagatacggtagggtctt

SEQ ID NO: 138 - DNA, 32 bp, Artificial sequence
  Description of artificial sequence Oligonucleotide
ctttgactactggggccagggaaccctggtca

SEQ ID NO: 139 - DNA, 30 bp, Artificial sequence
  Description of artificial sequence Oligonucleotide
ccgtctcctcaggtgagtggatcctctgcg

SEQ ID NO: 140 - DNA, 32 bp, Artificial sequence
  Description of artificial sequence Oligonucleotide
agggagggtttgtagttattggtaccgtcgta

SEQ ID NO: 141 - DNA, 32 bp, Artificial sequence
  Description of artificial sequence Oligonucleotide
acgtgtcacgtgatatggtgattcgatccttg

SEQ ID NO: 142 - DNA, 32 bp, Artificial sequence
  Description of artificial sequence Oligonucleotide
agagctcagcttcagggagaactggttcttgg

SEQ ID NO: 143 - DNA, 32 bp, Artificial sequence
  Description of artificial sequence Oligonucleotide
cagtaatacactgcagtgtccgcagcggtcac

SEQ ID NO: 144 - DNA, 32 bp, Artificial sequence
  Description of artificial sequence Oligonucleotide
agtagtcaaagaagaccctaccgtatctcgca

SEQ ID NO: 145 - DNA, 33 bp, Artificial sequence
  Description of artificial sequence Oligonucleotide
ctgaggagacggtgaccagggttccctggcccc

SEQ ID NO: 146 - DNA, 18 bp, Artificial sequence
  Description of artificial sequence Oligonucleotide
cgcagaggatccactcac

SEQ ID NO: 147 - DNA, 31 bp, Homo sapiens
ctggttactccatcagcggtggttatttatg

SEQ ID NO: 148 - DNA, 31 bp, Homo sapiens
cataaataaccaccgctgatggagtaaccag

SEQ ID NO: 149 - DNA, 31 bp, Homo sapiens
gggactggagtggatcgggtatatcagctac

SEQ ID NO: 150 - DNA, 31 bp, Homo sapiens
gtagctgatatacccgatccactccagtccc

SEQ ID NO: 151 - DNA, 31 bp, Homo sapiens
tccctcaaggatcgagtcaccatatcacgtg

SEQ ID NO: 152 - DNA, 31 bp, Homo sapiens
cacgtgatatggtgactcgatccttgaggga

SEQ ID NO: 153 - DNA, 39 bp, Homo sapiens
gatcgagtcaccatatcagtggacacgtccaagaaccag

SEQ ID NO: 154 - DNA, 39 bp, Homo sapiens
ctggttcttggacgtgtccactgatatggtgactcgatc

SEQ ID NO: 155 - DNA, 31 bp, Homo sapiens
gcttccagcagtgatattgtgatgactcagt

SEQ ID NO: 156 - DNA, 31 bp, Homo sapiens
actgagtcatcacaatatcactgctggaagc

(19) United States
(12) Patent Application Publication
(10) Pub. No.: US 2008/0193445 A1
(43) Pub. Date: Aug. 14, 2008

(54) Novel anti-IGF-IR antibodies and uses thereof

(76) Inventors: Liliane Goetsch, Ayse (FR); Nathalie Corvaia, Collonges sous Saleve (FR); Olivier Leger, Saint Sixt (FR); Nelly Kuklin, Collegeville, PA (US); Michael Chastain, Seattle, WA (US)

Correspondence Address: MERCK AND CO., INC, P O BOX 2000, RAHWAY, NJ, 07065-0907, US

(21) Appl. No.: 11/801,080

(22) Filed: May 8, 2007

Related U.S. Application Data
(63) Continuation-in-part of application No. 10/735,916, filed on Dec. 16, 2003, now Pat. No. 7,241,444.
Application No. 10/735,916 is a continuation-in-part of application No. PCT/FR03/00178, filed on Jan. 20, 2003.

(30) Foreign Application Priority Data
Jan. 18, 2002 (FR) FR 0200654
May 7, 2002 (FR) FR 0205753
Jul. 11, 2003 (FR) FR 0308538

(51) Int. Cl. A61K 39/395; A61P 35/00

(52) U.S. Cl. 424/133.1; 435/29

(57) ABSTRACT

The present invention relates to novel antibodies capable of binding specifically to the human insulin-like growth factor I receptor IGF-IR and/or capable of specifically inhibiting the tyrosine kinase activity of said IGF-IR receptor, especially monoclonal antibodies of murine, chimeric and humanized origin, as well as the amino acid and nucleic acid sequences coding for these antibodies. The invention likewise comprises the use of these antibodies as a medicament for the prophylactic and/or therapeutic treatment of cancers overexpressing IGF-IR or any pathology connected with the overexpression of said receptor as well as in processes or kits for diagnosis of illnesses connected with the overexpression of the IGF-IR receptor. The invention finally comprises products and/or compositions comprising such antibodies in combination with anti-EGFR antibodies and/or compounds and/or anti-cancer agents or agents conjugated with toxins and their use for the prevention and/or the treatment of certain cancers.

### CROSS REFERENCE TO RELATED APPLICATIONS

This application is a continuation-in-part of U.S. patent application Ser. No. 10/735,916, filed Dec. 16, 2003, which is a continuation-in-part of PCT/FR 03/00178 filed in France on Jan. 20, 2003, which claims priority from FR 0200653 filed in France on Jan. 18, 2002, FR 0200654 filed in France on Jan. 18, 2002, FR 0205753 filed in France on May 7, 2002, and FR 0308538 filed in France on Jul. 11, 2003, the entire contents of which are hereby incorporated by reference.

### BACKGROUND OF THE INVENTION

The present invention relates to novel antibodies capable of binding specifically to the human insulin-like growth factor I receptor IGF-IR and/or capable of specifically inhibiting the tyrosine kinase activity of said IGF-IR receptor, especially monoclonal antibodies of murine, chimeric and humanized origin, as well as the amino acid and nucleic acid sequences coding for these antibodies. The invention likewise comprises the use of these antibodies as a medicament for the prophylactic and/or therapeutic treatment of cancers overexpressing IGF-IR or any pathology connected with the overexpression of said receptor as well as in processes or kits for diagnosis of illnesses connected with the overexpression of the IGF-IR receptor. The invention finally comprises products and/or compositions comprising such antibodies in combination with anti-EGFR antibodies and/or compounds and/or anti-cancer agents or agents conjugated with toxins and their use for the prevention and/or the treatment of certain cancers.

The insulin-like growth factor I receptor called IGF-IR is a receptor with tyrosine kinase activity having 70% homology with the insulin receptor IR. IGF-IR is a glycoprotein of molecular weight approximately 350,000. It is a hetero-tetrameric receptor of which each half-linked by disulfide bridges—is composed of an extracellular α-subunit and of a transmembrane β-subunit (see FIG. 1). IGF-IR binds IGF I and IGF II with a very high affinity (Kd #1 nM) but is equally capable of binding to insulin with an affinity 100 to 1000 times less. Conversely, the IR binds insulin with a very high affinity although the IGFs only bind to the insulin receptor with a 100 times lower affinity. The tyrosine kinase domain of IGF-IR and of IR has a very high sequence homology although the zones of weaker homology respectively concern the cysteine-rich region situated on the α-subunit and the C-terminal part of the f-subunit. The sequence differences observed in the α-subunit are situated in the binding zone of the ligands and are therefore at the origin of the relative affinities of IGF-IR and of IR for the IGFs and insulin respectively. The differences in the C-terminal part of the β-subunit result in a divergence in the signalling pathways of the two receptors; IGF-IR mediating mitogenic, differentiation and antiapoptosis effects, while the activation of the IR principally involves effects at the level of the metabolic pathways (Baserga et al., Biochim. Biophys. Acta, 1332: F105-126, 1997; Baserga R., Exp. Cell. Res., 253:1-6, 1999).

The cytoplasmic tyrosine kinase proteins are activated by the binding of the ligand to the extracellular domain of the receptor. The activation of the kinases in its turn involves the stimulation of different intra-cellular substrates, including IRS-1, IRS-2, Shc and Grb 10 (Peruzzi F. et al., J. Cancer Res. Clin. Oncol., 125:166-173, 1999). The two major substrates of IGF-IR are IRS and Shc which mediate, by the activation of numerous effectors downstream, the majority of the growth and differentiation effects connected with the attachment of the IGFs to this receptor (FIG. 2). The availability of substrates can consequently dictate the final biological effect connected with the activation of the IGF-IR. When IRS-1 predominates, the cells tend to proliferate and to transform. When Shc dominates, the cells tend to differentiate (Valentinis B. et al., J. Biol. Chem. 274:12423-12430, 1999). It seems that the route principally involved for the effects of protection against apoptosis is the phosphatidyl-inositol 3-kinases (PI 3-kinases) route (Prisco M. et al., Horm. Metab. Res., 31:80-89, 1999; Peruzzi F. et al., J. Cancer Res. Clin. Oncol., 125:166-173, 1999).

The role of the IGF system in carcinogenesis has become the subject of intensive research in the last ten years. This interest followed the discovery of the fact that in addition to its mitogenic and antiapoptosis properties, IGF-IR seems to be required for the establishment and the maintenance of a transformed phenotype. In fact, it has been well established that an overexpression or a constitutive activation of IGF-IR leads, in a great variety of cells, to a growth of the cells independent of the support in media devoid of fetal calf serum, and to the formation of tumors in nude mice. This in itself is not a unique property since a great variety of products of overexpressed genes can transform cells, including a good number of receptors of growth factors. However, the crucial discovery which has clearly demonstrated the major role played by IGF-IR in the transformation has been the demonstration that the R— cells, in which the gene coding for IGF-IR has been inactivated, are totally refractory to transformation by different agents which are usually capable of transforming the cells, such as the E5 protein of bovine papilloma virus, an overexpression of EGFR or of PDGFR, the T antigen of SV 40, activated ras or the combination of these two last factors (Sell C. et al., Proc. Natl. Acad. Sci., USA, 90: 11217-11221, 1993; Sell C. et al., Mol. Cell. Biol., 14:3604-3612, 1994; Morrione A. J., Virol., 69:5300-5303, 1995; Coppola D. et al., Mol. Cell. Biol., 14:4588-4595, 1994; DeAngelis T et al., J. Cell. Physiol., 164:214-221, 1995).

IGF-IR is expressed in a great variety of tumors and of tumor lines and the IGFs amplify the tumor growth via their attachment to IGF-IR. Other arguments in favor of the role of IGF-IR in carcinogenesis come from studies using murine monoclonal antibodies directed against the receptor or using negative dominants of IGF-IR. In effect, murine monoclonal antibodies directed against IGF-IR inhibit the proliferation of numerous cell lines in culture and the growth of tumor cells in vivo (Arteaga C. et al., Cancer Res., 49:6237-6241, 1989; Li et al., Biochem. Biophys. Res. Com., 196:92-98, 1993; Zia F et al., J. Cell. Biol., 24:269-275, 1996; Scotlandi K et al., Cancer Res., 58:4127-4131, 1998). It has likewise been shown in the works of Jiang et al. (Oncogene, 18:6071-6077, 1999) that a negative dominant of IGF-IR is capable of inhibiting tumor proliferation.

Colon cancer which is also known as cancer of the large bowel and colorectal cancer, is among the leading causes of cancer-related morbidity and mortality in industrialized nations. It is second only to lung cancer as a cause of cancer death in the United States. It is a common malignant condition that generally occurs in individuals 50 years of age or older; and the overall incidence rate of colon cancer has not changed substantially during the past 40 years. (Harrison's Principles of Internal Medicine, 14/e, McGraw-Hill Companies, New York, 1998). In 1995, the American Cancer society estimated that 135,000 new cases of colon cancer were diagnosed; 71% were in the colon and 30% were in the rectum. Colon and rectal cancers are often silent and slowly progressive. Most patients exhibit symptoms such as rectal bleeding, pain, abdominal distension or weight loss only after the disease is advanced and not surgically curable. It thus seems to be of relevance to identify new therapeutic target molecules and to define patients likely to benefit from treatment at an early stage.

To date, researchers have found that Insulin-like growth factors (IGF-1 and IGF-2) and the IGF-1 membrane receptor (IGF-1R) are implicated as playing a critical role in the carcinogenesis of several tumors, among them colorectal cancer (CRC). The term colorectal cancer includes cancer of the colon and the rectum. See Peters, et al., IGF-1R, IGF-1 and IGf-2 expression as potential prognostic and predictive markers in colorectal-cancer, Virchows Arch, 443: 139-145 (2003).

The traditional method of colon cancer diagnosis is through the use of non-invasive or mildly invasive diagnostic tests such as, for example, fecal occult blood testing, more invasive visual examination, and histologic examination of biopsy. Although these tests may detect colon cancers, each has drawbacks that limit its effectiveness as a diagnostic tool. One primary source of difficulty with most of the currently available methods for diagnosing colorectal cancer, is patient reluctance to submit to, or follow through with the procedures, due to the uncomfortable or perceived embarrassing nature of the tests. As well, the usefulness of tests for occult blood is hampered by the intermittent bleeding patterns of colon cancers, which can result in a high percentage of false negative results. These limitations of the less-invasive tests for colon cancer may delay a patient's procurement of rapid diagnosis and appropriate colon cancer treatment.

Yet another method of colon cancer diagnosis is the detection of carcinoembryonic antigen (CEA) in a blood sample from a subject, which when present at high levels, may indicate the presence of advanced colon cancer. But CEA levels may also be abnormally high when no cancer is present. Thus, this test is not selective for colon cancer, which limits the test's value as an accurate and reliable diagnostic tool. In addition, elevated CEA levels are not detectable until late-stage colon cancer, when the cure rate is low, treatment options limited, and patient prognosis poor.

New methodology of immunological testing may be an improvement and has potential advantages over conventional diagnostic techniques. If colon cancer screening by immunological testing is more specific, the problem of false positive test results leading to unnecessary colonoscopic examination would be reduced leading to cost savings and improved safety. Although available diagnostic procedures for colon cancer may be partially successful, the methods for detecting colon cancer remain unsatisfactory.

If cancerous cells are discovered, the prognosis, or chance of recovery and choice of treatment depend on several factors, namely, the stage of the cancer (e.g. whether it is just in the inner lining of the colon or if it has spread to other places), and the patient's general state of health. After treatment, a blood test and x-rays may be done to see if the cancer is in remission.

The treatment of colon cancer once diagnosis is made depends on the extent of the cancer's invasion of the colon tissue, lymph nodes, and metastasis to other organs such as the liver. The survival rate for patients diagnosed with early-stage cancer is about 90% survival after 5 years. At present, only 41% of patients are diagnosed at an early stage. The five-year survival rate drops if the cancer is not detected until the cancer has spread beyond the mucosal layer of the colon, and drops significantly further if, when detected, the cancer has spread beyond the colon to the lymph nodes and beyond. Unfortunately, 55,000 Americans die each year due to recurrent or metastatic colon or rectal cancer. The key to enhanced survival is early diagnosis and treatment. Thus, it is critical to diagnose and treat colon cancer at the earliest possible stage to increase the likelihood of a positive prognosis and outcome.

The prognosis of colon cancer is clearly related to the degree of penetration of the tumor through the bowel wall and the presence or absence of nodal involvement. These two characteristics form the basis for all staging systems developed for this disease. Bowel obstruction and bowel perforation are indicators of poor prognosis. Elevated pretreatment serum levels of carcinoembryonic antigen (CEA) and carbohydrate antigen 19-9 (CA 19-9) also have negative prognostic significance.

There are currently three primary treatments available for patients with cancer of the colon. These treatments depend upon the stage of the cancer and the health of the individual seeking the treatment. Each one is briefly discussed.

Surgery is the primary treatment and results in cure in approximately 50% of patients. Recurrence following surgery is a major problem and often is the ultimate cause of death. Ultimately, 50% of patients thought to have undergone curative resections eventually develop recurrent disease. The remaining cases frequently undergo peri-operative radiation and/or chemotherapy to attempt to control the metastatic spread of disease. Radiation can be used alone or in addition to surgery and/or chemotherapy. Chemotherapy is generally the last possible treatment option. This procedure uses drugs to kill cancerous cells. Chemotherapy may be administered through capsules, or intravenously.

While these efforts alleviate, and in some instances, remove the threat of colon cancer in an individual, these treatments can be extremely costly and unpredictable. Moreover, these treatments can be dangerous, not to mention putting incredible amounts of physical strain upon the individual. Because of the high incidence of colon cancer, there is a dire need for a better treatment option for patients presenting with colon cancer.

Cancer of the ovary is the second most common cancer of the female reproductive organs and the fourth most common cause of cancer deaths among American women. Carcinoma of the ovary is most common in women over age 60. Because ovarian cancers are not readily detectable by diagnostic techniques (Siemens and Auersperg, 1988, J. Cellular Physiol., 134:347-356), diagnosis of carcinoma of the ovary is generally only possible when the disease has progressed to a late stage of development. As a result, two thirds of women with ovarian cancer have advanced (Stage III or IV) disease at the time of diagnosis. As a consequence, it is one of the most lethal of the gynecological malignancies. Indeed, it has the highest mortality of any of the gynecologic cancers. The overall 5-year survival rate is at least 75%, if the cancer is confined to the ovaries, and decreases to 17% in women diagnosed with distant metastases. Symptoms usually do not become apparent until the tumor compresses or invades adjacent structures, or ascites develops, or metastases become clinically evident.

Potential screening tests for ovarian cancer include the bimanual pelvic examination, the Papanicolaou (Pap) smear, tumor markers, and ultrasound imaging. The pelvic examination, which can detect a variety of gynecologic disorders, is of unknown sensitivity in detecting ovarian cancer. Although pelvic examinations can occasionally detect ovarian cancer, small, early-stage ovarian tumors are often not detected by palpation due to the deep anatomic location of the ovary. Ovarian cancers detected by pelvic examination are generally advanced and associated with poor survival. The pelvic examination, likewise, may also produce false positives when benign adnexal masses (e.g., functional cysts) are found. The Pap smear may occasionally reveal malignant ovarian cells, but it is not considered to be a valid screening test for ovarian carcinoma.

Management of the disease currently relies on a combination of early diagnosis and aggressive treatment, which may include one or more of a variety of treatments such as surgery, radiotherapy, chemotherapy and hormone therapy. The course of treatment for a particular cancer is often selected based on a variety of prognostic parameters, including an analysis of specific tumor markers. However, the use of established markers often leads to a result that is difficult to interpret, and high mortality continues to be observed in many cancer patients.

Mounting evidence suggests that insulin-like growth factors (IGF) play important roles in carcinogenesis and tumor progression. Many members of the IGF family, including IGF ligands (IGF-I and IGF-II), specific IGF-binding proteins (IGFBP), and IGF receptors (IGF-IR and IR-A), are expressed in ovarian epithelial cells. Indeed, blocking IGF signaling is under intense investigation as a potential therapeutic target for cancer treatment. See Lu, et al., The Relationship of Insulin-Like Growth Factor-II, Insulin-Like Growth Factor Binding Protein-3, and Estrogen Receptor-α Expression to Disease Progression in Epithelial Ovarian Cancer, Clinical Cancer Research Vol. 12, 1208-1214, (2006). See also Warrenfeltz et al., Gene expression profiling of epithelial ovarian tumours correlated with malignant potential, Mol Cancer; 3: 27 (2004); Mauro L et al., Role of the IGF-I receptor in the regulation of cell-cell adhesion: implications in cancer development and progression. J. cell Physiol., 194:108-116 (2003); Moschos et al., The role of the IGF system in cancer: from basic to clinical studies and clinical applications; Oncology. 63:317-332 (2002); and Lukanova et al., Circulating levels of insulin-like growth factor-I and risk of ovarian cancer; Int. J. Cancer, 101:549-554. (2000)_. doi: 10.1002/ijc.10613.

Consequently, immunotherapy has the potential to substantially improve cancer treatment and survival. Such therapy may include administering an antibody specific for ovarian cancer cell specific receptor polypeptides that are present in greater amounts in ovarian cancer than normal tissue.

Pediatric cancers such as rhabdomyosarcoma is the most common soft-tissue sarcoma of childhood and accounts for 4-8% of all pediatric malignancies. Rhabdomyosarcomas are histologically classified as embryonal (E-RMS), which is more common or alveolar (A-RMS) rhabdomyosarcoma. Although rhabdomyosarcomas display rhabdomyoblastic differentiation, the cell type of origin has not been identified yet. Most rhabdomyosarcoma of the alveolar subtype carry the characteristic t(2;13)(q35;q14) and t(1;13)(p36;q14) translocations which result in the formation of the chimeric PAX3-FKHR and PAX7-FKHR transcription factors. In addition, the TP53 tumor suppressor gene, among others like MDM2, CDKN2A and CDK4 is frequently mutated in sporadic embryonal rhabdomyosarcoma. A function for the TP53 pathway in rhabdomyosarcoma tumorigenesis is further indicated by the observation that rhabdomyosarcoma is the most common sarcoma type in patients with hereditary predisposition to cancer due to germ-line mutations in the TP53 gene. Rhabdomyosarcomas also develop in p53 heterozygous and homozygous mutant mice, although with low penetrance and long latency. Mutated TP53 may function through an inhibition of MYOD function, thereby blocking differentiation, or by loss of the normal transcriptional repression of IGF-2 by wildtype TP53. Anderson et al., Genes, Chromosomes & Cancer, 26; 275-285 (1999).

Ewing's sarcoma (ES), another pediatric cancer is a rare malignancy that most often presents as an undifferentiated primary bone tumor; less commonly, it arises in soft tissue (extraosseous Ewing's sarcoma, EES). Both are part of a spectrum of neoplastic diseases known as the Ewing's sarcoma family of tumors (EFT), which also includes the more differentiated peripheral primitive neuroectodermal tumor (PNET, previously called neuroepithelioma, adult neuroblastoma, and Askin's tumor of the chest wall). PNET can also present either in bone or soft tissue. Because these tumors share similar histological and immunohistochemical characteristics and unique nonrandom chromosomal translocations, they are considered to have a common origin. In addition to their immunohistochemical and cytogenetic similarities, the EFT share important clinical features. These include a peak incidence between the age of 10 and 20 (70 percent of affected patients are under the age of 20), a tendency towards rapid spread to lungs, bone, and bone marrow, and responsiveness to the same chemotherapeutic regimens and radiotherapy. As with osteosarcoma (the other major sarcoma affecting bone), advances in multidisciplinary management over the past 30 years have resulted in a marked improvement in long-term survival. In data derived from the Surveillance, Epidemiology, and End Results (SEER) program of the National Cancer Institute, five-year survival rates for patients with Ewing's sarcoma rose from 36 to 56 percent during the periods 1975 to 1984 and 1985 to 1994. (See “Bone sarcomas: Principles of surgical management”).

In certain embodiments, the antibodies disclosed herein will find use in treating pediatrics cancers, including, but not limited to, neuroblastoma, osteosarcoma, Ewing sarcoma, and rhabdomyosarcoma.

The object of the present invention is to be able to have available a murine monoclonal antibody, preferably a chimerized or humanized antibody, which will recognize IGF-IR specifically and with great affinity. This antibody will interact little or not at all with the IR receptor on insulin. Its attachment will be able to inhibit in vitro the growth of tumors expressing IGF-IR by interacting principally with the signal transduction pathways activated during IGF1/IGF-IR and IGF2/IGF-IR interactions. This antibody will be able to be active in vivo on all the types of tumors expressing IGF-IR including estrogen-dependent tumors of the breast and tumors of the prostate, which is not the case for the anti-IGF-IR monoclonal antibodies (written MAb or MAB) currently available. In effect, αIR3, which refers to the domain of IGF-IR, totally inhibits the growth of estrogen-dependent tumors of the breast (MCF-7) in vitro but is without effect on the corresponding model in vivo (Arteaga C. et al., J. Clin. Invest. 84:1418-1423, 1989). In the same way, the scFv-Fc fragment derived from the murine monoclonal 1H7 is only weakly active on the tumor of the breast MCF-7 and totally inactive on an androgen-independent tumor of the prostate (Li S. L. et al., Cancer Immunol. Immunother., 49:243-252, 2000).

In a surprising manner, the inventors have demonstrated a chimeric antibody (called C7C10) and two humanized antibodies respectively called h7C10 humanized form 1 and h7C10 humanized form 2, derivatives of the murine monoclonal antibody 7C10, recognising IGF-IR and corresponding to all of the criteria stated above, that is to say to a nonrecognition of the receptor on the insulin, to an in vitro blockage of the IGF1 and/or IGF2 proliferation induced but likewise to the in vivo inhibition of the growth of different tumors expressing IGF-IR among which are an osteosarcoma and a non-small cell lung tumor but likewise and more particularly the estrogen-dependent tumor of the breast MCF-7 and an androgen-independent tumor of the prostate DU-145. In the same way, and in a surprising manner, the intensity of inhibition of the tumor growth of MCF-7 cells in vivo by the antibody 7C10 is comparable, or even significantly superior, to that observed with tamoxifen, one of the reference compounds in the treatment of estrogen-dependent tumors of the breast. Furthermore, it has been shown that these antibodies inhibit the phosphorylation of the tyrosine of the beta chain of IGF-IR and of IRS1, the first substrate of the receptor. Moreover, it has likewise been established that these antibodies cause the internalization of said receptor and its degradation contrary to what is usually observed with natural ligands which allow the rapid recycling of the receptor on the surface of the cells. It has been possible to characterize these antibodies by their peptidic and nucleic sequence, especially by the sequence of their regions determining their complementarity (CDR) for IGF-IR.

Thus, according to a first embodiment, a subject of the present invention is an isolated antibody, or one of its functional fragments, said antibody or one of its said fragments being capable of binding specifically to the human insulin-like growth factor I receptor and, if necessary, preferably moreover capable of inhibiting the natural attachment of the ligands IGF1 and/or IGF2 of IGF-IR and/or capable of specifically inhibiting the tyrosine kinase activity of said IGF-IR receptor, characterized in that it comprises a light chain comprising at least one complementarity determining region CDR chosen from the CDRs of amino acid sequence SEQ ID Nos. 2, 4 or 6, or at least one CDR whose sequence has at least 80%, preferably 85%, 90%, 95% and 98% identity, after optimum alignment, with the sequence SEQ ID Nos. 2, 4 or 6, or in that it comprises a heavy chain comprising at least one CDR chosen from the CDRs of amino acid sequence SEQ ID Nos. 8, 10 and 12, or at least one CDR whose sequence has at least 80%, preferably 85%, 90%, 95% and 98% identity, after optimum alignment, with the sequence SEQ ID No. 8, 10 and 12.

In the present description, the terms “to bind” and “to attach” have the same meaning and are inter-changeable.

In the present description, the terms polypeptides, polypeptide sequences, peptides and proteins attached to antibody compounds or to their sequence are interchangeable.

It must be understood here that the invention does not relate to the antibodies in natural form, that is to say they are not in their natural environment but that they have been able to be isolated or obtained by purification from natural sources, or else obtained by genetic recombination, or by chemical synthesis, and that they can then contain unnatural amino acids as will be described further on.

By CDR region or CDR, it is intended to indicate the hypervariable regions of the heavy and light chains of the immunoglobulins as defined by Kabat et al. (Kabat et al., Sequences of proteins of immunological interest, 5th Ed., U.S. Department of Health and Human Services, NIH, 1991, and later editions). 3 heavy chain CDRs and 3 light chain CDRs exist. The term CDR or CDRs is used here in order to indicate, according to the case, one of these regions or several, or even the whole, of these regions which contain the majority of the amino acid residues responsible for the binding by affinity of the antibody for the antigen or the epitope which it recognizes.

By “percentage of identity” between two nucleic acid or amino acid sequences in the sense of the present invention, it is intended to indicate a percentage of nucleotides or of identical amino acid residues between the two sequences to be compared, obtained after the best alignment (optimum alignment), this percentage being purely statistical and the differences between the two sequences being distributed randomly and over their entire length. The comparisons of sequences between two nucleic acid or amino acid sequences are traditionally carried out by comparing these sequences after having aligned them in an optimum manner, said comparison being able to be carried out by segment or by “comparison window”. The optimum alignment of the sequences for the comparison can be carried out, in addition to manually, by means of the local homology algorithm of Smith and Waterman (1981) [Ad. App. Math. 2:482], by means of the local homology algorithm of Neddleman and Wunsch (1970) [J. Mol. Biol. 48: 443], by means of the similarity search method of Pearson and Lipman (1988) [Proc. Natl. Acad. Sci. USA 85:2444), by means of computer software using these algorithms (GAP, BESTFIT, FASTA and TFASTA in the Wisconsin Genetics Software Package, Genetics Computer Group, 575 Science Dr., Madison, Wis., or else by BLAST N or BLAST P comparison software).

The percentage of identity between two nucleic acid or amino acid sequences is determined by comparing these two sequences aligned in an optimum manner and in which the nucleic acid or amino acid sequence to be compared can comprise additions or deletions with respect to the reference sequence for an optimum alignment between these two sequences. The percentage of identity is calculated by determining the number of identical positions for which the nucleotide or the amino acid residue is identical between the two sequences, by dividing this number of identical positions by the total number of positions in the comparison window and by multiplying the result obtained by 100 in order to obtain the percentage of identity between these two sequences.

For example, it is possible to use the BLAST program, “BLAST 2 sequences” (Tatusova et al., “Blast 2 sequences—a new tool for comparing protein and nucleotide sequences”, FEMS Microbiol Lett. 174:247-250) available on the site http://www.ncbi.nlm.nih.gov/ gorf/b12.html, the parameters used being those given by default (in particular for the parameters “open gap penalty”: 5, and “extension gap penalty: 2; the matrix chosen being, for example, the matrix “BLOSUM 62” proposed by the program), the percentage of identity between the two sequences to be compared being calculated directly by the program.

By amino acid sequence having at least 80%, preferably 85%, 90%, 95% and 98% identity with a reference amino acid sequence, those having, with respect to the reference sequence, certain modifications, in particular a deletion, addition or substitution of at least one amino acid, a truncation or an elongation are preferred. In the case of a substitution of one or more consecutive or nonconsecutive amino acid(s), the substitutions are preferred in which the substituted amino acids are replaced by “equivalent” amino acids. The expression “equivalent amino acids” is aimed here at indicating any amino acid capable of being substituted with one of the amino acids of the base structure without, however, essentially modifying the biological activities of the corresponding antibodies and such as will be defined later, especially in the examples.

These equivalent amino acids can be determined either by relying on their structural homology with the amino acids which they replace, or on results of comparative trials of biological activity between the different antibodies capable of being carried out.

By way of example, mention is made of the possibilities of substitution capable of being carried out without resulting in a profound modification of the biological activity of the corresponding modified antibody. It is thus possible to replace leucine by valine or isoleucine, aspartic acid by glutamic acid, glutamine by asparagine, arginine by lysine, etc., the reverse substitutions being naturally envisageable under the same conditions.

The antibodies according to the present invention are preferably specific monoclonal antibodies, especially of murine, chimeric or humanized origin, which can be obtained according to the standard methods well known to the person skilled in the art.

In general, for the preparation of monoclonal antibodies or their functional fragments, especially of murine origin, it is possible to refer to techniques which are described in particular in the manual “Antibodies” (Harlow and Lane, Antibodies: A Laboratory Manual, Cold Spring Harbor Laboratory, Cold Spring Harbor N.Y., pp. 726, 1988) or to the technique of preparation from hybridomas described by Kohler and Milstein (Nature, 256:495-497, 1975).

The monoclonal antibodies according to the invention can be obtained, for example, from an animal cell immunized against the IGF-IR receptor, or one of its fragments containing the epitope specifically recognized by said monoclonal antibodies according to the invention. Said IGF-IR receptor, or one of its said fragments, can especially be produced according to the usual working methods, by genetic recombination starting with a nucleic acid sequence contained in the cDNA sequence coding for the IGF-IR receptor or by peptide synthesis starting from a sequence of amino acids comprised in the peptide sequence of the IGF-IR receptor.

The monoclonal antibodies according to the invention can, for example, be purified on an affinity column on which the IGF-IR receptor or one of its fragments containing the epitope specifically recognized by said monoclonal antibodies according to the invention has previously been immobilized. More particularly, said monoclonal antibodies can be purified by chromatography on protein A and/or G, followed or not followed by ion-exchange chromatography aimed at eliminating the residual protein contaminants as well as the DNA and the LPS, in itself followed or not followed by exclusion chromatography on Sepharose gel in order to eliminate the potential aggregates due to the presence of dimers or of other multimers. In an even more preferred manner, the whole of these techniques can be used simultaneously or successively.

Chimeric or humanized antibodies are likewise included in antibodies according to the present invention.

By chimeric antibody, it is intended to indicate an antibody which contains a natural variable (light chain and heavy chain) region derived from an antibody of a given species in combination with the light chain and heavy chain constant regions of an antibody of a species heterologous to said given species.

The antibodies or their fragments of chimeric type according to the invention can be prepared by using the techniques of genetic recombination. For example, the chimeric antibody can be produced by cloning a recombinant DNA containing a promoter and a sequence coding for the variable region of a nonhuman, especially murine, monoclonal antibody according to the invention and a sequence coding for the constant region of human antibody. A chimeric antibody of the invention encoded by such a recombinant gene will be, for example, a mouse-man chimera, the specificity of this antibody being determined by the variable region derived from the murine DNA and its isotype determined by the constant region derived from the human DNA. For the methods of preparation of chimeric antibodies, it is possible, for example, to refer to the document Verhoeyn et al. (BioEssays, 8:74, 1988).

By humanized antibody, it is intended to indicate an antibody which contains CDR regions derived from an antibody of nonhuman origin, the other parts of the antibody molecule being derived from one (or from several) human antibodies. Moreover, some of the residues of the segments of the skeleton (called FR) can be modified in order to conserve the affinity of the binding (Jones et al., Nature, 321:522-525, 1986; Verhoeyen et al., Science, 239:1534-1536, 1988; Riechmann et al., Nature, 332:323-327, 1988).

The humanized antibodies according to the invention or their fragments can be prepared by techniques known to the person skilled in the art (such as, for example, those described in the documents Singer et al., J. Immun. 150:2844-2857, 1992; Mountain et al., Biotechnol. Genet. Eng. Rev., 10:1-142, 1992; or Bebbington et al., Bio/Technology, 10:169-175, 1992). Such humanized antibodies according to the invention are preferred for their use in in vitro diagnostic methods, or in vivo prophylactic and/or therapeutic treatment.

By functional fragment of an antibody according to the invention, it is intended to indicate in particular an antibody fragment, such as Fv, scFv (sc for single chain), Fab, F(ab′)₂, Fab′, scFv-Fc fragments or diabodies, or any fragment of which the half-life time would have been increased by chemical modification, such as the addition of poly(alkylene) glycol such as poly(ethylene) glycol (“PEGylation”) (pegylated fragments called Fv-PEG, scFv-PEG, Fab-PEG, F(ab′)₂—PEG or Fab′-PEG) (“PEG” for Poly(Ethylene) Glycol), or by incorporation in a liposome, said fragments having at least one of the characteristic CDRs of sequence SEQ ID No. 2, 4, 6, 8, 10 or 12 according to the invention, and, especially, in that it is capable of exerting in a general manner an even partial activity of the antibody from which it is descended, such as in particular the capacity to recognize and to bind to the IGF-IR receptor, and, if necessary, to inhibit the activity of the IGF-IR receptor.

Preferably, said functional fragments will be constituted or will comprise a partial sequence of the heavy or light variable chain of the antibody from which they are derived, said partial sequence being sufficient to retain the same specificity of binding as the antibody from which it is descended and a sufficient affinity, preferably at least equal to 1/100, in a more preferred manner to at least 1/10, of that of the antibody from which it is descended, with respect to the IGF-IR receptor.

Such a functional fragment will contain at the minimum 5 amino acids, preferably 10, 15, 25, 50 and 100 consecutive amino acids of the sequence of the antibody from which it is descended.

Preferably, these functional fragments will be fragments of Fv, scFv, Fab, F(ab′)₂, F(ab′), scFv-Fc type or diabodies, which generally have the same specificity of binding as the antibody from which they are descended. According to the present invention, antibody fragments of the invention can be obtained starting from antibodies such as described above by methods such as digestion by enzymes, such as pepsin or papain and/or by cleavage of the disulfide bridges by chemical reduction. In another manner, the antibody fragments comprised in the present invention can be obtained by techniques of genetic recombination likewise well known to the person skilled in the art or else by peptide synthesis by means of, for example, automatic peptide synthesizers such as those supplied by the company Applied Biosystems, etc.

In a more preferred manner, the invention comprises the antibodies, or their functional fragments, according to the present invention, especially chimeric or humanized antibodies, obtained by genetic recombination or by chemical synthesis.

In a preferred embodiment, a subject of the invention is an antibody, or one of its functional fragments, according to the invention, characterized in that it comprises a heavy chain comprising at least one CDR of sequence SEQ ID No. 12 or a sequence having at least 80% identity after optimum alignment with the sequence SEQ ID No. 12.

Among the six short CDR sequences, the third CDR of the heavy chain (CDRH3) has a greater size variability (greater diversity essentially due to the mechanisms of arrangement of the genes which give rise to it). It can be as short as 2 amino acids although the longest size known is 26. Functionally, CDRH3 plays a role in part in the determination of the specificity of the antibody (Segal et al., PNAS, 71:4298-4302, 1974; Amit et al., Science, 233:747-753, 1986; Chothia et al., J. Mol. Biol., 196:901-917, 1987; Chothia et al., Nature, 342:877-883, 1989; Caton et al., J. Immunol., 144:1965-1968, 1990; Sharon et al., PNAS, 87:4814-4817, 1990; Sharon et al., J. Immunol., 144:4863-4869, 1990; Kabat et al., J. Immunol., 147:1709-1719, 1991).

It is known that only a low percentage of the amino acids of the CDRs contribute to the construction of an antibody binding site, but these residues must be maintained in a very specific tridimensional conformation.

In a more preferred manner, the present invention relates to an antibody or one of its functional fragments, according to the invention, characterized in that it comprises a heavy chain comprising at least two of the three CDRs or the three CDRs of sequence SEQ ID Nos. 8, 10 and 12, or at least two of three CDRs or three CDRs of sequence respectively having at least 80% identity after optimum alignment with the sequence SEQ ID No. 8, 10 and 12.

In a likewise preferred embodiment, a subject of the invention is an antibody or one of its functional fragments, according to the invention, characterized in that it comprises a light chain comprising at least one CDR chosen from the CDRs of sequence SEQ ID No. 2, 4 or 6, or a CDR whose sequence has at least 80% identity after optimum alignment with the sequence SEQ ID No. 2, 4 or 6.

In a more preferred embodiment, a subject of the invention is an antibody or one of its functional fragments according to the invention, characterized in that it comprises a light chain comprising at least two of the three CDRs or the three CDRs of sequence SEQ ID Nos. 2, 4 and 6, or at least two of three CDRs or three CDRs of sequence respectively having at least 80% identity after optimum alignment with the sequence SEQ ID No. 2, 4 and 6.

In a more preferred manner, the antibody or one of its functional fragments according to the invention is characterized in that it comprises a heavy chain comprising the three CDRs of sequence SEQ ID Nos. 8, 10 and 12, or three CDRs of sequence respectively having at least 80% of identity after optimum alignment with the sequence SEQ ID No. 8, 10 and 12 and in that it moreover comprises a light chain comprising the three CDRs of sequence SEQ ID Nos. 2, 4 and 6, or three CDRs of sequence respectively having at least 80% of identity after optimum alignment with the sequence SEQ ID No. 2, 4 and 6.

According to another aspect, a subject of the present invention is an antibody or one of its functional fragments, according to the invention, characterized in that it does not attach or it does not attach in a significant manner to the human insulin receptor IR.

In a preferred manner, said functional fragments according to the present invention will be chosen from the fragments Fv, scFv, Fab, (Fab′)₂, Fab′, scFv-Fc or diabodies, or any functional fragment whose half-life would have been increased by a chemical modification, especially by PEGylation, or by incorporation in a liposome.

According to another aspect, the invention relates to a murine hybridoma capable of secreting a monoclonal antibody according to the present invention, especially the hybridoma of murine origin such as deposited at the Centre National de Culture De Microorganisme (CNCM, National Center of Microorganism Culture) (Institut Pasteur, Paris, France) on Sep. 19, 2001 under the number I-2717.

The monoclonal antibody here called 7C10, or one of its functional fragments, characterized in that said antibody is secreted by the hybridoma deposited at the CNCM on Sep. 19, 2001 under the number 1-2717 is, of course, part of the present invention.

“h7C10” or “MK-0646” or “F50035” are used interchangeably to describe a humanized antibody that is characterized as binding IGF-1R as well as binding the IR/IGF-1 hybrid receptor. Such an antibody may include the antibody described, for example, in U.S. Ser. No. 10/735,916 (US20050084906), which is CIP of PCT/FR03/00178 and/or US20050249730, wherein said is a humanized antibody or a fragment thereof and comprises a light chain and/or a heavy chain in which the skeleton segments FR1 to FR4 of said light chain and/or heavy chain are respectively derived from skeleton segments FR1 to FR4 of human antibody light chain and/or heavy chain. The humanized antibody may comprise at least one light chain that comprises at least one or more complementary determining regions derived from a non-human source and having the amino acid sequence selected from the group consisting of SEQ ID NOs: 2, 4 or 6 and at least one heavy chain comprising at least one or more complementary determining regions having an amino acid sequence selected from the group consisting of SEQ ID NOs 8, 10, or 12. The light chain may comprise one or more of the amino acid sequences as set forth in one of SEQ ID No. 61 or 65, or a sequence having at least 80% identity after optimum alignment with the sequence SEQ ID No. 61 or 65. Likewise, the heavy chain comprises one or more amino acid sequences as set forth in one of SEQ ID No. 75, 79 or 83, or a sequence having at least 80% identity after optimum alignment with the sequence SEQ ID No. 75, 79 or 83. In certain embodiments, the antibody used to treat one of ovarian or colon cancer, wherein cells express IGF-1R on their surfaces, may be one which competes for binding IGF-1R with h7C10. In another embodiment, the methods of treatment include administering an antibody that binds the same epitope on IGF-1R as that bound by h7C10.

In a particular embodiment, the present invention relates to a murine antibody, or one of its functional fragments, according to the invention, characterized in that said antibody comprises a light chain of sequence comprising the amino acid sequence SEQ ID No. 54, or a sequence having at least 80% identity after optimum alignment with the sequence SEQ ID No. 54, or/and in that it comprises a heavy chain of sequence comprising the amino acid sequence SEQ ID No. 69, or a sequence having at least 80% identity after optimum alignment with the sequence SEQ ID No. 69.

According to a likewise particular aspect, the present invention relates to a chimeric antibody, or one of its functional fragments, according to the invention, characterized in that said antibody moreover comprises the light chain and heavy chain constant regions derived from an antibody of a species heterologous to the mouse, especially man, and in a preferred manner in that the light chain and heavy chain constant regions derived from a human antibody are respectively the kappa and gamma-1, gamma-2 or gamma-4 region.

According to a likewise particular aspect, the present invention relates to a humanized antibody or one of its functional fragments, according to the invention, characterized in that said antibody comprises a light chain and/or a heavy chain in which the skeleton segments FR1 to FR4 (such as defined below in examples 12 and 13, in tables 5 and 6) of said light chain and/or heavy chain are respectively derived from skeleton segments FR1 to FR4 of human antibody light chain and/or heavy chain.

According to a preferred embodiment, the humanized antibody or one of its functional fragments, according to the present invention is characterized in that said humanized antibody comprises a light chain comprising the amino acid sequence SEQ ID No. 61 or 65, or a sequence having at least 80% identity after optimum alignment with the sequence SEQ ID No. 61 or 65, or/and in that it comprises a heavy chain comprising the amino acid sequence SEQ ID No. 75, 79 or 83, or a sequence having at least 80% identity after optimum alignment with the sequence SEQ ID No. 75, 79 or 83.

Preferably, the humanized antibody, or one of its functional fragments, according to the invention is characterized in that said humanized antibody comprises a light chain comprising the amino acid sequence SEQ ID No. 65, and in that it comprises a heavy chain of sequence comprising the amino acid sequence SEQ ID No. 79 or 83, preferably SEQ ID No. 83.

According to a novel aspect, the present invention relates to an isolated nucleic acid, characterized in that it is chosen from the following nucleic acids:

a) a nucleic acid, DNA or RNA, coding for an antibody, or one of its functional fragments, according to the invention;

b) a complementary nucleic acid of a nucleic acid such as defined in a); and

c) a nucleic acid of at least 18 nucleotides capable of hybridizing under conditions of great stringency with at least one of the CDRs of nucleic acid sequence SEQ ID No. 1, 3, 5, 7, 9 or 11, or with a sequence having at least 80%, preferably 85%, 90%, 95% and 98%, identity after optimum alignment with the sequence SEQ ID No. 1, 3, 5, 7, 9 or 11.

By nucleic acid, nucleic or nucleic acid sequence, polynucleotide, oligonucleotide, polynucleotide sequence, nucleotide sequence, terms which will be employed indifferently in the present invention, it is intended to indicate a precise linkage of nucleotides, which are modified or unmodified, allowing a fragment or a region of a nucleic acid to be defined, containing or not containing unnatural nucleotides, and being able to correspond just as well to a double-stranded DNA, a single-stranded DNA as to the transcription products of said DNAs.

It must also be understood here that the present invention does not concern the nucleotide sequences in their natural chromosomal environment, that is to say, in the natural state. It concerns sequences which have been isolated and/or purified, that is to say that they have been selected directly or indirectly, for example by copy, their environment having been at least partially modified. It is thus likewise intended to indicate here the isolated nucleic acids obtained by genetic recombination by means, for example, of host cells or obtained by chemical synthesis.

By nucleic sequences having a percentage of identity of at least 80%, preferably 85%, 90%, 95% and 98%, after optimum alignment with a preferred sequence, it is intended to indicate the nucleic sequences having, with respect to the reference nucleic sequence, certain modifications such as, in particular, a deletion, a truncation, an elongation, a chimeric fusion and/or a substitution, especially point substitution. It preferably concerns sequences in which the sequences code for the same amino acid sequences as the reference sequence, this being connected to the degeneracy of the genetic code, or complementary sequences which are capable of hybridizing specifically with the reference sequences, preferably under conditions of high stringency, especially such as defined below.

A hybridization under conditions of high stringency signifies that the temperature conditions and ionic strength conditions are chosen in such a way that they allow the maintenance of the hybridization between two fragments of complementary DNA. By way of illustration, conditions of high stringency of the hybridization step for the purposes of defining the polynucleotide fragments described above are advantageously the following.

The DNA-DNA or DNA-RNA hybridization is carried out in two steps: (1) prehybridization at 42° C. for 3 hours in phosphate buffer (20 mM, pH 7.5) containing 5×SSC (1×SSC corresponds to a 0.15 M NaCl+0.015 M sodium citrate solution), 50% of formamide, 7% of sodium dodecyl sulfate (SDS), 10×Denhardt's, 5% of dextran sulfate and 1% of salmon sperm DNA; (2) actual hybridization for 20 hours at a temperature dependent on the size of the probe (i.e.: 42° C., for a probe size >100 nucleotides) followed by 2 washes of 20 minutes at 20° C. in 2×SSC+2% of SDS, 1 wash of 20 minutes at 20° C. in 0.1×SSC+0.1% of SDS. The last wash is carried out in 0.1×SSC+0.1% of SDS for 30 minutes at 60° C. for a probe size >100 nucleotides. The hybridization conditions of high stringency described above for a polynucleotide of defined size can be adapted by the person skilled in the art for oligonucleotides of greater or smaller size, according to the teaching of Sambrook et al., (1989, Molecular cloning: a laboratory manual. 2nd Ed. Cold Spring Harbor).

The invention likewise relates to a vector comprising a nucleic acid according to the present invention.

The invention aims especially at cloning and/or expression vectors which contain a nucleotide sequence according to the invention.

The vectors according to the invention preferably contain elements which allow the expression and/or the secretion of the nucleotide sequences in a determined host cell. The vector must therefore contain a promoter, signals of initiation and termination of translation, as well as appropriate regions of regulation of transcription. It must be able to be maintained in a stable manner in the host cell and can optionally have particular signals which specify the secretion of the translated protein. These different elements are chosen and optimized by the person skilled in the art as a function of the host cell used. To this effect, the nucleotide sequences according to the invention can be inserted into autonomous replication vectors in the chosen host, or be integrative vectors of the chosen host.

Such vectors are prepared by methods currently used by the person skilled in the art, and the resulting clones can be introduced into an appropriate host by standard methods, such as lipofection, electroporation, thermal shock, or chemical methods.

The vectors according to the invention are, for example, vectors of plasmidic or viral origin. They are useful for transforming host cells in order to clone or to express the nucleotide sequences according to the invention.

The invention likewise comprises the host cells transformed by or comprising a vector according to the invention.

The host cell can be chosen from prokaryotic or eukaryotic systems, for example bacterial cells but likewise yeast cells or animal cells, in particular mammalian cells. It is likewise possible to use insect cells or plant cells.

The invention likewise concerns animals, except man, which comprise at least one cell transformed according to the invention.

According to another aspect, a subject of the invention is a process for production of an antibody, or one of its functional fragments according to the invention, characterized in that it comprises the following stages:

a) culture in a medium and appropriate culture conditions of a host cell according to the invention; and

b) the recovery of said antibodies, or one of their functional fragments, thus produced starting from the culture medium or said cultured cells.

The cells transformed according to the invention can be used in processes for preparation of recombinant polypeptides according to the invention. The processes for preparation of a polypeptide according to the invention in recombinant form, characterized in that they employ a vector and/or a cell transformed by a vector according to the invention, are themselves comprised in the present invention. Preferably, a cell transformed by a vector according to the invention is cultured under conditions which allow the expression of said polypeptide and said recombinant peptide is recovered.

As has been said, the host cell can be chosen from prokaryotic or eukaryotic systems. In particular, it is possible to identify nucleotide sequences according to the invention, facilitating secretion in such a prokaryotic or eukaryotic system. A vector according to the invention carrying such a sequence can therefore advantageously be used for the production of recombinant proteins, intended to be secreted. In effect, the purification of these recombinant proteins of interest will be facilitated by the fact that they are present in the supernatant of the cell culture rather than in the interior of the host cells.

It is likewise possible to prepare the polypeptides according to the invention by chemical synthesis. Such a preparation process is likewise a subject of the invention. The person skilled in the art knows the processes of chemical synthesis, for example the techniques employing solid phases (see especially Steward et al., 1984, Solid phase peptide synthesis, Pierce Chem. Company, Rockford, 111, 2nd ed., (1984)) or techniques using partial solid phases, by condensation of fragments or by a classical synthesis in solution. The polypeptides obtained by chemical synthesis and being able to contain corresponding unnatural amino acids are likewise comprised in the invention.

The antibodies, or one of their functional fragments, capable of being obtained by a process according to the invention are likewise comprised in the present invention.

According to a second embodiment, the present invention concerns an antibody according to the invention such as described further above, characterized in that it is, moreover, capable of binding specifically to the human epidermal growth factor receptor EGFR and/or capable of specifically inhibiting the tyrosine kinase activity of said EGFR receptor.

In a general manner, the growth factors are small proteins involved in the regulation of the proliferation and of the differentiation of normal cells. Some of these growth factors likewise play an important role in the initiation and the maintenance of cell transformation, being able to function as autocrine or paracrine factors. This is especially the case, in addition to the IGF1 described further above, for the epidermal growth factor EGF, which seems particularly involved in the appearance of the tumor phenotype, the progression of tumors and the generation of metastases.

EGF and IGF1 exert their action through the intermediary of their respective receptor here called EGFR and IGF-IR. It concerns in the two cases membrane receptors with tyrosine kinase activity whose overexpression is described in numerous cancers. It must, however, be noted that the interaction of these two receptors is not clearly established and that the studies carried out by various teams in this connection give contradictory results as to the collaboration of these two receptors.

Studies carried out on prostate tumor cells show that the interruption of the autocrine loop EGF/EGFR by an anti-EGFR monoclonal antibody (here called “MAB” or “MAb”) is manifested by a complete loss of the response of the DU145 cells to IGF1 (Connolly J. M. and Rose D. P., Prostate, Apr. 24(4):167-75, 1994; Putz T. et al., Cancer Res., Jan. 1, 59(1):227-33, 1999). These results would suggest that a blockage of the receptor for the EGF would be sufficient in order to obtain a total inhibition of the transformation signals generated by the activation of the two receptors (EGFR and IGF-IR). On the other hand, other studies (Pietrzkowski et al., Cell Growth Differ, Apr., 3(4):199-205, 1992; Coppola et al., Mol Cell Biol., Jul., 14(7):4588-95, 1994) have shown that an over-expression of EGFR necessitates the presence of a functional IGF-IR in order to exert its mitogenic and transformant potential, although IGF-IR does not necessitate, for its part, the presence of functional EGFR in order to mediate its action. This second series of studies would be more in agreement with a strategy tending preferentially to block IGF-IR with the aim of simultaneously affecting the two receptors.

In a surprising manner, the inventors have, firstly, demonstrated that a coinhibition of the attachment of the IGF1 and/or IGF2 to the IGF-IR receptor and of the attachment of the EGF to the EGFR receptor allows a significant synergy of action of these two actions to be obtained against the in vivo tumor growth in nude mice carrying a tumor expressing these two receptors. One of the more probable hypotheses which is able to explain this synergy of action is that the two growth factors EGF and IGF1 (and/or IGF2) themselves act in synergy in the transformation of normal cells to cells with tumoral character and/or in the growth and/or the proliferation of tumor cells for certain tumors, especially for those overexpressing the two receptors EGFR and IGF-IR and/or having an overactivation of the transduction signal mediated by these two receptors, in particular at the level of the tyrosine kinase activity of these receptors.

As used herein, the term “IGF-1R mediated disorder” is intended to include diseases and other disorders in which the presence of high levels of IGF-IR in a subject suffering from the disorder has been shown to be or is suspected of being either responsible for the pathophysiology of the disorder or a factor that contributes to a worsening of the disorder. Alternatively, the disorder results from hyperactivation of the signaling pahway mediated by the interaction of IGF-1R with an endogenous ligand. Accordingly, an IGF-1R mediated disorder is a disorder in which inhibition of IGF-IR activity is expected to alleviate the symptoms and/or progression of the disorder. Such disorders may be evidenced, for example, by an increase in the levels of IGF-IR on the cell surface or in increased tyrosine autophosphorylation of IGF-IR in the affected cells or tissues of a subject suffering from the disorder. The increase in IGF-IR levels may be detected, for example, using an anti-IGF-IR antibody as described above.

According to a preferred aspect of this embodiment, the invention concerns an antibody such as described further above, characterized in that it consists of a bispecific antibody comprising a second motif specifically inhibiting the attachment of the EGF to the EGFR and/or specifically inhibiting the tyrosine kinase activity of said EGFR receptor.

The term “second motif” is intended to indicate above especially a sequence of amino acids comprising a fragment capable of specifically binding to EGFR, in particular a CDR region of a variable chain of an anti-EGFR antibody, or one of the fragments of this CDR region of sufficient length in order to exert this specific binding, or else several CDR regions of an anti-EGFR antibody.

The bispecific or bifunctional antibodies form a second generation of monoclonal antibodies in which two different variable regions are combined in the same molecule (Hollinger and Bohlen, 1999, Cancer and metastasis rev. 18: 411-419). Their use has been demonstrated both in the diagnostic field and in the therapy field from their capacity to recruit new effector functions or to target several molecules on the surface of tumor cells. These antibodies can be obtained by chemical methods (Glennie M. J. et al., 1987 J. Immunol. 139, 2367-2375; Repp R. et al., 1995, J. Hemat. 377-382) or somatic methods (Staerz U. D. and Bevan M. J. 1986 PNAS 83, 1453-1457; Suresh M. R. et al., 1986, Method Enzymol. 121: 210-228) but likewise and preferentially by genetic engineering techniques which allow the heterodimerization to be forced and thus facilitate the process of purification of the antibody sought (Merchand et al., 1998, Nature Biotech. 16:677-681).

These bispecific antibodies can be constructed as entire IgG, as bispecific Fab′2, as Fab′PEG or as diabodies or else as bispecific scFv but likewise as a tetravalent bispecific antibody or two attachment sites are present for each antigen targeted (Park et al., 2000, Mol. Immunol. 37 (18):1123-30) or its fragments as described further above.

In addition to an economic advantage from the fact that the production and the administration of a bispecific antibody are less onerous than the production of two specific antibodies, the use of such bispecific antibodies has the advantage of reducing the toxicity of the treatment. This is because the use of a bispecific antibody allows the total quantity of circulating antibodies to be reduced and, consequently, the possible toxicity.

In a preferred embodiment of the invention, the bispecific antibody is a bivalent or tetravalent antibody.

In practice, the interest in using a tetravalent bispecific antibody is that it has a greater avidity in comparison with a bivalent antibody on account of the presence of two attachment sites for each target, respectively IGF-IR and EGFR in the present invention.

In a similar manner to the selection of the functional fragments of the anti-IGF-IR antibody described above, said second motif is selected from the fragments Fv, Fab, F(ab′)₂, Fab′, scFv, scFv-Fc and the diabodies, or any form whose half-life would have been increased like the pegylated fragments such as Fv-PEG, scFv-PEG, Fab-PEG, F(ab′)₂—PEG or Fab′-PEG. According to an even more preferred aspect of the invention, said second anti-EGFR motif is descended from the mouse monoclonal antibody 225, its mouse-man chimeric derivative C225, or a humanized antibody derived from this antibody 225.

According to yet another aspect, a subject of the invention is an antibody, or one of its functional fragments, according to the invention as a medicament, preferably a humanized antibody such as defined above. Antibody, for the remainder of the present description, must be understood as an anti-IGF-IR antibody as well as a bispecific anti-IGF-IR/EGFR antibody.

The invention likewise concerns a pharmaceutical composition comprising by way of active principle a compound consisting of an antibody, or one of its functional fragments according to the invention, preferably mixed with an excipient and/or a pharmaceutically acceptable vehicle.

According to yet another embodiment, the present invention likewise concerns a pharmaceutical composition such as described further above which comprises a second compound chosen from the compounds capable of specifically inhibiting the attachment of the EGF to the human epidermal growth factor receptor EGFR and/or capable of specifically inhibiting the tyrosine kinase activity of said EGFR receptor.

In a preferred aspect of the invention, said second compound is chosen from the isolated anti-EGFR antibodies, or their functional fragments, capable of inhibiting by competition the attachment of the EGF to the EGFR. More particularly, said anti-EGFR antibody is chosen from the monoclonal, chimeric or humanized anti-EGFR antibodies, or their functional fragments. Even more particularly, said functional fragments of the anti-EGFR antibody are chosen from the fragments Fv, Fab, F(ab′)₂, Fab′, scFv-Fc or diabodies, or any fragment whose half-life would have been increased, like pegylated fragments. Said antibody can consist, in an even more preferred manner, of the mouse monoclonal antibody 225, its mouse-man chimeric derivative C225 (also called IMC-C225) or a humanized antibody derived from this antibody 225.

Another complementary embodiment of the invention consists in a composition such as described above which comprises, moreover, as a combination product for simultaneous, separate or sequential use, a cytotoxic/cytostatic agent and/or an inhibitor of the tyrosine kinase activity respectively of the receptors for IGF-I and/or for EGF.

“Simultaneous use” is understood as meaning the administration of the two compounds of the composition according to the invention in a single and identical pharmaceutical form.

“Separate use” is understood as meaning the administration, at the same time, of the two compounds of the composition according to the invention in distinct pharmaceutical forms.

“Sequential use” is understood as meaning the successive administration of the two compounds of the composition according to the invention, each in a distinct pharmaceutical form.

In a general fashion, the composition according to the invention considerably increases the efficacy of the treatment of cancer. In other words, the therapeutic effect of the anti-IGF-IR antibody according to the invention is potentiated in an unexpected manner by the administration of a cytotoxic agent. Another major subsequent advantage produced by a composition according to the invention concerns the possibility of using lower efficacious doses of active principle, which allows the risks of appearance of secondary effects to be avoided or to be reduced, in particular the effects of the cytotoxic agent.

In addition, this composition according to the invention would allow the expected therapeutic effect to be attained more rapidly.

In a particularly preferred embodiment, said composition as a combination product according to the invention is characterized in that said cytotoxic/cytostatic agent is chosen from the agents interacting with DNA, the antimetabolites, the topoisomerase I or II inhibitors, or else the spindle inhibitor or stabilizer agents or else any agent capable of being used in chemotherapy. Such cytotoxic/cytostatic agents, for each of the aforesaid classes of cytotoxic agents, are, for example, cited in the 2001 edition of VIDAL, on the page devoted to the compounds attached to the cancerology and hematology column “Cytotoxics”, these cytotoxic compounds cited with reference to this document are cited here as preferred cytotoxic agents.

In a particularly preferred embodiment, said composition as a combination product according to the invention is characterized in that said cytotoxic agent is coupled chemically to said antibody for simultaneous use.

In a particularly preferred embodiment, said composition according to the invention is characterized in that said cytotoxic/cytostatic agent is chosen from the spindle inhibitor or stabilizer agents, preferably vinorelbine and/or vinflunine and/or vincristine.

Immunoliposomes are liposomes capable of vehicling compounds, such as cytotoxic and/or cytostatic agents, such as described above, and of addressing them to tumour cells by means of antibodies or of antibody fragments attached to their surface. The antibodies or antibody fragments used are directed against antigens overexpressed at the surface of tumour cells and/or surface antigens the expression of which is restricted to tumour cells. They are preferably directed against tyrosine kinase receptors, and more particularly against the receptors for IGF-I, EGF or else VEGF. A preferred antibody is a monoclonal or polyclonal, preferably monoclonal, or even humanized, antibody which will recognize the IGF-IR specifically and with high affinity. Even more preferably, this antibody consists of the antibody which is the subject of the present invention.

The use of immunoliposomes for inhibiting tumour cell growth has been described in the literature. By way of example, mention may be made of the immunoliposomes which target proteins, such as ErbB2 (Hurwitz E. et al., Cancer Immunol. Immunother, 49:226-234, 2000; Park J. W. et al., Clinical Cancer Res., 8:1172-1181, 2002) or EGFR (Harding J. A. et al., Biochim. Biophys. Acta, 1327:181-192, 1997), or glycolipids such as the ganglioside GD2 (Pastorino F. et al., Cancer Res., 63:86-92, 2003).

Immunoliposomes combine the advantages of liposomes and of immunoconjugates. Liposomes in fact make it possible to encapsulate cytotoxic and/or cytostatic agents and thus to protect them against degradation. They also have the advantage of decreasing the toxicity of the vehiculed agents and of reducing the side effects that they induce. They may thus allow the use of agents which are much more toxic than the agents conventionally used in anticancer chemotherapies. The conjugation of antibodies or of antibody fragments to the surface of liposomes has the advantage of thus providing a system for specific targeting and addressing of the cytotoxic agent encapsulated in the liposome. In addition, unlike immunoconjugates, since the vehiculed agent is not covalently coupled to the antibody or to the antibody fragment, it will be completely active as soon as it is introduced into the target cell.

The antibodies or antibody fragments may be attached, without any limitation, covalently to the surface of the liposomes using conventional methods of bioconjugation. The coupling of these antibodies or of the fragments will be carried out on the lipids or lipids carrying a PEG which have been inserted into the liposomal membrane. In the case of a PEG-lipid, the coupling will be carried out on the PEG in the distal position with respect to the lipid. Liposomes carrying PEG groups (PEG-grafted liposomes) have the advantage of having longer half-lives than “naked” liposomes. By way of example, mention may be made of coupling of the antibody or of the fragment, via thiol groups, to the activated lipids or PEG-lipids exhibiting maleimide or bromoacetyl groups. The thiol groups for this type of coupling may come from 2 sources. They may be free cysteine residues introduced into a recombinant fragment of the antibody of interest, for example Fab′ or scFv fragments with an additional cysteine residue, or released after enzymatic hydrolysis of the antibody of interest and controlled reduction, which is the case, for example, during the preparation of Fab′ fragments from complete antibodies. Complete antibodies can also be coupled, after controlled oxidation of the oligosaccharides carried by the heavy chains, to lipids or PEG-lipids exhibiting free amine or hydrazide groups.

Since tumour cells overexpressing the IGF-IR generally possess the property of also overexpressing EGFR, it could also prove to be advantageous to claim bispecific immunoliposomes for targeting both the IGF-IR and the EGFR. Similarly, monospecific liposomes to the surface of which would be grafted one of the ligands for these two receptors, IGF-I, IGF-2 or EGF, or bispecific liposomes, would make it possible to target the same tumour cells overexpressing one of these receptors or both. This approach has been described for the EGFR (Kullberg E. B. et al., Pharm. Res., 20:229-236, 2003) but not for the IGF-IR.

Such immunoliposomes having antibodies anti-IGR-IR, or fragments thereof, attached covalently to the surface of the liposomes, are comprised in the present invention.

Method for the treatment of cancer wherein such immunoliposomes are administrated to patient in need of such treatment forms also part of the present invention.

In order to facilitate the coupling between said cytotoxic agent and said antibody according to the invention, it is especially possible to introduce spacer molecules between the two compounds to be coupled, such as poly(alkylene) glycols like polyethylene glycol, or else amino acids, or, in another embodiment, to use active derivatives of said cytotoxic agents into which would have been introduced functions capable of reacting with said antibody according to the invention. These coupling techniques are well known to the person skilled in the art and will not be expanded upon in the present description.

In another preferred embodiment, said inhibitor of the tyrosine kinase activity of the receptors for IGF-I and/or for EGF is selected from the group consisting of derived natural agents, dianilinophthalimides, pyrazolo- or pyrrolopyridopyrimidines or else quinazilines. Such inhibitory agents are well known to the person skilled in the art and described in the literature (Ciardiello F., Drugs 2000, Suppl. 1, 25-32).

Other inhibitors of EGFR can, without any limitation, consist of the anti-EGFR monoclonal antibodies C225 and 22Mab (ImClone Systems Incorporated), ABX-EGF (Abgenix/Cell Genesys), EMD-7200 (Merck KgaA) or the compounds ZD-1834, ZD-1838 and ZD-1839 (AstraZeneca), PKI-166 (Novartis), PKI-166/CGP-75166 (Novartis), PTK 787 (Novartis), CP 701 (Cephalon), leflunomide (Pharmacia/Sugen), CI-1033 (Warner-Lambert Parke-Davis), CI-1033/PD 183, 805 (Warner-Lambert Parke-Davis), CL-387, 785 (Wyeth-Ayerst), BBR-1611 (Boehringer Mannheim GmbH/Roche), Naamidine A (Bristol-Myers Squibb), RC-3940-II (Pharmacia), BIBX-1382 (Boehringer Ingelheim), OLX-103 (Merck & Co), VRCTC-310 (Ventech Research), EGF fusion toxin (Seragen Inc.), DAB-389 (Seragen/Lilgand), ZM-252808 (Imperial Cancer Research Fund), RG-50864 (INSERM), LFM-A12 (Parker Hughes Cancer Center), WHI-P97 (Parker Hughes Cancer Center), GW-282974 (Glaxo), KT-8391 (Kyowa Hakko) or the “EGFR Vaccine” (York Medical/Centro de Immunologia Molecular).

According to yet another embodiment of the invention, the composition such as described above can likewise comprise another antibody compound directed against the extracellular domain of the HER2/neu receptor, as a combination product for simultaneous, separate or sequential use, intended for the prevention and for the treatment of cancer, especially the cancers overexpressing said HER2/neu receptor and the receptor IGF-IR and/or EGFR, such as especially cancer of the breast.

Reference can be made especially to the publications of Albanell et al. (J. of the National Cancer Institute, 93(24):1830-1831, 2001) and of Lu et al. (J. of the National Cancer Institute, 93(24):1852-1857, 2001) justifying the unexpected interest in combining an anti-HER2/neu antibody with an anti-IGF-IR antibody according to the present invention.

In a particular manner, said anti-HER2/neu antibody of the composition according to the invention is the antibody called Trastuzumab (also called Herceptin).

The invention relates, in another aspect, to a composition characterized in that one, at least, of said antibodies, or one of their functional fragments, is conjugated with a cell toxin and/or a radioelement.

Preferably, said toxin or said radioelement is capable of inhibiting at least one cell activity of cells expressing the IGF-IR and/or EGFR receptor, in a more preferred manner capable of preventing the growth or the proliferation of said cell, especially of totally inactivating said cell.

Preferably also, said toxin is an enterobacterial toxin, especially Pseudomonas exotoxin A.

The radioelements (or radioisotopes) preferably conjugated to the antibodies employed for the therapy are radioisotopes which emit gamma rays and preferably iodine¹³¹, yttrium⁹⁰, gold¹⁹⁹, palladium¹⁰⁰, copper⁶⁷, bismuth²¹⁷ and antimony²¹¹. The radioisotopes which emit beta and alpha rays can likewise be used for the therapy.

By toxin or radioelement conjugated to at least one antibody, or one of its functional fragments, according to the invention, it is intended to indicate any means allowing said toxin or said radioelement to bind to said at least one antibody, especially by covalent coupling between the two compounds, with or without introduction of a linking molecule.

Among the agents allowing binding in a chemical (covalent), electrostatic or noncovalent manner of all or part of the components of the conjugate, mention may particularly be made of benzoquinone, carbodiimide and more particularly EDC (1-ethyl-3-[3-dimethyl-aminopropyl]-carbodiimide hydrochloride), dimaleimide, dithiobis-nitrobenzoic acid (DTNB), N-succinimidyl S-acetyl thio-acetate (SATA), the bridging agents having one or more phenylazide groups reacting with the ultraviolets (U.V.) and preferably N-[-4-(azidosalicylamino)butyl]-3′-(2′-pyridyldithio)propionamide (APDP), N-succinimid-yl 3-(2-pyridyldithio)propionate (SPDP), 6-hydrazino-nicotinamide (HYNIC).

Another form of coupling, especially for the radioelements, can consist in the use of a bifunctional ion chelator.

Among these chelates, it is possible to mention the chelates derived from EDTA (ethylenediaminetetraacetic acid) or from DTPA (diethylenetriaminepentaacetic acid) which have been developed for binding metals, especially radioactive metals, and immunoglobulins. Thus, DTPA and its derivatives can be substituted by different groups on the carbon chain in order to increase the stability and the rigidity of the ligand-metal complex (Krejcarek et al. (1977); Brechbiel et al. (1991); Gansow (1991); U.S. Pat. No. 4,831,175).

For example diethylenetriaminepentaacetic acid (DTPA) and its derivatives, which have been widely used in medicine and in biology for a long time either in their free form, or in the form of a complex with a metallic ion, have the remarkable characteristic of forming stable chelates with metallic ions and of being coupled with proteins of therapeutic or diagnostic interest such as antibodies for the development of radioimmunoconjugates in cancer therapy (Meases et al., (1984); Gansow et al. (1990)).

Likewise preferably, said at least one antibody forming said conjugate according to the invention is chosen from its functional fragments, especially the fragments amputated of their Fc component such as the scFv fragments.

The present invention moreover comprises the use of the composition according to the invention for the preparation of a medicament.

More particularly, according to another embodiment, the invention concerns the use of an antibody, or one of its functional fragments, and/or of a composition for the preparation of a medicament intended for the prevention or for the treatment of an illness induced by an overexpression and/or an abnormal activation of the IGF-IR and/or EGFR receptor, and/or connected with a hyperactivation of the transduction pathway of the signal mediated by the interaction of the 1-IGF 1 or IGF2 with IGF-IR and/or of EGF with EGFR and/or HER2/neu.

In the present specification, by the object of the invention “use of a product or a composition for the preparation of a medicament intended for the prevention or for the treatment of a disease”, it is also comprised “a method of preventing or treatment of such disease comprising the administration of said product or composition in a patient in need of such treatment”.

Preferably, said use according to the invention is characterized in that the administration of said medicament does not induce or induces only slightly secondary effects connected with inhibition of the insulin receptor IR, that is to say inhibition of the interaction of the IR receptor with its natural ligands due to the presence of said medicament, especially by a competitive inhibition connected with the attachment of said medicament to the IR.

The present invention moreover comprises the use of an antibody, or one of its functional fragments, preferably humanized, and/or of a composition according to the invention for the preparation of a medicament intended to inhibit the transformation of normal cells into cells with tumoral character, preferably IGF-dependent, especially IGF1- and/or IGF2-dependent and/or EGF-dependent and/or HER2/neu-dependent cells.

The present invention likewise relates to the use of an antibody, or one of its functional fragments, preferably humanized, and/or of a composition according to the invention for the preparation of a medicament intended to inhibit the growth and/or the proliferation of tumor cells, preferably IGF-dependent, especially IGF1- and/or IGF2-dependent and/or EGF-dependent and/or estrogen-dependent, and/or HER2/neu-dependent cells.

In a general manner, a subject of the present invention is the use of an antibody, or one of its functional fragments, preferably humanized, and/or of a composition according to the invention, for the preparation of a medicament intended for the prevention or for the treatment of cancer preferably expressing IGF-IR and/or EGFR, and/or of cancer preferably having a hyperactivation of the transduction pathway of the signal mediated by the interaction of IGF1 or IGF2 with IGF-IR, such as, for example, the overexpression of IRS1 and/or of EGF with EGFR.

The subject of the present invention is likewise the use of an antibody, or one of its functional fragments, preferably humanized, and/or of a composition according to the invention, for the preparation of a medicament intended for the prevention or for the treatment of psoriasis, psoriasis whose epidermal hyperproliferation can be connected with the expression or the overexpression of IGF-IR and/or EGFR, and/or with the hyperactivation of the transduction pathway of the signal mediated by the interaction of IGF-IR with its natural ligands (Wraight C. J. et al. Nat. Biotechnol., 2000, 18(5):521-526. Reversal of epidermal hyperproliferation in psoriasis by insulin-like growth factor I receptor antisense oligonucleotides) and/or of EGFR with its natural ligands.

Among the cancers which can be prevented and/or treated, prostate cancer, osteosarcomas, lung cancer, breast cancer, endometrial cancer or colon cancer or any other cancer overexpressing IGF-IR is preferred.

According to yet another aspect, a subject of the present invention is a method of diagnosis, preferably in vitro, of illnesses connected with an overexpression or an underexpression, preferably an overexpression, of the IGF-IR and/or EGFR receptor starting from a biological sample in which the abnormal presence of IGF-IR and/or EGFR receptor is suspected, characterized in that said biological sample is contacted with an antibody, or one of its functional fragments, according to the invention, it being possible for said antibody to be, if necessary, labeled.

Preferably, said illnesses connected with the overexpression of the IGF-IR and/or EGFR receptor in said diagnosis method will be cancers.

Said antibody, or one of its functional fragments, can be present in the form of an immunoconjugate or of a labeled antibody so as to obtain a detectable and/or quantifiable signal.

The antibodies labeled according to the invention or their functional fragments include, for example, antibodies called immunoconjugates which can be conjugated, for example, with enzymes such as peroxidase, alkaline phosphatase, α-D-galactosidase, glucose oxydase, glucose amylase, carbonic anhydrase, acetylcholinesterase, lysozyme, malate dehydrogenase or glucose 6-phosphate dehydrogenase or by a molecule such as biotin, digoxygenin or 5-bromodeoxyuridine. Fluorescent labels can be likewise conjugated to the antibodies or to their functional fragments according to the invention and especially include fluorescein and its derivatives, fluorochrome, rhodamine and its derivatives, GFP (GFP for “Green Fluorescent Protein”), dansyl, umbelliferone etc. In such conjugates, the antibodies of the invention or their functional fragments can be prepared by methods known to the person skilled in the art. They can be coupled to the enzymes or to the fluorescent labels directly or by the intermediary of a spacer group or of a linking group such as a polyaldehyde, like glutaraldehyde, ethylenediaminetetraacetic acid (EDTA), diethylene-triaminepentaacetic acid (DPTA), or in the presence of coupling agents such as those mentioned above for the therapeutic conjugates. The conjugates containing labels of fluorescein type can be prepared by reaction with an isothiocyanate.

Other conjugates can likewise include chemoluminescent labels such as luminol and the dioxetanes, bio-luminescent labels such as luciferase and luciferin, or else radioactive labels such as iodine¹²³, iodine¹²⁵, iodine¹²⁶, iodine¹³³, bromine⁷⁷, technetium^{99m}, indium¹¹¹, indium^{113m}, gallium⁶⁷, gallium⁶⁸, ruthenium⁹⁵, ruthenium⁹⁷, ruthenium¹⁰³, ruthenium¹⁰⁵, mercury¹⁰⁷, mercury²⁰³, rhenium^{99m}, rhenium¹⁰¹, rhenium¹⁰⁵, scandium⁴⁷, tellurium^{121m}, tellurium^{122m}, tellurium^{125m}, thulium¹⁶⁵, thulium¹⁶⁷, thulium¹⁶⁸, fluorine¹⁸, yttrium¹⁹⁹, iodine¹³¹. The methods known to the person skilled in the art existing for coupling the therapeutic radioisotopes to the antibodies either directly or via a chelating agent such as EDTA, DTPA mentioned above can be used for the radioelements which can be used in diagnosis. It is likewise possible to mention labeling with Na[I¹²⁵] by the chloramine T method [Hunter W. M. and Greenwood F. C. (1962) Nature 194:495] or else with technetium^{99m} by the technique of Crockford et al. (U.S. Pat. No. 4,424,200) or attached via DTPA as described by Hnatowich (U.S. Pat. No. 4,479,930).

Thus, the antibodies, or their functional fragments, according to the invention can be employed in a process for the detection and/or the quantification of an overexpression or of an underexpression, preferably an overexpression, of the IGF-IR and/or EGFR receptor in a biological sample, characterized in that it comprises the following steps:

a) the contacting of the biological sample with an antibody, or one of its functional fragments, according to the invention; and

b) the demonstration of the IGF-IR and/or EGFR/antibody complex possibly formed.

In a particular embodiment, the antibodies, or their functional fragments, according to the invention, can be employed in a process for the detection and/or the quantification of the IGF-IR and/or EGFR receptor in a biological sample, for the monitoring of the efficacy of a prophylactic and/or therapeutic treatment of IGF- and/or EGF-dependent cancer or else of psoriasis.

More generally, the antibodies, or their functional fragments, according to the invention can be advantageously employed in any situation where the expression of the IGF-IR and/or EGFR receptor must be observed in a qualitative and/or quantitative manner.

Preferably, the biological sample is formed by a biological fluid, such as serum, whole blood, cells, a tissue sample or biopsies of human origin.

Any procedure or conventional test can be employed in order to carry out such a detection and/or dosage. Said test can be a competition or sandwich test, or any test known to the person skilled in the art dependent on the formation of an immune complex of antibody-antigen type. Following the applications according to the invention, the antibody or one of its functional fragments can be immobilized or labeled. This immobilization can be carried out on numerous supports known to the person skilled in the art. These supports can especially include glass, polystyrene, poly-propylene, polyethylene, dextran, nylon, or natural or modified cells. These supports can be either soluble or insoluble.

By way of example, a preferred method brings into play immunoenzymatic processes according to the ELISA technique, by immunofluorescence, or radio-immunoassay (RIA) technique or equivalent.

Thus, the present invention likewise comprises the kits or sets necessary for carrying out a method of diagnosis of illnesses induced by an overexpression or an underexpression of the IGF-IR and/or EGFR receptor or for carrying out a process for the detection and/or the quantification of an overexpression or of an underexpression of the IGF-IR and/or EGFR receptor in a biological sample, preferably an overexpression of said receptor, characterized in that said kit or set comprises the following elements:

a) an antibody, or one of its functional fragments, according to the invention;

b) optionally, the reagents for the formation of the medium favorable to the immunological reaction;

c) optionally, the reagents allowing the demonstration of IGF-IR and/or EGFR/antibody complexes produced by the immunological reaction.

The invention moreover relates to the use of a composition as a combination product according to the invention, for the preparation of a medicament intended for the prevention or for the treatment of cancer, especially cancers for which said cytotoxic agent or said anti-HER2/neu antibody is generally prescribed and, especially, for which cancers the tumor cells express or overexpress the IGF-IR and/or EGFR receptor.

A subject of the invention is likewise the use of an antibody according to the invention for the preparation of a medicament intended for the specific targeting of a biologically active compound to cells expressing or overexpressing the IGF-IR and/or EGFR receptor.

It is intended here by biologically active compound to indicate any compound capable of modulating, especially of inhibiting, cell activity, in particular their growth, their proliferation, transcription or gene translation.

A subject of the invention is also an in vivo diagnostic reagent comprising an antibody according to the invention, or one of its functional fragments, preferably labeled, especially radiolabeled, and its use in medical imaging, in particular for the detection of cancer connected with the expression or the overexpression by a cell of the IGF-IR and/or EGFR receptor.

The invention likewise relates to a composition as a combination product or to an anti-IGF-IR and/or EGFR/toxin conjugate or radioelement, according to the invention, as a medicament.

Preferably, said composition as a combination product or said conjugate according to the invention will be mixed with an excipient and/or a pharmaceutically acceptable vehicle.

In the present description, pharmaceutically acceptable vehicle is intended to indicate a compound or a combination of compounds entering into a pharmaceutical composition not provoking secondary reactions and which allows, for example, facilitation of the administration of the active compound(s), an increase in its lifespan and/or in its efficacy in the body, an increase in its solubility in solution or else an improvement in its conservation. These pharmaceutically acceptable vehicles are well known and will be adapted by the person skilled in the art as a function of the nature and of the mode of administration of the active compound(s) chosen.

Preferably, these compounds will be administered by the systemic route, in particular by the intravenous route, by the intramuscular, intradermal, intraperitoneal or subcutaneous route, or by the oral route. In a more preferred manner, the composition comprising the antibodies according to the invention will be administered several times, in a sequential manner.

Their modes of administration, dosages and optimum pharmaceutical forms can be determined according to the criteria generally taken into account in the establishment of a treatment adapted to a patient such as, for example, the age or the body weight of the patient, the seriousness of his/her general condition, the tolerance to the treatment and the secondary effects noted.

For the first time, data illustrating the recognition of IGF-IR and Insulin/IGF-I hybrid receptor by the same monoclonal antibody able to inhibit specifically, in vitro and in vivo, the tumoral growth, thus allowing to treat cancer, more particularly breast cancer, able to conjointly express the two receptor types are shown in the present example (see particularly example 26). Actually, the capacity of 7C10 and h7C10 to recognize and/or inhibit the tyrosine kinase activity of IGF-IR and Insulin/IGF-I receptor allow to avoid the escape of tumor consequent upon the expression of this hybrid receptor. Such an antibody could be an innovative therapeutic compound of essential interest for the treatment of cancer.

Cancer pathologies are characterized by an uncontrolled cellular growth. In several cancers, growth factors are specifically binding with their receptors and then transmit growth, transformation and/or survival signals to the tumoral cell. The growth factor receptors over-expression at the tumoral cell surface is largely described (Salomon D S et al., Crit. Rev. Oncol. Hematol. 1995. 19: 183; Burrow S. et al., J. Surg. Oncol., 1998. 69: 21; Hakam A. et al. Hum. Pathol, 1999. 30: 1128; Railo M. J. et al., Eur. J. Cancer, 1994. 30: 307; Happerfield L. C. et al., J. Pathol., 1997. 183: 412). This over-expression, leading to a direct perturbation of cellular growth regulation mechanisms, can also affect the cell sensibility to induced apoptose by classical chemotherapies or radiotherapies.

During last few years, it has been show that the targeting of growth factor receptors, like EGF-R (for Epidermal growth factor receptor) or Her2/neu over-expressed on the tumoral cell surface, with respectively humanized (Herceptin®) or chimeric (C225) antibodies results in an significant inhibition of the tumoral growth on patients and in a significant increase of the efficacity of classical chemotherapy treatments (Carter P. Nature Rev. Cancer, 2001. 1(2): 118; Hortobagyi G. N. Semin. Oncol., 2001. 28: 43; Herbst R. S. et al. Semin. Oncol., 2202. 29: 27). Other receptors like IGF-IR (for Insulin like growth factor receptor) or VEGF-R (for vascular endothelial growth factor receptor) have been identified as potential target in several preclinical studies.

More particularly, IGF-IR is part of the tyrosine kinase receptors. It shows a high homology with the Insulin receptor (IR) which exists under two isoforms, A and B.

The IGF-IR and IR are tetrameric glycoproteins composed of two extracellular α- and two transmembrane β-subunits linked by disulfide bonds. Each α-subunit, containing the ligand-binding site is approximately 130- to 135-kDa, whereas each β-subunit containing the tyrosine kinase domain is approximately 90- to 95-kDa. These receptors share more than 50% overall amino acid sequence similarity and 84% similarity in the tyrosine kinase domain. After ligand binding, phosphorylated receptors recruit and phosphorylate docking proteins, including the insulin receptor substrate-I protein family (IRS1), Gab1 and Shc (Avruch 1998, Roth et al. 1988, White 1998, Laviola et al. 1997, Cheatham et al. 1995), leading to the activation of different intracellular mediators. Although both the IR and IGF-IR similarly activate major signalling pathways, differences exist in the recruitment of certain docking proteins and intracellular mediators between both receptors (Sasaoka et al. 1996, Nakae et al. 2001, Dupont and Le Roith 2001, Koval et al. 1998). These differences are the basis for the predominant metabolic effects elicited by IR activation and the predominant mitogenic, transforming and anti-apoptotic effects elicited by IGF-IR activation (De Meyts et al. 1995, Singh et al. 2000, Prisco et al. 1999, Kido et al. 2001). Insulin binds with high affinity to the IR (100-fold higher than to the IGF-IR), whereas insulin-like growth factors (IGF-I and IGF-2) bind to the IGF-IR with 100-fold higher affinity than to the IR.

The human IR exists in two isoforms, IR-A and IR-B, generated by alternative splicing of the IR gene that either excludes or includes 12 amino acid residues encoded by a small exon (exon 11) at the carboxy-terminus of the IR α-subunit. The relative abundance of IR isoforms is regulated by tissue specific and unknown factors (Moller et al. 1989, Mosthaf et al. 1990). IR-B is the predominant IR isoform in normal adult tissues (adipose tissue, liver and muscle) that are major target tissues for the metabolic effects of insulin (Moller et al. 1989, Mosthaf et al. 1990). IR-A is the predominant isoform in fetal tissues and mediates fetal growth in response to IGF-2 (Frasca et al. 1999), as also suggested by genetic studies carried out in transgenic mice (DeChiara et al. 1990, Louvi et al. 1997). Moreover, when cells transform and become malignant, dedifferentiation is often associated with an increased IR-A relative abundance (Pandini et al. 2002).

Given the high degree of homology, the insulin and IGF-I half-receptors (composed of one α- and one β-subunit) can heterodimerize, leading to the formation of insulin/IGF-I hybrid receptors (Hybrid-Rs) (Soos et al. 1990, Kasuya et al. 1993, Seely et al. 1995, Bailyes et al. 1997).

Both IR isoforms are equally able to form hybrids with IGF-IR. Hybrid-RsA and Hybrid-RsB, however, have different functional characteristics. Hybrid-RsB has reduced affinity for IGF-I and especially for IGF-2. In contrast, Hybrid-RsA has a high affinity for IGF-I and bind also IGF-2 and insulin at a physiological concentration range. The expression of Hybrid-RsA up-regulates the IGF system by two different mechanisms i) binding (with high affinity) and activation by both IGF-I and IGF-2 (which do not occur with the Hybrid-RsB), ii) activation of the IGF-IR pathway after insulin binding. Insulin binding to Hybrid-RsA phosphorylates the IGF-IR β-subunit and activates an IGF-1R-specific substrate (CrkII) so that Hybrid-RsA shifts insulin to IGF-IR signaling (Pandini et al. 2002).

In several tissues, like liver, spleen or placenta, Hybrid-Rs are more represented than IGF-IR (Bailyes et al. 1997). As tumor tissues overexpress both IGF-IR and IR-A (Frasca et al. 1999, Sciacca et al. 1999, Vella et al. 2001), Hybrid-RsA may also be overexpressed in a variety of human malignancies, including thyroid and breast cancers providing a selective growth advantage to malignant cells able to respond by a type IGF-IR signalisation following a stimulation by IGF-I and/or IGF-2 but also by insulin at physiological concentrations (Bailyes et al. 1997, Pandini et al. 1999, Belfiore et al. 1999, Frasca et al. 1999, Sciacca et al. 1999, Vella et al. 2001).

The realisation of such “therapeutic tools” able to block in the same time the two receptors is of particular interest as they will allow to avoid the escape phenomena mediated by the expression in a same tumor of IGF-IR and hybrid receptors.

The present invention allows to jointly block the Insulin/IGF-I receptor and IGF-IR activity by generating a compound, and more particularly an antibody, of high affinity able to bind to said two receptors and also to block their activation by IGF-I, IGF-II or Insulin.

The present invention also deals with the use of an isolated antibody according to the present invention, or a fragment thereof, said antibody or fragment being able to bind to i) human IGF-IR and/or to inhibit the natural binding of its ligands IGF-I and/or IGF-II, and/or also able to inhibit specifically the tyrosine kinase activity of said IGF-IR and ii) insulin/IGF-I hybrid receptors, and/or to inhibit the natural binding of their ligands IGF-I, IGF-II and/or Insulin, and/or also able to specifically inhibit the tyrosine kinase activity of said Insulin/IGF-I receptors.

More particularly, in a preferred embodiment, said antibody is characterized in that it comprises the sequences of the 7C10 and h7C10 antibodies anti-IGF-IR, and fragment thereof, of the present invention, notably the antibodies anti-IGF-IR according to the present invention having a light chain comprising at least a CDR region selected in the group consisting in SEQ ID) No. 2, 4 or 6 (or at least a CDR with at least 80% of homology after optimal alignment with SEQ ID No. 2, 4 or 6), and/or a heavy chain comprising at least a CDR region selected in the group consisting in SEQ ID No. 8, 10 or 12 (or at least a CDR with at least 80% of homology after optimal alignment with SEQ ID No. 8, 10 or 12).

According to another preferred embodiment, said antibody is used for cancer therapy, more particularly breast cancer therapy.

Actually, it is known that breast tumoral cells specifically present on their surface IGF-IR but also a great number of Insulin receptor and, as a consequence, a great number of Insulin/IGF-1Hybrid receptors (Frasca et al. 1999, Sciacca et al. 1999, Vella et al. 2001).

The antibody, or fragments thereof, could be use alone or in association with another antibody able to target another growth factor implied in the proliferation or dissemination of tumoral cells. It could also be used in association with a chemotherapeutic agent or another tyrosine kinase inhibitor in co-administration or in the form of an immuno-conjugate, said agent being chemical, biological and/or natural. Fragments of said antibody could also be use in bispecific antibodies obtained by recombinant mechanisms or biochemical coupling, and then associating the specificity of the above described antibody with the specificity of other antibodies able to recognise other receptors involved in the proliferation, the angiogenese or any other mechanisms involved in the tumoral development.

Particular aspect of the present invention: Cytotoxic and/or cytostatic-active agent coupled to an addressing system, particularly to the antibodies 7C10, C7C10 or h7C10, or fragment thereof, according to the present invention capable of binding specifically to the human insulin-like growth factor-1 receptor IGF-IR and Insulin/IGF-I hybrid receptor

The present invention relates also to novel compounds comprising a cytotoxic and/or cytostatic active agent coupled to an addressing system. More particularly, the present invention relates to a compound comprising a Vinca alkaloid coupled to an antibody capable of binding specifically to the human insulin-like growth factor-1 receptor IGF-IR and/or capable of specifically inhibiting the tyrosine kinase activity of said IGF-IR receptor, in particular a monoclonal antibody of murine, chimeric, primatized, humanized and human origin. The invention also relates to the mode of coupling of the elements of said compound and also comprises the use of these compounds as a medicinal product for the prophylactic and/or therapeutic treatment of cancer, more particularly of cancers overexpressing IGF-IR, or of any pathological condition associated with overexpression of said receptor.

Currently, along with surgery and radiotherapy, chemotherapy represents one of the most effective means of combating cancer. Many cytotoxic and/or cytostatic agents have been isolated or synthesized and make it possible to destroy or reduce, if not definitively, at least significantly, the tumour cells. However, the toxic activity of these agents is not limited to tumour cells, and the non-tumour cells are also effected and can be destroyed. More particularly, side effects are observed on rapidly renewing cells, such as haematopoietic cells or cells of the epithelium, in particular of the mucous membranes. By way of illustration, the cells of the gastrointestinal tract are largely affected by the use of cytotoxic agents.

One of the aims of the present invention is also to be able to provide a compound which makes it possible to limit the side effects on normal cells while at the same time conserving a high cytotoxicity on tumour cells.

According to an original approach, the applicant, rather than developing new molecules, has sought to overcome the problem of toxicity of known molecules by limiting to tumour cells the access of said molecules. To do this, the applicant has developed an antibody-type addressing system for targeting only tumour cells.

One of the advantages of this approach is to be able to use known cytotoxic agents which are well defined in pharmacological and pharmacokinetic terms. In addition, it is then possible to use strong cytotoxic agents which until now have been neglected in favour of cytotoxic agents which are less strong but which have a better therapeutic index (and therefore exhibit fewer side effects).

Another advantage lies in the use of an antibody, i.e. of a product of biological origin which does not add any toxicity to that of the cytotoxic agent. In addition, as will be subsequently developed, the choice of the antibody makes it possible to accumulate with the action of the cytotoxic agent its own biological activity.

The applicant has demonstrated that the use of a Vinca alkaloid coupled to an addressing device is of value in chemotherapy.

According to a first aspect, a subject of the present invention is a compound comprising at least one molecule of active agent coupled to an addressing system, said at least one molecule of active agent being a strong cytotoxic and/or cytostatic compound chosen from Vinca alkaloids, and said addressing system being a polyclonal or monoclonal antibody, which may be bispecific, or a functional fragment thereof, capable of targeting, preferably specifically, tumour cells.

An advantage of a compound according to the invention is that the active agent is directly brought to the target cells by the antibody and, besides the fact that it does not degrade the other cells, its biological activity is not decreased.

One of the advantages associated with using antibodies as an addressing system is that it is possible to couple several active agents to them, thus increasing the efficacy of the compound. Specifically, since the compound is brought directly to the target cells, the fact that there are several active agents will not lead to an increase in side effects, but only to an increase in the desired in situ effect on the tumour cells.

By way of non-limiting examples of targeting antibodies which can be used according to the invention, mention may be made, without any limitation, of the CeaVac antibodies directed against colorectal tumour cells, and the Y Theragyn/pemtumomab and OvaRex antibodies directed against ovarian tumour cells.

The present invention relates to a compound as described above, which comprises from 1 to 50 molecules of active agent, preferably from 1 to 10, and better still from 1 to 6. The choice of the number of molecules of active agent depends, inter alia, on the molecular weight of each of the elements. For example, by way of indication, for an antibody of IgG1 type with a molecular weight of 150 000 Da, it is preferred to couple from 4 to 6 molecules of vinblastine with a molecular weight of 900 Da (Petersen et al., Cancer Res., 1991, 51:2286). If the antibody is conjugated with too large an amount of cytotoxic agents, there is a risk that said agents will mask the recognition site for the antigen and decrease its activity.

In practice, the compound which is the subject of the invention is used as a medicinal product, and more particularly as a medicinal product intended for the treatment of cancer.

The present invention differs from the prior art not only in the sense that the choice of the antibody is aimed at targeting tumour cells as described above, but also in that said antibody exhibits an intrinsic activity on the tumour cells.

According to another embodiment of the invention, the compound as described above is also capable of inhibiting tumour cell proliferation and/or apoptotic function restoration by blocking transduction signals, the progression of cells in the cell cycle and/or membrane-bound receptor availability (phenomena of internalization and of degradation of said receptor), or of reverting an apoptosis-resistant phenotype in the case of an antibody directed against the IGF-IR, insofar as it is widely described that overexpression of this receptor confers on tumour cells a means of withstanding apoptosis and in particular apoptosis induced by chemotherapy compounds (Beech D. J. et al., Oncology reports, 2001, 8:325-329; Grothe A. et al., J. Cancer Res. din Oncol., 1999, 125:166-173). Another mechanism of action of the compound as described above may be associated with the Fc portion of the antibody, if a whole antibody is used, and may consist of the setting up of effector mechanisms such as ADCC (antibody-dependent cellular cytotoxicity) and CDC (complement-dependent cytotoxicity).

By way of non-limiting example of antibodies, mention may be made of Avastin/Bevacizumab which acts on colorectal cancers by interfering with tumour angiogenesis, Rituxan/rituximab, the activity of which is mainly related to the effector functions of the molecule, and in particular ADCC, and also Herceptin/trastuzumab which acts by inhibition of signal transduction and inhibition of cell progression in the cell cycle, and also, in large part, by initiating ADCC mechanisms.

Vinca alkaloids correspond to the family of natural compounds of which vinblastine, vincristine, anhydrovinblastine and leurosine, which are present in considerable amounts in plants, are demonstrative examples.

The term “Vinca alkaloids” should also be understood to mean all the derivatives present in small amounts, such as deoxyvinblastine or leurosidine, taken by way of non-limiting examples. It should also be understood to mean derivatives of natural structure but which are obtained by synthesis, such as, without any limitation, anhydrovinblastine.

The term “Vinca alkaloid” should also be understood to mean all the compounds derived from these natural compounds by chemical or biochemical modification in one or more steps. These modifications may affect the “vindoline” component or the “velbanamine” component or both components simultaneously. The Vinca alkaloids, as such, are known to those skilled in the art (Antitumor Bisindole Alkaloids from Catharanthus roseus (L.)). The Alkaloids, Brossi A. et al., M. Ed. Academic Press Inc. San Diego, Vol. 37, 1990; Jacquesy J. C. et al., Biomedical Chemistry: Applying Chemical Principles to the Understanding and Treatment of Disease, edited by Torrence, P. F., John Wiley and Sons Inc.: New York, 2000, pp. 227-246; Fahy J. et al., J. Current Pharm. Des., 2001, 7:1181-97; Duflos A. et al., Novel Aspects of Natural and Modified Vinca Alkaloids, Curr. Med. Chem.—Anti-Cancer Agents, 2002, 2:55-70).

The preferred derivatives according to the present invention are those which exhibit a pharmacological advantage established by virtue of cytotoxicity assays or activity assays on certain specific targets, such as tubulin, or which have demonstrated advantages in in vivo tests on animals. Among these compounds, mention may be made of the derivatives currently used in anticancer chemotherapy: vinblastine, vincristine, vindesine and vinorelbine, and also the derivatives which have demonstrated an advantage in clinical studies, such as vinepidine, vinfosiltine, vinzolidine and vinflunine.

The invention is therefore partly based on the choice of an original cytotoxic agent without any bias from the prior art.

More particularly, a subject of the present invention is a compound as described above, in which said Vinca alkaloid is selected from vinblastine, deoxyvinblastine, deoxyleurosidine, vincristine, vindesine, vinorelbine, vinepidine, vinfosiltine, vinzolidine and vinflunine.

The subject of the invention has, more specifically, been demonstrated and exemplified using deoxyvinblastine and its 4′-S isomer, commonly known as deoxyleurosidine.

The structure of each of these two compounds has been described for many years, but their pharmacological activity is considered to be moderate or weak (Neuss N. et al., Tetrahedron Letters, 1968, No. 7, pp 783-7; U.S. Pat. No. 4,143,041, Eli Lilly and Company, Filed Nov. 25, 1977; and recently, Kuehne M. E. et al., J. Org. Chem., 1989, 54, 14:3407-20; Kuehne M. E., Org. Biomol. Chem., 2003 1:2120-36). Their real advantage as a compound with unquestionable antitumour pharmacological activity has never been described and demonstrated by in vivo experiments on murine tumour models.

The present invention therefore relates to a compound as described above, in which said Vinca alkaloid is (4′-R) deoxyvinblastine and/or (4′-S) deoxyleurosidine.

The greater activity of these two derivatives has been demonstrated against P388 murine leukaemia grafted intravenously on day 0. The compound is administered intraperitoneally in a single dose on day 1. The protocol for this test is described by Kruczynski A. et al., Cancer Chemotherapy and Pharmacology, 1998, volume 41, pages 437 to 447.

Conventionally, the in vivo activity of cytotoxic compounds is expressed by the T/C at a dose expressed in mg per kg. The T/C corresponds to the ratio, multiplied by 100, of the median of the survival time of the treated animals to the median of the survival time of the control animals.

By way of example, for cytotoxic agents used to date, the maximum activity of vinblastine sulphate is expressed at the dose of 5 mg/kg, with T/C=143. The maximum activity of vincristine sulphate is expressed at the doses of 1.25 and 2.5 mg/kg, with T/C=143 in both cases.

Unexpectedly, the maximum activity of deoxyvinblastine ditartrate is expressed at the dose of 20 mg/kg, with T/C=214 and the maximum activity of deoxyleurosidine ditartrate is expressed at the dose 2.5 mg/kg, with T/C=200.

In view of these results, the present invention therefore relates to the use of (4′-R) deoxyvinblastine and/or (4′-S) deoxyleurosidine, collectively referred to as deoxyvinblastine in the remainder of the description, for treating cancer.

According to a preferred form, as described above, the present invention envisages the coupling of deoxyvinblastine to a compound of the monoclonal or polyclonal, preferably monoclonal, antibody type.

More particularly, as will subsequently be described, a preferred antibody making up the compound which is the subject of the present invention is a monoclonal or polyclonal, preferably monoclonal, antibody which will recognize the IGF-IR specifically and with high affinity, and which will have the ability to inhibit the growth of tumours, more particularly of tumours expressing the IGF-IR.

The cytoplasmic protein tyrosine kinases are activated by binding of the ligand to the extracellular domain of the receptor. Activation of the kinases leads, in turn, to stimulation of various intracellular substrates, including IRS-1, ISR-2, Shc and Grb 10 (Peruzzi F. et al., J. Cancer Res. Clin. Oncol., 125:166-173, 1999). The two major substrates for the IGF-IR are IRS and Shc, which mediate, by activation of many downstream effectors, most of growth and differentiation effects associated with the binding of IGFs to this receptor. Substrate availability can, consequently, dictate the final biological effect associated with activation of the IGF-IR. When IRS-I predominates, the cells tend to proliferate and to transform. When Shc dominates, the cells tend to differentiate (Valentinis B. et al., J. Biol. Chem., 274:12423-12430, 1999). It appears that the pathway mainly implicated for the effects of protection against apoptosis is the phosphatidylinositol 3-kinases (PI 3-kinases) pathway (Prisco M. et al., Horm. Metab. Res., 31:80-89, 1999; Peruzzi F. et al., J. Cancer Res. Clin. Oncol., 125:166-173, 1999).

According to a preferred embodiment, a subject of the present invention is a compound as described above (cytotoxic and/or cytostatic active agent coupled to an addressing system), comprising an antibody capable of recognizing the IGF-IR specifically and with high affinity. This antibody will interact little or not at all with the insulin receptor IR. Its binding should inhibit, in vitro, the growth of tumours expressing the IGF-IR by interacting mainly with the signal transduction pathways activated during IGF1/IGF-IR and IGF2/IGF-IR interactions. This antibody should be active in vivo on all tumour types expressing the IGF-IR, including oestrogen-dependent breast tumours and prostate tumours, which is not the case for the anti-IGF-IR monoclonal antibodies (referred to as MAb or MAB) currently available. In fact, αIR3, which is a reference in the IGF-IR field, completely inhibits the growth of oestrogen-dependent breast tumours (MCF-7) in vitro, but has no effect on the corresponding in vivo model (Artega C. et al., J. Clin Invest., 84:1418-1423, 1989). Similarly, the scFv-Fc fragment derived from the murine monoclonal 1H7 is only weakly active on the MCF-7 breast tumour and completely inactive on an androgen-independent prostate tumour (Li S. L. et al., Cancer Immunol. Immunother., 49:243-252, 2000).

According to a preferred embodiment, a subject of the present invention is a compound (cytotoxic and/or cytostatic active agent coupled to an addressing system) as described above, comprising an antibody, or one of its functional fragments, said antibody or one of its said fragments being capable of binding specifically to the human insulin-like growth factor-I receptor IGF-IR and, where appropriate, capable of inhibiting the natural binding of the IGF-IR ligands IGF1 and/or IGF2, and/or capable of specifically inhibiting the tyrosine kinase activity of said IGF-IR receptor.

Such a compound has a double advantage.

Firstly, it makes it possible, as described above, to bring the cytotoxic agent directly to tumour cells, more particularly tumour cells overexpressing the IGF-IR, and thus to decrease the side effects in normal cells.

Secondly, its mode of action is not limited to targeting. The compound which is the subject of the present invention cumulates the action of the cytotoxic agent which makes it possible to destroy the tumour cells and the action of the antibody which will inhibit the growth of tumour cells, preferably of tumour cells expressing the IGF-IR, by interacting with the signal transduction pathways, and will make it possible to decrease the resistance to apoptosis of cells overexpressing the receptor for IGF-I and, consequently, to improve the activity of chemotherapy drugs, part of the mechanism of action of which lies in the induction of apoptosis.

According to a preferred embodiment of the compound (cytotoxic and/or cytostatic active agent coupled to an addressing system) which is the subject of this particularly object of the present invention, the monoclonal antibody, or one of its functional fragments, is the 7C10, a C7C10 or a h7C10, or fragment thereof, or their derived antibodies, as described in the first part of the present specification directed to the antibodies anti-IGR-IR of the present invention

In this respect, the applicant filed a French patent application FR 03/08538 on Jul. 11, 2003 for “Novel antitumour immunoconjugates”. The content of this patent application is incorporated herein by way of reference.

Immunoliposomes containing such particular cytotoxic and/or cytostatic agents, such as described above, such as the vinca alkaloids, and of addressing them to tumour cells by means of antibodies or of antibody fragments attached to their surface are comprised in the present invention.

Method of treatment of cancer, particularly the preferred cancers cited above, comprising the administration of the present immunoliposomes forms also part of the present invention.

The antibodies or antibody fragments used are directed against antigens overexpressed at the surface of tumour cells and/or surface antigens the expression of which is restricted to tumour cells. They are preferably directed against tyrosine kinase receptors, and more particularly against the receptors for IGF-I, EGF or else VEGF. A preferred antibody is a monoclonal or polyclonal, preferably monoclonal, or even humanized, antibody which will recognize the IGF-IR specifically and with high affinity. Even more preferably, this antibody consists of the antibody anti-IGR-IR which is the subject of the present invention described in the first part of the specification.

According to another embodiment of the compound (cytotoxic and/or cytostatic active agent coupled to an addressing system) which is a subject of the present invention, the monoclonal antibody as described above is also capable of binding specifically to the human epidermal growth factor receptor, EGFR, and/or capable of specifically inhibiting the tyrosine kinase activity of said EGFR receptor.

According to a preferred aspect of this embodiment of the compound(cytotoxic and/or cytostatic active agent coupled to an addressing system), the coupled monoclonal antibody consists of a bispecific antibody comprising a second unit which specifically inhibits the binding of EGF to the EGFR and/or which specifically inhibits the tyrosine kinase activity of said EGFR receptor.

In a preferred embodiment of the invention, the bispecific antibody which can be used here for cytotoxic and/or cytostatic active agent coupled to an addressing system according to this invention are those as described in the first part of the present specification related to bispecific antibodies of the invention.

Another aspect of the invention concerns the mode of coupling between the antibody and the cytotoxic agent. Whatever the nature of the coupling, which may be direct or indirect, stable or labile, it should in no way impair the respective biological functions of the antibody and of the cytotoxic agent. It is clearly understood that any coupling satisfying this characteristic, and known to those skilled in the art, is included in the scope of the present patent application. In addition, the coupling, and more particularly the linkage used, must allow release of the deoxyvinblastine, in the 4-deacetylated or 3-acid, or 4-deacetylated and 3-acid, form, or in the form of one of these forms carrying all or part of said linkage used, in the target cells.

According to a preferred embodiment, the coupling is chemical coupling. More particularly, said chemical coupling is composed of an anchorage on the Vinca alkaloid, an anchorage on the antibody and a linkage connecting these two anchorages.

The term “linkage” should be understood to mean any structure capable of providing a bond of whatever possible nature between the two elements of the compound, namely a chemical molecule and an antibody.

In terms of the anchorage on the Vinca alkaloid, several possibilities are envisaged. Mention may, for example, be made of an anchorage on the alcohol function in the 4-position after deacetylation of the 4-acetoxy group of said Vinca alkaloid.

In another embodiment, the anchorage on the Vinca alkaloid is effected on the acid function in the 3-position after deacetylation of the 4-acetoxy group and demethylation of the ester function in the 3-position of said Vinca alkaloid.

According to yet another embodiment of the invention, the anchorage on the Vinca alkaloid is effected on the acid function in the 3-position directly by reaction on the ester function in the 3-position of said Vinca alkaloid.

According to yet another embodiment of the invention, the anchorage on the Vinca alkaloid is effected via an ester or thioester function on the hydroxyl function in the 3-position.

An additional embodiment consists in effecting the anchorage on the Vinca alkaloid via an amide function or an ester function or a hydrazide function on the acid function in the 4-position.

As regards the anchorage on the antibody, it should in no way denature the antibody, so as not to decrease its ability to recognize and interact with the tumour cells.

To do this, it is preferable for the anchorage on the antibody to be effected on the oligosaccharides, the lysines and/or the aspartic acid and glutamic acid residues.

The Vinca alkaloid may also be coupled on the carboxylic functions of the antibody, carried by the aspartic acid and glutamic acid residues of the antibody. For example, an amine, hydrazide or hydrazine derivative of the Vinca alkaloid will be coupled on these residues in the presence of a compound of carbodiimide type, such as N-(3-dimethylaminopropyl)-N′-ethylcarbodiimide (or EDAC).

In practice, it is even more preferable to effect the anchorage on the oligosaccharides present on the antibody. Specifically, there are no oligosaccharides in the recognition sites of the antibody and, as a result, there is no risk of impairing the recognition/biological activity capacities of said antibody. According to a preferred embodiment of the invention, the anchorage is effected on the oligosaccharides present on the asparagines (Asn) which are followed by a consensus sequence consisting of an amino acid and a serine or a threonine. For example, without any limitation, a preferred anchorage on the IgG1 antibody used in the invention is on Asn297.

A combined anchorage, i.e. an anchorage on oligosaccharides, lyines and/or aspartic acid and glutamic acid, is also covered.

An additional embodiment consists in greatly increasing the density of the Vinca alkaloid in order to attain 10 to 50 mol per mole of antibody. Mention may be made of the coupling of a hemisuccinate derivative of the Vinca alkaloid on a lysine polymer (Poly-L-Lys or Poly-D-Lys). The conjugate thus obtained is then coupled on the oligosaccharides of the antibody, oxidized beforehand with meta-periodate.

In another embodiment, a hydrazide derivative of the Vinca alkaloid may be coupled on a dextran oxidized beforehand with meta-periodate. The conjugate obtained is then coupled to the antibody via the lysine residues.

According to yet another embodiment, a hemisuccinate derivative of the Vinca alkaloid may be coupled on a dextran activated beforehand by controlled oxidation with meta-periodate and then substituted with a compound of diamide type. The conjugate obtained is then coupled on the lysine residues of the antibody.

According to a preferred embodiment of the invention, the anchorage on the antibody is effected by reaction of an amine function, a hydrazine function, a hydrazide function or an acid function which has been activated.

More particularly, the anchorage on the antibody is effected by reaction of an epoxide function or of a disulphide function, a sulphide function or an acid function which has been activated, with a nitrogen-containing residue or with a hydroxyl residue or with a thiol residue of said antibody.

Mention may also be made, in a nonlimiting manner, of other linkages which may also be used to covalently attach the Vinca alkaloids to the antibodies or to their functional fragments (Garnett et al., Adv. Drug Deliv. Rev., 2001, 171-216), such as aldehydes which make it possible to form Schiff bases, which can then be stabilized by reduction with sodium borohydride or cyanoborohydride; disulphides which have the advantage of being able to release the Vinca alkaloids inside the tumour cell by virtue of the intracytoplasmic reducing environment; more stable thioethers; more labile thioesters; linkages which are labile in acidic medium, which have the advantage of allowing release of the cytotoxic agent in the tumour, which is generally more acid, or during the passage from the endosome (pH 6.0 6.8) to the lysosome (pH 4.5 5.5), or else enzyme-degradable linkages which have the advantage of being stable in the serum and of releasing the cytotoxic agent in the intracellular medium of the tumour cell.

Mention may also be made of peptide sequences of the Ala Leu type, which can be cleaved by lysosomal hydrolases (Masquelier et al., J. Med. Chem., 1980, 23:1166-1170) or else linkages of the hydrazone type, such as those used in the gemtuzumab ozogamicin immunoconjugate used in the treatment of certain types of leukaemia and sold under the name Mylotarg (Hamann et al., Bioconjugate Chem., 2002, 13:47).

As described above, a preferred form of the invention uses a linkage which allows release of the deoxyvinblastine in the tumour cells.

A first means for achieving this consists in using a linkage connecting the two anchorages which consists of a peptide chain. In fact, such a peptide linkage will be degraded/hydrolysed in the target cells by the enzymes of the endosomes and of the lysosomes.

According to another embodiment of the invention, the linkage connecting the two anchorages consists of a linear or branched carbon-based chain. In the latter case, it is envisaged that one or more aromatic, ethylenic or acetylenic groups and also one or more ketone, amide, ester, hydrazide, hydrazone, amine, ether, sulphide or disulphide groups are included in the carbon chain in a distinct or combined manner. For example, in the case of an attachment via a disulphide bridge, it is the reducing medium which will allow cleavage of the linkage and release of the deoxyvinblastine.

In all cases, only the linkage is destroyed in order to release the active principle, said active principle and the antibody themselves remaining intact.

According to yet another embodiment, there is no linkage, but the Vinca alkaloid is coupled directly with a nitrogen-containing residue or with a hydroxyl residue or with a thiol residue of the antibody.

The advantage of such a direct coupling lies in the absence of anchorage linkage and, consequently, in the absence of an immune reaction by the patient against this linkage. The appearance of anti-linkage antibodies secreted by the body in response to the intrusion of said linkage is thus, for example, avoided.

More particularly, the compound according to the invention is characterized in that the acid function in the 4-position of the Vinca alkaloid is coupled, via a hydrazide function, with an aldehyde residue of the antibody, generated beforehand.

The invention also relates to a pharmaceutical composition comprising, as active principle, a compound consisting of a Vinca alkaloid coupled to an antibody, or one of its functional fragments, according to the invention, to which a pharmaceutically acceptable excipient and/or vehicle is preferably added.

The present invention also comprises the use of the compound according to the invention for preparing a medicinal product.

More particularly, according to another embodiment, the invention relates to the use of a compound as described above and/or of a composition comprising such a compound, for preparing a medicinal product intended for the prevention or treatment of cancers, in particular cancers induced by overexpression and/or activation of the IGF-IR and/or EGFR receptor which is abnormal, and/or associated with hyperactivation of the signal transduction pathway mediated by the interaction of IGF1 or IGF2 with IGF-IR and/or of EGF with EGFR.

Among the cancers which may be prevented and/or treated, prostate cancer, osteosarcomas, lung cancer, breast cancer, endometrial cancer or colon cancer, or any other cancer overexpressing IGF-IR, is preferred.

Certain embodiments of the invention include the detection, diagnosis, monitor, treatment, killing etc of colon cancer and ovarian cancer cells that are characterized as expressing aberrant levels of a common receptor—IGF-1R, whose aberrant expression, as noted, supra, has been linked to the underlying disease. In yet another embodiment, the invention includes the detection, diagnosis, treatment, monitor, killing etc. of colon and ovarian cancer cells that may express greater amounts of IGF-1R (increased expression or overexpression of IGF-1R) relative to normal. For the purposes of this invention, it is understood that increased expression of IGF-1R is not limited to increased receptor expression but may also result from increased ligand expression or by other genes which are part of the IGF-1R signaling pathway.

As used herein, the term “colon cancer” “ovarian cancer” “pancreatic cancer” etc., are intended to include diseases and other disorders in which the presence of high levels of IGF-IR in a subject suffering from the disorder has been shown to be or is suspected of being either responsible for the pathophysiology of the disorder or a factor that contributes to a worsening of the disorder. Accordingly, a disorder in which high levels of IGF-IR activity is detrimental is a disorder in which inhibition of IGF-IR activity is expected to alleviate the symptoms and/or progression of the disorder. Such disorders may be evidenced, for example, by an increase in the levels of IGF-IR on the cell surface or in increased tyrosine autophosphorylation of IGF-IR in the affected cells or tissues of a subject suffering from the disorder. The increase in IGF-IR levels may be detected, for example, using an anti-IGF-IR antibody as described above.

The term “Ewing's sarcoma (ES)” refers to a rare malignancy that most often presents as an undifferentiated primary bone tumor; less commonly, it arises in soft tissue (extraosseous Ewing's sarcoma, EES). Accordingly, the present invention includes methods for treating or preventing all types and stages of Ewing's sarcoma in a subject comprising administering to the subject a therapeutically effective amount of an IGF-1R antibody disclosed herein, optionally in association with a further chemotherapeutic agent.

The term “neuroblastoma” includes all types and stages of neuroblastoma. Neuroblastoma is a cancer of specialised nerve cells called neural crest cells. Neuroblastoma can occur anywhere in the body but often occurs in the adrenal glands. Accordingly, the present invention includes methods for treating or preventing all types and stages of neuroblastoma in a subject comprising administering to the subject a therapeutically effective amount of an IGF-1R antibody of the invention, optionally in association with a further chemotherapeutic agent. One type of neuroblastoma expresses the TRK-A neurotrophin receptor, is hyperdiploid, and tends to spontaneously regress. Another type of neuroblastoma expresses the TRK-B neurotrophin receptor; has gained an additional chromosome, 17q; has loss of heterozygosity of 14q; and is genomically unstable. In a third type of neuroblastoma, chromosome 1 p is lost and the N-MYC gene becomes amplified (Maris et al., J Clin Oncol 17 (7): 2264-79 (1999); Lastowska et al., J. Clin. Oncol. 19 (12): 3080-90 (2001).

The term “rhabdomyosarcoma” includes all types and stages of rhabdomycsarcoma. Accordingly, the present invention includes methods for treating or preventing all types and stages of rhabdomyosarcoma, in a subject, comprising administering, to the subject, a therapeutically effective amount of an IGF-1R described herein, optionally in association with a further chemotherapeutic agent. For example, subtypes of rhabdomyosarcoma include: embryonal rhabdomyosarcomas, alveolar rhabdomyosarcomas, undifferentiated rhabdomyosarcoma, botryoid rhabdomyosarcoma and pleomorphic rhabdomyosarcoma. In general, embryonal rhabdomyosarcoma (ERMS) tends to occur in the head and neck area, bladder, vagina, and in or around the prostate and testes. These usually affect infants and young children. In general, alveolar rhabdomyosarcoma (ARMS), occurs more often in large muscles of the trunk, arms, and legs and typically affects older children or teenagers. This type is called alveolar because the malignant cells form little hollow spaces, or alveoli. In general, botryoid rhabdomyosarcoma, a subset of embryonal rhabdomyosarcoma arises under the mucosal surfaces of body orifices, and is commonly observed in areas such as the vagina, bladder, and nares. Typically, it is distinguished by the formation of polypoid grapelike tumor masses, and it histologically demonstrates malignant cells in an abundant myxoid stroma. In general, pleomorphic rhabdomyosarcoma often occurs in patients aged 30-50 years. Its cells are irregularly arranged and vary in size, thus its pleomorphic distinction. Cross striations are rare.

The term “osteosarcoma” includes all types and stages of osteosarcoma. Accordingly, the present invention includes methods for treating or preventing all types and stages of osteosarcoma, in a subject, comprising administering, to the subject, a therapeutically effective amount of an IGF-1R antibody described herein, optionally in association with a further chemotherapeutic agent. For example, three types of osteosarcoma include high-grade osteosarcomas such as osteoblastic osteosarcoma, chondroblastic osteosarcoma, osteosarcoma fibroblastic, mixed osteosarcoma, small cell osteosarcoma, telangiectatic osteosarcoma and high grade surface osteosarcoma; intermediate-grade osteosarcomas such as periosteal osteosarcoma; and low-grade osteosarcomas such as parosteal osteosarcoma and intramedullary low grade osteosarcoma.

Towards these specific ends, the invention provided specific embodiments, in addition to those enumerated above that are drawn to the use of the antibodies described herein to treat etc. one of colon cancer, ovarian cancer, pancreatic cancer and pediatric cancers exemplified by rhabdomyosarcoma, Ewing's sarcoma, neuroblastoma and osteosarcoma. In other specific embodiments, the invention provides a combination therapy as detailed in the examples set forth below wherein the antibodies described herein are used to treat IGF-1R mediated cell proliferative disorders in combination with other anti-cancer agents.

In accordance with the above, the invention, in a broad aspect provides, inter alia, diagnostic assays and methods, both quantitative and qualitative for detecting, diagnosing, monitoring, staging, and prognosticating colon cancer by comparing levels of IGF-1R with those of IGF-1R in a normal human control. What is meant by “levels of IGF-1R” as used herein, means levels of the native protein or a functionally equivalent fragment thereof that is specifically recognized and bound by the antibody of the invention. In the alternative, what is meant by “levels of IGF-1R” as used herein, means levels of the native mRNA encoded by any of the genes comprising any of the polynucleotide sequences encoding native or mutant IGF-1R that is specifically recognized by the antibodies or antigen-binding fragments of the invention. Such levels are preferably measured in at least one of, cells, tissues and/or bodily fluids, including determination of normal and abnormal levels. Thus, for instance, a diagnostic assay in accordance with the invention for diagnosing over-expression of IGF-1R compared to normal control bodily fluids, cells, or tissue samples may be used to diagnose the presence of cancers, including colon cancer. Any of the known IGF-1R's may be measured in the methods of the invention.

In its broadest aspect, the present invention is directed to a monoclonal antibody, or binding fragment thereof, which specifically binds to cell surface receptors sharing a common epitope present on the surface of human colon cancer cells and ovarian cancer cells. A non-limiting example of the monoclonal antibody is IGF-IR specific antibody designated 7C10 or MK-0646. Preferably, the shared cell surface receptor is one which is expressed at levels higher than those found in cells from non-cancerous tissue and is preferably IGF-1R.

A broad method in accordance with the invention encompasses a method for treating or preventing a medical condition, in a subject, selected from the group consisting of neuroblastoma, rhabdomyosarcoma, Ewings sarcoma, osteosarcoma, pancreatic cancer, ovarian cancer, and colon cancer comprising administering a therapeutically effective amount of one or more of the IGF-1R specific antibodies of the invention or pharmaceutical compositions thereof to the subject.

In certain aspects, the invention is directed to antibodies that are capable of binding to the same antigenic determinant as does the monoclonal antibody MK-0646; and to binding fragments of said MK-0646. In accordance with still another aspect of the present invention there are provided diagnostic assays for detecting micrometastases/metastasis of colon cancer in a host. While applicant does not wish to limit the reasoning of the present invention to any specific scientific theory, it is believed that the presence of altered levels of expression of IGF-1R relative to normal in cells of the host is indicative of colon cancer metastases. This is true because, IGF-1R expression is higher in cancerous tissue than normal tissue. Thus, if colon cancer is present, colon cancer cells will express greater or higher levels (aberrant or altered expression) of IGF-1R than is normally found in non-diseased individuals, i.e., expression is higher than found in non-colon tissues in healthy individuals. It is the detection of this enhanced transcription or enhanced protein expression in cells, relative to normal, which is indicative of metastases of colon cancer. The same holds true for ovarian cancer.

Thus, in certain embodiments, cancers mediated by IGF-1R, IGF-1 and/or IGF-2 on certain cell types relative to normal, such as colon cancer, pancreatic, ovarian or pediatric cancers in subjects may be diagnosed or monitored by determining the presence of an IGF-1R in, for example, colon cells or cells derived from the ovary. Elevated levels of the colon specific IGF-1R indicates active transcription and expression of the corresponding colon specific IGF-1R. The presence of active transcription, which is greater than that normally found, of IGF-1R on cells derived from the colon, for example, by the presence of an altered level of mRNA, cDNA or expression products is an important indication of the presence of a colon cancer which has metastasized. Accordingly, this phenomenon may have important clinical implications since the method of treating a localized, as opposed to a metastasized, tumour is entirely different. As such, these embodiments aim to satisfy a long felt need for diagnostic tests that can detect colon or ovarian cancer at its early stages, when appropriate treatment may substantially increase the likelihood of positive outcome for the patient. Assays used to detect levels of the colon specific IGF-1R encoding gene or receptor polypeptide in a sample derived from a host are well-known to those skilled in the art and include radioimmunoassays, competitive-binding assays, Western blot analysis, ELISA assays and “sandwich” assays.

In its broadest aspect, the proposed method of diagnosis includes obtaining a biological sample from a subject, contacting the sample with an IGF-1R specific antibody or antigen-binding fragment thereof, that binds specifically to a cells expressing IGF-1R, and determining specific binding between the antibody or antigen-binding fragment thereof and IGF-1R in the sample, wherein the presence of specific binding is diagnostic for cancer in the subject. As used herein, a biological sample, relative to colon cancer, includes, but is not limited to: tissue, body fluid (e.g. blood), bodily exudate, mucus such as colonic mucosa, and stool specimen. The tissue may be obtained from a subject or may be grown in culture (e.g. from a cell line).

As used herein, a colorectal tissue sample is tissue obtained (e.g., from a colorectal tissue biopsy) using methods well-known to those of ordinary skill in the related medical arts. The phrase “suspected of being cancerous” as used herein means a colon cancer tissue sample believed by one of ordinary skill in the medical arts to contain cancerous cells. Methods for obtaining the sample from the biopsy include gross apportioning of a mass, microdissection, laser-based microdissection, or other art-known cell-separation methods.

By “control” it is meant a human patient without cancer and/or non-cancerous samples from the patient, also referred to herein as a normal human control; in the methods for diagnosing or monitoring for metastasis, control may also include samples from a human patient that is determined by reliable methods to have colon cancer which has not metastasized. In the embodiments related to diagnosis, monitoring or treating cancer, such as colon cancer or ovarian cancer, the invention provides an improved method of diagnosing one of colon or ovarian cancer, which relies on the ability of the IGF-1R specific antibodies disclosed herein to specifically bind to IGF-1R expressing cells attendant the particular cancer.

Methods for identifying subjects suspected of having colon cancer may include fecal occult blood examination, digital examination, CEA testing, endoscopic or radiographic techniques, biopsy, subject's family medical history, subject's medical history, or imaging technologies, such as magnetic resonance imaging (MRI). Such methods for identifying subjects suspected of having colon cancer are well-known to those of skill in the medical arts.

Generally, the level of a particular metastatic marker expression product (IGF-1R) in a body sample can be quantitated. Quantitation can be accomplished, for example, by comparing the level of expression product detected in the body sample with the amounts of product present in a standard curve. A comparison can be made visually or using a technique such as densitometry, with or without computerized assistance. For use as controls, body samples can be isolated from other humans, other non-cancerous organs of the patient being tested, or non-metastatic breast or colon cancer from the patient being tested.

The antagonist monoclonal antibodies of the invention may also be employed to treat colon cancer, since they interact with the function of colon specific IGF-1R polypeptides in a manner sufficient to inhibit natural function which is necessary for the viability of colon cancer cells. In this respect, the IGF-1R antagonists or antagonistic antibodies, e.g., IGF-1R specific monoclonal antibodies described herein may be employed in a composition with a pharmaceutically acceptable carrier, e.g., as hereinafter described.

Antibodies specific to IGF-1R expressing colon cancer cells, for example, the h7C10 monoclonal antibody (mAb) may also be used to target colon cancer cells, for example, in a method of homing anti-cancer therapeutic agents which, when contacting colon cancer cells, destroy them. This is true since the antibodies are specific for colon cancer cell specific IGF-1R which are primarily expressed in the colon.

Antibodies of the type described and claimed herein may also be used to conduct in vivo imaging, for example, by labeling the antibodies of the invention to facilitate scanning of the pelvic area and the colon. One method for imaging comprises contacting any tumor cells of the colon to be imaged with an anti-colon specific antibody, humanized 7C10 labeled with a detectable marker. The method is performed under conditions such that the labeled antibody binds to any colon specific IGF-1R polypeptides. In a specific example, the antibodies interact with the colon, for example, colon cancer cells, and fluoresce upon such contact such that imaging and visibility of the colon is enhanced to allow a determination of the diseased or non-diseased state of the colon.

According to another aspect of the invention, methods for determining onset, progression, or regression, of cancer in a subject are provided. In general, the presence of differentially expressed colon cancer specific receptor polypeptides, e.g., IGF-1R whose expression is generally higher in colon cancer cells relative to a control or normal sample, are measured in mucus or fecal/stool samples. Measurement of the presence of IGF-IR expression in subject's samples over time by sequential determinations at temporal intervals permits monitoring of the disease and/or the effects of a course of treatment. In general, an increase in expression of IGF-1R in cells derived from the colon is predictive of the subject presenting with colon cancer. Expression of IGF-1R may include determining DNA or mRNA levels or alternatively protein levels. As noted by Peters et al, supra, colon cancer cells express IGF-1R at levels that are higher than those present in non-cancerous cells. According to the authors, altered (higher than normal) levels of expression of IGF-1R has been observed “during the transition from normal to adenomatous and to carcinomatous tissue.” Peters et al., supra at 142. This observation corroborates the earlier findings by Ardeshir et al., “Expression of insulin-like growth factor-1 receptor in human colorectal cancer,” Hum. Pathol., 10:1128-1133 (1999). It thus stands to reason that increase in expression levels of IGF-1R in normal cells derived from the colon is a predictive marker for colorectal cancer (CRC).

The invention also provides, within other aspects, methods for monitoring the progression of a cancer in a patient. Such methods comprise the steps of: (a) contacting a biological sample obtained from a patient at a first point in time with a binding agent that binds to a polypeptide expressed by a colon cancer cell as recited above; (b) detecting in the sample an amount of polypeptide that binds to the binding agent; (c) repeating, steps (a) and (b) using a biological sample obtained from the patient at a subsequent point in time; and (d) comparing the amount of polypeptide detected in step (c) with the amount detected step (b) and thereafter monitoring the progression of the cancer in the patient. In all embodiments, the polypeptide expressed by colon cancer cells is IGF-1R and the binding agent is an anti-IGF-1R specific antibody, e.g., h7C10 or 7C10 or an antigen-binding fragment thereof.

Within further aspects, the present invention provides a method of predicting propensity for metastatic spread of a colon tumor. Towards this end, the method proposes determining the level of IGF-1R expression at a first time point in a colon tumor sample followed by measuring the same IGF-1R levels at subsequent time points. Following these iterative steps, a colon tumor sample which shows an upward progression or an increased expression of IGF-1R relative to normal or control sample over time is characterized as having a high propensity to metastasize.

Yet another object of the present invention is to provide a method of monitoring the change in stage of colon cancer in a patient which comprises identifying a patient having colon cancer, periodically measuring levels of IGF-1R in a sample of cells, tissue, or bodily fluid obtained from the patient, and comparing the measured IGF-1R levels with levels of IGF-1R in preferably the same cells, tissues, or bodily fluid type of a control wherein an increase in measured IGF-1R levels versus the control IGF-1R levels is associated with a cancer which is progressing and a decrease in the measured IGF-IR levels versus the control IGF-1R levels is associated with a cancer which is regressing or in remission.

According to another aspect of the invention, methods for selecting a course of treatment of a subject having or suspected of having colon cancer are provided. The methods include obtaining from the subject a biological sample, contacting the sample with antibodies or antigen-binding fragments thereof that bind specifically to IGF-1R, determining specific binding between the IGF-1R in the sample and the antibodies or antigen-binding fragments thereof, and selecting a course of treatment appropriate to the cancer of the subject. In the diagnostic and monitoring embodiments, the antibody used may be 7C10 or h7C10 or any other antibody that binds the same epitope on IGF-1R as do these two antibodies. Likewise, in preferred embodiments, the treatment comprises administering antibodies that specifically bind to the IGF-IR, exemplified by humanized 7C10 or any other human or humanized antibody that binds the same epitope on IGF-1R as does one of 7C10 or h7C10. In some embodiments, the antibodies are labelled with one or more cytotoxic agents.

The present invention is also directed to methods of inhibiting the growth of, or killing, one of colon cancer or ovarian cancer tumor cells in a patient by administering the monoclonal antibody, or a binding fragment as described herein, under conditions sufficient for the binding of the monoclonal antibody, or the binding fragment, to the ovarian cancer cells to inhibit the growth of, or to kill, the cells. In another aspect, a method for inhibiting or killing ovarian cancer cells proposes administering the monoclonal antibody, or binding fragment as described above, wherein the antibody or fragment thereof is conjugated with a cytotoxic moiety, under conditions sufficient for the binding of the monoclonal antibody, or binding fragment, to the cancer cells to inhibit the growth of, or to kill, the cells. The cytotoxic moiety may be, by way of non-limiting example, a chemotherapeutic agent, a photo-activated toxin, or a radioactive agent.

In yet another aspect, the invention is also directed to anti-idiotypic antibodies which mirror the binding site of the monoclonal antibody of the invention, e.g., h7C10/MK-0646 and are specific to the colon and/or ovarian cancer conformational epitope recognized by the antibody of the invention. The invention is further directed to the use of the aforementioned anti-idiotypic antibodies for the treatment of colon cancer or ovarian cancer.

In yet another aspect of the invention, a method is provided for localizing ovarian cancer cells in a patient by administering the monoclonal antibody, or binding fragment, described above, allowing the monoclonal antibody, or binding fragment thereof, to bind to ovarian cancer cells within said patient, and determining the location of said monoclonal antibody, or binding fragment thereof, within said patient. In another related aspect, the monoclonal antibody, or binding fragment, is detectably labeled, for example, with a radionuclide.

In another aspect, the invention provides methods for staging an IGF-1R mediated cancer by comparing the level of IGF-1R from a test sample to a control sample, wherein an increase over time in IGF-1R expression relative to normal or control sample aids the physician in staging the disease status. Monitoring the effectiveness of an anti-cancer therapy is also within the confines of the invention. Thus, a decrease in IGF-1R after treatment with the herein disclosed antibodies is indicative of a good prognosis and suggest that the treatment protocol is effective in reducing IGF-1R expression. On the other hand, an increase in IGF-1R expression or no change in expression following treatment with the herein disclosed antibodies is suggestive of a poor prognosis in that the treatment protocol in not effective or poorly effective in treating the underling disease.

The present invention is also directed to therapeutic methods for the treatment of ovarian cancer and related dysproliferative diseases in humans, using the antibodies of the present invention. The therapeutic and diagnostic uses described herein embrace primary tumors as well as metastases. For example, a method for inhibiting or killing ovarian cancer cells in a patient may be carried out by administering to the patient, in a single dose or in successive doses, the monoclonal antibody, or antibody binding fragment as described herein, under conditions sufficient for the binding of the monoclonal antibody, or binding fragment, to tumor cells in the patient. Binding of antibodies to the tumor cells induces the growth inhibition and/or killing of the tumor cells by the antibody.

The aforementioned therapy may be accompanied by other treatments directed at the tumor cells, such as chemotherapy, radiation, etc., as well as by adjunctive therapies to enhance the immune system's attack on the opsonized tumor cells following the procedure described above. For example, a growth factor such as erythropoietin and/or GM-CSF can be co-administered to the patient for stimulating the white blood cells and supporting the immunocompetence status of the patient.

Other characteristics and advantages of the invention appear in the continuation of the description with the examples and the figures whose legends are represented below.

### LEGENDS TO THE FIGURES

FIG. 1: Schematic representation of IGF-IR.

FIG. 2: Scheme of the transduction of the signals mediated by IGF-IR during the attachment of IGFs.

FIGS. 3A, 3B and 3C: Recognition of native IGF-IR expressed on the surface of MCF-7 cells by the monoclonal antibody 7C10.

For this experiment, the MCF-7 cells are incubated with the 7C10 antibody or with a negative control antibody, then recovered with the aid of a fluorescent anti-species secondary antibody. The labeling is read on a FACS. The first histogram (FIG. 3A) corresponds to the MCF-7 cells alone. In the second histogram (FIG. 3B), the unshaded curve corresponds to the nonspecific labeling by a control isotype murine antibody. In the third histogram (FIG. 3C), the unshaded curve shows the recognition of IGF-IR by MAB 7C10.

FIGS. 4A, 4B and 4C: Labeling of Sf9 insect cells respectively expressing IGF-IR or IR.

FIG. 4A shows the labeling of nontransfected cells alone (1) or cells labeled with control commercial monoclonal antibodies respectively recognizing IGF-IR (2) or IR (3). In FIG. 4B, Sf9 cells uniquely expressing IGF-IR are labeled with αIR3 (2) or anti-IR(3), the peak (1) representing the single cells. In FIG. 4C, Sf9 cells uniquely expressing IR are labeled with an anti-IR (3) or αIR3 (2), the peak (1) representing the single cells.

FIG. 5: Inhibitor effect of 7C10 antibody on the proliferation of MCF-7 cells induced by IGF-I.

The MCF-7 cells are incubated in the presence of increasing concentrations of IGF1 in the presence or in the absence of the MAB to be tested. The cell proliferation is evaluated by following the incorporation of ³H thymidine. The commercial antibody αIR3 is used as a positive control of the experiment. The 7G3 is a murine anti-IGF-IR IgG1 without activity on proliferation and used as a control isotype.

FIGS. 6A, 6B and 6C:

FIG. 6A: in vivo effect of the monoclonal antibody 7C10 on the growth of MCF-7 tumors established in nude mice;

FIGS. 6B and 6C: figures respectively from publications of Arteaga et al. (J. Clin. Invest., 84, 1418-1423, 1989) and from Li et al. (Cancer Immunol. Immunother., 49, 243-252), and showing for FIG. 6B the effect of murine αIR3 (likewise written aIR3) and for FIG. 6C the effect of a recombinant scFv-Fc derived from the 1H7 antibody on tumor growth.

FIG. 7: Comparative study of the effect of the MAb 7C10 and of tamoxifen on the growth in vivo of the tumor MCF-7.

FIGS. 8A, 8B and 8C: Study of the antitumor activity of the murine antibody 7C10 in different xenograft models of tumor cells in vivo.

FIG. 8A shows the results obtained on an osteosarcoma model SK-ES-1, FIG. 8B concerns an androgen-independent tumor of the prostate DU-145 and FIG. 8C a model of non-small cell tumor of the lung A549. In these three models, the treatment was carried out twice per week i.p. at a rate of 250 μg/dose/mouse. The curves 7G3, EC2 and 9G4 correspond respectively to three murine IgG1 used as an experiment control isotype in each of the models.

FIG. 9: Study of the antitumor effect of the MAb 7C10 compared to navelbine (vinorelbine) as well as the synergy of the two compounds on the growth in vivo of the line A549.

FIG. 10: Comparative activity of MAb αIR3, 7C10 and 1H7 on the IGF-2 proliferation induced by MCF-7 cells.

FIG. 11: Comparison of the murine 7C10 and chimeric C7C10 MAb for the inhibition of the IGF1 proliferation of MCF-7 cells in vitro. The antibody 9G4 is a murine IgG1 used as an experiment control isotype.

FIG. 12: Comparative effect of the 7C10 and h7C10 MAb (humanized 1, written here 7H2HM) on the in vitro model of IGF1-induced proliferation of MCF-7 cells.

FIG. 13: Effect of the 7C10 and h7C10 MAb (humanized 1, written here 7H2HM) on the transduction of the signal induced by IGF1. The first line of spots corresponds to the revelation, by an antiphospho-tyrosine antibody, of the phosphorylation of the immunoprecipitated β chain from the cells incubated in the presence of IGF1 alone or of IGF1 mixed with various antibodies to be tested. The 9G4 and the hIgG1 are respectively the control isotypes of the forms 7C10 and h7C10 (likewise written 7H2HM). The second line of spots corresponds to the revelation of the β chain and shows that the quantity deposited in all of the wells is perfectly equivalent.

FIG. 14: Sequence of the cDNA (SEQ ID No. 48), of its complementary strand (SEQ ID No. 50) and its translation into amino acids (SEQ ID No. 49), of the PCR fragment amplified from the mouse hybridoma 7C10 with the primers MKV-1 and MKC and which codes for the 3′ end of the leader peptide and 7C10 VL.

FIG. 15: Sequence of the cDNA (SEQ ID No. 51), of its complementary strand (SEQ ID No. 53) and its translation into amino acids (SEQ ID No. 52), of the PCR fragment amplified from the mouse hybridoma 7C10 with the primers MHV-12 and MHC-1, or MHV-8 and MHC-1 and which codes for the 3′ end of the leader peptide and 7C10 VH.

FIG. 16: Recognition of the IGF-I receptor by the chimeric antibody 7C10, likewise called C7C10 (supernatant of cos7-transfected cell culture).

FIG. 17: Comparison of the amino acid sequence of mouse 7C10 VL (SEQ ID No. 54) with cells of other mouse antibodies having the greatest sequence homology.

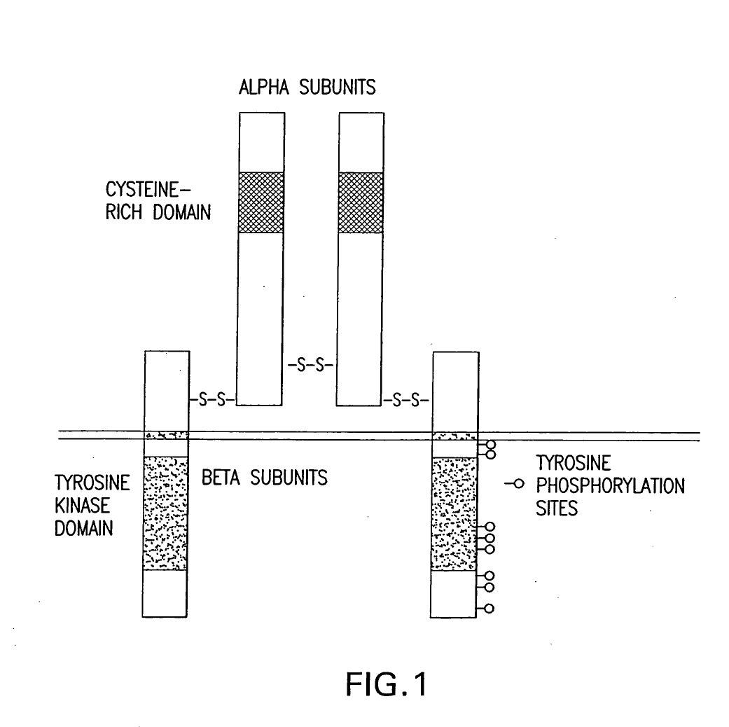

The numbering of the amino acids is that of Kabat et al. (1991). The residues in the framework regions (outside CDRs) which differ between 7C10 VL and Kabat mouse subgroup II (SEQ ID No. 57) are underlined. A dot indicates that the residue is identical at this position in comparison with the sequence of 7C10 VL. DRB1-4.3 (SEQ ID No. 55) represents the sequence of the light chain of an anti-human mouse antibody MHC CLASS II B-Chain (access number in the Kabat databank is N011794). C94-5B11′CL (SEQ ID No. 56) represents the sequence of the light chain of a mouse antibody (access number in the Kabat databank is P019314).

FIG. 18: Comparison of amino acid sequences of mouse 7C10 VL (SEQ ID No. 54) with cells of human light chains belonging to Kabat human subgroup II (SEQ ID No. 60) and having the greatest sequence homology.

The amino acid sequences are aligned and compared with that of mouse 7C10 VL. A dot indicates that the residue is identical at this position in comparison with the sequence of 7C10 VL. GM607 (SEQ ID No. 58) represents the sequence of the kappa light chain secreted by the human lymphoblastoid line GM607 (Klobeck et al., Nucleic Acids Res., 12:6995-7006, 1984a and Klobeck et al., Nature, 309:73-76, 1984b, the access number in the Kabat databank is N011606). DPK15/A19 (SEQ ID No. 59) represents the sequence of the human V germinal line kappa II.

FIG. 19: Comparison of amino acid sequences of variable regions of the light chains (VL) of mouse 7C10 (SEQ ID No. 54), of human antibody GM 607 (SEQ ID No. 58) and of two versions of humanized 7C10 1 and 2 (SEQ ID Nos. 61 and 65).

The amino acid sequences are aligned and compared with that of mouse 7C10 VL. A dot indicates that the residue is identical at this position in comparison with the sequence of 7C10 VL. GM607 represents the sequence of the kappa light chain secreted by the human lymphoblastoid line GM607 (Klobeck et al., 1984a and 1984b, access number in the Kabat database: N011606).

FIG. 20: cDNA sequence (SEQ ID No. 62), its complementary strand (SEQ ID No. 64) and its translation into amino acids (SEQ ID No. 63), of the gene constructed by de novo assembly coding for the leader peptide and the humanized version 1 of 7C10 VL.

FIG. 21: cDNA sequence (SEQ ID No. 66), its complementary strand (SEQ ID No. 68) and its translation into amino acids (SEQ ID No. 67), of the gene constructed by de novo assembly coding for the leader peptide and the humanized version 2 of 7C10 VL.

FIG. 22: Comparison of the amino acid sequences of mouse 7C10 VH (SEQ ID No. 69) with those of human mouse heavy chains belonging to Kabat mouse subgroup I(A) and having the greatest sequence homology.

The numbering of the amino acids is that of Kabat et al. (1991). The residues in the framework regions (outside CDRs) which differ between 7C10 VH and Kabat mouse subgroup I(A) (SEQ ID No. 71) are underlined. A dot indicates that the residue is identical at this position in comparison with the sequence of mouse 7C10 VH. AN03′CL (SEQ ID No. 70) represents the sequence of the heavy chain of a mouse antibody (access number in the Kabat databank: P001289).

FIG. 23: Comparison of amino acid sequences of mouse 7C10 VH (SEQ ID No. 69) with those of human heavy chains belonging to the Kabat human subgroup II (SEQ ID No. 72) and having the greatest sequence homology.

The underlined residues are part of the canonical structures defined by Chothia et al. (1989). A dot indicates that the residue is identical at this position in comparison with the mouse 7C10 VH sequence. Human VH FUR1′CL (SEQ ID No. 73) represents the sequence of the heavy chain of a human anti-lamin B antibody IgM/K of autoimmune origin (Mariette et al., Arthritis and Rheumatism, 36:1315-1324, 1993; access number in Kabat: N020619). Human germline (SEQ ID No. 74) represents the sequence of the human germinal line 4.22 VH IV (Sanz et al., EMBO. J. 8:3741-3748, 1989).

FIG. 24: Comparison of the amino acid sequences of the variable regions of the heavy chains (VH) of mouse 7C10 (SEQ ID No. 69) and of the three versions humanized by CDR-grafting humanized VH 1, 2 and 3 (respectively SEQ ID Nos. 75, 79 and 83).

The numbering of the residues corresponds to that of Kabat. The sequences are aligned and compared with that of mouse 7C10 VH. A dot indicates that the residue is identical at this position in comparison with the sequence of mouse 7C10 VH.

FIG. 25: cDNA sequence (SEQ ID No. 76), its complementary strand (SEQ ID No. 78) and its translation into amino acids (SEQ ID No. 77), of the gene constructed by de novo assembly coding for the leader peptide and the humanized version 1 of 7C10 VH.

FIG. 26: cDNA sequence (SEQ ID No. 80), its complementary strand (SEQ ID No. 82) and its translation into amino acids (SEQ ID No. 81), of the gene constructed by de novo assembly coding for the leader peptide and the humanized version 2 of 7C10 VH.

FIG. 27: cDNA sequence (SEQ ID No. 84), its complementary strand (SEQ ID No. 86) and its translation into amino acids (SEQ ID No. 85), of the gene constructed by de novo assembly coding for the leader peptide and the humanized version 3 of 7C10 VH.

FIG. 28: Comparison of the recognition activity of the IGF-I receptor by the chimeric antibody 7C10 (called “C7C10”) and its humanized version 1 (7C10 hum 1) in ELISA.

FIG. 29: Influence on the recognition activity of the IGF-I receptor of the humanized versions 1 and 2 of the light chain of the 7C10 antibody in ELISA.

FIG. 30: Comparison of the recognition activity of the IGF-I receptor by the chimeric antibody 7C10 and three humanized versions of the heavy chain (7C10 hum 1, 2 and 3) in combination with humanized 7C10 VL 2 in ELISA.

FIG. 31: Antitumor activity of the 7C10 antibody in an orthotopic model A549.

FIGS. 32A, 32B, 32C and 32D: Study of the ADCC observed at the level of A549 and MCF-7 cells cultured during 4 hours in the presence of the antibody 7H2HM (respectively FIGS. 32C and 32D). The antibody h4D5 is used in parallel as an experiment positive control for the cells A549 and MCF-7 (respectively FIGS. 32A and 32B).

FIGS. 33A, 33B and 33C: Effects of the antibodies 7C10 and 7H2HM on the cell cycle of the MCF-7 cells.

FIG. 33A represents the proportion of MCF-7 cells in the G0/G1, S and G2/M phase in the absence of IGF1, expressed as a significant percentage of total MCF-7 cells observed.

FIG. 33B represents the proportion of MCF-7 cells in the G0/G1, S and G2/M phase in the presence of IGF1, expressed as a percentage of total MCF-7 cells observed.

FIG. 33C represents the proportion of MCF-7 cells in the S (▪) and G2/M (γ) phase, expressed as a percentage of total MCF-7 cells observed, in the presence of the compounds indicated in the figure compared with a control sample in the absence of IGF1 (“0”).

FIGS. 34A and 34B: Comparative effect of the antibodies 7C10 and 7H2HM on the growth of A549 cells in vitro (FIG. 34A) and on the growth of MCF-7 cells in vivo (FIG. 34B).

FIGS. 35A and 35B: Study of the synergy of the antibody 7H2HM combined with navelbine (NA) on the model A549 in vivo, compared with the control samples. FIG. 35A represents the development of the volume of the implanted tumor as a function of the treatment carried out starting from the commencement of the treatment and over approximately 50 days (FIG. 35A). FIG. 35B represents in a particular manner the results obtained for this development compared at approximately 48 days. In this figure, the results obtained with the antibody 7C10 have been introduced by way of comparison (the asterisks (*) correspond to the comparison control group/group (7C10+Na) or control group/group (7H2HM+Na) in a t-test).

FIG. 36: Study of the effect of the antibodies 7C10 and 7H2HM on apoptosis.

This figure represents the potentiation of the effect of doxorubicin by the antibodies 7C10 and 7H2HM (doxorubicin 2 μg/ml).

FIGS. 37A to 37D: Demonstration by labeling in FACS of the presence of EGFR and of IGF-IR on the surface of A549 cells.

FIG. 38: Effect of a coadministration of the MAB 7C10 and 225 on the in vivo growth of the tumor A549.

FIG. 39: Effect of a coadministration of the MAB 7C10 and 225 on the survival of mice orthotopically implanted with A549 cells.

FIGS. 40A and 40B: Demonstration of the inhibition of tyrosine phosphorylation of the beta chain of IGF-IR and of IRS-1 by the MAB 7C10 and 7H2HM.

FIG. 41: Demonstration of the induction of the internalization of IGF-IR by the MAB 7C10 and 7H2HM.

FIGS. 42A to 42C: Demonstration of the degradation of IGF-IR by the MAB 7C10 and 7H2HM.

FIGS. 43A and 43B: Immuno-blotting with an anti-IGF-IR β-subunit and anti-IR β-subunit on filters containing cellular lysates obtained after immunoprecipitation and SDS-PAGE for two independent experiments (A and B).

FIG. 44: Immunocapture of R+ cell lysates IGF-IR in Maxisorb plates coated with 17-69 antibody and binding by ¹²⁵-IGF-I in the absence or the presence of increasing concentrations of unlabeled ligand (IGF-I) or antibodies (7C10, h7C10, 1H7, 9G4).

FIG. 45: Immunocapture of R−/IR-A cell lysates Hybrid-R^{A} in Maxisorb plates coated with 83-7 antibody and binding by ¹²⁵I-IGF-I in the absence or the presence of increasing concentrations of unlabeled ligand (IGF-I) or antibodies (7C10, h7C10, 1H7, 9G4).

FIG. 46: Immunocapture of R−/IR-B cell lysates Hybrid-R^{B} in Maxisorb plates coated with 83-7 antibody and binding by ¹²⁵-IGF-I in the absence or the presence of increasing concentrations of unlabeled ligand (IGF-1) or antibodies (7C10, h7C10, 1H7, 9G4).

FIGS. 47A and 47B: Immuno-blotting analysis of antibody induced degradation of the IGF-IR in A549 (A) and MCF-7 (B) cells.

FIG. 48: Immuno-blotting analysis of antibody degradation pathway of IGF-IR in MCF-7 cells.

FIG. 49: Anti-tumoral activity of the murine antibody 7C10 co-administrated with an anti-VEGF antibody on mice orthopically implanted with A549 cells.

FIGS. 50 and 51: Comparison of the in vivo anti-tumoral activity of the 7C10 and h7C10 antibodies on the A549 (FIG. 50) and MCF-7 (FIG. 51) models.

FIGS. 52 and 53: Comparison of the anti-leukaemia activity of vinblastine and vincristine (FIG. 52) and of 4′ R and 4′ S deoxyvinblastines (FIG. 53).

FIG. 54: In vivo antitumour activity of 4′ R— and 4′ S-deoxyvinblastines conjugated with IGR-IR antibodies on human tumours of various origins.

FIG. 55: In vivo anti-tumoral effect of the MK-0646 Ab combined with Avastin® Ab on the orthotopic A549 model.

FIG. 56: In vivo anti-tumoral activity of monoclonal antibody MK-0646 alone or combined with Avastin, on A549 xenograft model.

FIG. 57: In vivo anti-tumoral effect of the MK-0646 Ab combined with Herceptin® Ab on the orthotopic A549 model.

FIG. 58: Effect of combination of MK-0646 and gemcitabine in xenograft BxPC-3 pancreatic model in athymic nude mice.

FIG. 59: Evaluation of weight loss in mice treated with Gemcitabine 138.5 mg/kg alone or combined with MK-0646 antibody 12.5 μg/dose.

FIG. 60: In vivo anti-tumoral activity of monoclonal antibody MK-0646 alone or combined with Irinotecan, on COLO 205 xenograft model.

FIG. 61: Weight follow-up of MK-0646 and/or Irinotecan treated, COLO 205 xenografted mice.

FIG. 62: Combination of the MK-0646 antibody and Doxorubicin in xenograft MCF-7 model.

FIG. 63: Evaluation of weight loss in mice treated with either 5 mg/kg of Doxorubicin alone or combined to the MK-0646 antibody

FIG. 64: Combination of the MK-0646 antibody and Docetaxel in xenograft MCF-7 model.

FIG. 65: Evaluation of weight loss in mice treated with MK-0646 alone or in combination with Docetaxel.

FIG. 66: Combination of the MK-0646 antibody and Paclitaxel in xenograft MCF-7 model.

FIG. 67: SKOPV3ip (ovarian) mouse xenograft model.

FIG. 68: Dose response of Herceptin in ovarian SKOV31p orthotopic model.

FIG. 69: Combination studies of MK-0646 and Herceptin in SKOV31p model.

FIG. 70: Combination studies of MK-0646 and Herceptin in SKOV31p model.

FIG. 71: Inhibition of colon tumor xenografts by MK-0646.

FIG. 72: Enhanced efficacy of MK-0646 in combination with Erbitux in HT29 colon tumor xenograft model.

FIG. 73-74: Reported data represent the average of the primary tumor weight recorded in a group of athymic mice (n=10) treated with either 100 or 400 μg of MK-0646. (n=10).

FIG. 75: Reported data represent the total volume of ascites (ml) and the total number of metastases counted in mice from the same group (n=10).

FIG. 76: Antitumor efficacy of MK-0646 in nude mice bearing the OVXF 899 ovarian carcinoma xenograft showing the group mean tumor volumes over time.

FIG. 77: Antitumor efficacy of MK-0646 in nude mice bearing the BxPC-3 pancreatic carcinoma xenograft showing mean tumor volumes.

FIG. 78: Effect of 7C10 (h7C10; 10 ug/mL) on a rhabdomyosacrcoma cell line (RMS) and xenografts as measured by MTT assay. The data show that IGF-IR antibody (h7C10; 10 ug/mL) decreases Rh30 cell number (78A) and decreases phosphorylation of downstream targets of IGF-1R (78B). (A) MTT proliferation assay of h7C10 treated Rh30 and RD cells. Rh30 and RD Cells were treated with either complete RPMI medium alone or h7C10 (10 ng/mL) in complete RPMI medium. Cell growth and survival was determined by MTT assay. (B) Western blot analysis of h7C10 treated Rh30 and RD cells. Rh30 and RD cells were treated with h7C10 (10 ng/mL) for 48 hrs or 96 hrs in complete RPMI and then lysed in lysis buffer for western blot analysis for phosphorylation and expression of p-AKT and p-p-MAPK p44/p42.

FIG. 79: Paten A show the effect of 7C10 as measured in a proliferation assay of 7C10 treated Rh1, Rh4 and RD4 cells. Briefly, cells were treated with either complete RPMI medium alone or h7C10 (10 ng/mL) in complete RPMI medium. Cell growth and survival was determined by MTS assay. (B) Details the Western blot analysis of h7C10 treated Rh1, Rh41, and RD4A cells. The cells were treated with h7C10 (10 ng/mL) for 48 hrs or 96 hrs in complete RPMI and then lysed in lysis buffer for western blot analysis for phosphorylation and expression of P-AKT and p-p42/44 MAPK.

FIG. 80: IGF-IR antibody (h7C10) alone and in combination with rapamycin decreases primary tumor growth in Rh30-Luc xenografts. (A) Mice bearing Rh30 xenografts were treated IP with h7C10 (12.5 mg/kg) q4d alone, rapamycin (5 mg/kg) q3d alone, the combination of h7C10+rapamycin, or vehicle for 57 days. All mice were imaged weekly by D-luciferin to monitor primary tumor growth. (B) Caliper Measurements of the average primary tumor size of the mice for each group.

FIG. 81: Details the anti-tumor effects of 7C10 alone or in combination with an mTOR pathway inhibitor as evidenced by chemiluminescent measurement.

### DETAILED DESCRIPTION OF THE INVENTION

Although advances have been made in detection and therapy of ovarian or colon is cancer, no universally successful method for prevention or treatment is currently available. Accordingly, there is a need in the art for improved methods for identifying ovarian cancer and for treating said cancer.

The present invention fulfills these needs and further provides other related advantages. It is understood that the general techniques detailed here after apply equally to colon and ovarian cancer as well as any other cancer whose cells express IGF-1R or a protein that is recognized by the antibodies described herein.

### Methods for Detecting Cancer

Assay techniques that can be used to determine levels of gene expression, such as IGF-1R, in a sample derived from a host are well-known to those of skill in the art. Such assay methods include radioimmunoassays, reverse transcriptase PCR(RT-PCR) assays, immunohistochemistry assays, in situ hybridization assays, competitive-binding assays, Western Blot analyses and ELISA assays. Among these, ELISAs are frequently preferred to diagnose a gene's expressed protein in biological fluids. An ELISA assay initially comprises preparing an antibody, if not readily available from a commercial source, specific to IGF-1R, preferably a monoclonal antibody, e.g., h7C10 or 7C10. In addition a reporter antibody generally is prepared which binds specifically to IGF-1R. The reporter antibody is attached to a detectable reagent such as radioactive, fluorescent or enzymatic reagent, for example horseradish peroxidase enzyme or alkaline phosphatase.

In general, a cancer may be detected in a patient based on the presence of one or more ovarian carcinoma proteins and/or polynucleotides encoding such proteins in a biological sample (such as blood, sera, urine and/or tumor biopsies) obtained from the patient. In other words, such proteins may be used as markers to indicate the presence or absence of a cancer such as ovarian cancer. A non-limiting example of such a protein is IGF-IR whose expression levels are generally higher than normal in cells derived from cancerous ovarian tissue. In addition, such proteins may be useful for the detection of other cancers, e.g. colon cancer or other IGF-1R mediated cell proliferative disorders.

There are a variety of assay formats known to those of ordinary skill in the art for using a binding agent to detect polypeptide markers in a sample. See, e.g., Harlow and Lane, Antibodies: A Laboratory Manual, Cold Spring Harbor Laboratory, 1988. In general, the presence or absence of a cancer in a patient may be determined by (a) contacting a biological sample obtained from a patient with a binding agent; (b) detecting in the sample a level of polypeptide that binds to the binding agent; and (c) comparing the level of polypeptide with a predetermined cut-off value.

In a preferred embodiment, the assay involves the use of binding agent immobilized on a solid support to bind to and remove the polypeptide from the remainder of the sample. The bound polypeptide may then be detected using a detection reagent that contains a reporter group and specifically binds to the binding agent/polypeptide complex. Such detection reagents may comprise, for example, a binding agent that specifically binds to the polypeptide or an antibody or other agent that specifically binds to the binding agent, such as an anti-immunoglobulin, protein G, protein A or a lectin.

Alternatively, a competitive assay may be utilized, in which a polypeptide is labeled with a reporter group and allowed to bind to the immobilized binding agent after incubation of the binding agent with the sample. The extent to which components of the sample inhibit the binding of the labeled polypeptide to the binding agent is indicative of the reactivity of the sample with the immobilized binding agent. Suitable polypeptides for use within such assays include full length ovarian carcinoma proteins and portions thereof to which the binding agent binds, as described above.

The solid support may be any material known to those of ordinary skill in the art to which the tumor protein may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681. The binding agent may be immobilized on the solid support using a variety of techniques known to those of skill in the art, which are amply described in the patent and scientific literature. In the context of the present invention, the term “immobilization” refers to both noncovalent association, such as adsorption, and covalent attachment (which may be a direct linkage between the agent and functional groups on the support or may be a linkage by way of a cross-linking agent). Immobilization by adsorption to a well in a microtiter plate or to a membrane is preferred. In such cases, adsorption may be achieved by contacting the binding agent, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of binding agent ranging from about 10 ng to about 10 .mμ.g, and preferably about 100 ng to about 1 .mμ.g, is sufficient to immobilize an adequate amount of binding agent.

Covalent attachment of binding agent to a solid support may generally be achieved by first reacting the support with a bifunctional reagent that will react with both the support and a functional group, such as a hydroxyl or amino group, on the binding agent. For example, the binding agent may be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at A12-A13).

In certain embodiments, the assay is a two-antibody sandwich assay. This assay may be performed by first contacting an antibody that has been immobilized on a solid support, commonly the well of a microtiter plate, with the sample, such that polypeptides within the sample are allowed to bind to the immobilized antibody. Unbound sample is then removed from the immobilized polypeptide-antibody complexes and a detection reagent (preferably a second antibody capable of binding to a different site on the polypeptide) containing a reporter group is added. The amount of detection reagent that remains bound to the solid support is then determined using a method appropriate for the specific reporter group.

More specifically, once the antibody is immobilized on the support as described above, the remaining protein binding sites on the support are typically blocked. Any suitable blocking agent known to those of ordinary skill in the art, such as bovine serum albumin or Tween 20™. (Sigma Chemical Co., St. Louis, Mo.). The immobilized antibody is then incubated with the sample, and polypeptide is allowed to bind to the antibody. The sample may be diluted with a suitable diluent, such as phosphate-buffered saline (PBS) prior to incubation. In general, an appropriate contact time (i.e., incubation time) is a period of time that is sufficient to detect the presence of polypeptide within a sample obtained from an individual with ovarian cancer. Preferably, the contact time is sufficient to achieve a level of binding that is at least about 95% of that achieved at equilibrium between bound and unbound polypeptide. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 20™. The second antibody, which contains a reporter group, may then be added to the solid support. Preferred reporter groups include those groups recited above.

The detection reagent is then incubated with the immobilized antibody-polypeptide complex for an amount of time sufficient to detect the bound polypeptide. An appropriate amount of time may generally be determined by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of an IGF-1R mediated cell proliferative disorder such as colon ovarian cancer, the signal detected from the reporter group that remains bound to the solid support is generally compared to a signal that corresponds to a predetermined cut-off value.

In one embodiment, the cut-off value for the detection of a cancer is the average mean signal obtained when the immobilized antibody is incubated with samples from patients without the cancer. In general, a sample generating a signal that is three standard deviations above the predetermined cut-off value is considered positive for the cancer. In an alternate preferred embodiment, the cut-off value is determined using a Receiver Operator Curve, according to the method of Sackett et al., Clinical Epidemiology: A Basic Science for Clinical Medicine, Little Brown and Co., 1985, p. 106-7. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for a cancer.

In a related embodiment, the assay is performed in a flow-through or strip test format, wherein the binding agent is immobilized on a membrane, such as nitrocellulose. In the flow-through test, polypeptides within the sample bind to the immobilized binding agent as the sample passes through the membrane. A second, labeled binding agent then binds to the binding agent-polypeptide complex as a solution containing the second binding agent flows through the membrane. The detection of bound second binding agent may then be performed as described above. In the strip test format, one end of the membrane to which binding agent is bound is immersed in a solution containing the sample. The sample migrates along the membrane through a region containing second binding agent and to the area of immobilized binding agent. Concentration of second binding agent at the area of immobilized antibody indicates the presence of a cancer. Typically, the concentration of second binding agent at that site generates a pattern, such as a line, that can be read visually. The absence of such a pattern indicates a negative result. In general, the amount of binding agent immobilized on the membrane is selected to generate a visually discernible pattern when the biological sample contains a level of polypeptide that would be sufficient to generate a positive signal in the two-antibody sandwich assay, in the format discussed above. Preferred binding agents for use in such assays are antibodies and antigen-binding fragments thereof. Preferably, the amount of antibody immobilized on the membrane ranges from about 25 ng to about 1 .mμ.g, and more preferably from about 50 ng to about 500 ng. Such tests can typically be performed with a very small amount of biological sample.

Of course, numerous other assay protocols exist that are suitable for use with the tumor proteins or binding agents of the present invention. The above descriptions are intended to be exemplary only. For example, it will be apparent to those of ordinary skill in the art that the above protocols may be readily modified to use ovarian carcinoma polypeptides to detect antibodies that bind to such polypeptides in a biological sample. The detection of such ovarian carcinoma protein specific antibodies may correlate with the presence of a cancer.

As noted above, a cancer may also, or alternatively, be detected based on the level of mRNA encoding an ovarian carcinoma protein (IGF-1R) in a biological sample. For example, at least two oligonucleotide primers may be employed in a polymerase chain reaction (PCR) based assay to amplify a portion of an ovarian carcinoma protein cDNA derived from a biological sample, wherein at least one of the oligonucleotide primers is specific for (i.e., hybridizes to) a polynucleotide encoding the ovarian carcinoma protein. The amplified cDNA is then separated and detected using techniques well known in the art, such as gel electrophoresis. Similarly, oligonucleotide probes that specifically hybridize to a polynucleotide encoding an ovarian carcinoma protein may be used in a hybridization assay to detect the presence of polynucleotide encoding the tumor protein in a biological sample.

To permit hybridization under assay conditions, oligonucleotide primers and probes should comprise an oligonucleotide sequence that has at least about 60%, preferably at least about 75% and more preferably at least about 90%, identity to a portion of a polynucleotide encoding an ovarian carcinoma protein that is at least 10 nucleotides, and preferably at least 20 nucleotides, in length. Preferably, oligonucleotide primers and/or probes hybridize to a polynucleotide encoding a polypeptide described herein under moderately stringent conditions, as known to one skilled in the art. The oligonucleotide primers comprise at least 10 contiguous nucleotides, more preferably at least 15 contiguous nucleotides, of a DNA molecule encoding IGF-1R recognized by the antibodies describe herein. Techniques for both PCR based assays and hybridization assays are well known in the art (see, for example, Mullis et al., Cold Spring Harbor Symp. Quant. Biol., 51:263, 1987; Erlich ed., PCR Technology, Stockton Press, New York, 1989).

One preferred assay employs RT-PCR, in which PCR is applied in conjunction with reverse transcription. Typically, RNA is extracted from a biological sample such as a biopsy tissue and is reverse transcribed to produce cDNA molecules. PCR amplification using at least one specific primer generates a cDNA molecule, which may be separated and visualized using, for example, gel electrophoresis. Amplification may be performed on biological samples taken from a test patient and from an individual who is not afflicted with a cancer. The amplification reaction may be performed on several dilutions of cDNA spanning two orders of magnitude. A two-fold or greater increase in expression in several dilutions of the test patient sample as compared to the same dilutions of the non-cancerous sample is typically considered positive.

In another embodiment, ovarian carcinoma proteins and polynucleotides encoding such proteins may be used as markers for monitoring the progression of cancer. In this embodiment, assays as described above for the diagnosis of a cancer may be performed over time, and the change in the level of reactive polypeptide(s) evaluated. For example, the assays may be performed every 24-72 hours for a period of 6 months to 1 year, and thereafter performed as needed. In general, a cancer is progressing in those patients in whom the level of polypeptide detected by the binding agent increases over time. In contrast, the cancer is not progressing when the level of reactive polypeptide either remains constant or decreases with time.

Certain in vivo diagnostic assays may be performed directly on a tumor. One such assay involves contacting tumor cells with a binding agent. The bound binding agent may then be detected directly or indirectly via a reporter group. Such binding agents may also be used in histological applications. Alternatively, polynucleotide probes may be used within such applications.

Any antibody which binds to IGF-1R may be used for quantitation of IGF-1R levels as an IGF-1R related cancer screen. In some embodiments of the foregoing methods, the antibodies are single chain antibodies or antigen-binding fragments are F(ab′)₂, Fab, Fd, or Fv fragments. In preferred embodiments of the foregoing methods, the cancer is one of colon cancer or ovarian cancer or pancreatic cancer. The foregoing cancer cells express aberrant levels of IGF-1R (colon cancer specific polypeptide) and the antibody is an IGF-1R specific antibody or an antigen-binding fragment thereof. In preferred embodiments of the foregoing methods, the antibodies are monoclonal or polyclonal antibodies, chimeric, human, or humanized antibodies. A representative monoclonal antibody includes 7C10, h7C10/Mk-0646 or an antigen-binding fragment thereof any other antibody that competes for binding IGF-1R with 7C10 or h7C10 (humanized). Optionally, the preferred antibody can be linked to one or more detectable markers, antitumor agents or immunomodulators. Antitumor agents can include cytotoxic agents and agents that act on tumor neovasculature. Detectable markers include, for example, radioactive or fluorescent markers. Cytotoxic agents include cytotoxic radionuclides, chemical toxins and protein toxins. For treatment purposes, a subject suspected of or presenting with one of a colon cancer, ovarian cancer or pancreatic cancer can be administered a pharmaceutical composition comprising h7C10 or 7C10 in a pharmaceutically acceptable excipient alone or in combination with another anti-cancer or cytotoxic agent.

A monoclonal antibody which binds to IGF-1R may be obtained by isolation of IGF-1R from a conventional cell line or produced recombinantly or from a tissue known to express IGF-1R. The antibody for IGF-1R antigen is reacted with the antigen to form a complex of the antibody and the IGF-1R antigen. In a preferred embodiment, a combination of one IgG antibody and one IgM antibody is used. Any means available for facilitation of antibody-antigen binding may be used in the disclosed method including but not limited to tubes, filters, beads, multiwell plates and a mixture thereof. Preferred embodiments use either ELISA plate technology or slot dot assays.

Means to quantitate the extent of binding include detection using colorimetric assays as well as radioimmunoassay. In certain embodiments, the complex of the antibody and the IGF-1R antigen is exposed to a second antibody which is labeled such that the level of IGF-1R antigen in the sample may be detected and quantitated by reference to a standard curve prepared from dilutions of purified IGF-1R. Such labels include, but are not limited to, radioactive and calorimetric methods including absorption, bioluminescence and fluorescence labeling means. In certain embodiments, the second antibody is biotinylated and is subsequently treated with peroxidase conjugated streptavidin to produce a quantifiable colorimetric signal. ELISA methodology may also be used to detect the IGF-1R polypeptide. A cut off value for detection of colon cancer in μ.gm/ml will be based upon values obtained from normal/control individuals.

### Assay Techniques

To carry out the ELISA, antibody specific to IGF-1R is incubated on a solid support, e.g., a polystyrene dish, that binds the antibody. Any free protein binding sites on the dish are then covered by incubating with a non-specific protein such as bovine serum albumin. Next, the sample to be analyzed is incubated in the dish, during which time IGF-1R binds to the specific antibody attached to the polystyrene dish. Unbound sample is washed out with buffer. A reporter antibody specifically directed to IGF-1R and linked to horseradish peroxidase is placed in the dish resulting in binding of the reporter antibody to any monoclonal antibody bound to IGF-1R. Unattached reporter antibody is then washed out. Reagents for peroxidase activity, including a calorimetric substrate are then added to the dish. Immobilized peroxidase, linked to IGF-1R antibodies, produces a colored reaction product. The amount of color developed in a given time period is proportional to the amount of IGF-1R protein present in the sample. Quantitative results typically are obtained by reference to a standard curve.

A competition assay may be employed wherein antibodies specific to IGF-1R attached to a solid support and labeled IGF-1R and a sample derived from the host are passed over the solid support and the amount of label detected attached to the solid support can be correlated to a quantity of IGF-1R in the sample.

Nucleic acid methods may be used to detect IGF-1R mRNA as a marker for colon cancer. Polymerase chain reaction (PCR) and other nucleic acid methods, such as ligase chain reaction (LCR) and nucleic acid sequence based amplification (NASABA), can be used to detect malignant cells for diagnosis and monitoring of various malignancies. For example, reverse-transcriptase PCR (RT-PCR) is a powerful technique which can be used to detect the presence of a specific mRNA population in a complex mixture of thousands of other mRNA species. In RT-PCR, an mRNA species is first reverse transcribed to complementary DNA (cDNA) with use of the enzyme reverse transcriptase; the cDNA is then amplified as in a standard PCR reaction. RT-PCR can thus reveal by amplification the presence of a single species of mRNA. Accordingly, if the mRNA is highly specific for the cell that produces it, RT-PCR can be used to identify the presence of a specific type of cell.

Hybridization to clones or oligonucleotides arrayed on a solid support (i.e., gridding) can be used to both detect the expression of and quantitate the level of expression of that gene. In this approach, a cDNA encoding the IGF-1R gene is fixed to a substrate. The substrate may be of any suitable type including but not limited to glass, nitrocellulose, nylon or plastic. At least a portion of the DNA encoding the IGF-1R gene is attached to the substrate and then incubated with the analyte, which may be RNA or a complementary DNA (cDNA) copy of the RNA, isolated from the tissue of interest.

### EXAMPLE 1

### Generation and Selection of the Murine Monoclonal Antibody (MAB)

With the aim of generating MAb specifically directed against IGF-IR and not recognizing the IR, a protocol comprising 6 screening stages was envisaged.

It consisted in:
immunizing mice with recombinant IGF-IR, in order to generate hybridomas,
screening the culture supernatants by ELISA on the recombinant protein which served for immunization,
testing all the supernatants of hybridomas positive by ELISA on the native receptor overexpressed on the surface of MCF-7 tumor cells,
evaluating the supernatants of hybridomas positive in the two first screenings in terms of differential recognition of IGF-IR and of IR on insect cells infected with baculoviruses respectively expressing IGF-IR or IR,
verifying that the antibodies selected at this stage were capable of inhibiting in vitro the induced IGF1 proliferation of the MCF-7 cells,
ensuring the in vivo activity, in nude mice, of the candidate retained in terms of impact on the growth of the tumor MCF-7.

All of these different stages and results obtained will be briefly described below in example 1.

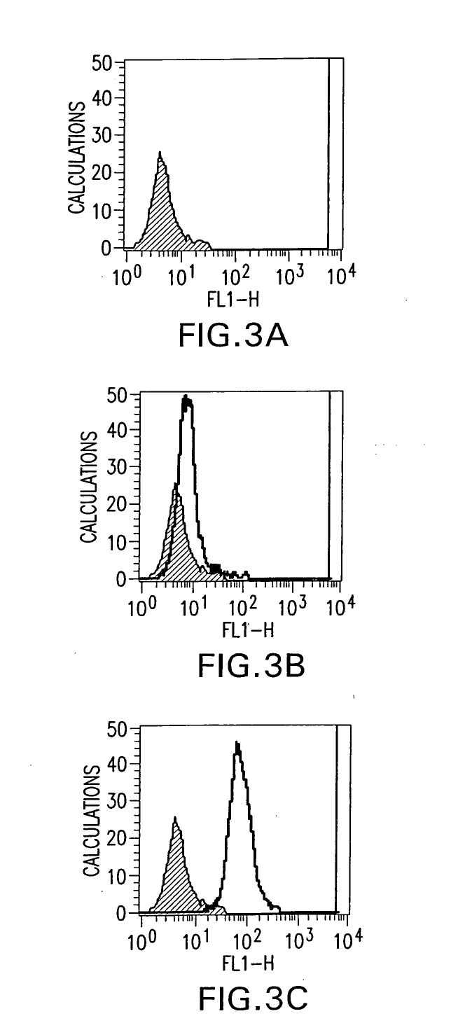

For the immunization stage, mice were injected twice, by the subcutaneous route, with 8 μg of recombinant IGF-IR. Three days before the fusion of the cells of the female rat with the cells of the murine myeloma Sp2OAg14, the mice were stimulated by an intravenous injection of 3 μg of the recombinant receptor. Fourteen days after the fusion, the supernatants of hybridomas were screened by ELISA, on plates sensitized by recombinant IGF-IR. The hybridomas whose supernatants were found positive were conserved and amplified before being tested on the FACScan so as to verify that the antibodies produced were likewise capable of recognizing native IGF-IR. In order to do this, MCF-7 cells from an estrogen-dependent tumor of the breast overexpressing IGF-IR were incubated with each of the culture supernatants produced by the hybridomas selected in ELISA. The native/MAb receptor complexes on the surface of the cell were revealed by a secondary anti-species antibody coupled to a fluorochrome. FIGS. 3A to 3C show a histogram type obtained with the supernatant of the hybridoma 7C10 (FIG. 3C) compared with a cell labeling alone+secondary antibody (FIG. 3A) or with a labeling utilizing a control isotype (FIG. 3B).

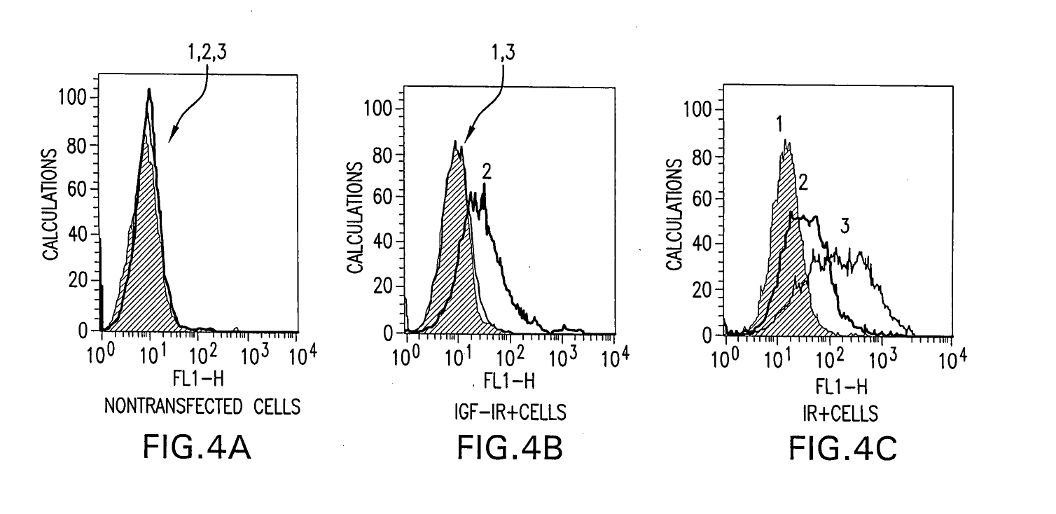

At this stage of the selection, only the hybridomas secreting MAb at the same time recognizing the recombinant receptor and the native receptor were selected and cloned. The MAb secreted by these hybridomas were produced and then purified before being tested on the FACScan, according to the method described above, on Sf9 insect cells expressing IGF-IR or IR in order to eliminate the hybridomas at the same time recognizing the two receptors. FIG. 4A shows a total recovery of the histograms 1, 2, 3 respectively corresponding to the noninfected cells+secondary antibodies (1), to the noninfected cells labeled by αIR3+ secondary antibodies (2) and to the noninfected cells labeled by an anti-IR antibody+ secondary antibodies (3). This first result shows well the absence of IGF-IR and of IR detectable on the surface of these noninfected insect cells. FIG. 4B shows a labeling of infected cells by a baculovirus expressing IGF-IR. In this second figure, the αIR3, used as a positive control, labels well, as expected, the cells (peak 2), while the anti-IR (peak 3) is superimposed on the peak of single cells. Finally, in FIG. 4C, it is shown that the anti-IR labels well, as expected, the Sf9 cells expressing the IR (peak 3), but in an unexpected manner, the αIR3 described in the literature as specific for IGF-IR seems likewise to recognize the IR (peak 2).

The results obtained in this third screening system are summarized in table 1 and show the generation of an MAb: 7C10, satisfying the criteria of recognition of the IGF-IR and of nonrecognition of the IR. The isotyping of the Mab 7C10 has shown that it involves an IgG1.

TABLE 1
Comparative reactivity of MAb 7C10 on Sf9
insect cells expressing IGF-IR or IR

| | MFI | | |
|---|---|---|---|
| | (Mean fluorescence intensity) | | |
| | Noninfected cells | IGF1R + cells | IR + cells |
| Cells | 8 | 8 | 7 |
| Anti-IR | 4.6 | 9 | 91 |
| Anti-IGF-IR (αIR3) | 9 | 35 | 32 |
| EC2 | 8 | 13 | 11 |
| Anti-mouse FITC | 4.3 | 9 | 13 |
| UltraCulture medium | 9 | 10 | 11 |
| 15B9 | 7.5 | 25 | 77.8 |
| 9F5D | 8 | 41 | 40 |
| 13G5 | 7.8 | 37 | 24 |
| 7C10 | 8.6 | 49 | 13 |

The two last screenings provided for the selection of the MAb consisted in verifying that the latter was very capable of inhibiting the cell proliferation induced by the IGF-I in vitro and in vivo on the cell line MCF-7.

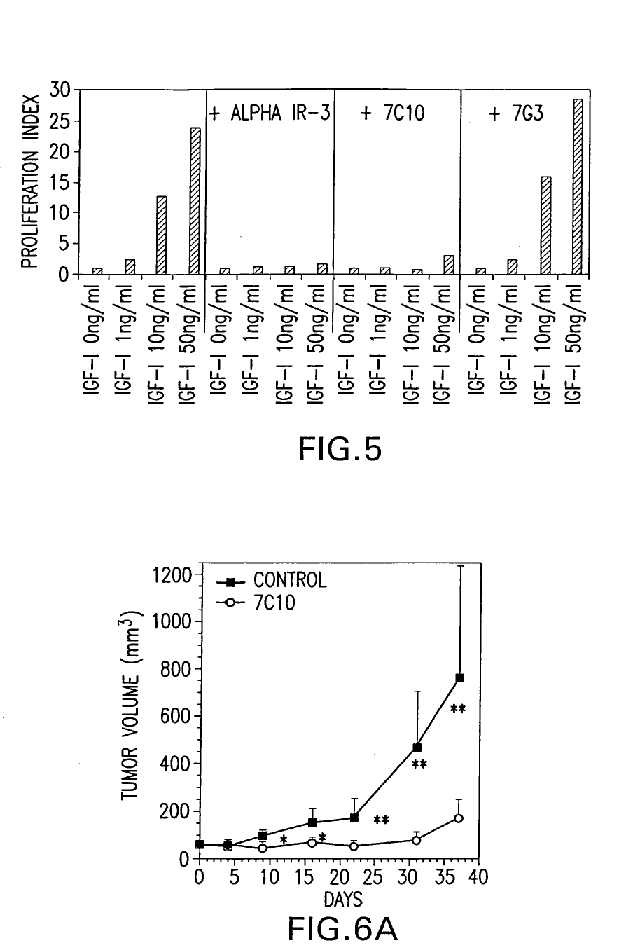

For the in vitro selection, the MCF-7 cells were inoculated, deprived of fetal calf serum, then incubated in the presence of increasing concentrations of IGF-I (from 1 to 50 ng/ml) in the presence or in the absence of the 7C10 antibody to be tested added to a final concentration of 10 μg/ml. In this experiment, the commercial αIR3MAb was introduced as a positive control and the 7G3 MAb (isolated in parallel to the 7C10 and weakly recognizing the native receptor (MFI on the FACS of 50 compared with 200 for the MAb 7C10)) as a control isotype. The cell proliferation is estimated by following on the β counter the incorporation of tritiated thymidine by the cells. The results are expressed as a proliferative index. The data presented in FIG. 5 show that IGF1 is capable of stimulating in a dose-dependent manner the proliferation of the MCF-7 cells. The MAb αIR3, used as a positive control, completely inhibits the proliferation of the MCF-7 cells induced by the IGF-I. In the same manner, the MAb 7C10 significantly inhibits the growth of the MCF-7 cells induced by IGF-I. Finally, the MAb 7G3 used as an isotype control turns out well, as expected, without effect on the tumor cell growth in vitro of the MCF-7 cell.

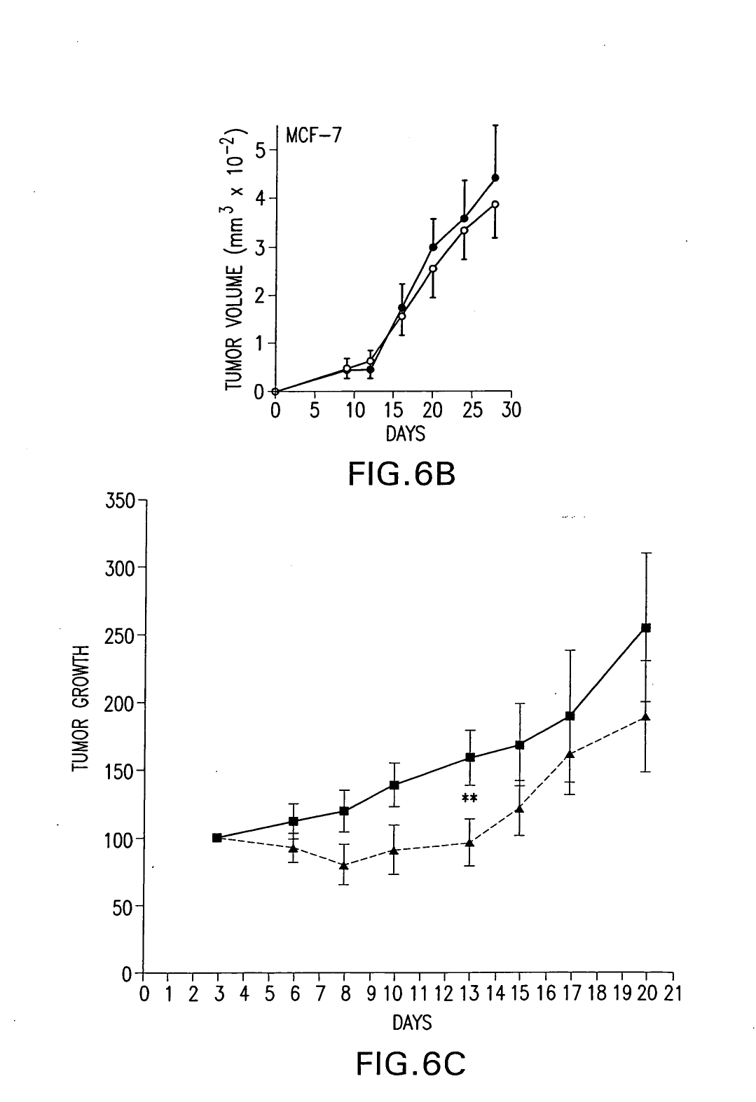

The in vivo selection was carried out in an established tumor model. In order to do this, nude mice received a subcutaneous implant of slow-release estrogen, indispensable for the taking of the tumor in a murine model. Twenty-four hours after implantation of the estrogens, 5.10⁶ MCF-7 cells are grafted onto the right flank of the mouse subcutaneously. Five days after this cell graft, the tumors are measurable and batches of 6 mice are formed at random. The treatment of the mice is carried out twice per week, during 5 to 6 weeks, at the dose of 250 μg/dose/mouse. In the control group, the mice are treated in the same fashion with a murine control isotype. The results presented in FIG. 6A show a very significant inhibition of the tumor growth induced by the antibody 7C10. This activity is particularly unexpected if reference is made to the data available concerning αIR3, always used as a reference in the domain of the receptor for IGF1, and known for not having any activity in vivo on the growth of estrogen-dependent tumors (see FIG. 6B). In the same way, compared with the results obtained with the recombinant antibody scFv-Fc derived from the murine MAb 1H7 (see FIG. 6C), the MAb 7C10 is much more efficacious in the in vivo inhibition of the growth of the MCF-7 cells.

### EXAMPLE 2

### Comparison of the Effect of 7C10 and of Tamoxifen on the In Vivo Growth of the Tumor MCF-7

With the aim of determining the effectiveness of the treatment by the antibody 7C10 in the context of estrogen-dependent cancer of the breast, 7C10 was compared with the tamoxifen compound currently used for the treatment of mammary carcinoma in the context of developed forms with local and/or metastatic progression and in the context of the prevention of recurrences (see VIDAL 2000, pages 1975-1976).

In hormone-dependent cancers of the breast, a significant correlation exists between the expression of the receptors for estrogens (ER) and that of the IGF-IR (Surmacz E. et al., Breast Cancer Res. Treat., Feb., 47(3):255-267, 1998). Furthermore, it seems that the estrogens (E2) act in synergy with IGF 1 (sometimes written IGF-I) in order to stimulate cell proliferation. It has in effect been shown that a treatment with E2 increases by approximately 10 times the mRNA level of IGF-IR as well as the expression level of the protein (Lee A. V. et al., Mol. Endocrinol., May, 13(5):787-796, 1999). This increase is manifested by a significant increase in the phosphorylation of the IGF-IR. In addition, the E2 significantly stimulates the expression of IRS-I (“IRS-1” for “Insulin Receptor Substrate-1”) which is one of the substrates of the phosphorylated IGF-IR.

Tamoxifen has been widely used for many years in hormone therapy for the treatment of patients suffering from E2-dependent breast cancers (Forbes J. F., Semin. Oncol., Feb., 24 (1st Suppl. 1):S1-5-S1-19, 1997). This molecule enters into competition with the estradiol and inhibits the attachment of this to its receptor (Jordan V. C., Breast Cancer Res. Treat., 31(1):41-52, 1994). It has in addition been demonstrated that tamoxifen is capable of inhibiting the IGF-1R-dependent proliferation by inhibiting the expression of the receptor and its phosphorylation (Guvakova M. A. et al., Cancer Res., July 1, 57(13):2606-2610, 1997). These data as a whole seem to indicate that IGF-IR is an important mediator of the proliferation induced by the E2/ER interaction.

The long-term use of tamoxifen is associated with a significant increase in the risk of endometrial cancer (Fisher et al., J. of National Cancer Institute, 86, 7:527-537, 1994; VIDAL 2000, 1975-1976) and of collateral recurrence of E2-independent cancer of the breast (Li C. I. et al., J. Natl. Cancer Inst., July 4, 93(13):1008-1013, 2001). In this context, a comparison of the in vivo antitumor effect of the antibody 7C10 and of tamoxifen has been carried out on the MCF-7 model so as to determine the part of the activity connected with IGF-IR in the mediated ER proliferation. In order to do this, 7. 10⁶ MCF-7 cells were implanted sc (subcutaneously) in nude mice, 24 hours after implantation in these same mice of a grain of estradiol with prolonged release (0.72 mg/tablet liberated over 60 days), indispensable for the establishment of any E2-dependent human tumor in this animal species. Five days after this implantation, the tumors are measured and groups of 6 mice are formed. These groups are treated respectively with 1) the 7C10 antibody injected ip (intraperitoneally) at a rate of 250 μg/mouse, twice per week, 2) 10/g of tamoxifen taken in PBS containing 3% of hydroxypropyl-cellulose (HPC) ip or 3) the solvent in which the tamoxifen is taken up (hydroxypropylcellulose). The tamoxifen is administered daily for 4 weeks except at the weekend. The mice treated with the MAb 7C10 likewise daily receive an injection of PBS with 3% HPC. A study was previously carried out in order to verify that the solvent alone is without influence on the tumor growth.

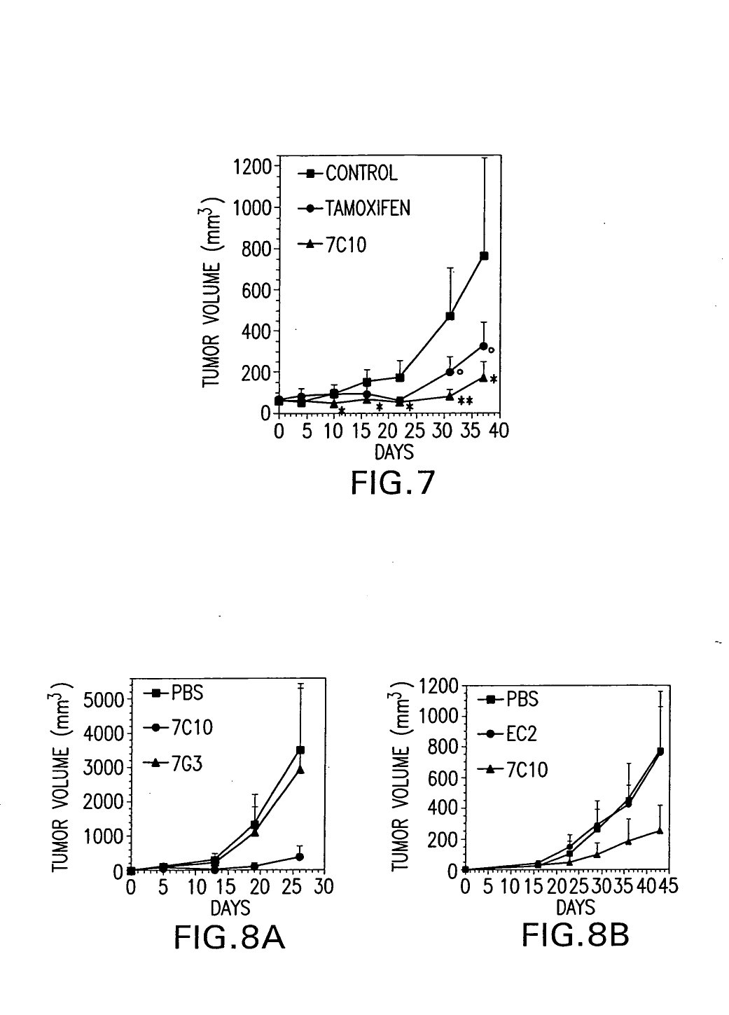

The results presented in FIG. 7 shown that the MAb 7C10 is capable of significantly inhibiting the growth of the tumor MCF-7 in vivo (the asterisks (*) correspond to the comparison control group/7C10 group in a t-test). In a surprising fashion, the antibody 7C10 seems to be significantly more efficacious than tamoxifen for the inhibition of the tumor growth (the circles (°) correspond to the comparison tamoxifen group/7C10 group in a t-test) suggesting that this type of treatment by MAB might be substituted for treatment with tamoxifen.

### EXAMPLE 3

### Demonstration of the Antitumor Activity of the MAB 7C10 In Vivo on Human Tumors of Different Origins

a) In Vivo Activity of the Antibody 7C10 in Three Tumor Models

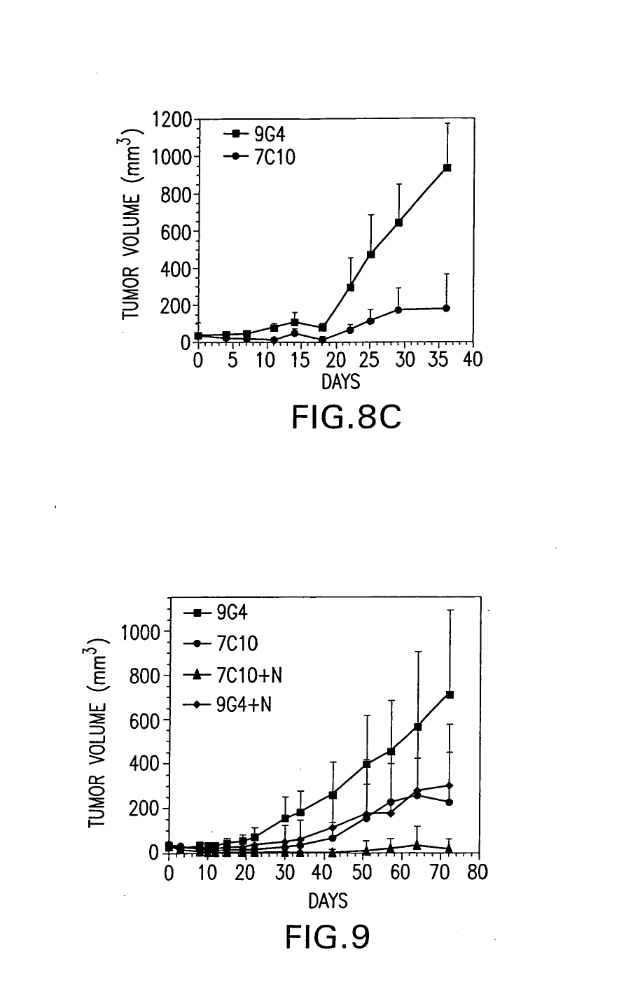

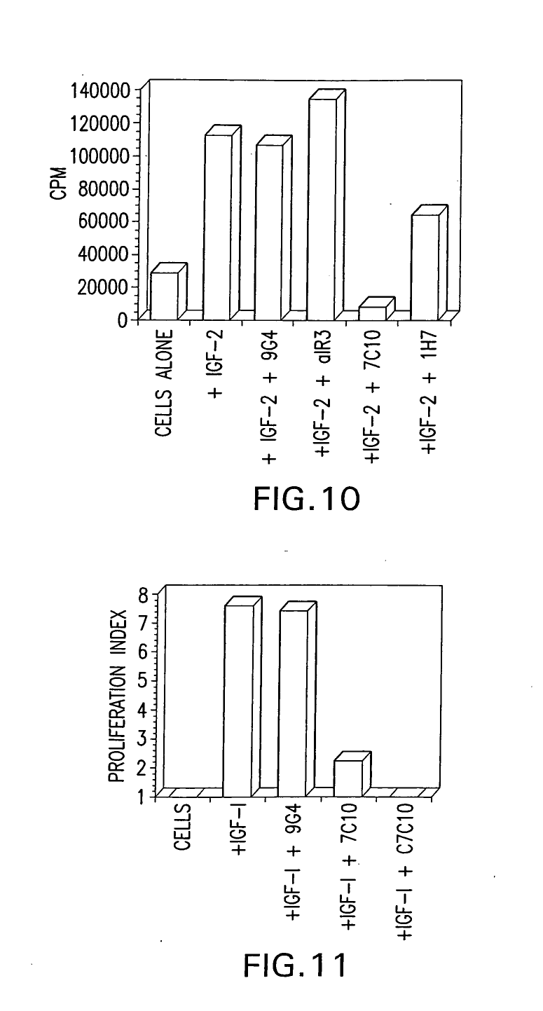

In order to generalize the activity of the 7C10 antibody to other tumors expressing the receptor for IGF 1, 7C10 was tested in vivo in an androgen-independent model of tumor of the prostate DU145 (likewise written DU-145), in an SKES-1 osteosarcoma model and in a model of non-small cell tumor of the lung A549. The protocol is comparable to that described above for MCF-7 and the results presented in FIGS. 8A to 8C show a significant activity of this MAB in the 3 tumor models. The activity observed in the model of tumor of the prostate is to be noted very particularly inasmuch as the single chain scFv of the MAB 1H7 is without activity in an androgen-independent model of tumor of the prostate (Li et al., 2000).

b) In Vivo Activity of the Antibody 7C10 in an Orthotopic Model A549

The conventional xenograft models as described above do not allow the study of drugs on metastatic dissemination. In effect, the tumors implanted s.c. (subcutaneously) remain localized at the sight of injection and are therefore not really a reflection of the situation in man. In order to evaluate our antibody in a model closer to reality, the A549 cells were implanted in an intrapleural location. This model, which is well described (Clin. Cancer Res. 2000 January; 6(1):297-304) allows a metastatic dissemination close to that observed in man to be observed, with mediastinal, pulmonary, cardiac and vertebral metas-tases. In the study which was carried out, 106 A549 cells were injected intrapleurally into female nude mice. 7 days after implantation, the mice were divided into 2 batches of 22. One of these batches received a challenge dose of 500 μg/mouse and was then treated twice per week at a rate of 250 μg of 7C10/dose. The second batch was treated according to the same scheme with the control isotype 9G4. FIG. 31 shows a significant extension of survival in the mice treated with the MAB 7C10 indicating that this antibody is capable of having an action on metastatic dissemination.

### EXAMPLE 4

### Comparison of the MAB 7C10 with Navelbine In Vivo; Effect of a Coadministration of the Two Treatments

Navelbine is a chemotherapy compound indicated in non-small cell cancer of the lung and in metastatic cancer of the breast. The comparative study of 7C10 and of navelbine and the possible synergy between the two products was studied on the tumor model A549. For this study, 5.106 A549 cells were grafted subcutaneously on the right flank of the mouse. Five days after the cell graft, the tumors are measurable and the treatments with MAb and/or navelbine are commenced. The MAb dose is always 250 μg/dose/mouse, twice per week, intra-peritoneally. Concerning navelbine, it will be administered at the maximum dose tolerated by the mouse or 10 mg/kg, intraperitoneally. For this treatment three injections will be carried out at intervals of 7 days. During the coadministrations, the two products are mixed before injection.

The results presented in FIG. 9 show in a surprising fashion that, in this model, the antibody 7C10 is as active as the conventional treatment with navelbine. A very significant synergy of the two products is likewise observed with five mice out of seven not having measurable tumors on day 72.

### EXAMPLE 5

### Study of the In Vitro Inhibition of the IGF2-Induced Growth of the MCF-7 Tumors

As indicated above, IGF-IR is overexpressed by numerous tumors but it has furthermore been described that in a good part of the cancers of the breast and of the colon especially, the proliferation signal is given to this receptor via IGF2 (sometimes written IGF-II or I). It is therefore essential to ensure that the MAb 7C10 is likewise capable of inhibiting the IGF2 growth induced on the MCF-7 tumor in vitro. In order to do this, cells were inoculated into 96-well plates, deprived of fetal calf serum and stimulated by the addition of 200 ng of IGF2 per ml, final concentration, of medium, in the presence and in the absence of the MAb to be tested introduced at a concentration of 10 μg/ml. The results presented in F1G. 10 show that IGF2, like IGF1, significantly stimulates the growth of MCF-7 cells. The addition of a control isotype, 9G4, remains without effect on this stimulation. As already described by De Leon et al. (Growth Factors, 6:327-334, 1992), no effect is observed during the addition of the MAb αIR3. On the other hand, 7C10 totally inhibits the growth induced by IGF2. Its activity is significantly better than that of 1H7.

### EXAMPLE 6

### Biological Activity of the Chimeric 7C10 (C7C10) and Humanized (H7C10) Antibodies 7C10

a) 7C10/C7C10 and 7C10/h7C10 Comparison on the MCF-7 Model In Vitro

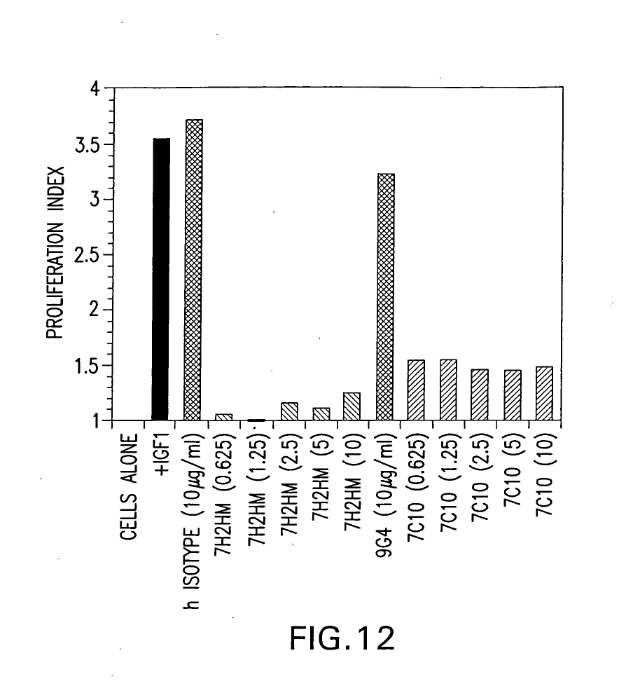

The chimeric form of the MAb 7C10 and the purified humanized form 1 (written here 7H2HM) were tested in vitro in the MCF-7 model as described above. The results presented respectively in FIGS. 11 and 12 show that these two forms have perfectly preserved their properties of inhibiting the IGF1-induced growth of the MCF-7 tumor.

b) Comparative Effect of the MAb 7C10 and h7C10 on the Transduction of the signal induced by the attachment of IGF1 to its receptor

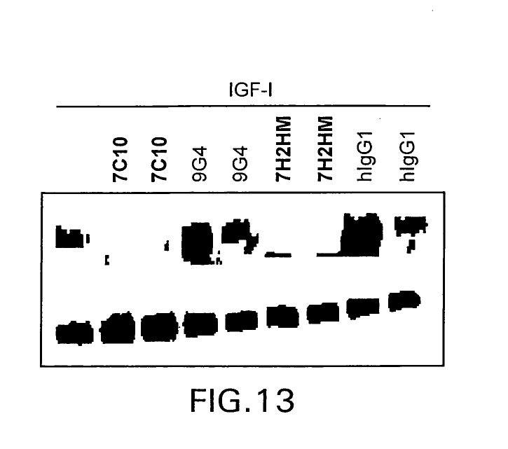

The activity of the inhibition of the IGF1 growth induced in vitro on the line MCF-7 ought to be the translation of an inhibition of the transduction of the signal mediated by IGF1 during the attachment of the MAb 7C10 to the receptor. In order to verify this hypothesis, MCF-7 cells were incubated with or without IGF1, in the presence or in the absence of the antibodies to be tested. After a short incubation time, the cells were lyzed, the β chain immunoprecipitated and the phosphorylation of this subunit estimated with the aid of an antiphosphotyrosine kinase antibody. The results presented in FIG. 13 show that the attachment of the 7C10 or of the h7C10 significantly inhibits the phosphorylation of the β subunit of IGF-IR contrary to an irrelevant murine (9G4) or human antibody (written IgG1 on the scheme).

c) Involvement of the 7H2HM Antibody in the Mechanisms of ADCC

The inhibition of the transduction of the signal described above in paragraph b) is the principal mechanism of action involved in the biological activity of the antibodies 7C10 and 7H2HM. It is, however, probable that during its administration in man, the antibody 7H2HM, of isotype IgG1, is capable of inducing cell lysis by a mechanism of ADCC type (Antibody Dependent Cellular Cytotoxicity). In order to verify this point, NK (Natural Killer) cells coming from the peripheral blood of human donors are placed in the presence of A549 or MCF-7 cells previously incubated for 4 hours with 10 μg of 7H2HM antibody per 5.10⁵ cells and labeled with ⁵¹Cr (50 μg). In this experiment, herceptin (written h4D5 on FIGS. 32A and 32B) is used as an experiment positive control. FIGS. 32A to 32D show that, as expected, herceptin induces a significant ADCC on the two cells A549 and MCF-7 (see respectively FIGS. 32A and 32B). 7H2HM is likewise capable of inducing an ADCC on the A549 cells (see FIG. 32C), but this phenomenon is of smaller amplitude on the MCF-7 cells (see FIG. 32D).

d) Effects of the Antibodies 7C10 and 7H2HM on the Cell Cycle

The inhibition of the cell growth observed in vitro on the line MCF-7 should be manifested by an effect on the cell cycle. In order to reply to this question, 4.10⁵ cells are inoculated into 6-well plates. 24 hours after inoculation, the calf serum is removed and added in the presence or in the absence of the antibodies to be tested. After incubation for 24 hours, the cells are recovered for the study of the cell cycle. FIG. 33B demonstrates the effect of IGF1 on the entry into the cycle and the growth of the MCF-7 cells compared with the entry into the cycle and the growth of the MCF-7 cells in the absence of IGF1 (see FIG. 33A). After addition of the growth factor, a significant decrease in the G0/G1 phase (from 88.2% to 56.3%) to the benefit of the S (from 7.8% to 31%) and G2/M (from 4% to 12.7%) phases is observed. During the addition of the antibodies 7C10 and 7H2HM (see FIG. 33C), a significant inhibition of the entry into the cycle is observed. In it is to be noted that the murine antibody and its humanized homolog have a comparable activity on the cell cycle. The αIR3, introduced as a positive control, seems slightly less active than the 7C10 and the 7H2HM in this test. The antibody 9G4 used as a control isotype is without effect on the cell cycle.

e) Comparative Activity In Vivo of the Antibodies 7C10 and 7H2HM on the Model A549

In order to confirm the activity of the humanized antibody 7H2HM in vivo, the latter was compared with 7C10 in the model of non-small cell tumor of the lung A549. This experiment was carried out exactly as described above except for the dose of antibody which is 125 μg/dose twice per week in place of 250 μg/dose twice per week and that of the fact of the nonavailability of great quantities of 7H2HM. The antibody 9G4 was used as an isotype control for 7C10 and an irrelevant human immunoglobulin of isotype IgG1 (below called HIgG1) was used as a control for the humanized antibody 7H2HM.

FIG. 34A shows that there are no significant differences between the 9G4 and HIgG1 control curves. As expected, a significant inhibition of the tumor growth is observed with the murine antibody 7C10. Concerning the humanized antibody 7H2HM, the activity observed is of exactly the same intensity as that observed with its murine counterpart. This data, in addition to the observations described above in vitro, indicates that the humanization has not modified the properties of the antibody generated. On the other hand, in the xenograft models in the mouse, the activity of the humanized antibody seems to be integrally connected with a mechanism of inhibition of the transduction of the signal. In effect, if an ADCC part was in play in the inhibition of the tumor growth in the Nude mouse, a difference ought to be observed between the activity of the murine and humanized antibodies.

An in vivo experiment was likewise carried out on the MCF-7 breast tumor model and shows that, as expected, the antibody 7H2HM is perfectly comparable with the murine antibody 7C10 for the inhibition of the growth of this tumor in vivo (FIG. 34B).

f) Demonstration of a Synergy Between the 7H2HM and Navelbine

The protocol described in example 4 was repeated with the aim of reproducing the results obtained with 7C10 with its humanized homolog: the antibody 7H2HM.

The results presented in FIGS. 35A and 35B show that, as in the case of 7C10, a significant synergy is demonstrated between the humanized antibody 7H2HM and navelbine.

g) Effect of the Antibodies 7C10 and 7H2HM on the Apoptosis of MCF-7 cells in vitro

As indicated above, IGF-IR is capable of conferring protection against apoptosis when it is overexpressed on the surface of cells. Furthermore, it has been demonstrated in these examples that the antibodies 7C10 and 7H2HM were capable of potentiating an active compound in chemotherapy. In order to test the power of the antibodies 7C10 and 7H2HM to induce apoptosis, and to explain in part their synergy potential with the chemotherapy, experiments were conducted on the MCF-7 cells in the presence or in the absence of doxorubicin, a medicament known to induce the apoptosis of this cell line in vitro. In these experiments, the MCF-7 cells are inoculated at 2.10⁴/cm² in Petri dishes and cultured for 24 h in RPMI without phenol red supplemented with 10% of fetal calf serum (FCS). The cells are then washed twice with PBS and put back into culture in medium with 0% FCS. They are allowed an adaptation time of 10 minutes at 37° C. before the addition of the antibodies at 10 μg/ml. After an extra 10 minutes at 37° C., recombinant IGF-I (Sigma) is added to the culture medium to a final concentration of 50 ng/ml. The cells are left at 37° C. again for one hour in order to allow the attachment of the antibodies and of the IGF-I. Finally, the doxorubicin (Sigma) is added to the culture medium at 2 μg/ml and the cells are incubated for 24 hours at 37° C.

The experiments have likewise been conducted with navelbine at a concentration of 10 μg/ml.

The analysis of the cell viability is carried out by flow cytometric analysis after labeling with the annexin V-FITC (20 minutes, 4° C.) and DAPI (2 μg/ml). The percentage of dead cells considered is the labeled population Annexin+/DAPI+. The antibody 5C2 is used as a control isotype.

The results represented in FIG. 36 show that doxorubicin induces apoptosis in 8% of the MCF-7 cells. When the cells are treated conjointly with the antibody 7C10 and the doxorubicin a significant increase in cell death is observed. The same effect is shown with the antibody 7H2HM. The same type of results was observed when the antibody is combined with navelbine.

### EXAMPLE 7

### Cloning Strategy of Genes Coding for the Variable Regions of the Heavy and Light Chains of the Monoclonal Antibody (Mab) 7C10

The total RNA was extracted from 10⁷ cells of hybridomas secreting the antibody 7C10 by using the TRI REAGENT™ (according to the instructions given by the supplier, SIGMA, T9424). The first cDNA strand was synthesized with the aid of the ‘First strand cDNA synthesis’ kit of Amersham-Pharmacia (#27-9621-01, according to the instructions given by the supplier). For the two chains, the reaction was primed with the oligonucleotide Not I-d(T)18, comprised in the Kit.

The cDNA:mRNA hybrid thus obtained was used for the amplification by PCR of the genes coding for the heavy and light chains of the Mab 7C10. The PCR were carried out by using a combination of oligonucleotides specific for the heavy and light (Kappa) chains of mouse immunoglobulins. The primers corresponding to the 5′ ends hybridize in the region corresponding to the signal peptides (Table 2 for heavy chains, Table 3 for light chains). These primers were compiled from a large number of mouse antibody sequences found in the databanks (Jones S. T. et al., Bio/Technology 9:88-89, 1991). The primers corresponding to the 3′ ends hybridize in the constant regions of the heavy chains (CH1 domain of the subclass IgG1, not far from the V-C junction, MHC-1 primer Table 4) and light chains (Kappa domain not far from the V-C junction, MKC primer Table 4).

TABLE 2

Oligonucleotide primers for the 5′ region of the variable
domains of the heavy chains of mouse immunoglobulin (MHV)
(“MHV” for “Mouse Heavy Variable”)

| | | |
|---|---|---|
| MHV-1: | 5′ ATGAAATGCAGCTGGGTCATSTTCTT 3′ | (SEQ ID No. 13) |
| MHV-2: | 5′ ATGGGATGGAGCTRTATCATSYTCTT 3′ | (SEQ ID No. 14) |
| MHV-3: | 5′ ATGAAGWTGTGGTTAAACTGGGTTTT 3′ | (SEQ ID No. 15) |
| MHV-4: | 5′ ATGRACTTTGGGYTCAGCTTGRT 3′ | (SEQ ID No. 16) |
| MHV-5: | 5′ ATGGACTCCAGGCTCAATTTAGTTTT 3′ | (SEQ ID No. 17) |
| MHV-6: | 5′ ATGGCTGTCYTRGSGCTRCTCTTCTG 3′ | (SEQ ID No. 18) |
| MHV-7: | 5′ ATGGRATGGAGCKGGRTCTTTMTCTT 3′ | (SEQ ID No. 19) |
| MHV-8: | 5′ ATGAGAGTGCTGATTCTTTTGTG 3′ | (SEQ ID No. 20) |
| MHV-9: | 5′ ATGGMTTGGGTGTGGAMCTTGCTATT 3′ | (SEQ ID No. 21) |
| MHV-10: | 5′ ATGGGCAGACTTACATTCTCATTCCT 3′ | (SEQ ID No. 22) |
| MHV-11: | 5′ ATGGATTTTGGGCTGATTTTTTTTATTG 3′ | (SEQ ID No. 23) |
| MHV-12: | 5′ ATGATGGTGTTAAGTCTTCTGTACCT 3′ | (SEQ ID No. 24) |

NB KEY: R = A/G, Y = T/C, W = A/T, K = T/G, M = A/C, S = C/G.

TABLE 3

Oligonucleotide primers for the 5′ region of the variable
domains of kappa (light) chains of mouse immunoglobulin
(MKV) (“MKV” for “Mouse Kappa Variable”)

| | | |
|---|---|---|
| MKV-1: | 5′ ATGAAGTTGCCTGTTAGGCTGTTGGTGCT 3′ | (SEQ ID No. 25) |
| MKV-2: | 5′ ATGGAGWCAGACACACTCCTGYTATGGGT 3′ | (SEQ ID No. 26) |
| MKV-3: | 5′ ATGAGTGTGCTCACTCAGGTCCT 3′ | (SEQ ID No. 27) |
| MKV-4: | 5′ ATGAGGRCCCCTGCTCAGWTTYTTGG 3′ | (SEQ ID No. 28) |
| MKV-5: | 5′ ATGGATTTWCAGGTGCAGATTWTCAGCTT 3′ | (SEQ ID No. 29) |
| MKV-5A: | 5′ ATGGATTTWCARGTGCAGATTWTCAGCTT 3′ | (SEQ ID No. 30) |
| MKV-6: | 5′ ATGAGGTKCYYTGYTSAGYTYCTGRG 3′ | (SEQ ID No. 31) |
| MKV-7: | 5′ ATGGGCWTCAAGATGGAGTCACA 3′ | (SEQ ID No. 32) |
| MKV-8: | 5′ ATGTGGGGAYCTKTTTYCMMTTTTTCAAT 3′ | (SEQ ID No. 33) |
| MKV-9: | 5′ ATGGTRTCCWCASCTCAGTTCCTT 3′ | (SEQ ID No. 34) |
| MKV-10: | 5′ ATGTATATATGTTTGTTGTCTATTTC 3′ | (SEQ ID No. 35) |
| MKV-11: | 5′ ATGGAAGCCCCAGCTCAGCTTCTCTT 3′ | (SEQ ID No. 36) |
| MKV-12A: | 5′ ATGRAGTYWCAGACCCAGGTCTTYRT 3′ | (SEQ ID No. 37) |
| MKV-12B: | 5′ ATGGAGACACATTCTCAGGTCTTTGT 3′ | (SEQ ID No. 38) |
| MKV-13: | 5′ ATGGATTCACAGGCCCAGGTTCTTAT 3′ | (SEQ ID No. 39) |

NB KEY: R = A/G, Y = T/C, W = A/T, K = T/G, M = A/C, S = C/G.

TABLE 4

Oligonucleotide primers for the 3′ ends of the mouse
V_{H} and V_{L} genes

Light chain (MKC):

| | |
|---|---|
| 5′ ACTGGATGGTGGGAAGATGG 3′ | (SEQ ID No. 40) |
| Constant region of the mouse Kappa domain: | |
| ADAAPTVSIFPPSS | (SEQ ID No. 41) |
| GCTGATGCTGCACCAACTGTATCCATCTTCCCACCATCCAGT | (SEQ ID No. 42) |
| (MKC) CCATCTTCCCACCATCCAGT | (SEQ ID No. 43) |
| Heavy chain (MHC-1): | |
| 5′ CCAGTGGATAGACAGATG 3′ | (SEQ ID No. 44) |
| GCC AAA ACG ACA CCC CCA TCT GTC TAT CCA CTG | (SEQ ID No. 45 |
| CH1 domain of mouse gamma-1 (IgG1 subclass): | |
| AKTTPPSVYPL | (SEQ ID No. 46) |
| (MHC-1) CCCCCATCTGTCTATCCACTG | (SEQ ID No. 47) |

### EXAMPLE 8

### Sequences of Immunoglobulins Cloned from the Mouse Hybridoma 7C10

By following the amplification strategy described above, PCR products corresponding to the variable regions of the heavy (VH) and light (VL) chains were cloned by using the “pGEM®-T Easy Vector Systems” (Promega). For 7C10 VL, PCR products were obtained with the MKC primer in combination with the MKV1 and MKV2 primers. For 7C10 VH, PCR products were obtained with the MHC-1 primer in combination with the MHV8 and MHV12 primers. A thorough sequencing of the PCR products cloned in the pGem-T easy vectors revealed two different sequences for the light chain and one unique sequence for the heavy chain.

a) Variable Region Isolated from the Oligo MKV1

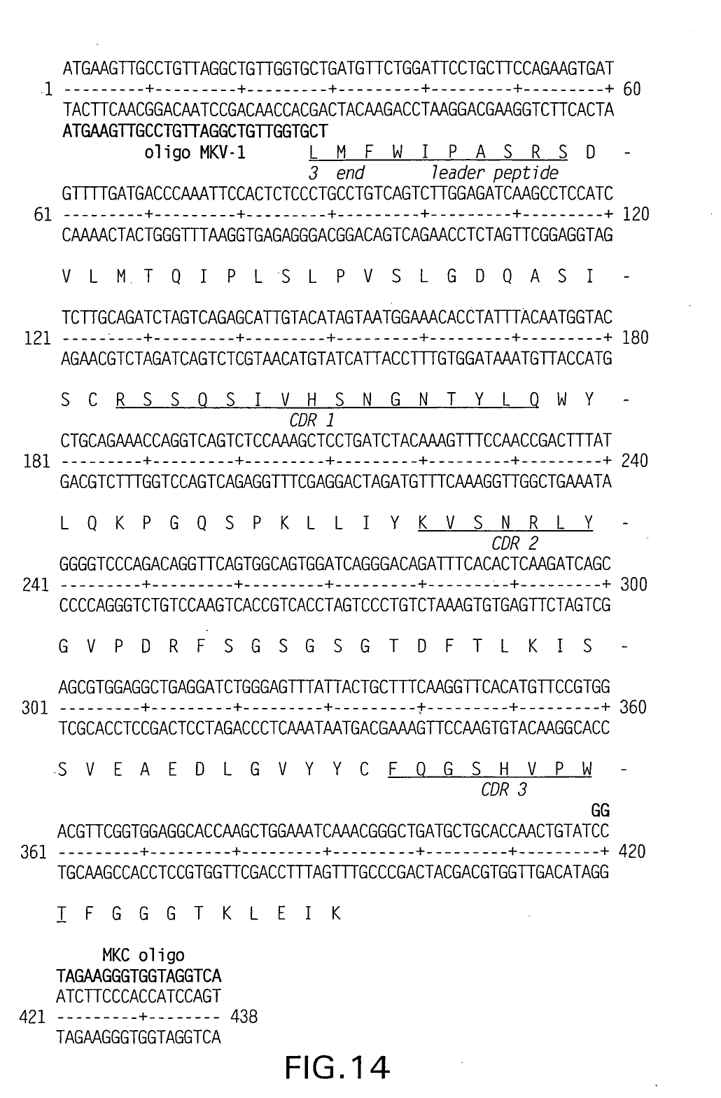

The DNA sequence obtained is characteristic of a variable region of functional Ig. This novel sequence is therefore presumed to be that coding for 7C10 VL. The DNA (SEQ ID Nos. 48 and 50) and amino acid (SEQ ID No. 49) sequences of the cDNA coding for 7C10 VL are represented in FIG. 14.

b) Variable Region Isolated from the Oligo MKV2

The gene coding for this light chain comes from an aberrant mRNA transcript which is present in all the standard fusion partners derived from the original MOPC-21 tumor of which the mouse myeloma Sp2/Oag14, which was used in order to produce the 7C10 hybridoma, is part. This sequence contains an aberrant recombination between the V and J genes (deletion of four nucleotide bases involving a change in the reading frame) and a mutation of the invariable cysteine in position 23 to tyrosine. These changes suggest that this light chain would be nonfunctional although nevertheless transcribed to messenger RNA. The DNA sequence of this pseudo light chain is not shown.

c) Variable Region Isolated from the Oligos MHV8 and MHV12

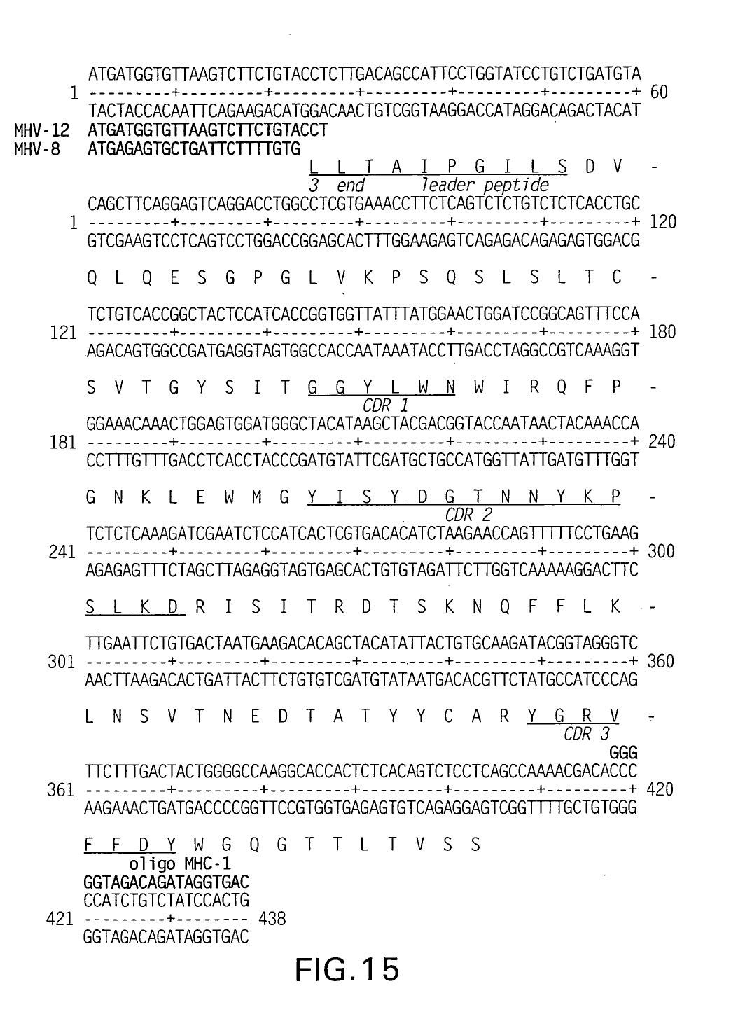

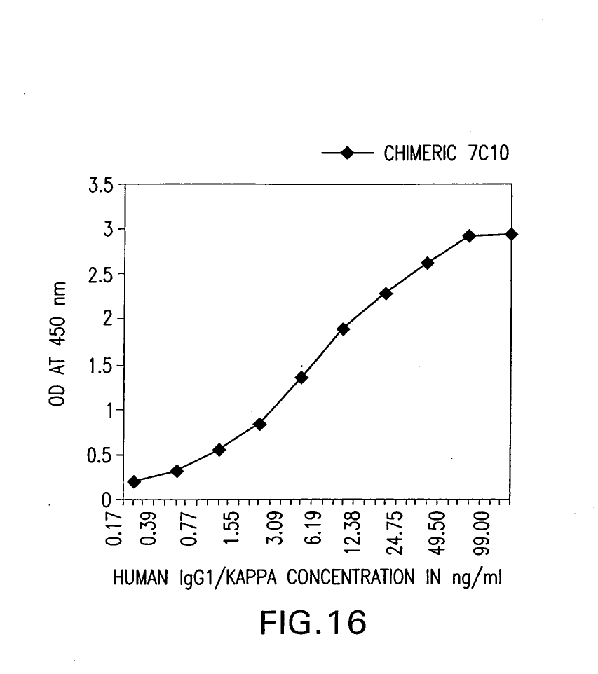

The DNA sequences obtained with these two oligos are identical, apart from the sequence encoded by the oligo itself. This sequence is a novel sequence coding for a functional heavy chain presumed to be that of the monoclonal antibody 7C10. The DNA (SEQ ID Nos. 51 and 53) and amino acid (SEQ ID No. 52) sequences of the cDNA coding for 7C10 VH are represented in FIG. 15.

### EXAMPLE 9

### Construction of Chimeric Mouse-Man Genes

The chimeric antibody 7C10 was constructed so as to have the mouse 7C10 regions VL and VH connected to the human constant regions kappa and gamma-1, respectively. Oligos were used in order to modify the 5′ and 3′ ends of the sequences flanking the DNA coding for 7C10 VL and VH in order to allow their cloning in vectors for expression in mammalian cells. These vectors use the strong promoter HCMV in order effectively to transcribe the heavy and light chains of the chimeric antibody 7C10. On the other hand, these vectors likewise contain the replication origin of SV40 allowing an effective replication of the DNA and, as a consequence, as a transitory expression of the proteins in cos cells.

### EXAMPLE 10

### Expression and Evaluation of the Recognition Activity of the IGF-I Receptor of the Chimeric Antibody 7C10

The two plasmids containing the DNA coding for the chimeric 7C10 antibody were cotransfected in cos-7 cells (ATCC number CRL-1651) in order to study the transitory expression of the recombinant antibody. After incubation for 72 hours, the culture medium was removed, centrifuged in order to eliminate the cell debris and analyzed by the ELISA technique for the production of human IgG1 (see Example 16) and the recognition of the receptor for IGF-I (see Example 17).

The ELISA tests for measurement of concentrations of human IgG1/Kappa showed that the expression of the chimeric antibody 7C10 in the cos-7 cells was between 300 and 500 ng/mm, which is comparable to the values obtained with the majority of antibodies.

The ELISA tests for recognition of the receptor for IGF-I show that the chimeric antibody recognizes it specifically and with a good relative avidity (see FIGS. 3A, 3B and 3C). This provides the functional proof that the good VH and VL of the 7C10 antibody have been identified. In addition, this chimeric form of 7C10 appears as being an indispensable tool in the evaluation of the affinity of the humanized forms.

### EXAMPLE 11

### Molecular Modeling of the Variable Regions of the Mouse Antibody 7C10

In order to assist and to refine the humanization process by “CDR grafting”, a molecular model of the VL and VH regions of the mouse antibody 7C10 was constructed. The model is based on the crystallographic structure of the heavy chain 1AY1 and of the light chain 2PCP.

### EXAMPLE 12

### Process of Humanization by CDR Grafting of the Variable Region of The Light Chain of the Antibody 7C10 (7C10 VL)

a) Comparison of the Amino Acid Sequence of 7C10 VL with all the Known Mouse VL Sequences

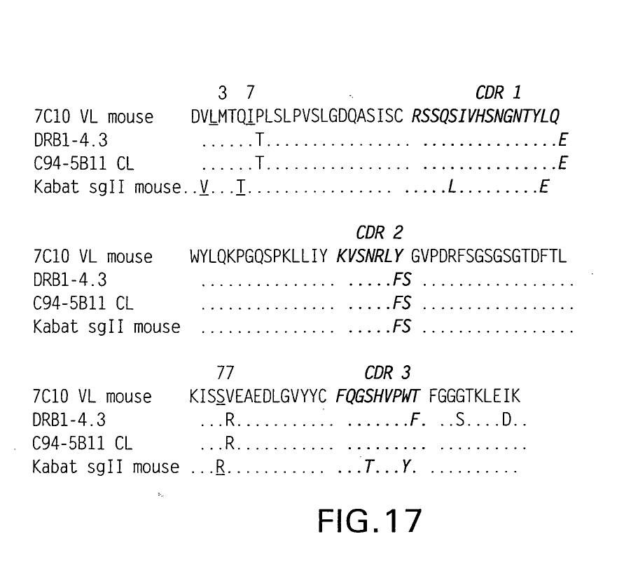

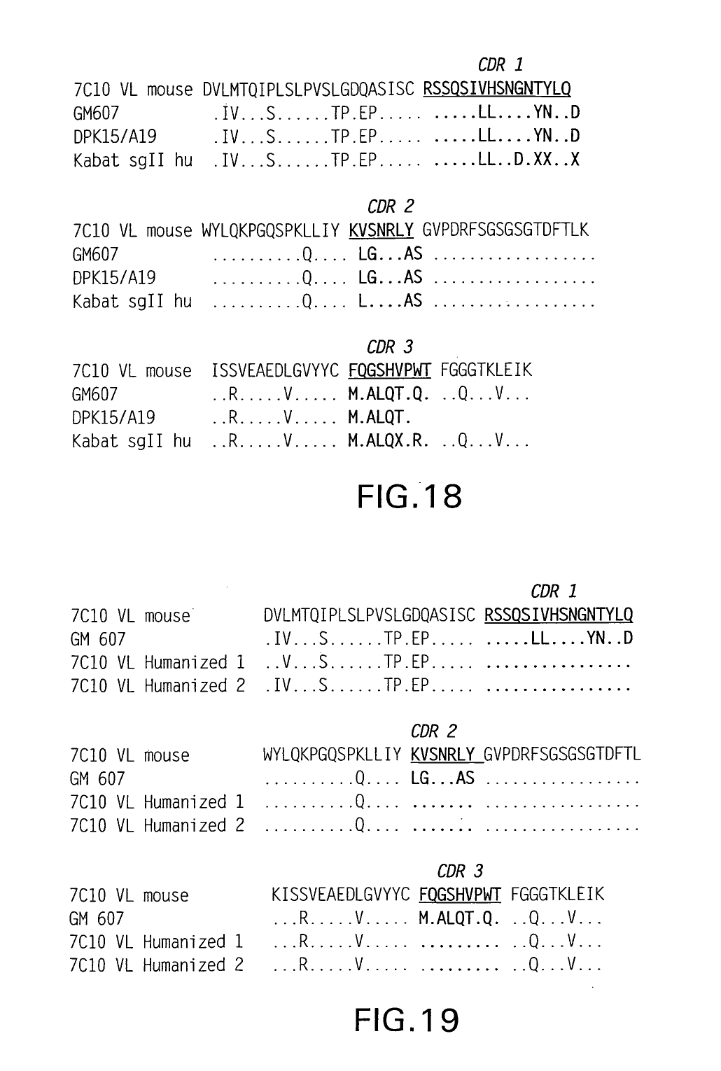

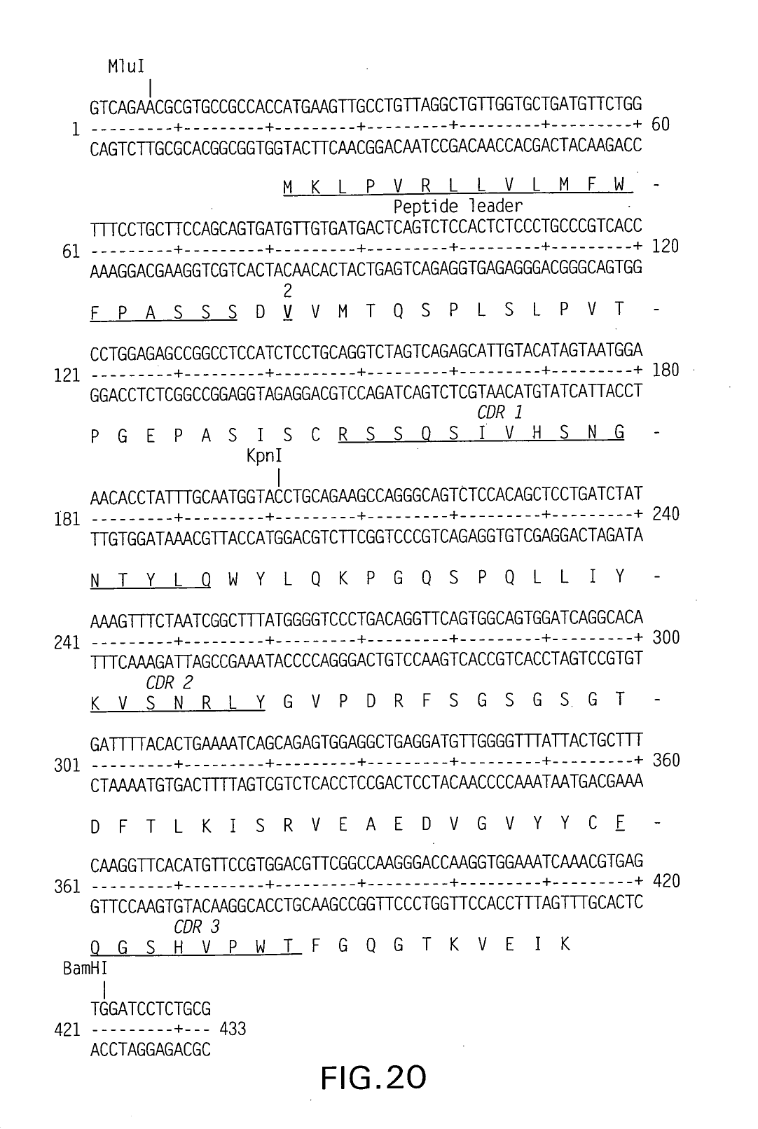

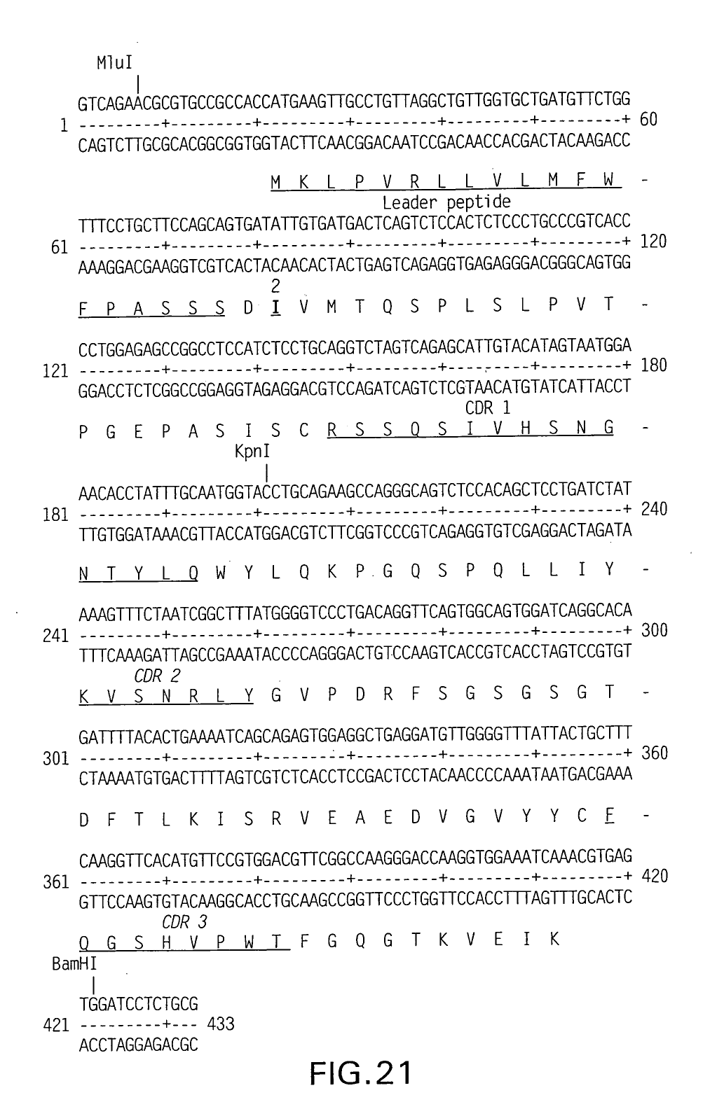

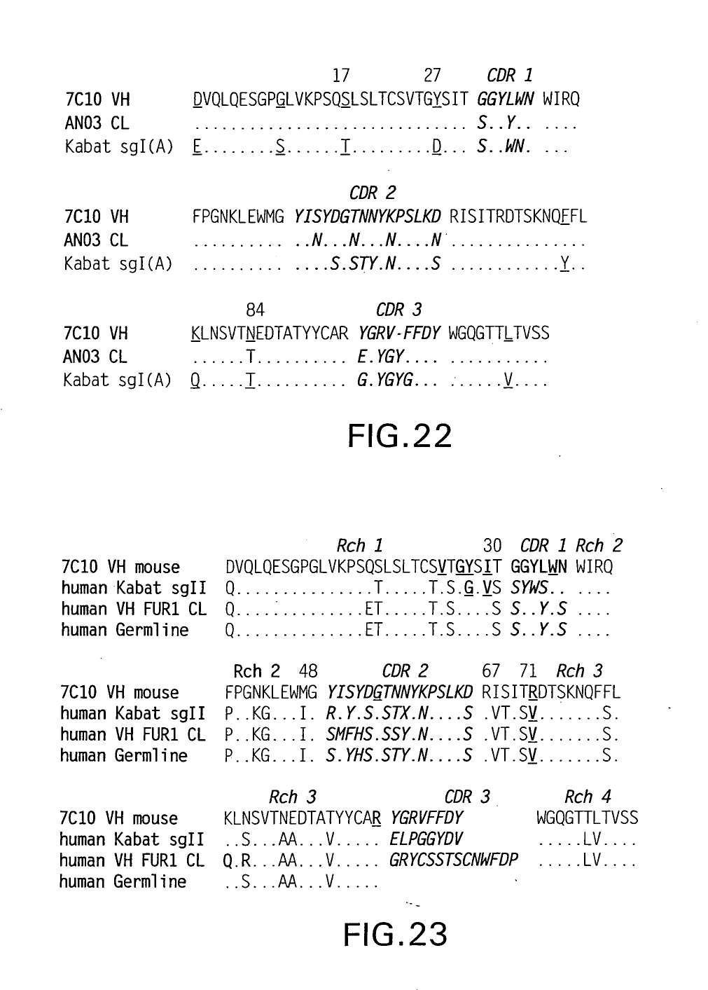

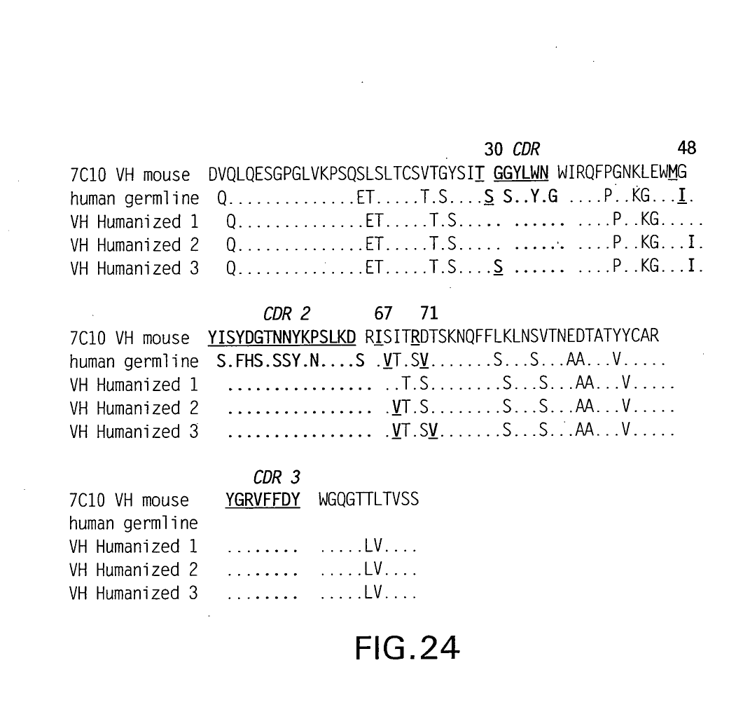

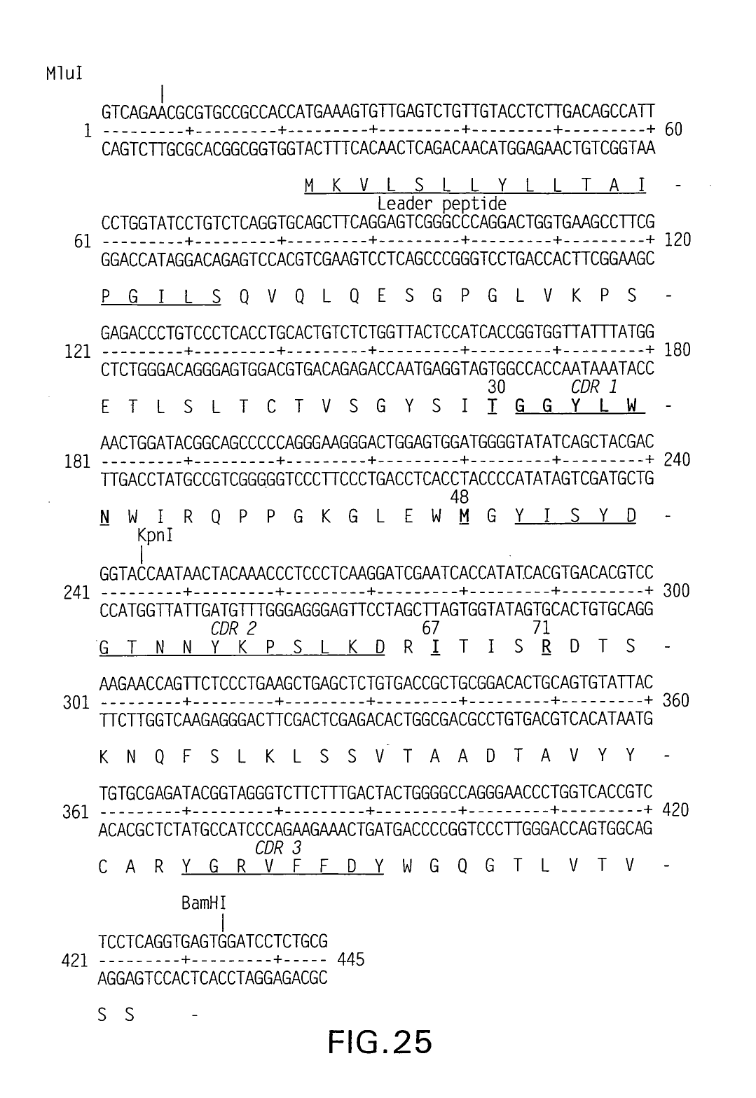

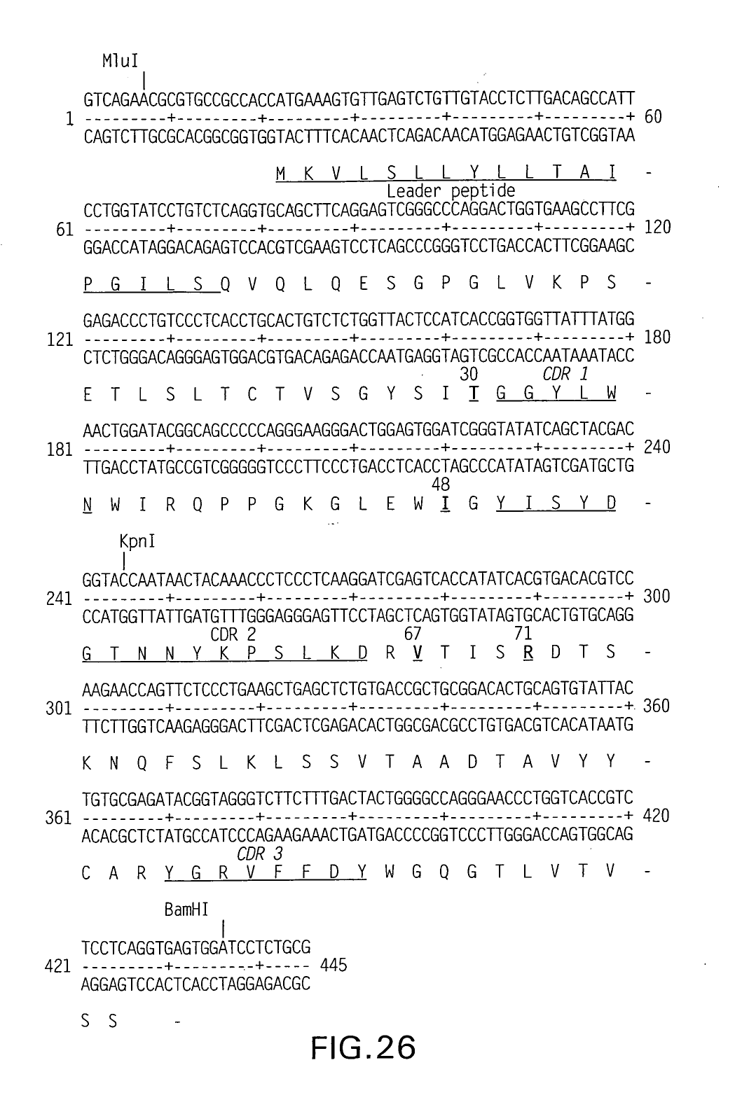

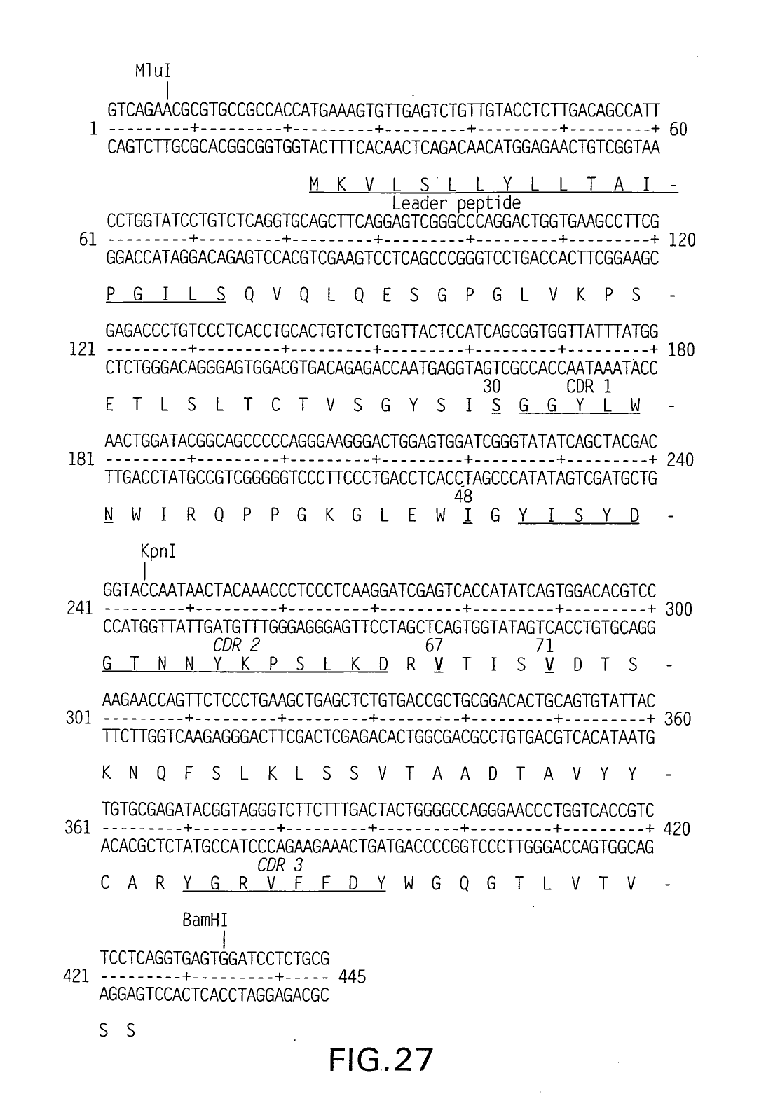

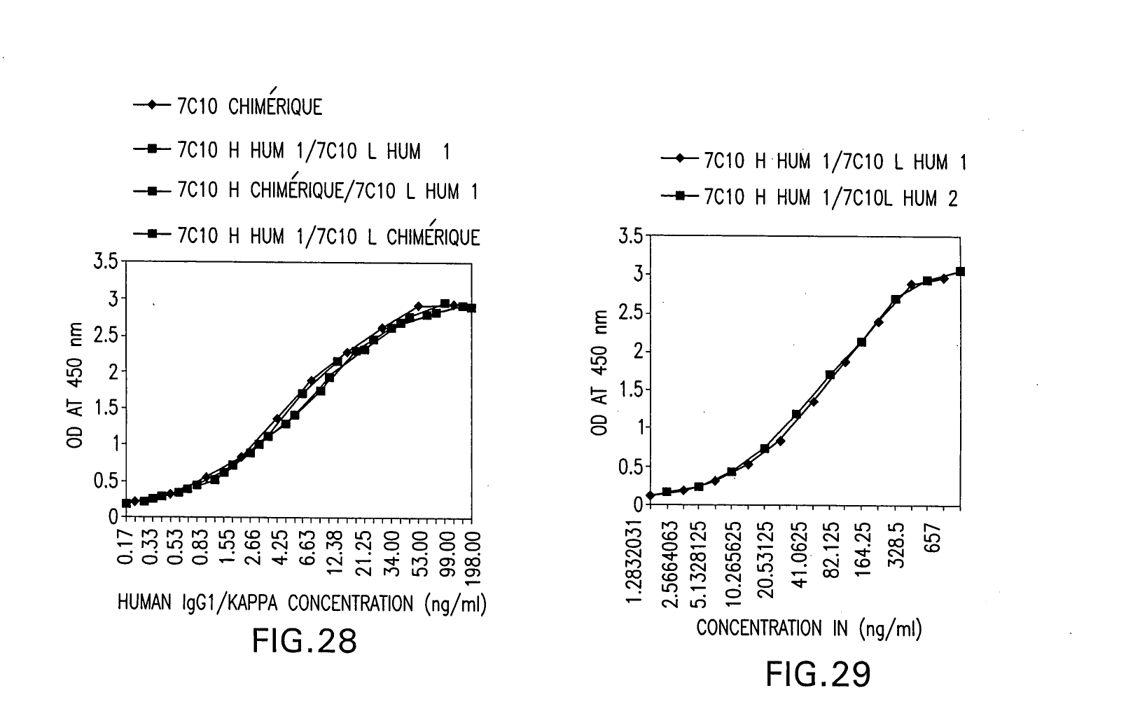

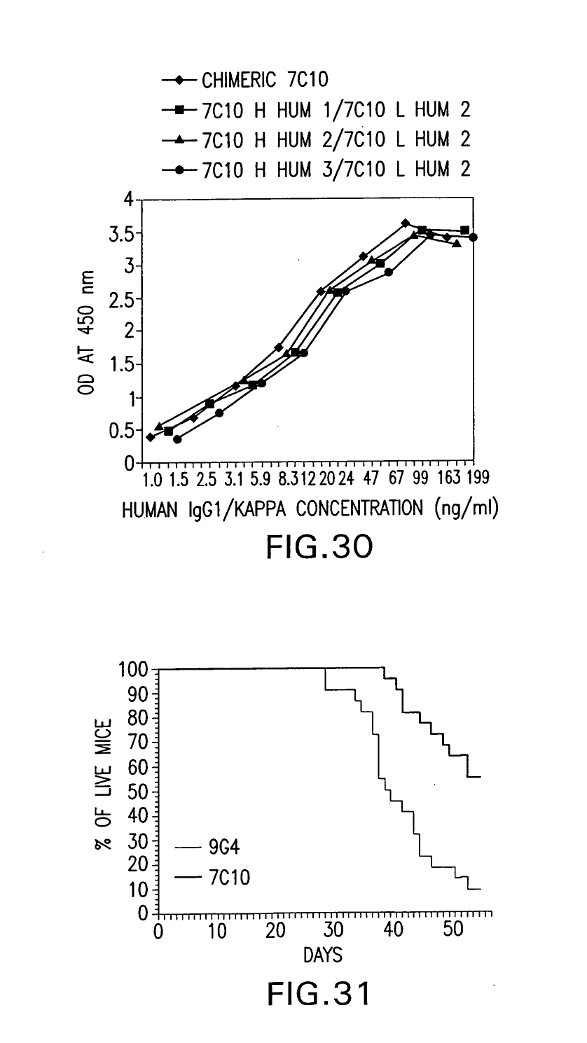

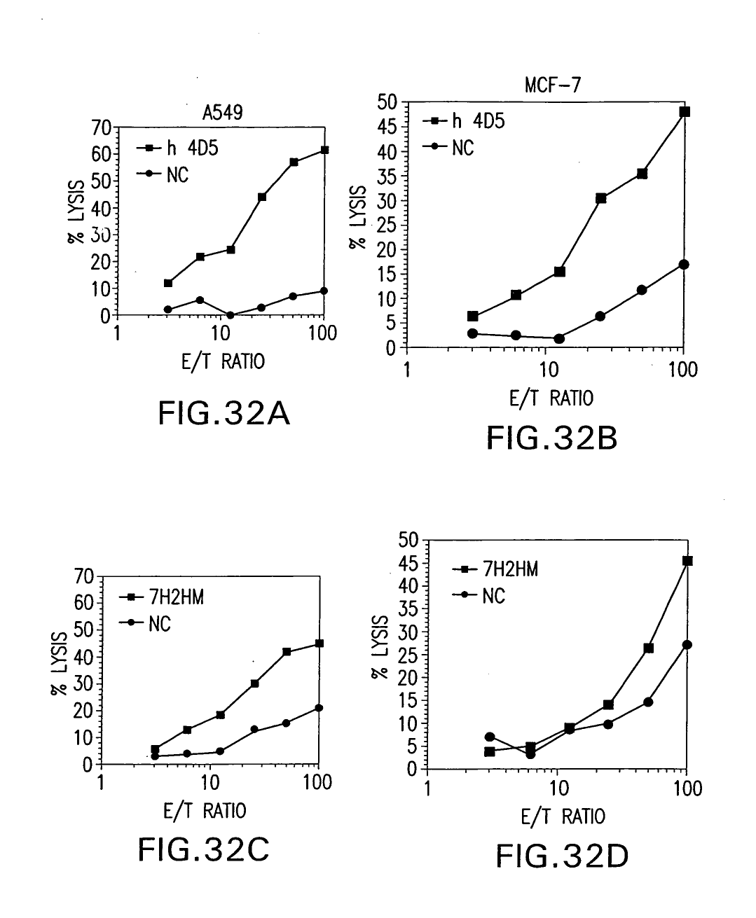

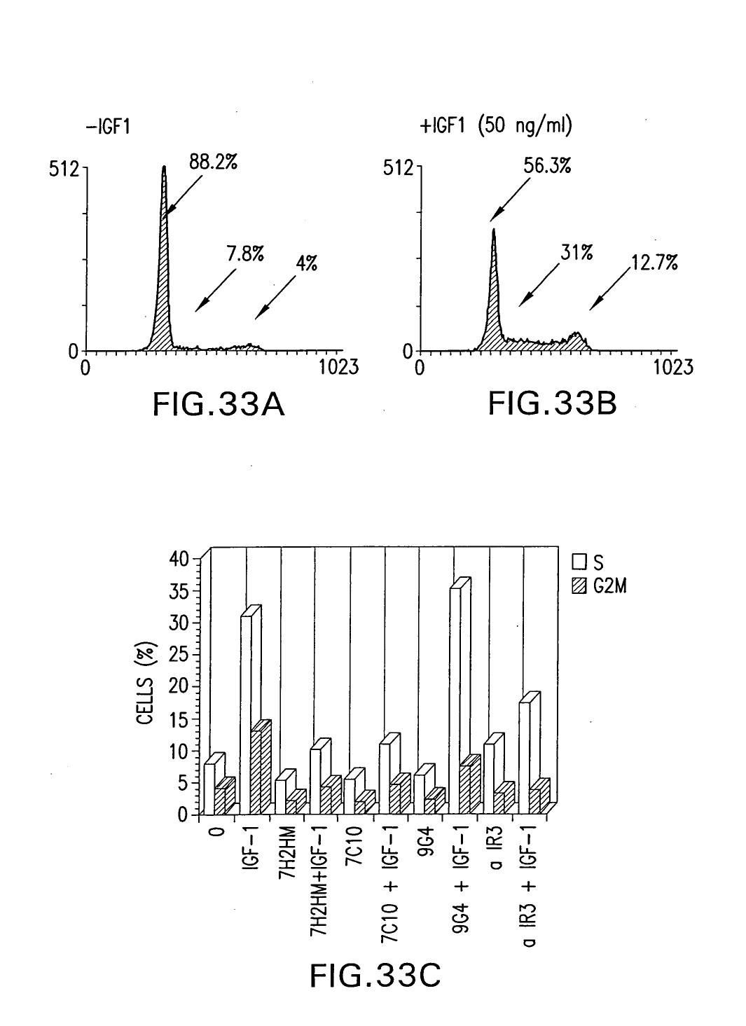

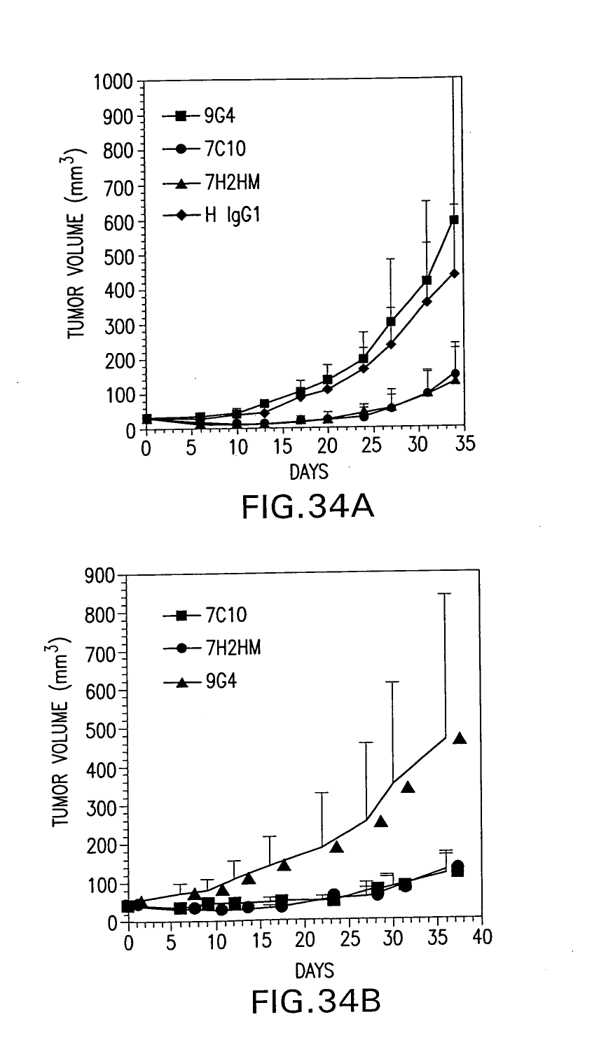

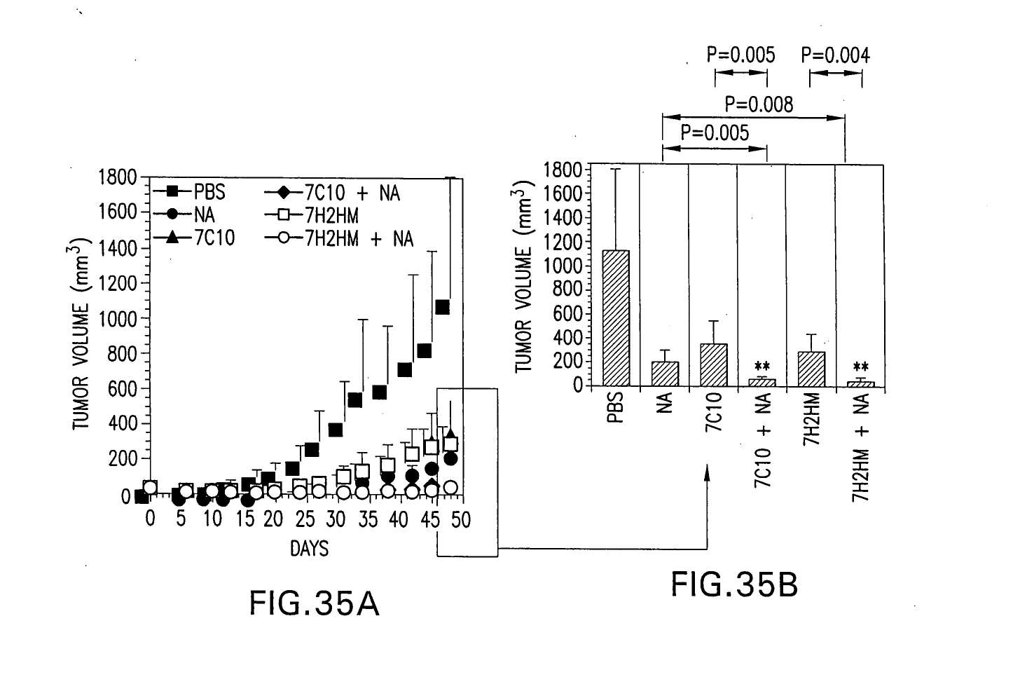

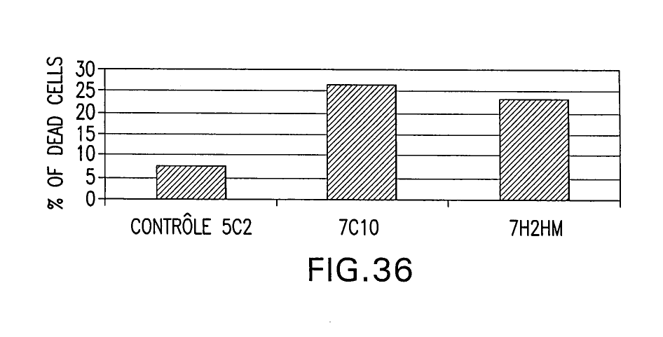

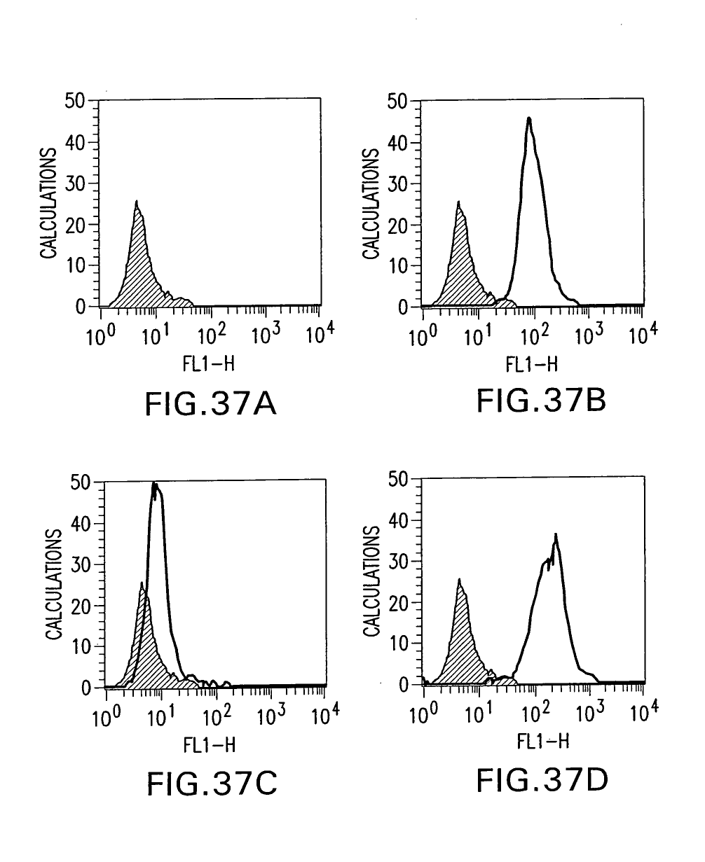

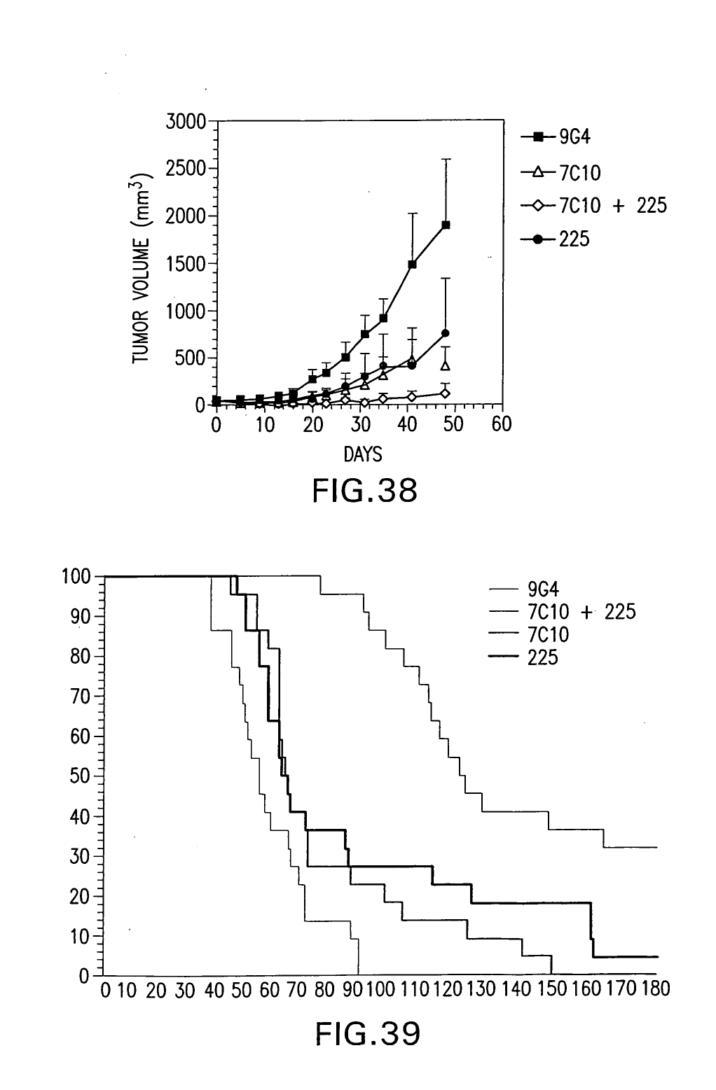

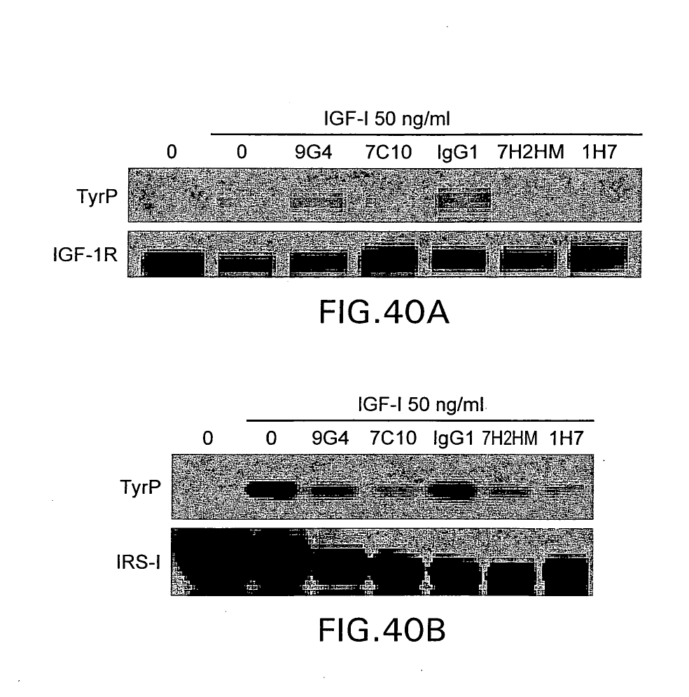

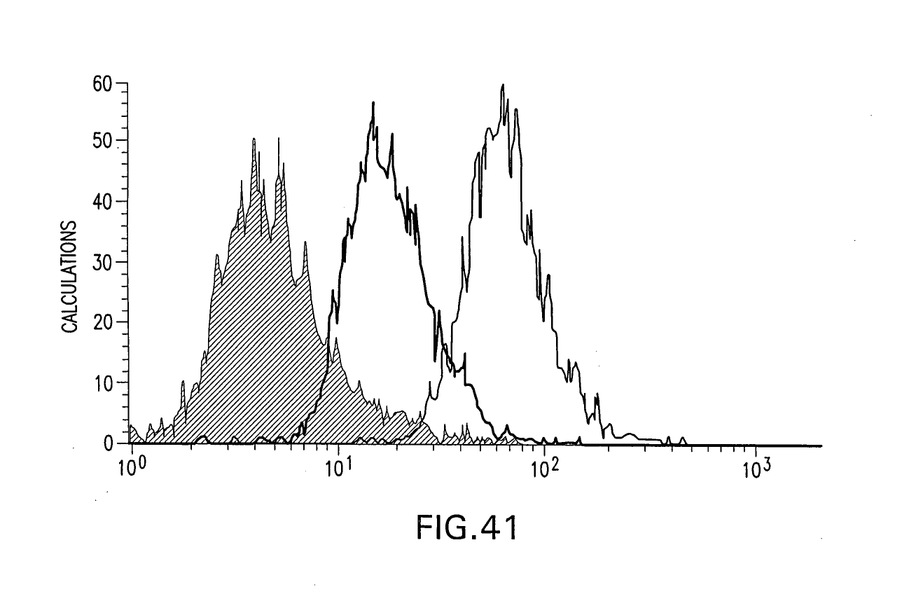

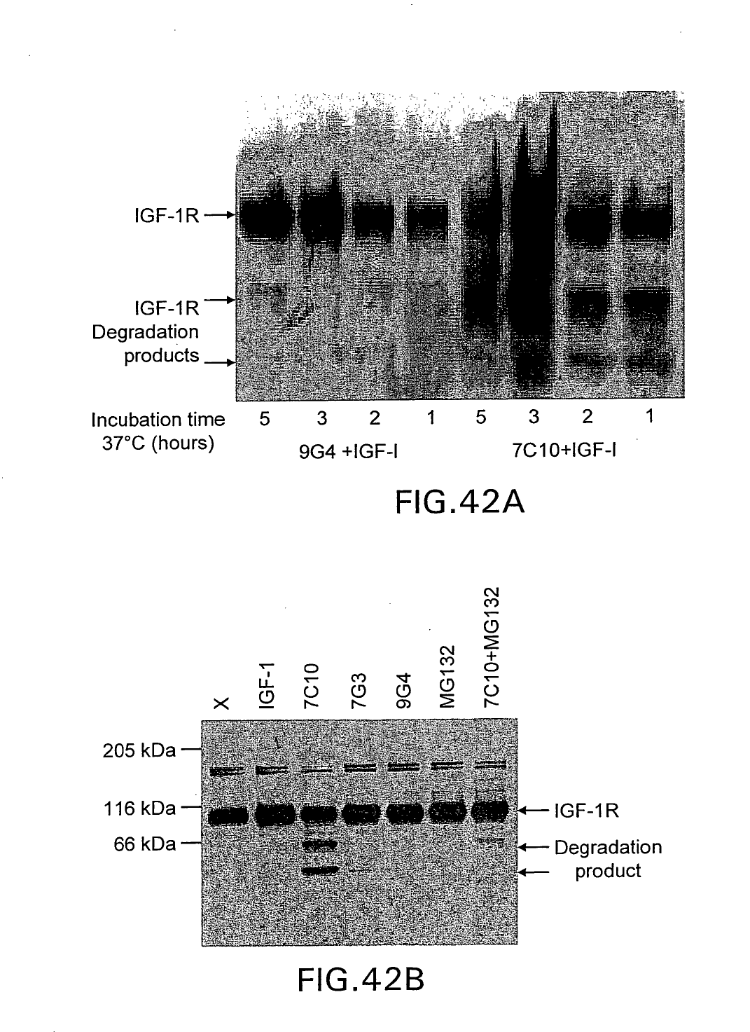

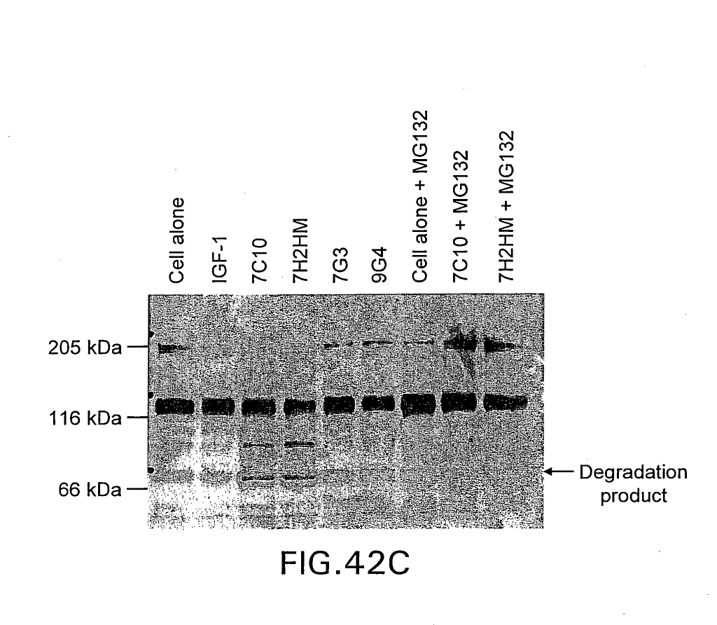

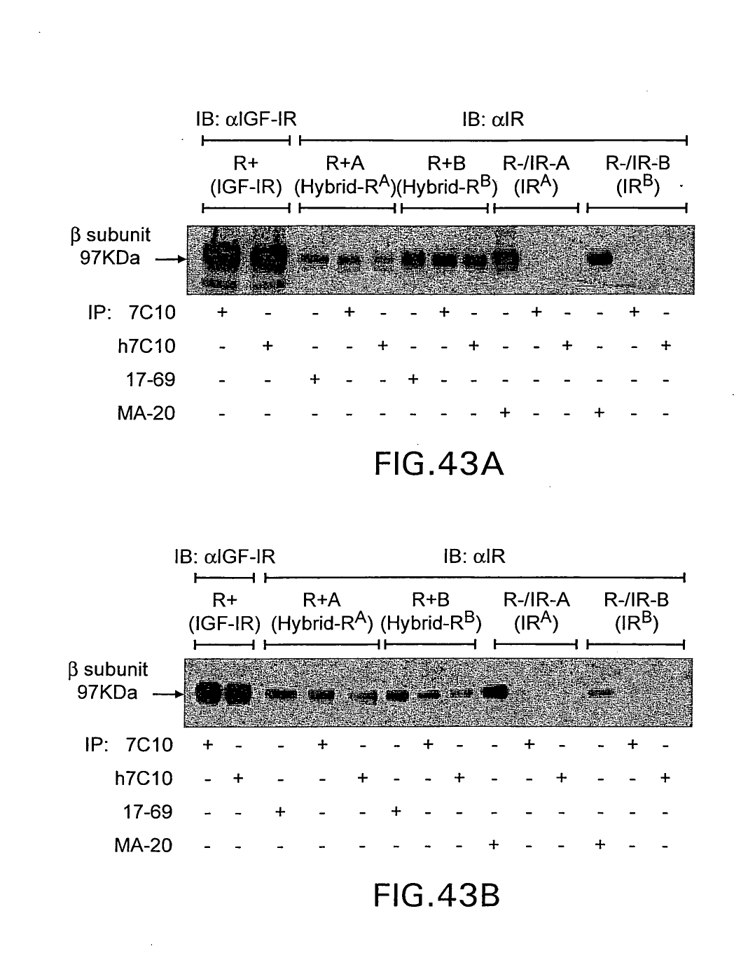

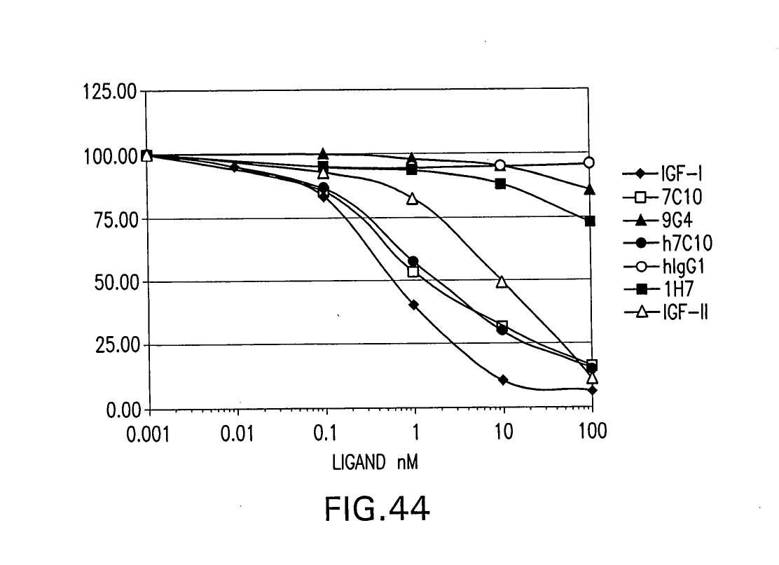

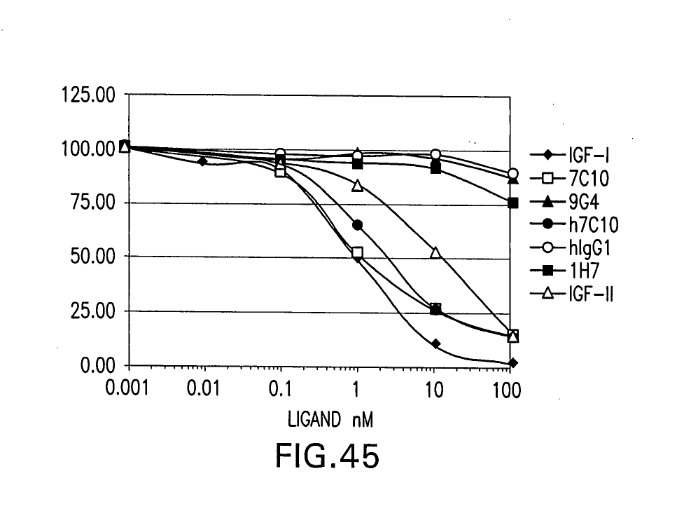

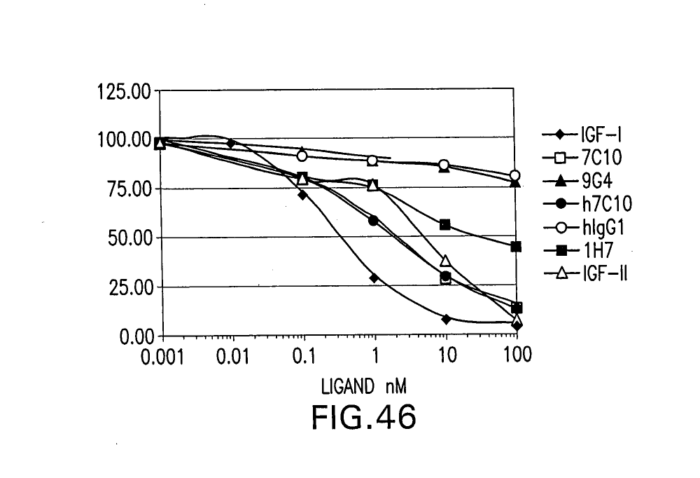

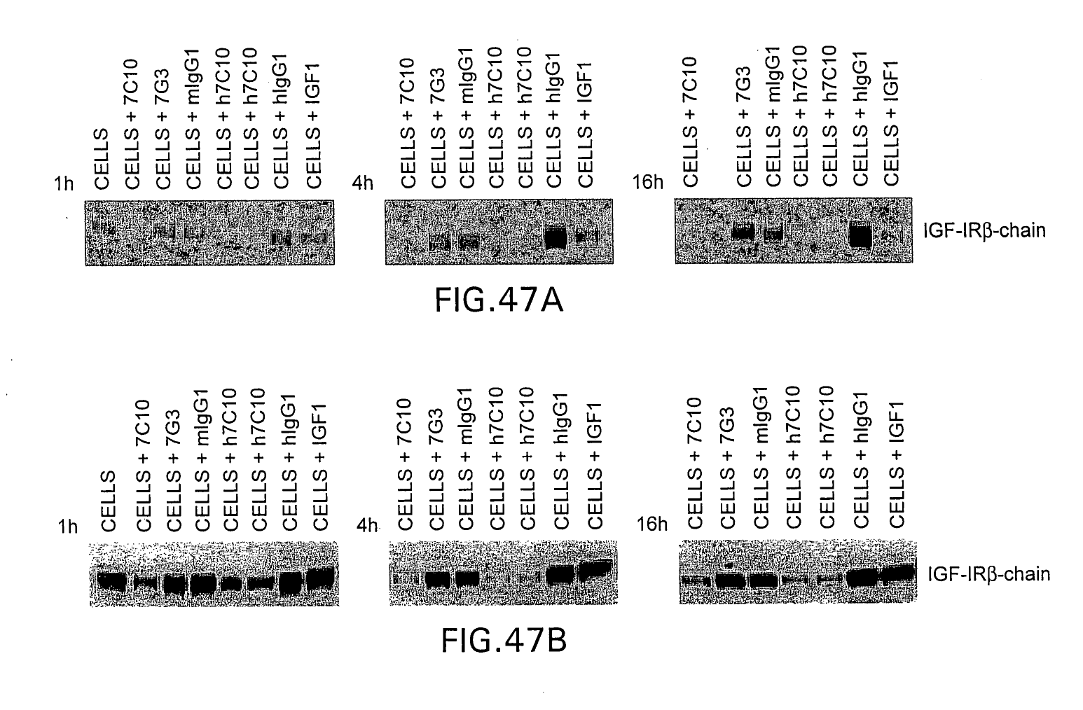

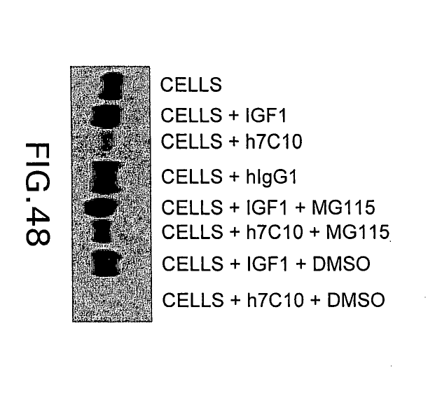

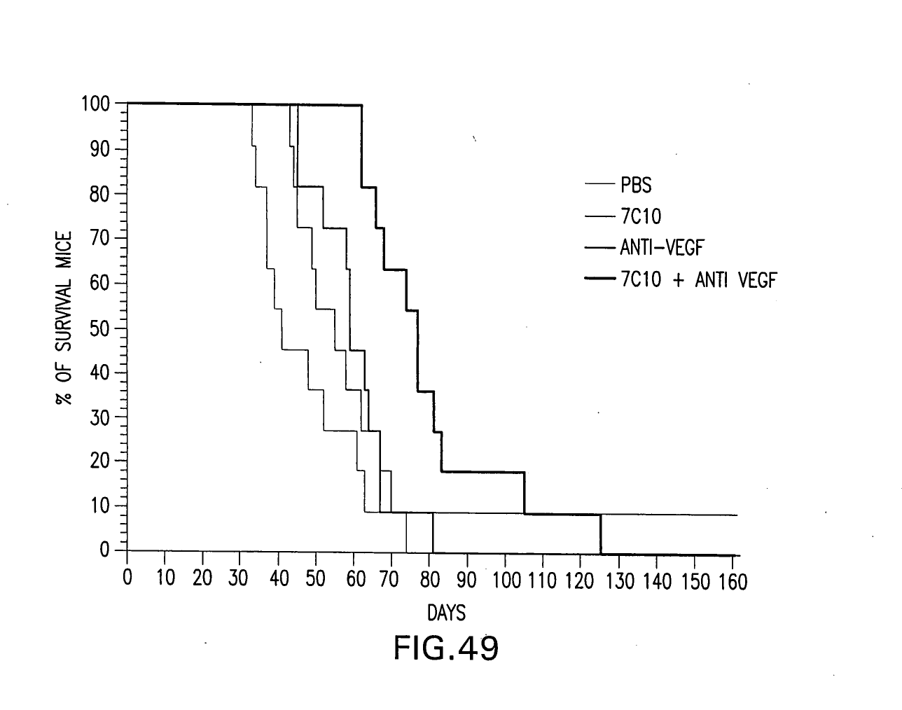

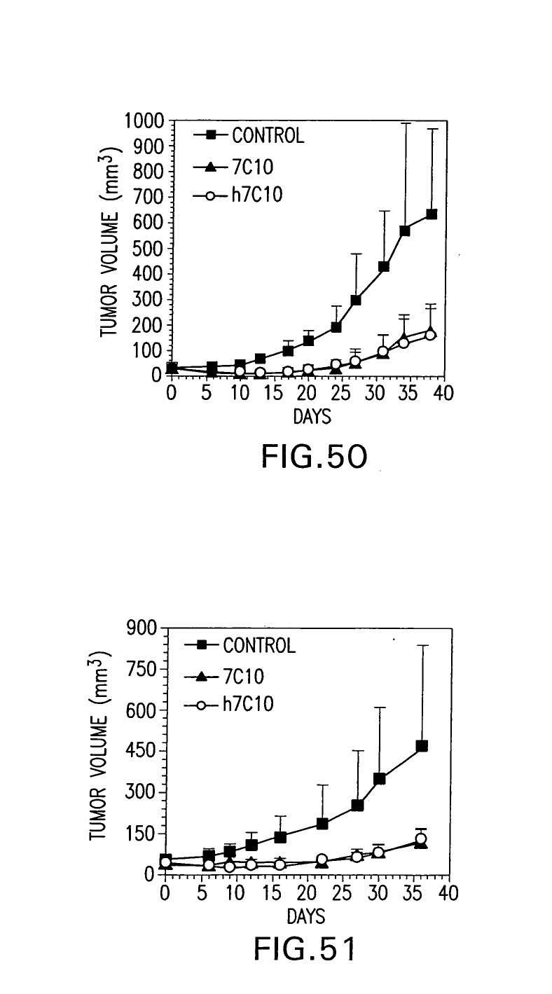

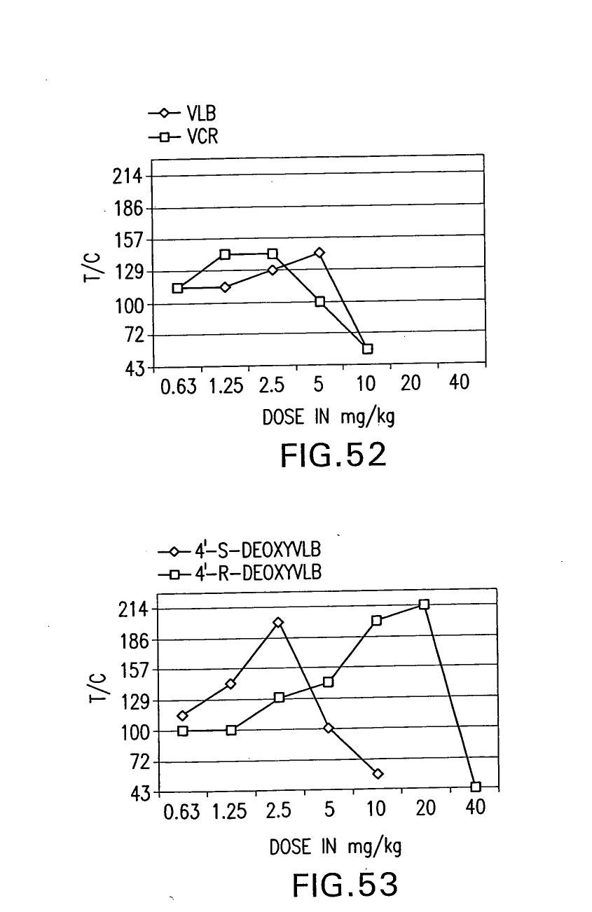

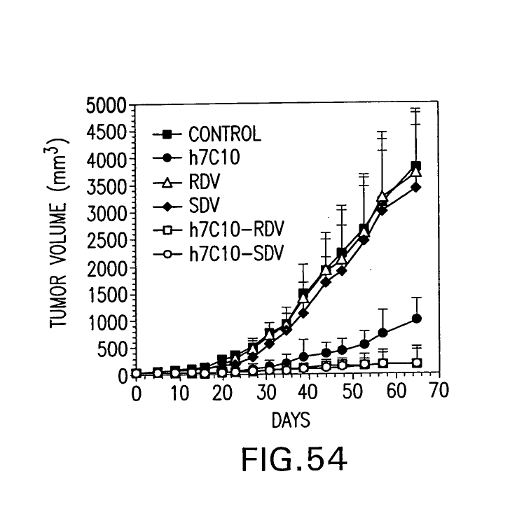

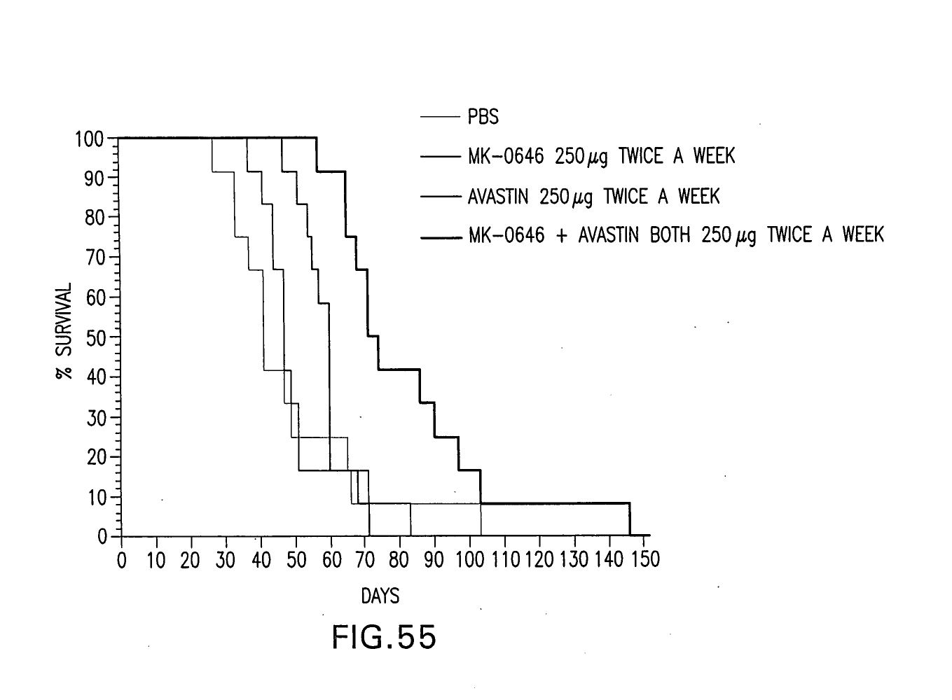

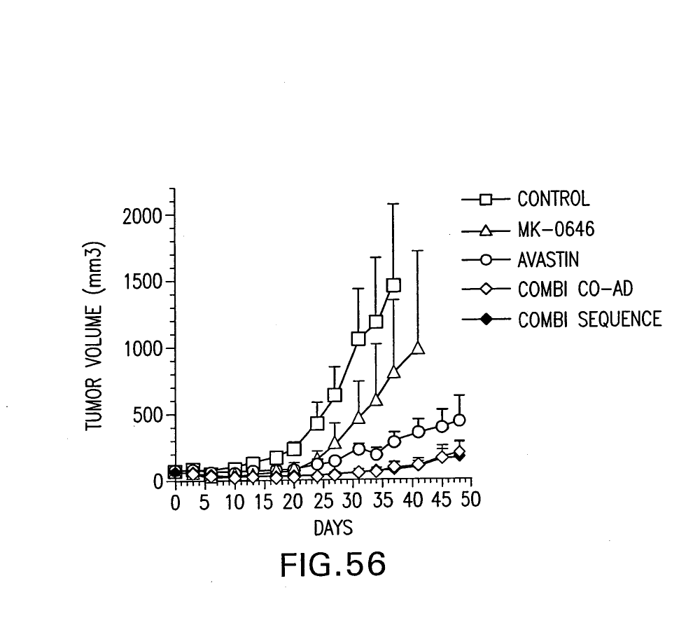

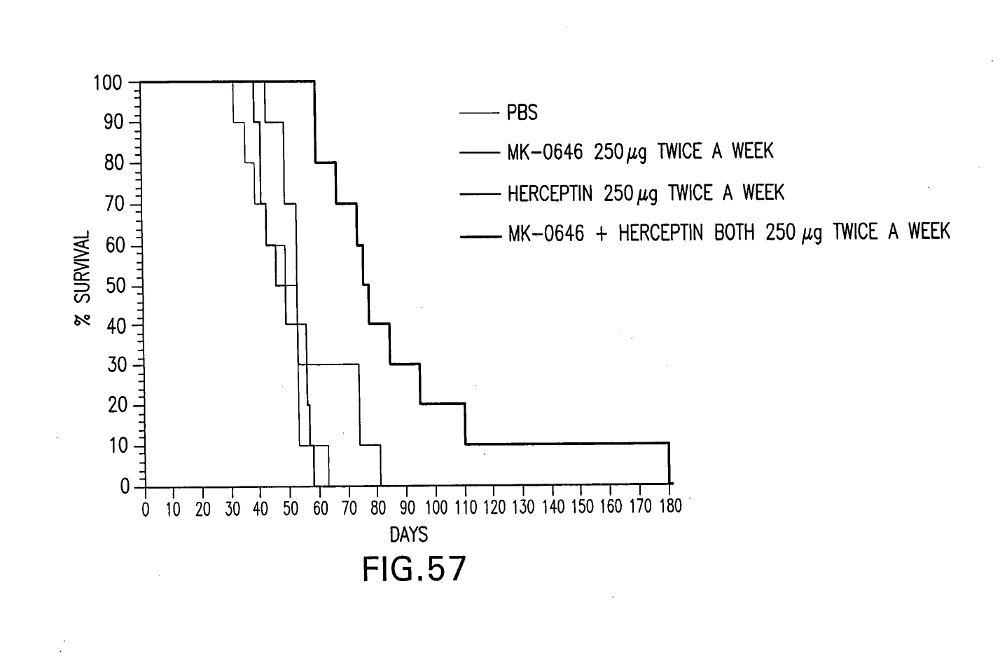

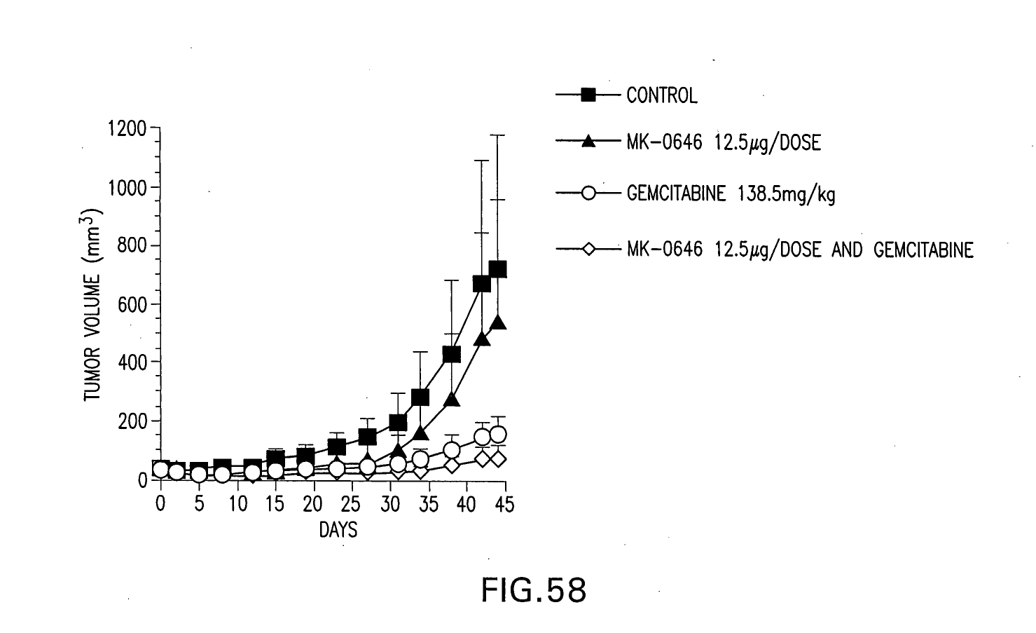

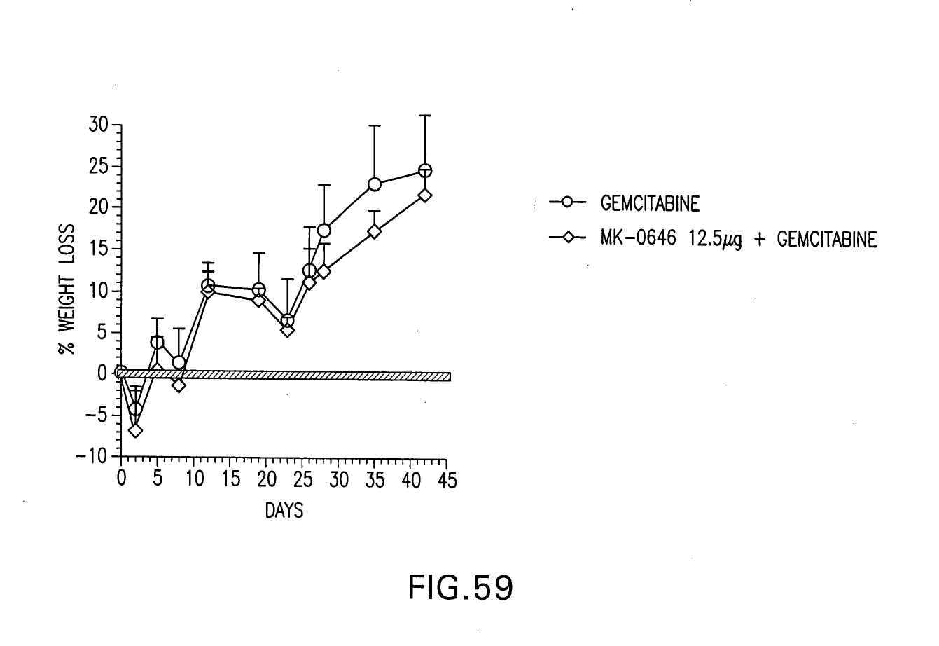

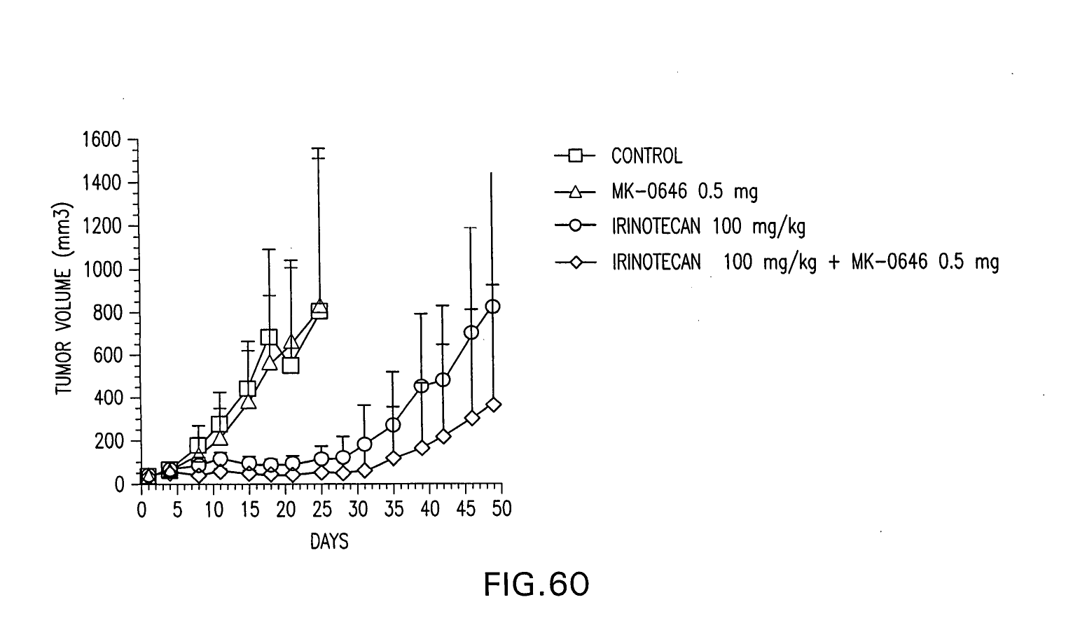

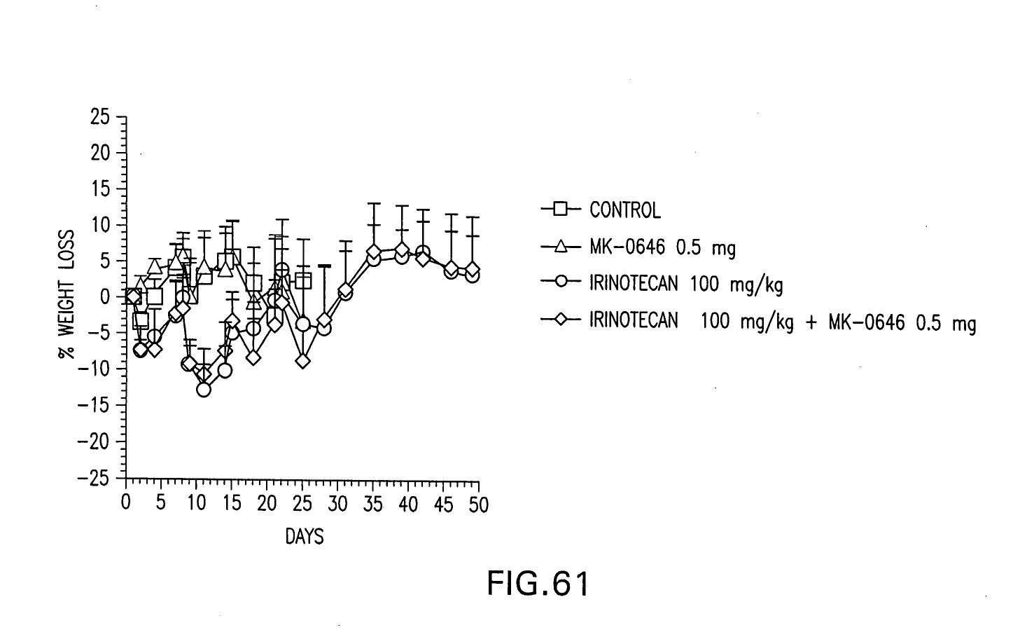

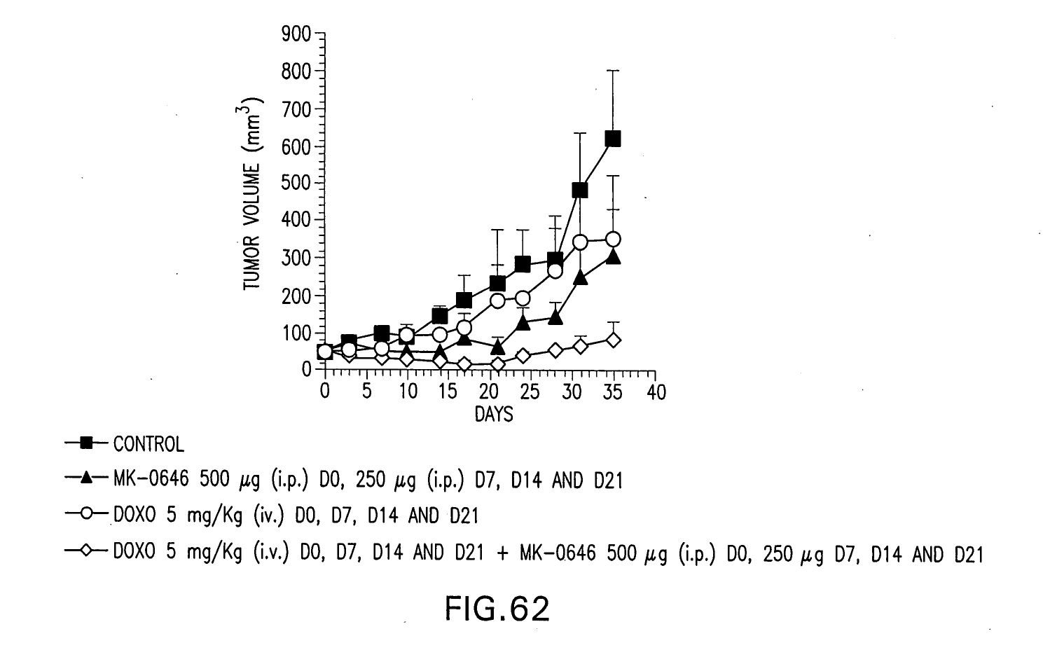

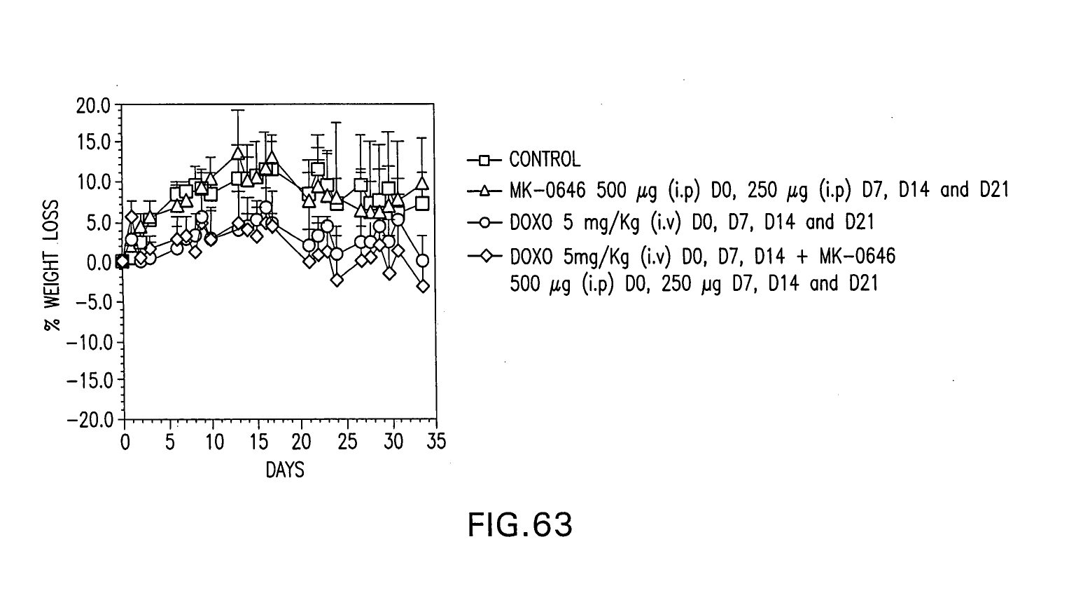

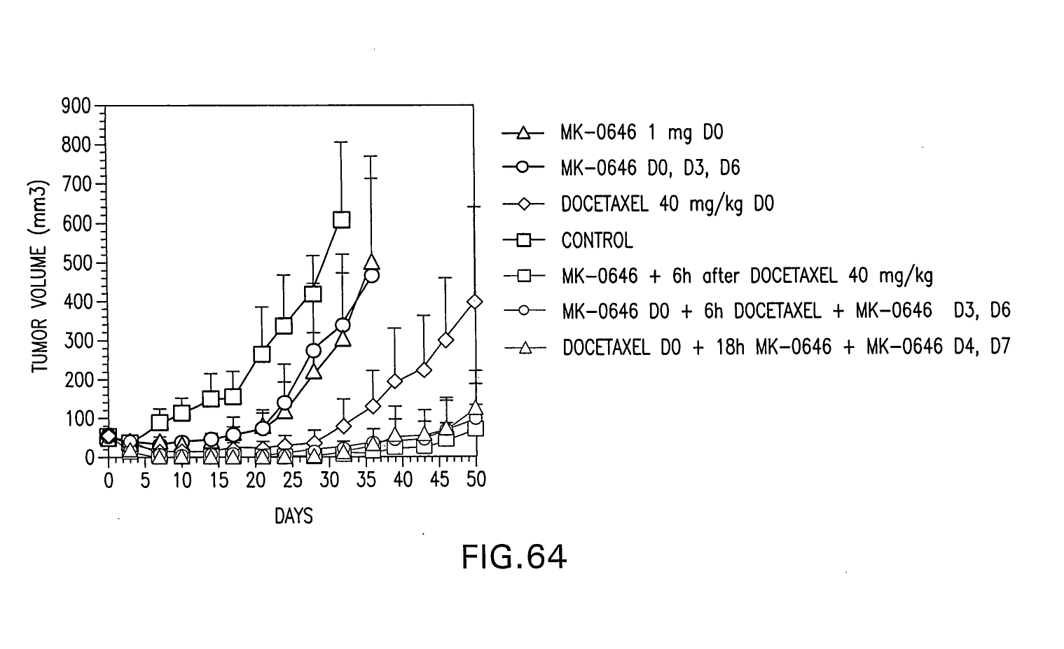

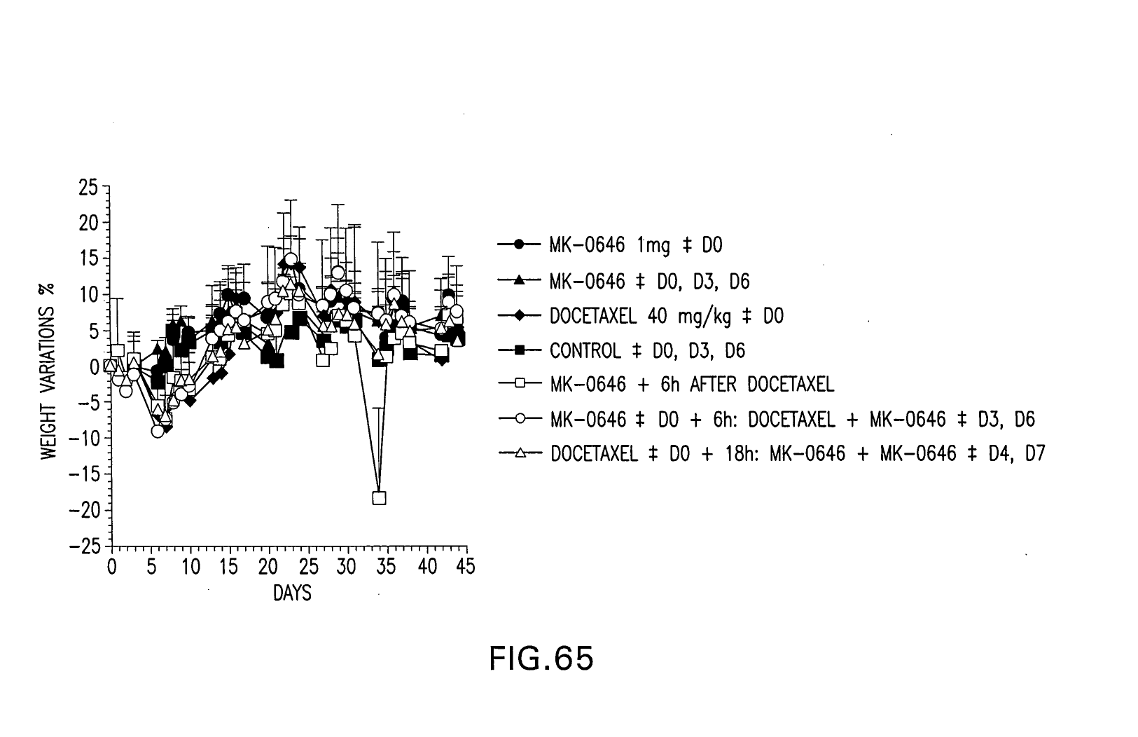

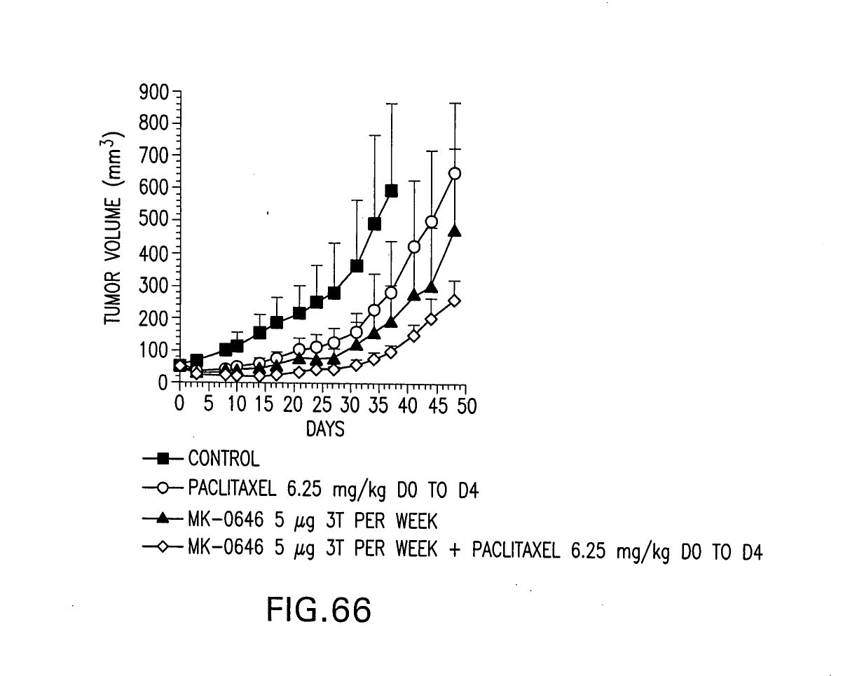

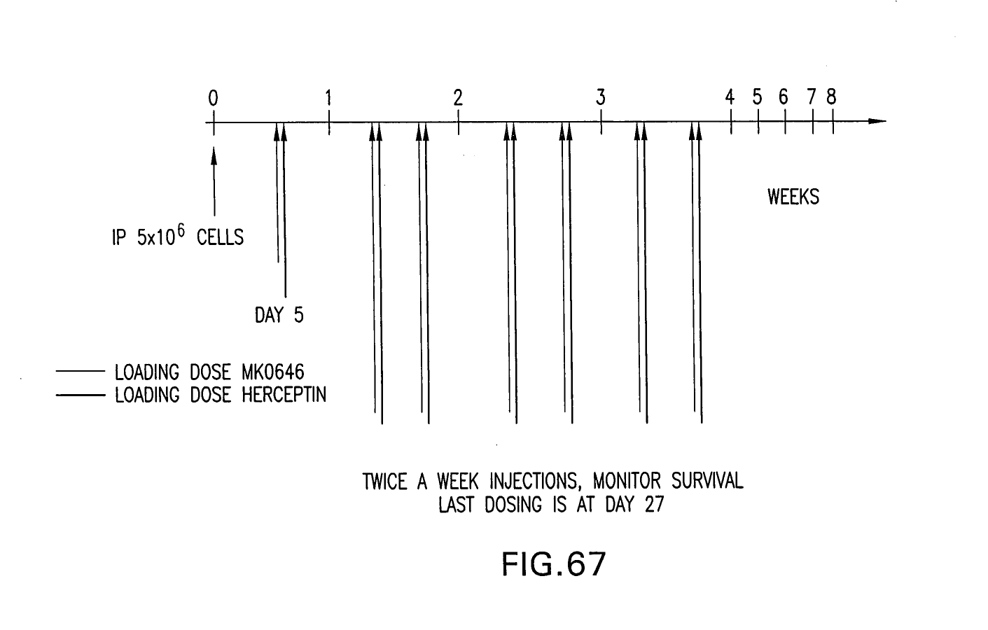

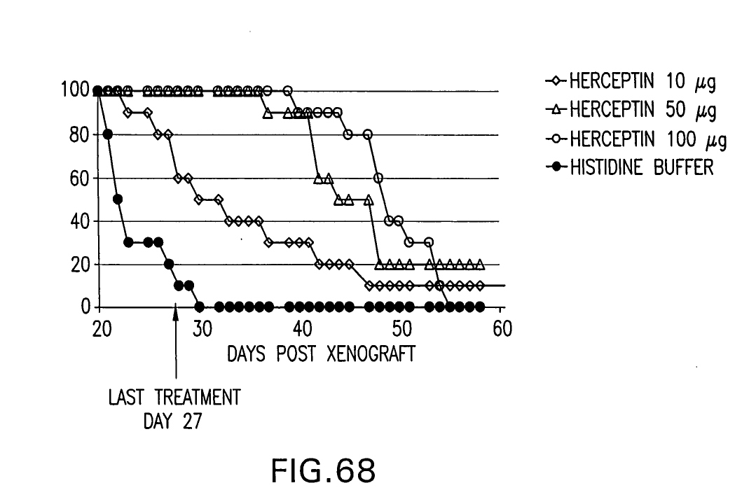

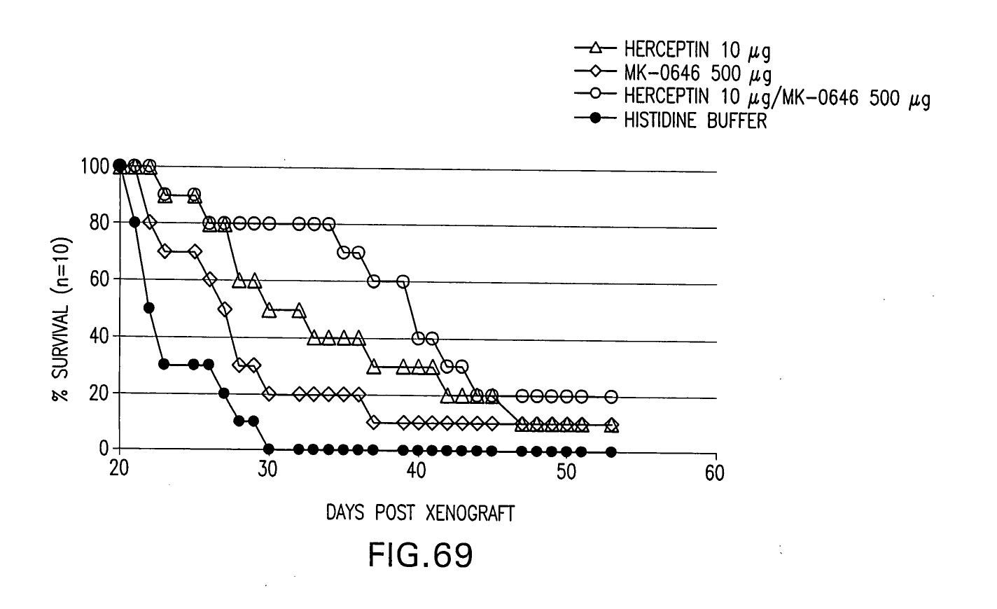

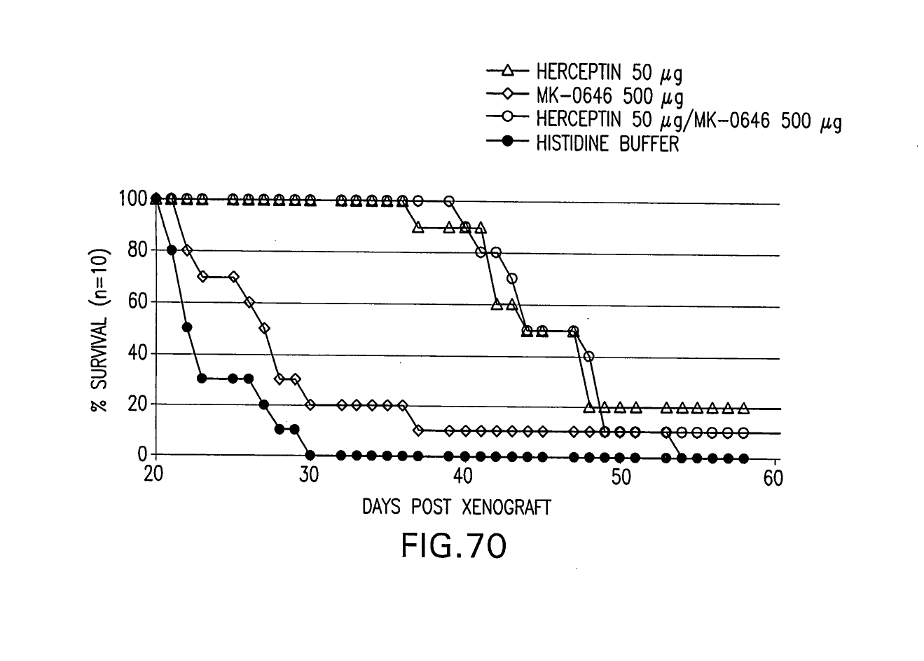

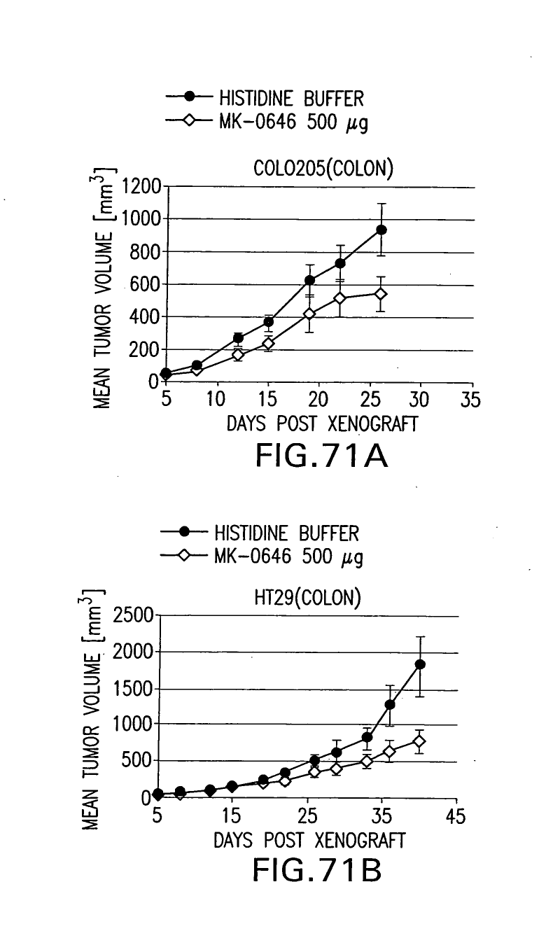

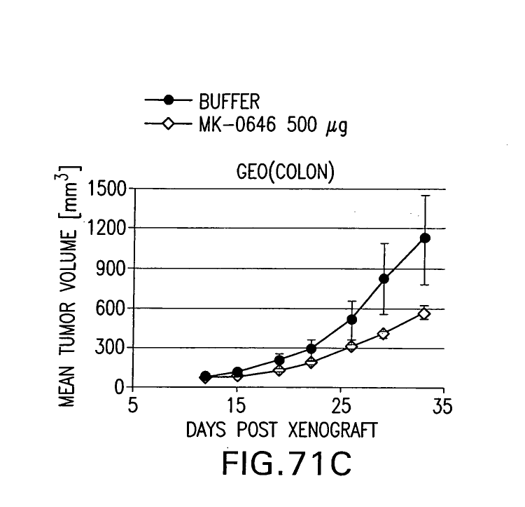

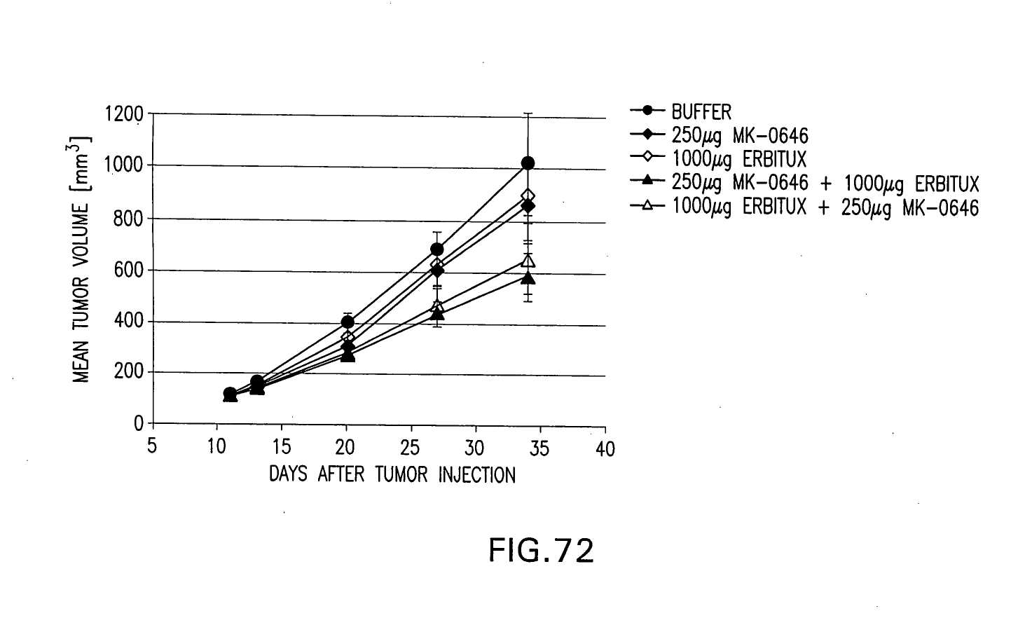

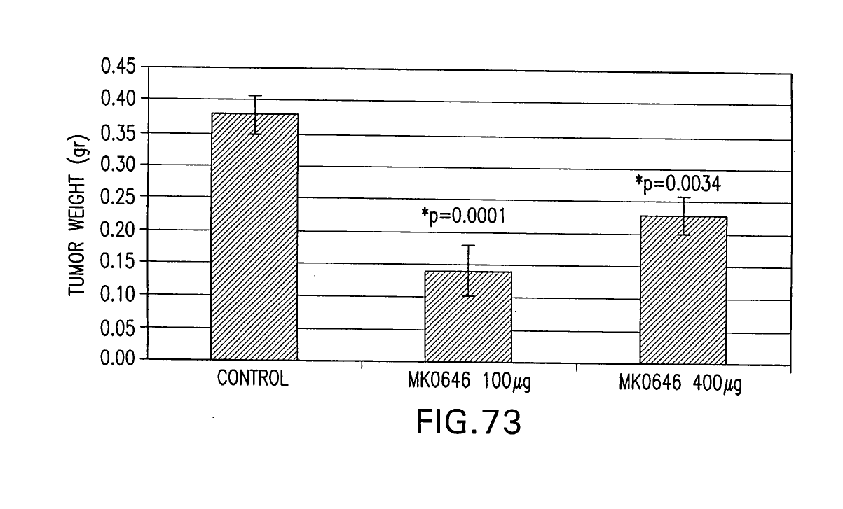

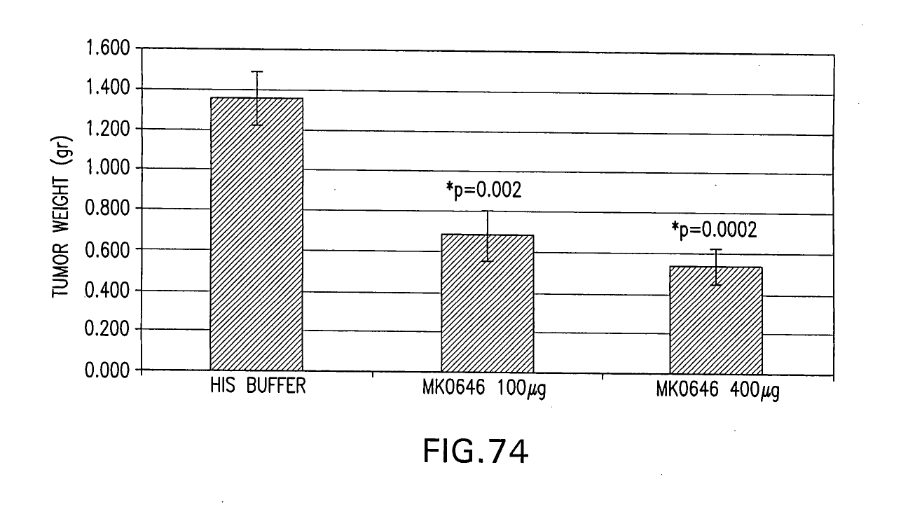

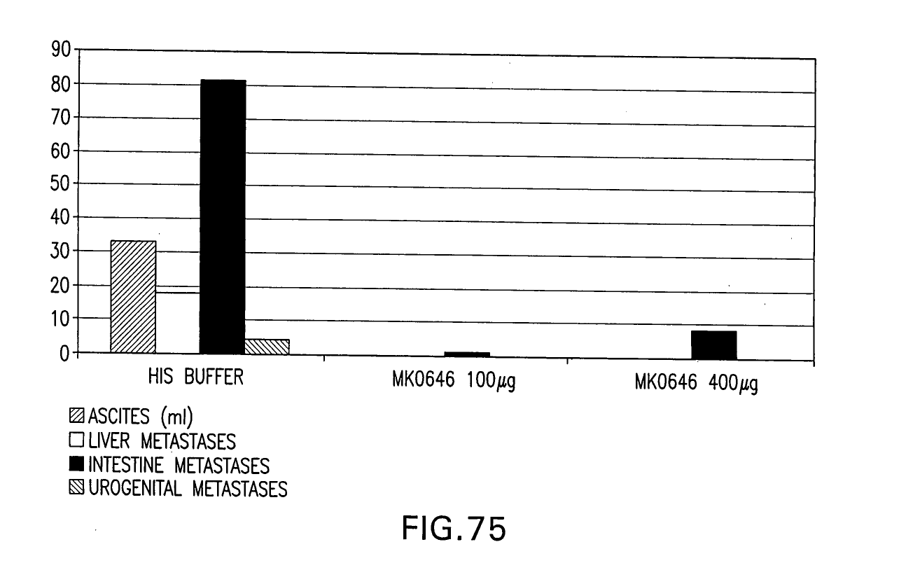

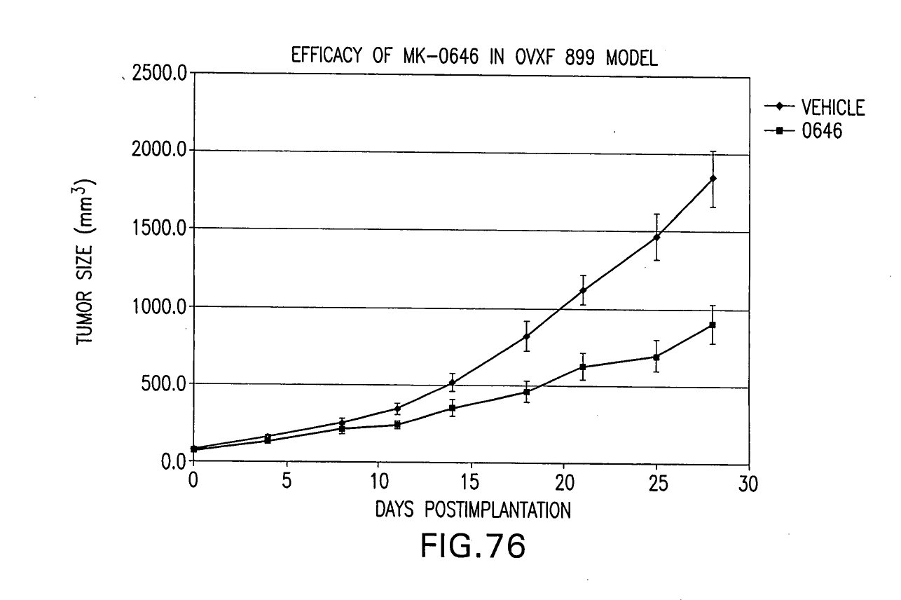

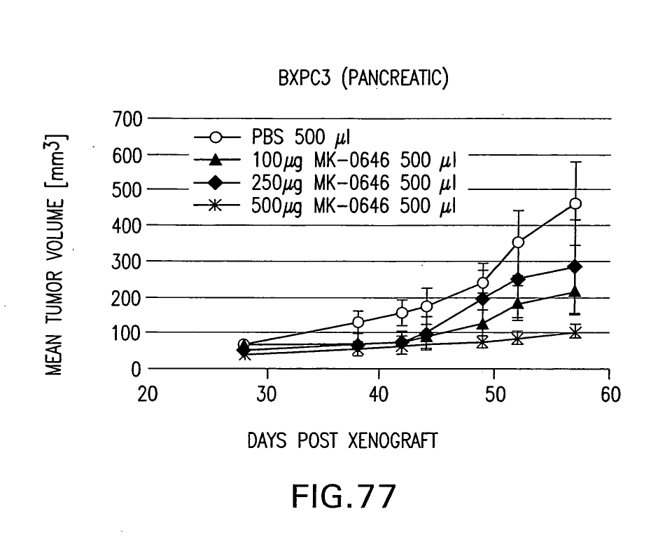

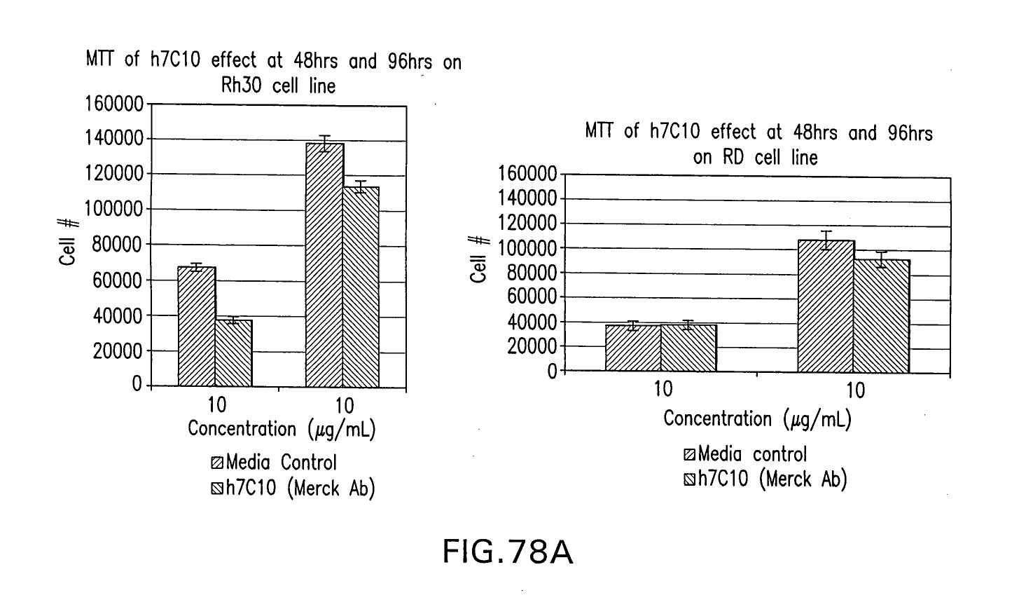

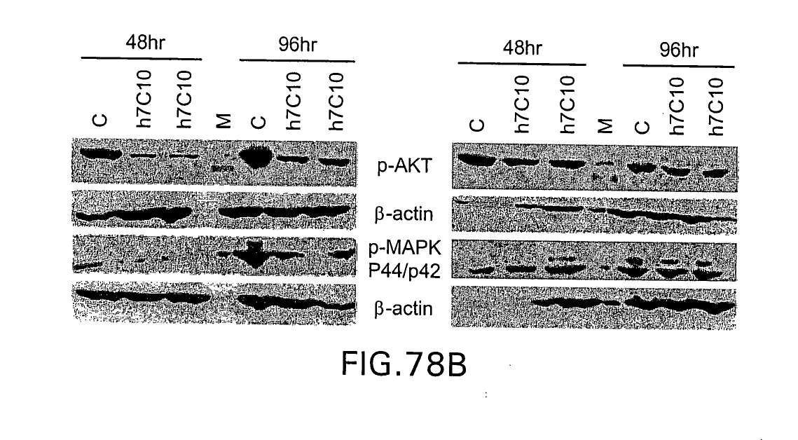

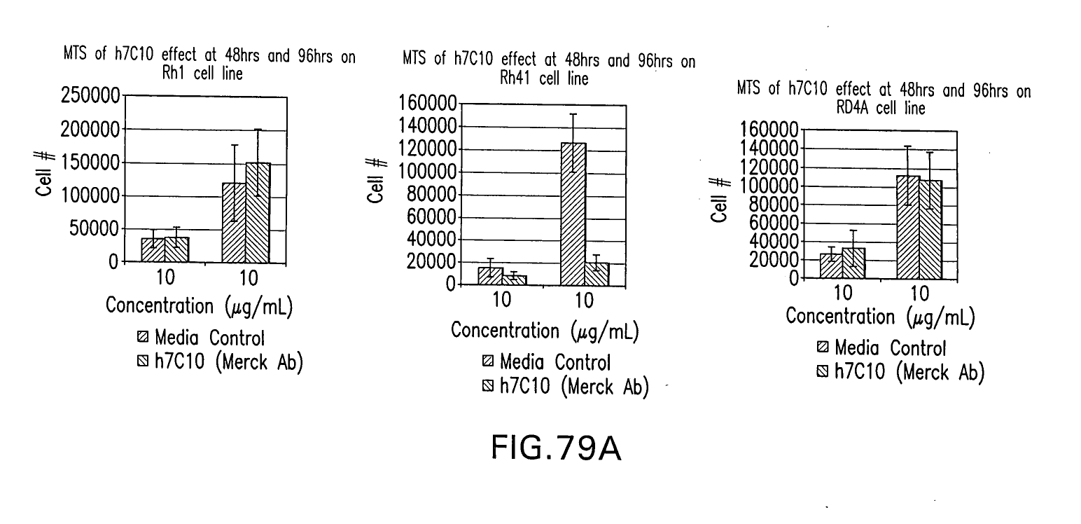

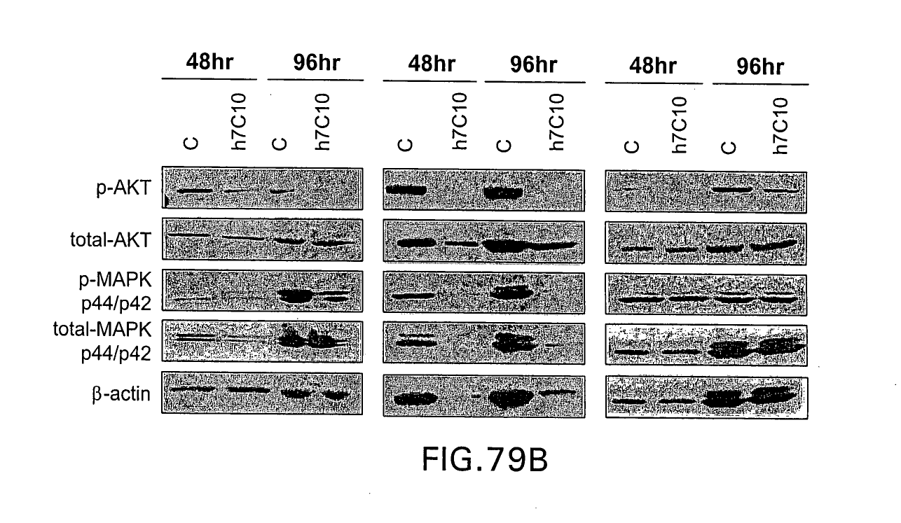

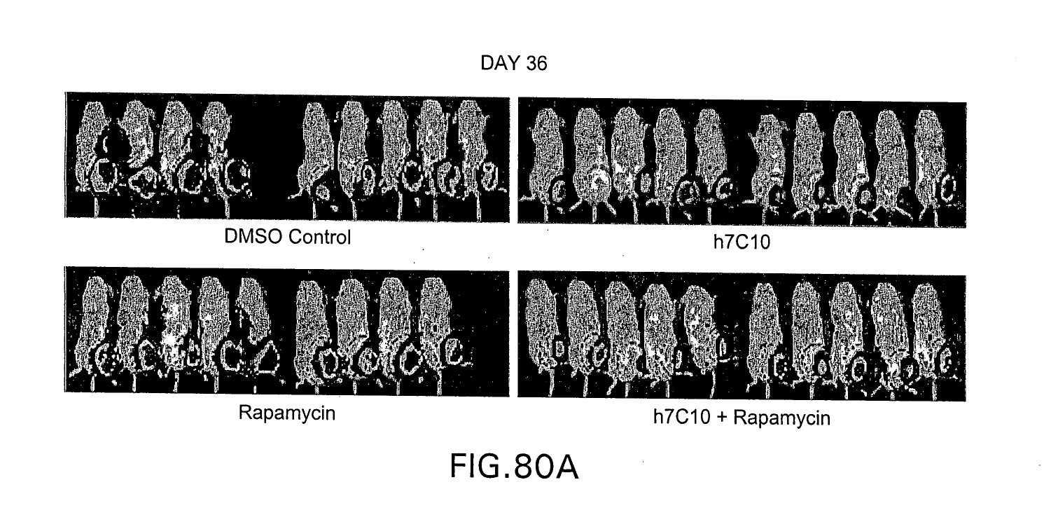

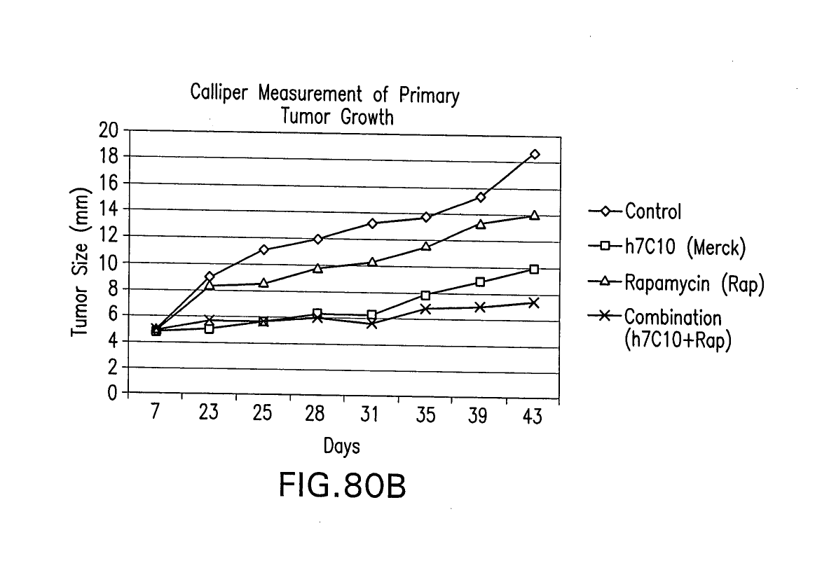

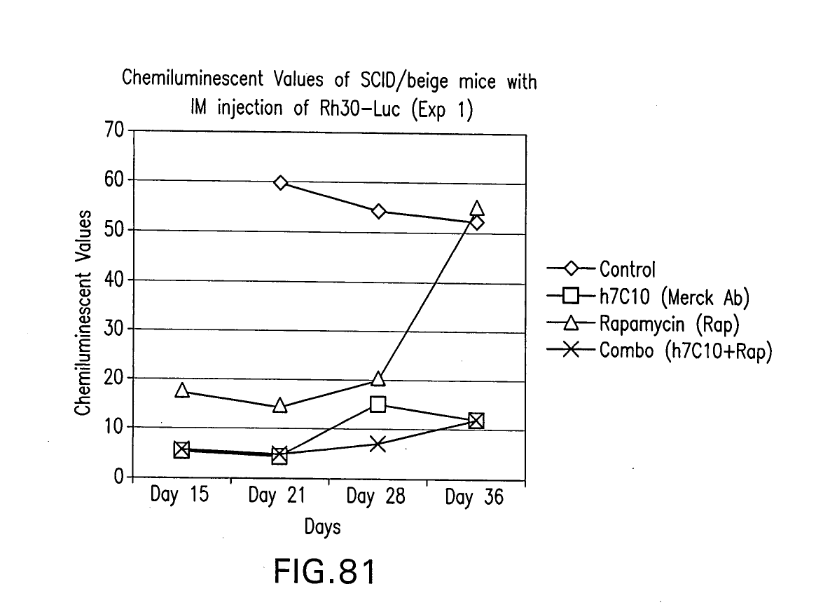

As a preliminary step to humanization by CDR grafting, the amino acid sequence of 7C10 VL was first compared with all the mouse VL sequences present in the databank of Kabat (Internet address: ftp://ftp.ebi.ac.uk/pub/ database/kabat/fasta_format/, last update of data dates from 1999). 7C10 VL has thus been identified as belonging to the subgroup II of the Kappa light chains as defined by Kabat et al. (In Sequences of proteins of immunological interest (5^{th} edn.), NIH publication No. 91-3242, US Department of Health and Human Services, Public Health Service, National Institutes of Health, Bethesda, 1991). The VL regions of monoclonal antibodies of mice having a sequence identity ranging up to 95% have been identified (DRB1-4.3 (SEQ ID No. 55): 95% and C94-5B11′CL (SEQ ID No. 56): 95%, see FIG. 17). In order to attempt to identify the out of the ordinary residues in the 7C10 VL sequence, the amino acid sequence of 7C10 VL (SEQ ID No. 54) was aligned with the consensus sequence of the subgroup II of the mouse kappa chains (SEQ ID No. 57) as defined by Kabat (see FIG. 17).

In the Kabat position number 3, the valine (V) normally present in the subgroup II of the Kappa light chains according to Kabat (71%) is replaced by a leucine (L). A leucine in this position is not rare since it is found, for example, in DRB1-4.3 and C94-5B11′CL. According to the molecular model, this residue does not seem to play a particular role. Consequently, the conservation of this residue in the humanized form will not be envisaged.

In the Kabat position number 7, the threonine (T) normally present in the subgroup II of the Kappa light chains according to Kabat (66%) is replaced by an iso-leucine (I). An isoleucine in this position is relatively rare since it is only found 15 times among all the mouse VL sequences known and never among human VL sequences. The molecular model shows that this residue (17) points toward the surface of the molecule but does not contact the CDRs (the residue of a CDR which is the closest would be the arginine in Kabat position number 42). In addition, it does not seem very probable that this residue 17 directly contacts the antigen. Consequently, the conservation of this residue in the humanized form will not be envisaged, at any rate at first.

In the Kabat position number 77, the arginine (R) normally present in the subgroup II of the Kappa light chains according to Kabat (95.5%) is replaced by a serine (S). A serine in this position is not rare.

b) Comparison of the Amino Acid Sequence of 7C10 VL with all the Known Human VL Sequences

In order to identify the best human candidate for the “CDR grafting”, the Kappa VL region of human origin having the greatest homology possible with 7C10 VL was sought. To this end, the amino acid sequence of mouse kappa 7C10 VL was compared with all the human Kappa VL sequences present in the database of Kabat. Mouse 7C10 VL had the greatest sequence homology with the human kappa VL regions of subgroup II as defined by Kabat et al. (1991). VH regions of monoclonal antibodies of human origin have been identified having a sequence identity ranging up to 75.9% (GM607 (SEQ ID No. 58), see FIG. 18) over the whole of the 112 amino acids composing the variable region. A germinal line of human origin, DPK15/A19 (SEQ ID No. 59), having a sequence identity of 76% (see FIG. 18) was also identified, GM607 (Klobeck et al., 1984). GM607 was therefore chosen as a human sequence receptive of CDRs (according to the definition of Kabat) of mouse 7C10 VL. By comparing the GM607 sequences with that of the consensus sequence of the human subgroup II (SEQ ID No. 60) (FIG. 18), no particular residue in the framework regions (Rch) could be identified, indicating by the same fact that GM607 was a good candidate for CDR grafting.

c) Humanized Versions of 7C10 VL

The following stage in the humanization process consisted in joining the CDRs of mouse 7C10 VL to the framework regions (Rch) of the human light chain selected, GM607 (Klobeck et al., 1984). At this stage of the process, the molecular model of the mouse Fv regions of 7C10 is particularly useful in the choice of the mouse residues to be conserved as being able to play a role either in the maintenance of the tridimensional structure of the molecule (canonical structure of the CDRs, VH/VL interface, etc.) or in the binding to the antigen. In the Rchs, each difference between the mouse (7C10 VL) and human (GM607) amino acids was examined scrupulously (see Table 5). In addition, the particular residues in the mouse sequence 7C10 VL which were identified (see example 12.a) were taken into account if needed.

In the first version humanized by “CDR grafting” of 7C10 VL, human 1, a single change in the framework regions (Rch) of GM607 was carried out. This change concerns the residue 2 (nomenclature of Kabat) situated in Rch 1. This residue enters in effect into the composition of the canonical structure of the CDR 1 of 7C10 VL and could therefore be critical for maintaining this loop in its good conformation. The valine present in this position in the mouse 7C10 VL sequence is thus conserved in this same position in the humanized form (see Table 5 and FIG. 19 for the amino acid sequence (SEQ ID No. 61) and FIG. 20 for the DNA sequence (SEQ ID Nos. 62 and 64) and the amino acid sequence comprising the peptide signal (SEQ ID No. 63).

In the second version humanized by “CDR grafting” of 7C10 VL, human 2, no change in the Rchs of the human light chain GM607 has been made. All the residues of the Rchs are thus of human origin including the residue 2 which has therefore been mutated in order to replace the valine present in mouse 7C10 VL by an isoleucine found in this same position in the human light chain GM607 (see Table 5 and FIG. 19 for the amino acid sequence (SEQ ID No. 65) and FIG. 21 for the DNA sequence (SEQ ID Nos. 66 and 68) and the amino acid sequence comprising the peptide signal (SEQ ID No. 67)). This human form 2 is therefore totally humanized (apart from, of course, CDRs themselves) since all the residues of the Rchs are those of the light chain of human origin, GM607.

TABLE 5
Alignment of the amino acid sequences leading to
the design of the remodeled human 7C10 V_{L} regions

| | | | | Human | | | | |
|---|---|---|---|---|---|---|---|---|
| | | | Mouse | germinal | | | | |
| | | | light | line | | Remodeled | Remodeled | |
| | | FR or | chain | DPK15/ | GM | human | human | |
| Kabat | # | CDR | 7C10 | A19 | 607 | 7C10 1 | 7C10 2 | Comments |
| 1 | 1 | FR1 | D | D | D | D | D | |
| 2 | 2 | \| | V* | I* | I* | V* | I* | Cano L1 |
| | | | | | | | | 4(16) |
| | | | | | | | | Vernier |
| | | | | | | | | zone |
| 3 | 3 | \| | L | V | V | V | V | |
| 4 | 4 | \| | M | M | M | M | M | Vernier |
| | | | | | | | | zone |
| 5 | 5 | \| | T | T | T | T | T | |
| 6 | 6 | \| | Q | Q | Q | Q | Q | |
| 7 | 7 | \| | I | S | S | S | S | |
| 8 | 8 | \| | P | P | P | P | P | |
| 9 | 9 | \| | L | L | L | L | L | |
| 10 | 10 | \| | S | S | S | S | S | |
| 11 | 11 | \| | L | L | L | L | L | |
| 12 | 12 | \| | P | P | P | P | P | |
| 13 | 13 | \| | V | V | V | V | V | |
| 14 | 14 | \| | S | T | T | T | T | |
| 15 | 15 | \| | L | P | P | P | P | |
| 16 | 16 | \| | G | G | G | G | G | |
| 17 | 17 | \| | D | E | E | E | E | |
| 18 | 18 | \| | Q | P | P | P | P | |
| 19 | 19 | \| | A | A | A | A | A | |
| 20 | 20 | \| | S | S | S | S | S | |
| 21 | 21 | \| | I | I | I | I | I | |
| 22 | 22 | \| | S | S | S | S | S | |
| 23 | 23 | FR1 | C | C | C | C | C | |
| 24 | 24 | CDR1 | R | R | R | R | R | |
| 25 | 25 | \| | S* | S* | S* | S* | S* | Cano L1 |
| | | | | | | | | 4(16) |
| 26 | 26 | \| | S | S | S | S | S | |
| 27 | 27 | \| | Q | Q | Q | Q | Q | |
| 27A | 28 | \| | S | S | S | S | S | |
| 27B | 29 | \| | I* | L* | L* | i* | i* | Cano L1 |
| | | | | | | | | 4(16) |
| 27C | 30 | \| | V | L | L | i | I | |
| 27D | 31 | \| | H | H | H | H | H | |
| 27E | 32 | \| | S | S | S | S | S | |
| 28 | 33 | \| | N | N | N | N | N | |
| 29 | 34 | \| | G | G | G | G | G | |
| 30 | 35 | \| | N | Y | Y | n | N | |
| 31 | 36 | \| | T | N | N | t | T | |
| 32 | 37 | \| | Y | Y | Y | Y | Y | |
| 33 | 38 | \| | L* | L* | L* | L* | L* | Cano L1 |
| | | | | | | | | 4(16) |
| 34 | 39 | CDR1 | Q | D | D | q | Q | |
| 35 | 40 | FR2 | W | W | W | W | W | Vernier |
| | | | | | | | | zone |
| 36 | 41 | \| | Y | Y | Y | Y | Y | VH/VL |
| | | | | | | | | inter |
| | | | | | | | | Vernier |
| | | | | | | | | zone |
| 37 | 42 | \| | L | L | L | L | L | |
| 38 | 43 | \| | Q | Q | Q | Q | Q | VL/VH |
| | | | | | | | | inter |
| 39 | 44 | \| | K | K | K | K | K | |
| 40 | 45 | \| | P | P | P | P | P | |
| 41 | 46 | \| | G | G | G | G | G | |
| 42 | 47 | \| | Q | Q | Q | Q | Q | |
| 43 | 48 | \| | S | S | S | S | S | |
| 44 | 49 | \| | P | P | P | P | P | VL/VH |
| | | | | | | | | inter (+) |
| 45 | 50 | \| | K | Q | Q | Q | Q | |
| 46 | 51 | \| | L | L | L | L | L | VL/VH |
| | | | | | | | | inter |
| | | | | | | | | Vernier |
| | | | | | | | | zone |
| 47 | 52 | \| | L | L | L | L | L | Vernier |
| | | | | | | | | zone |
| 48 | 53 | \| | I | I | I | I* | I* | Cano L2 |
| | | | | | | | | 1(7) |
| | | | | | | | | Vernier |
| | | | | | | | | zone |
| 49 | 54 | FR2 | Y | Y | Y | Y | Y | Vernier |
| | | | | | | | | zone |
| 50 | 55 | CDR2 | K | L | L | k | K | |
| 51 | 56 | \| | v* | G* | G* | v* | v* | Cano L2 |
| | | | | | | | | 1(7) |
| 52 | 57 | \| | S* | S* | S* | S* | S* | Cano L2 |
| | | | | | | | | 1(7) |
| 53 | 58 | \| | N | N | N | N | N | |
| 54 | 59 | \| | R | R | R | R | R | |
| 55 | 60 | \| | L | A | A | l | L | |
| 56 | 61 | CDR2 | Y | S | S | y | Y | |
| 57 | 62 | FR3 | G | G | G | G | G | |
| 58 | 63 | \| | V | V | V | V | V | |
| 59 | 64 | \| | P | P | P | P | P | |
| 60 | 65 | \| | D | D | D | D | D | |
| 61 | 66 | \| | R | R | R | R | R | |
| 62 | 67 | \| | F | F | F | F | F | |
| 63 | 68 | \| | S | S | S | S | S | |
| 64 | 69 | \| | G* | G* | G* | G* | G* | Cano L2 |
| | | | | | | | | 1(7) |
| | | | | | | | | Vernier |
| | | | | | | | | zone |
| 65 | 70 | \| | S | S | S | S | S | |
| 66 | 71 | \| | G | G | G | G | G | Vernier |
| | | | | | | | | zone |
| 67 | 72 | \| | S | S | S | S | S | |
| 68 | 73 | \| | G | G | G | G | G | Vernier |
| | | | | | | | | zone |
| 69 | 74 | \| | T | T | T | T | T | Vernier |
| | | | | | | | | zone |
| 70 | 75 | \| | D | D | D | D | D | |
| 71 | 76 | \| | F* | F* | F* | F* | F* | Cano L1 |
| | | | | | | | | 4(16) |
| | | | | | | | | Vernier |
| | | | | | | | | zone |
| 72 | 77 | \| | T | T | T | T | T | |
| 73 | 78 | \| | L | L | L | L | L | |
| 74 | 79 | \| | K | K | K | K | K | |
| 75 | 80 | \| | I | I | I | I | I | |
| 76 | 81 | \| | S | S | S | S | S | |
| 77 | 82 | \| | S | R | R | R | R | |
| 78 | 83 | \| | V | V | V | V | V | |
| 79 | 84 | \| | E | E | E | E | E | |
| 80 | 85 | \| | A | A | A | A | A | |
| 81 | 86 | \| | E | E | E | E | E | |
| 82 | 87 | \| | D | D | D | D | D | |
| 83 | 88 | \| | L | V | V | V | V | |
| 84 | 89 | \| | G | G | G | G | G | |
| 85 | 90 | \| | V | V | V | V | V | |
| 86 | 91 | \| | Y | Y | Y | Y | Y | |
| 87 | 92 | \| | Y | Y | Y | Y | Y | VL/VH |
| | | | | | | | | inter |
| 88 | 93 | FR3 | C | C | C | C | C | |
| 89 | 94 | CDR3 | F | M | M | f | F | VL/VH |
| | | | | | | | | inter |
| 90 | 95 | \| | Q* | Q* | Q* | Q* | Q* | Cano L3 |
| | | | | | | | | 1(9) |
| 91 | 96 | \| | G | A | A | g | G | VL/VH |
| | | | | | | | | inter |
| 92 | 97 | \| | S | L | L | s | S | |
| 93 | 98 | \| | H | Q | Q | h | H | |
| 94 | 99 | \| | V | T | T | v | V | |
| 95 | 100 | \| | P* | P* | P* | P* | P* | Cano L3 |
| | | | | | | | | 1(9) |
| 96 | 101 | \| | W | | Q | w | W | VL/VH |
| | | | | | | | | inter (+) |
| 97 | 102 | CDR3 | T | | T | T | T | |
| 98 | 103 | FR4 | F | | F | F | F | VL/VH |
| | | | | | | | | inter (+) |
| | | | | | | | | Vernier |
| | | | | | | | | zone |
| 99 | 104 | \| | G | | G | G | G | |
| 100 | 105 | \| | G | | Q | Q | Q | |
| 101 | 106 | \| | G | | G | G | G | |
| 102 | 107 | \| | T | | T | T | T | |
| 103 | 108 | \| | K | | K | K | K | |
| 104 | 109 | \| | L | | V | V | V | |
| 105 | 110 | \| | E | | E | E | E | |
| 106 | 111 | \| | I | | I | I | I | |
| 107 | 112 | FR4 | K | | K | K | K | |

Legend: The first column (Kabat) indicates the position of the amino acid residue according to Kabat et al. (1991); the second column (#) indicates the position of the amino acid residue in the regular sequence; the third column (FR or CDR) was made in order easily to identify the segments of the skeleton (FR1, FR2, FR3 and FR4) and the CDR segments (CDR1, CDR2 and CDR3) (“CDR” for “Complementarity-Determining Region”) with the three CDRs separating the four FRs; the fourth column (Mouse light chain 7C10) represents the amino acid sequence (SEQ ID No. 54) of the V_{L} region of mouse antibody 7C10; the fifth column (Human germinal line DPK15/A19) represents the amino acid sequence (SEQ ID No. 59) of the kappa II human V light chain of the germinal line; the sixth column (GM607) represents the amino acid sequence (SEQ ID No. 58) of the V_{L} region of the human antibody GM607; the seventh and eighth columns (remodeled human 7C10 1 and 2) represent the amino acid sequences of the humanized 1 and 2 antibody 7C10 VL (respectively SEQ ID Nos. 61 and 65). “*” indicates the parts of the canonical structure of the CDR loop such as defined by Chothia et al. (Nature, 342, 877-883, 1989).

### EXAMPLE 13

### Process of Humanization by CDR Grafting of the Variable Region of The Heavy Chain of the Antibody 7C10 (7C10 VH)

a) Comparison of the Amino Acid Sequence of 7C10 VH with all of the Known Mouse VH Sequences

As a preliminary stage in humanization by CDR grafting, the amino acid sequence of 7C10 VH was first compared with all the mouse VH sequences present in the Kabat databank (Internet address: ftp://ftp.ebi.ac.uk/ pub/database/kabat/fasta_format/, last update of data dates from 1999). 7C10 VH has thus been identified as belonging to the subgroup I(A) of the heavy chains as defined by Kabat et al. (1991). VH regions of mouse monoclonal antibodies having a sequence identity ranging up to 90.5% were identified (AN03′CL (SEQ ID No. 70), see FIG. 22). In order to attempt to identify the out of the ordinary residues in the sequence of 7C10 VH, we aligned the amino acid sequence of 7C10 VH (SEQ ID No. 69) with the consensus sequence (SEQ ID No. 71) of the subgroup I(A) of the mouse heavy chains as defined by Kabat (see FIG. 22).

Residue 17 (Kabat's numbering), Thr for the consensus sequence of subgroup I(A) and Ser in 7C10 VH, is located on the surface of the molecule with respect to the interface with the constant region. This residue does not seem to be important.

Residue 27 (Kabat's numbering), Asp for the consensus sequence of subgroup I(A) and Tyr in 7C10 VH, is a canonical residue for the CDR 1. Tyr in this position is not rare and is probably critical for maintaining CDR 1 in its good conformation.

Residue 84 (Kabat's numbering), Thr for the consensus sequence of the subgroup I(A) and Asn in 7C10 VH. Asn was found 93 times in mouse VH and 3 times in human VH. According to the molecular model, it is a surface residue remote from the paratope.

The numbering of the amino acids is that of Kabat et al. (1991). The residues in the framework regions (apart from CDRs) which differ between 7C10 VH and Kabat mouse subgroup I(A) are underlined. AN03'CL represents the sequence of the heavy chain of a mouse antibody (access number in the Kabat databank is P001289).

b) Comparison of the amino acid sequence of 7C10 VH with all of the known human VH sequences

In order to identify the best human candidate for the “CDR grafting”, the VH region of human origin having the greatest possible homology with 7C10 VH was sought. To this end, the amino acid sequence of mouse 7C10 VH was compared with all the human VH sequences present in the Kabat databank. Mouse 7C10 VH had the greatest sequence homology with the human VH regions of the subgroup II as defined by Kabat et al. (1991). VH regions of monoclonal antibodies of human origin were identified having a sequence identity ranging up to 67.3% (human VH FUR1 'CL (SEQ ID No. 73, see FIG. 23) over the whole of the 98 amino acids encoded by the variable gene (that is to say apart from CDR3 and region J). A germinal line of human origin, 4.22 VH IV (Sanz et al., 1989), having a sequence identity of 68.4%, according to the same criteria as for VH FUR1 'CL, was also identified (human Germ-line (SEQ ID No. 74), see FIG. 23). The sequence encoded by the germinal line 4.22 VH IV was chosen as a human sequence receptive of the CDRs (according to the definition of Kabat) of mouse 7C10 VH rather than VH FUR1 'CL because in comparing the sequences of 4.22 VH IV and VH FUR1 'CL with that of the consensus sequence of the human subgroup II (human Kabat sg II (SEQ ID No. 72), see FIG. 23 and table 6), no atypical residue in the framework regions (Rch) could be identified for 4.22 VH IV although the presence of two atypical residues (Gln and Arg in positions 81 and 82A according to the nomenclature of Kabat, respectively) were identified in the sequence encoded by VH FUR1 'CL.

c) Humanized Versions of 7C10 VH

The following stage in the humanization process consisted in joining the CDRs of mouse 7C10 VH to the framework regions (Rch) of the human germinal line 4.22 VH IV (Sanz et al., 1989). At this stage of the process, the molecular model of the mouse Fv regions of 7C10 is particularly useful in the choice of the mouse residues to be conserved as being able to play a role in the maintenance of the tridimensional structure of the molecule (canonical structure of the CDRs, VH/VL interface, etc.) or in the binding to the antigen (belonging to the paratope). In the Rchs, each difference between the mouse (7C10 VH) and human (4.22 VH IV) amino acids was examined scrupulously (see Table 6). In addition, the particular residues in the mouse 7C10 VH sequence which had been identified (see Example 8.a) were taken into account if needed.

In the first version of 7C10 VH humanized by “CDR grafting”, humanized 1, four changes in the framework regions (Rch) of 4.22 VH IV were carried out (see Table 6, FIG. 24 for the amino acid sequence (SEQ ID No. 75) and FIG. 25 for the DNA sequence (SEQ ID Nos. 76 and 78) and the amino acid sequence comprising the peptide signal (SEQ ID No. 77)). These four changes concern:

Residue 30 (Kabat's nomenclature) situated in Rch 1. This residue enters in effect into the structural composition of the CDR1 of 7C10 VH (as defined by Chothia et al., 1989) and could therefore be critical for maintaining this loop in its correct conformation. The Thr present in this position in the mouse sequence 7C10 VH is therefore conserved in this same position in the humanized form.

Residue 48 (Kabat's nomenclature) situated in Rch 2. This residue is close to the CDRs, although according to the molecular model not in direct contact with the latter, and could influence their ultimate conformation. The methionine present in this position in the mouse sequence 7C10 VH is therefore conserved in this same position in the humanized form 1.
Residue 67 (Kabat's nomenclature) situated in Rch 3. This residue is close to the CDRs and according to the molecular model could contact Lysine 60 (Kabat's nomenclature) in the CDR 2. The isoleucine present in this position in mouse sequence 7C10 VH is therefore conserved in this position in the humanized form 1.
Residue 71 (Kabat's nomenclature) situated in Rch 3. This residue is part of the canonical structure of the CDR 2 and should therefore be critical for maintaining this loop in its correct conformation. The arginine present in this position in the mouse sequence 7C10 VH is therefore conserved in this position in the humanized form 1.

In the second version of 7C10 VH humanized by “CDR grafting”, humanized 2, two changes in the framework regions (Rch) of 4.22 VH IV were carried out. These two changes concern the residues 30 and 71 (Kabat's nomenclature), already described in the humanized form 1 (see Table 6, FIG. 24 for the amino acid sequence (SEQ ID No. 79) and FIG. 26 for the DNA sequence (SEQ ID Nos. 80 and 82) and the amino acid sequence comprising the peptide signal (SEQ ID No. 81)).

In the third form of 7C10 VH humanized by “CDR grafting”, humanized 3, no change in the framework regions (Rch) of 4.22 VH IV was carried out. All the residues of the Rchs are therefore of human origin including the residues 30, 48, 67 and 71 (Kabat's nomenclature) which have been conserved (see Table 6, FIG. 24 for the amino acid sequence (SEQ ID No. 83) and FIG. 27 for the DNA sequence (SEQ ID Nos. 84 and 86) and the amino acid sequence comprising the peptide signal (SEQ ID No. 85)). This humanized form 3 is therefore totally humanized (apart, of course, from the CDRs themselves as defined by Kabat) since all the residues of the Rchs are those encoded by the VH gene of the germinal line 4.22 VH IV.

TABLE 6
Alignment of the amino acid sequences leading to the design of the remodeled human 7C10 V_{H} regions

| | | | Germinal | Human | Remodeled | Remodeled | Remodeled | |
|---|---|---|---|---|---|---|---|---|
| | FR or | Mouse heavy | line | FUR1′CL | Human | Human | Human | |
| Kabat | CDR | chain 7C10 | 4.22 VH IV | VH | 7C10 H 1 | 7C10 H 2 | 7C10 H 3 | Comments |
| 1 | FR1 | D | Q | Q | Q | Q | Q | |
| 2 | \| | V | V | V | V | V | V | Vernier Zone |
| 3 | \| | Q | Q | Q | Q | Q | Q | |
| 4 | \| | L | L | L | L | L | L | |
| 5 | \| | Q | Q | Q | Q | Q | Q | |
| 6 | \| | E | E | E | E | E | E | |
| 7 | \| | S | S | S | S | S | S | |
| 8 | \| | G | G | G | G | G | G | |
| 9 | \| | P | P | P | P | P | P | |
| 10 | \| | G | G | G | G | G | G | |
| 11 | \| | L | L | L | L | L | L | |
| 12 | \| | V | V | V | V | V | V | |
| 13 | \| | K | K | K | K | K | K | |
| 14 | \| | P | P | P | P | P | P | |
| 15 | \| | S | S | S | S | S | S | |
| 16 | \| | Q | E | E | E | E | E | |
| 17 | \| | S | T | T | T | T | T | |
| 18 | \| | L | L | L | L | L | L | |
| 19 | \| | S | S | S | S | S | S | |
| 20 | \| | L | L | L | L | L | L | |
| 21 | \| | T | T | T | T | T | T | |
| 22 | \| | C | C | C | C | C | C | |
| 23 | \| | S | T | T | T | T | T | |
| 24 | \| | V | V | V | V* | V* | V* | canonical H1 2(6) |
| 25 | \| | T | S | S | S | S | S | |
| 26 | \| | G* | G* | G* | G* | G* | G* | canonical H1 2(6) |
| 27 | \| | Y* | Y* | Y* | Y* | Y* | Y* | canonical H1 2(6) |
| | | | | | | | | Vernier Zone |
| 28 | \| | S | S | S | S | S | S | Vernier Zone |
| 29 | \| | I* | I* | I* | I* | I* | I* | canonical H1 2(6) |
| | | | | | | | | Vernier Zone |
| 30 | FR1 | T | S | S | t | T | S | Vernier Zone |
| | | | | | | | | Close to the CDRs |
| 31 | CDR1 | G | S | S | g | G | q | |
| 32 | \| | G | G | G | G | G | G | |
| 33 | \| | Y | Y | Y | Y | Y | Y | |
| 34 | \| | L | Y | Y | I | L | I | |
| 35 | \| | W* | W* | W* | W* | W* | W* | canonical H1 2(6) |
| | | | | | | | | VH/VL interface |
| 35A | CDR1 | N | G | G | n | N | n | |
| 36 | FR2 | W | W | W | W | W | W | |
| 37 | \| | I | I | I | I | I | I | VH/VL interface |
| 38 | \| | R | R | R | R | R | R | |
| 39 | \| | Q | Q | Q | Q | Q | Q | VH/VL interface |
| 40 | \| | F | P | P | P | P | P | |
| 41 | \| | P | P | P | P | P | P | |
| 42 | \| | G | G | G | G | G | G | |
| 43 | \| | N | K | K | K | K | K | |
| 44 | \| | K | G | G | G | G | G | |
| 45 | \| | L | L | L | L | L | L | VH/VL interface (+) |
| 46 | \| | E | E | E | E | E | E | |
| 47 | \| | W | W | W | W | W | W | VH/VL interface |
| | | | | | | | | Vernier Zone |
| 48 | \| | M | I | I | m | I | I | Vernier Zone |
| | | | | | | | | Close to the CDRs |
| 49 | FR2 | G | G | G | G | G | G | Vernier Zone |
| 50 | CDR2 | Y | S | S | y | Y | y | Vernier Zone |
| 51 | \| | I | I | M | I | I | I | |
| 52 | \| | S | Y | F | s | S | s | |
| 53 | \| | Y | H | H | y | Y | y | |
| 54 | \| | D | S | S | d | D | d | |
| 55 | \| | G* | G* | G* | G* | G* | G* | canonical H2 1(16) |
| 56 | \| | T | S | S | t | T | t | |
| 57 | \| | N | T | S | n | N | n | |
| 58 | \| | N | Y | Y | n | N | n | |
| 59 | \| | Y | Y | Y | Y | Y | Y | |
| 60 | \| | K | N | N | k | K | k | |
| 61 | \| | P | P | P | P | P | P | |
| 62 | \| | S | S | S | S | S | S | |
| 63 | \| | L | L | L | L | L | L | |
| 64 | \| | K | K | K | K | K | K | |
| 65 | CDR2 | D | S | S | d | d | d | |
| 66 | FR3 | R | R | R | R | R | R | |
| 67 | \| | I | V | V | i | V | V | Vernier Zone |
| | | | | | | | | Close to the CDRs |
| 68 | \| | S | T | T | T | T | T | |
| 69 | \| | I | I | I | I | I | I | Vernier Zone |
| 70 | \| | T | S | S | S | S | S | |
| 71 | \| | R* | V* | V* | r* | r* | V* | canonical H2 1(16) |
| | | | | | | | | Vernier Zone |
| 72 | \| | D | D | D | D | D | D | |
| 73 | \| | T | T | T | T | T | T | Vernier Zone |
| 74 | \| | S | S | S | S | S | S | |
| 75 | \| | K | K | K | K | K | K | |
| 76 | \| | N | N | N | N | N | N | |
| 77 | \| | Q | Q | Q | Q | Q | Q | |
| 78 | \| | F | F | F | F | F | F | Vernier Zone |
| 79 | \| | F | S | S | S | S | S | |
| 80 | \| | L | L | L | L | L | L | |
| 81 | \| | K | K | Q | K | K | K | |
| 82 | \| | L | L | L | L | L | L | |
| 82A | \| | N | S | R | S | S | S | |
| 82B | \| | S | S | S | S | S | S | |
| 82C | \| | V | V | V | V | V | V | |
| 83 | \| | T | T | T | T | T | T | |
| 84 | \| | N | A | A | A | A | A | |
| 85 | \| | E | A | A | A | A | A | |
| 86 | \| | D | D | D | D | D | D | |
| 87 | \| | T | T | T | T | T | T | |
| 88 | \| | A | A | A | A | A | A | |
| 89 | \| | T | V | V | V | V | V | |
| 90 | \| | Y | Y | Y | Y | Y | Y | |
| 91 | \| | Y | Y | Y | Y | Y | Y | VH/VL interface |
| 92 | \| | C | C | C | C | C | C | |
| 93 | \| | A | A | A | A | A | A | VH/VL interface |
| | | | | | | | | Vernier Zone |
| 94 | FR3 | R* | R* | R* | R* | R* | R* | canonical H1 2(6) |
| | | | | | | | | Vernier Zone |
| 95 | CDR3 | Y | | G | y | y | y | VH/VL interface |
| 96 | \| | G | | R | g | g | g | |
| 97 | \| | R | | Y | r | r | r | |
| 98 | \| | V | | C | v | v | v | |
| 99 | \| | F | | S | f | f | f | |
| 100 | \| | | | S | | | | |
| 100A | \| | | | T | | | | |
| 100B | \| | | | S | | | | |
| 100C | \| | | | C | | | | |
| 100D | \| | | | N | | | | |
| 100E | \| | | | W | | | | |
| 100K | \| | F | | F | f | f | f | VH/VL interface (+) |
| 101 | \| | D | | D | d | d | d | |
| 102 | CDR3 | Y | | P | y | y | y | |
| 103 | FR4 | W | | W | W | W | W | VH/VL interface (+) |
| | | | | | | | | Vernier Zone |
| 104 | \| | G | | G | G | G | G | |
| 105 | \| | Q | | Q | Q | Q | Q | |
| 106 | \| | G | | G | G | G | G | |
| 107 | \| | T | | T | T | T | T | |
| 108 | \| | T | | L | L | L | L | |
| 109 | \| | L | | V | V | V | V | |
| 110 | \| | T | | T | T | T | T | |
| 111 | \| | V | | V | V | V | V | |
| 112 | \| | S | | S | S | S | S | |
| 113 | FR4 | S | | S | S | S | S | |

Legend: The first column (Kabat) indicates the position of the amino acid residue according to Kabat et al. (1991); the second column (FR or CDR) was made in order easily to identify the segments of the skeleton (FR1, FR2, FR3 and FR4) and the CDR segments (CDR1, CDR2 and CDR3) with the three CDRs separating the four FRs; the third column (Mouse heavy chain 7C10) represents the amino acid sequence (SEQ ID No. 69) of the VH region of the mouse antibody 7C10; the fourth column (Germinal line 4.22 VH IV) represents the amino acid sequence of the gene 4.22 VH IV (Sanz et al., 1989) (SEQ ID No. 74); the fifth column (human FUR1 'CL VH, kabat accession number N₀₂₀₆₁₉) represents the amino acid sequence (SEQ ID No. 73) [lacuna] IgMK antilamin B of human origin (Mariette et al., 1993); the sixth, seventh and eighth columns (remodeled human 7C10 1, 2 and 3) represent the amino acid sequences of the VH region of remodeled human 7C10 respectively for the versions 1 (SEQ ID No. 75), 2 (SEQ ID No. 79) and 3 (SEQ ID No. 83). “*” indicates the parts of the canonical structure of the CDR loop such as defined by Chothia et al. (1989).

### EXAMPLE 14

### Construction of the Genes Coding for the Humanized Versions 1 of 7C10 VL and VH by Assembly of Oligonucleotides

a) Principle

The genes (leader peptide+variable regions VDJ for VH or VJ for VK) coding for the humanized variable regions were synthesized by solid-phase assembly on magnetic beads coated with streptavidin. The genes coding for humanized 7C10 VH (445 base pairs) and humanized 7C10 VL (433 base pairs) are constructed by fusing two fragments of DNA owing to the presence of a KpnI restriction site present in the two sequences and situated almost halfway along the gene (at 200 and 245 nucleotides with respect to the 5′ end of the gene for VL and VH, respectively). The two fragments which are fused together are themselves assembled by an assembly technique which consists in using phosphorylated oligonucleotides (approximately 30-35 mer) hybridized two by two (one oligo sense and the other antisense, with a homology of approximately 50%) in such a way that they overlap during elongation. A first oligonucleotide biotinylated in the 5′ position is attached to the magnetic beads and then the pairs of phosphorylated oligonucleotides are added one by one. The phosphodiester linkage between the juxtaposed phosphorylated oligonucleotides is produced by the enzyme T4 DNA ligase.

The genes thus synthesized de novo can be cloned directly (by digestion with restriction enzymes compatible with the expression vector chosen) or amplified by PCR in order to obtain more material as a prelude to directional cloning by enzymatic digestion. The sequence of the gene thus constructed by de novo assembly is then verified by automatic sequencing of the DNA.

b) Experimental Protocol of the De Novo Assembly Technique

Oligonucleotides phosphorylated in the 5′ position or biotinylated in the 5′ position whose concentration was adjusted to 100 μM were ordered from MWG Biotech (see the sequences of the oligonucleotides used in Table 7 for the construction of humanized 7C10 VL, and Table 8 for the construction of humanized 7C10 VH). The oligonucleotides were hybridized in pairs (an equimolar mixture, 500 μmol, of a sense oligo and of an antisense oligo in the buffer T4 DNA ligase is heated to 95° C. for 5 minutes and then allowed to cool on the bench to ambient temperature) according to a scheme described in Table 9.

The first biotinylated oligonucleotide is attached to magnetic beads coated with streptavidin (Dynabeads M-280 streptavidin, Dynal product No. 112-05). For this, 500 μmol of the biotinylated oligonucleotide in a 15 mM NaCl solution are added to 50 μl of the decanted beads (use of a magnet holder) previously washed twice with 100 μl of TE 1× buffer (Tris-EDTA 100× buffer: 1 M Tris-HCl, pH 8, 0.1 M EDTA, Sigma T-9285). After incubation at 37° C. for 15 min, the beads are washed twice with the wash buffer (10 mM Tris-HCl pH 7.6, 10 mM EDTA and 50 mM NaCl) and the pairs of hybridized oligo-nucleotides are then added one by one. On each readdition of a pair of oligonucleotides, the mixture is heated to 95° C. for 5 min and then allowed to cool on the bench to ambient temperature. Once ambient temperature is reached, 2 μl of 10 U/μl T4 DNA ligase (Biolabs) are added and the mixture is incubated for 20 min at 37° C. The beads are then washed (wash buffer) and the following pairs of oligonucleotides are then added in succession.

The last unpaired oligo (antisense) is assembled in the following fashion. 5 μl of oligo (500 μmol) and 43 μl of T4 DNA ligase buffer are added to the decanted beads, then the mixture is heated to 95° C. for 5 min and allowed to cool on the bench to ambient temperature. Once ambient temperature is reached, 2 μl of T4 DNA ligase are added and the mixture is incubated at 37° C. for 20 min. The beads are then washed twice with wash buffer and then twice with TE 1× buffer.

The beads can then be conserved at 4° C. before proceeding to the cloning and sequencing of the gene assembled de novo.

TABLE 7

DNA sequence of oligonucleotides used for the construction of humanized
7C10 VL 1 by de novo assembly

| | | |
|---|---|---|
| LeaderMluI.biotin | 5′-GTCAGAACGCGTGCCGCC | (SEQ ID No. 87) |
| 7C10Lresh.1sense | 5′-ACCATGAAGTTGCCTGTTAGGCTGTTGGTGCT | (SEQ ID No. 88) |
| 7C10Lresh.2sense | 5′-GATGTTCTGGTTTCCTGCTTCCAGCAGTGATG | (SEQ ID No. 89) |
| 7C10Lresh.3sense | 5′-TTGTGATGACTCAGTCTCCACTCTCCCTGCCC | (SEQ ID No. 90) |
| 7C10Lresh.4sense | 5′-GTCACCCCTGGAGAGCCGGCCTCCATCTCCTG | (SEQ ID No. 91) |
| 7C10Lresh.5sense | 5′-CAGGTCTAGTCAGACCATTATACATAGTAATG | (SEQ ID No. 92) |
| 7C10Lresh.6sense | 5′-GAAACACCTATTTGGAATGGTACCTGCAGA | (SEQ ID No. 93) |
| 7C10Lresh.7anti | 5′-GGCAACTTCATGGTGGCGGCACGCGTTCTGAC | (SEQ ID No. 94) |
| 7C10Lresh.8anti | 5′-GAAACCAGAACATCAGCACCAACAGCCTAACA | (SEQ ID No. 95) |
| 7C10Lresh.9anti | 5′-CTGAGTCATCACAACATCACTGCTGGAAGCAG | (SEQ ID No. 96) |
| 7C10Lresh.10anti | 5′-TCTCCAGGGGTGACGGGCAGGGAGAGTGGAGA | (SEQ ID No. 97) |
| 7C10Lresh.11anti | 5′-TCTGACTAGACCTGCAGGAGATGGAGGCCGGC | (SEQ ID No. 98) |
| 7C10Lresh.12anti | 5′-AAATAGGTGTTTCCATTACTATGTACAATGC | (SEQ ID No. 99) |
| 7C10Lresh.13sense | 5′-CAGGGCAGTCTCCACAGCTCCTGATCTATAAA | (SEQ ID No. 100) |
| 7C10Lresh.14sense | 5′-GTTTCTAATCGGCTTTATGGGGTCCCTGACAG | (SEQ ID No. 101) |
| 7C10Lresh.15sense | 5′-GTTCAGTGGCAGTGGATCAGGCACAGATTTTA | (SEQ ID No. 102) |
| 7C10Lresh.16sense | 5′-CACTGAAAATCAGCAGAGTGGAGGCTGAGGAT | (SEQ ID No. 103) |
| 7C10Lresh.17sense | 5′-GTTGGGGTTTATTACTGCTTTCAAGGTTCACA | (SEQ ID No. 104) |
| 7C10Lresh.18sense | 5′-TGTTCCGTGGACGTTCGGCCAAGGGACCAAGG | (SEQ ID No. 105) |
| 7C10Lresh.19sense | 5′-TGGAAATCAAACGTGAGTGGATCCTCTGCG | (SEQ ID No. 106) |
| 7C10Lresh.KpnIREV | 5′-TCTGCAGGTACCATTGC | (SEQ ID No. 107) |
| 7C10Lresh.KpnIbiotin | 5′-TGCAATGGTACCTGCAGAAGC | (SEQ ID No. 108) |
| 7C10Lresh.20anti | 5′-AGACTGCCCTGGCTTCTGCAGGTACCATTGCA | (SEQ ID No. 109) |
| 7C10Lresh.21anti | 5′-CGATTAGAAACTTTATAGATCAGGAGCTGTGG | (SEQ ID No. 110) |
| 7C10Lresh.22anti | 5′-TGCCACTGAACCTGTCAGGGACCCCATAAAGC | (SEQ ID No. 111) |
| 7C10Lresh.23anti | 5′-GATTTTCAGTGTAAAATCTGTGCCTGATCCAC | (SEQ ID No. 112) |
| 7C10Lresh.24anti | 5′-TAAACCCCAACATCCTCAGCCTCCACTCTGCT | (SEQ ID No. 113) |
| 7C10Lresh.25anti | 5′-TCCACGGAACATGTGAACCTTGAAAGCAGTAA | (SEQ ID No. 114) |
| 7C10Lresh.26anti | 5′-TTTGATTTCCACCTTGGTCCCTTGGCCGAAC | (SEQ ID No. 115) |
| 7C10Lresh.BamHIantisense | 5′-CGCAGAGGATCCACTCACG | (SEQ ID No. 116) |

TABLE 8

DNA sequence of oligonucleotides used for the construction of humanized
7C10 VH 1 by de novo assembly

| | | |
|---|---|---|
| LeaderMluI.biotin | 5′-GTCAGAACGCGTGCCGCC | (SEQ ID No. 117) |
| 7C10Hresh.1sense | 5′-ACCATGAAAGTGTTGAGTCTGTTGTACCTCTTGA | (SEQ ID No. 118) |
| 7C10Hresh.2sense | 5′-CAGCCATTCCTGGTATCCTGTCTCAGGTGCAGCT | (SEQ ID No. 119) |
| 7C10Hresh.3 sense | 5′-TCAGGAGTCGGGCCCAGGACTGGTGAAGCCTTCG | (SEQ ID No. 120) |
| 7C10Hresh.4sense | 5′-GAGACCCTGTCCCTCACCTGCACTGTCTCTGGT | (SEQ ID No. 121) |
| 7C10Hresh.5sense | 5′-TACTCCATCACCGGTGGTTATTTATGGAACTGG | (SEQ ID No. 122) |
| 7C10Hresh.6sense | 5′-ATACGGCAGCCCCCAGGGAAGGGACTGGAGTGG | (SEQ ID No. 123) |
| 7C10Hresh.7sense | 5′-ATGGGGTATATCAGCTACGACGGTACCAATAAC | (SEQ ID No. 124) |
| 7C10Hresh.8antisense | 5′-TCAACACTTTCATGGTGGCGGCACGCGTTCTGAC | (SEQ ID No. 125) |
| 7C10Hresh.9antisense | 5′-ATACCAGGAATGGCTGTCAAGAGGTACAACAGAC | (SEQ ID No. 126) |
| 7C10Hresh.10antisense | 5′-TGGGCCCGACTCCTGAAGCTGCACCTGAGACAGG | (SEQ ID No. 127) |
| 7C10Hresh.11antisense | 5′-TGAGGGACAGGGTCTCCGAAGGCTTCACCAGTCC | (SEQ ID No. 128) |
| 7C10Hresh.12antisense | 5′-CCACCGGTGATGGAGTAACCAGAGACAGTGCAGG | (SEQ ID No. 129) |
| 7C10Hresh.13antisense | 5′-CCCTGGGGGCTGCCGTATCCAGTTCCATAAATAA | (SEQ ID No. 130) |
| 7C10Hresh.14antisense | 5′-TAGCTGATATACCCCATCCACTCCAGTCCCTT | (SEQ ID No. 131) |
| 7C10Hresh.KpnIREV | 5′-GTTATTGGTACCGTCG | (SEQ ID No. 132) |
| 7C10Hresh.KpnIbiotin | 5′-TACGACGGTACCAATAACTAC | (SEQ ID No. 133) |
| 7C10Hresh.15sense | 5′-AAACCCTCCCTCAAGGATCGAATCACCATATC | (SEQ ID No. 134) |
| 7C10Hresh.16sense | 5′-ACGTGACACGTCCAAGAACCAGTTCTCCCTGA | (SEQ ID No. 135) |
| 7C10Hresh.17sense | 5′-AGCTGAGCTCTGTGACCGCTGCGGACACTGCA | (SEQ ID No. 136) |
| 7C10Hresh.18sense | 5′-GTGTATTACTGTGCGAGATACGGTAGGGTCTT | (SEQ ID No. 137) |
| 7C10Hresh.19sense | 5′-CTTTGACTACTGGGGCCAGGGAACCCTGGTCA | (SEQ ID No. 138) |
| 7C10Hresh.20sense | 5′-CCGTCTCCTCAGGTGAGTGGATCCTCTGCG | (SEQ ID No. 139) |
| 7C10Hresh.21antisense | 5′-AGGGAGGGTTTGTAGTTATTGGTACCGTCGTA | (SEQ ID No. 140) |
| 7C10Hresh.22antisense | 5′-ACGTGTCACGTGATATGGTGATTCGATCCTTG | (SEQ ID No. 141) |
| 7C10Hresh.23antisense | 5′-AGAGCTCAGCTTCAGGGAGAACTGGTTCTTGG | (SEQ ID No. 142) |
| 7C10Hresh.24antisense | 5′-CAGTAATACACTGCAGTGTCCGCAGCGGTCAC | (SEQ ID No. 143) |
| 7C10Hresh.25antisense | 5′-AGTAGTCAAAGAAGACCCTACCGTATCTCGCA | (SEQ ID No. 144) |
| 7C10Hresh.26antisense | 5′-CTGAGGAGACGGTGACCAGGGTTCCCTGGCCCC | (SEQ ID No. 145) |
| 7C10Hresh.BamHIantisense | 5′-CGCAGAGGATCCACTCAC | (SEQ ID No. 146) |

TABLE 9
Oligonucleotide pairing protocol for the de novo assembly
of genes coding for the humanized forms of 7C10 VH and VL

| de novo assembly | de novo assembly |
|---|---|
| of the M1UI-KpnI fragment | of the KpnI-BamHI fragment |
| of 7C10 VL humanized 1 | of 7C10 VL humanized 1 |
| Biotinylated oligo leader | Biotinylated oligo 7C10 L |
| M1UI 7C10 VL | KpnI |
| Oligo pair 1 and 7 | Oligo pair 13 and 20 |
| Oligo pair 2 and 8 | Oligo pair 14 and 21 |
| Oligo pair 3 and 9 | Oligo pair 15 and 22 |
| Oligo pair 4 and 10 | Oligo pair 16 and 23 |
| Oligo pair 5 and 11 | Oligo pair 17 and 24 |
| Oligo pair 6 and 12 | Oligo pair 18 and 25 |
| Antisense oligo 7C10 | Oligo pair 19 and 26 |
| VL KpnI | Antisense oligo 7C10 L |
| | BamHI |
| Biotinylated oligo leader | Biotinylated oligo 7C10 H |
| M1UI 7C10 VH | KpnI |
| Oligo pair 1 and 8 | Oligo pair 15 and 21 |
| Oligo pair 2 and 9 | Oligo pair 16 and 22 |
| Oligo pair 3 and 10 | Oligo pair 17 and 23 |
| Oligo pair 4 and 11 | Oligo pair 18 and 24 |
| Oligo pair 5 and 12 | Oligo pair 19 and 25 |
| Oligo pair 6 and 13 | Oligo pair 20 and 26 |
| Oligo pair 7 and 14 | Antisense oligo 7C10 VH |
| Antisense oligo 7C10 | BamHI |
| VH KpnI | |

### EXAMPLE 15

### Construction of the Genes Coding for the Humanized Versions 2 Of 7C10 VL and 7C10 VH and 3 Of 7C10 VH by Directed Mutagenesis

The humanized version 2 of 7C10 VH was obtained by directed mutagenesis of the residues 48 and 67 (according to Kabat's nomenclature) of version 1. This directed mutagenesis was carried out with the aid of the system QuikChange™ Site-directed mutagenesis of Stratagene (kit #200518) according to the protocol described by the manufacturer. The construction is carried out in two stages, first the residue 48 on version 1 was mutated with the aid of the pair of primers 7C10Hhumanized1QCM48 sense and antisense (see Table 10) and subsequently this version mutated at the residue 48 was itself mutated at the residue 67 with the aid of the pair of primers 7C10Hhumanized1 QCI67 sense and antisense (see Table 10).

The humanized version 3 of 7C10 VH was obtained by site-directed mutation of the residues 30 and 71 (according to Kabat's nomenclature) of version 2 likewise using the system QuikChange™. This construction is carried out in two stages. At first, the residue 30 on version 2 was mutated with the aid of the primers 7C10HhumanizedQCT30 sense and antisense (see Table 10). Subsequently, this version mutated at the residue 30 was itself mutated at the residue 71 by using the pair of primers 7C10Hhumanized1V67QCR71 sense and antisense (see Table 10).

The humanized version 2 of 7C10 VL was obtained by site-directed mutation of the residue 2 (according to Kabat's nomenclature) of version 1 by using the system QuikChange™. The residue 2 on version 1 was mutated by using the pair of primers 7C10Lhumanized1QCV2 sense and antisense (see Table 10).

TABLE 10

List of the oligonucleotides used for the directed mutagenesis by the
stratagene QuikChange ™ system

| | | |
|---|---|---|
| 7C10Hhumanized1QCT30.sense | 5′-CTGGTTACTCCATCAGCGGTGGTTATTTATG | (SEQ ID No. 147) |
| 7C10Hhumanized1QCT30.antisense | 5′-CATAAATAACCACCGCTGATGGAGTAACCAG | (SEQ ID No. 148) |
| 7C10Hhumanized1QCM48.sense | 5′-GGGACTGGAGTGGATCGGGTATATCAGCTAC | (SEQ ID No. 149) |
| 7C10Hhumanized1QCM48.antisense | 5′-GTAGCTGATATACCCGATCCACTCCAGTCCC | (SEQ ID No. 150) |
| 7C10Hhumanized1QCI67.sense | 5′-TCCCTCAAGGATCGAGTCACCATATCACGTG | (SEQ ID No. 151) |
| 7C10Hhumanized1QCI67.antisense | 5′-CACGTGATATGGTGACTCGATCCTTGAGGGA | (SEQ ID No. 152) |
| 7C10Hhumanized1V67QCR71.sense | 5′-GATCGAGTCACCATATCAGTGGACACGTCCAAGAACCAG | (SEQ ID No. 153) |
| 7C10Hhumanized1V67QCR71.antisense | 5′-CTGGTTCTTGGACGTGTCCACTGATATGGTGACTCGATC | (SEQ ID No. 154) |
| 7C10Lhumanized1QCV2.sense | 5′-GCTTCCAGCAGTGATATTGTGATGACTCAGT | (SEQ ID No. 155) |
| 7C10Lhumanized1QCV2.antisense | 5′-ACTGAGTCATCACAATATCACTGCTGGAAGC | (SEQ ID No. 156) |

### EXAMPLE 16

### Transfection of the COS7 Cells by Electroporation

The mammalian expression vectors containing the chimeric or humanized versions of the heavy and light chains of the antibody 7C10 were tested in cos7 cells for the transitory expression of the recombinant antibodies 7C10. The DNA was introduced into the cos cells by electroporation with the aid of a BioRad instrument (Gene Pulsar). The DNA (10 μg of each vector) is added to aliquots of 0.8 ml of cos cells at a concentration of 1×10⁷ cells per ml in PBS buffer (without Ca++ and Mg++). A pulsation of 1900 volts and a capacity of 25 μF was delivered. The transfected cos cells are then added to 8 ml of DMEM medium containing 5% of calf serum and incubated at 37° C. for 72 hours. The supernatant is then collected, centrifuged in order to eliminate the cell debris and tested by ELISA for the measurement of its concentration of recombinant antibody 7C10 of IgG1 /human Kappa type.

### EXAMPLE 17

### ELISA Method for Measuring the Concentrations of Recombinant Antibody IGG1/Human Kappa Present in the Supernatant of the COS Transfectants

The supernatants produced by transitory expression in cos7 cells were tested for the presence of 7C10 antibody of IgG1/human Kappa type. For the detection of the IgG1/human Kappa immunoglobulin, 96-well ELISA plates (Maxisorb, Nunc) were coated with a goat anti-human IgG polyclonal antibody (specific for the gamma Fc fragment, Jackson Immuno-Research Laboratories Inc., #109-005-098). The supernatants of cos cells were diluted in series and added to the coated wells. After incubation for one hour at 37° C. and washing, a goat anti-human light Kappa chain polyclonal antibody conjugated to peroxidase (HRP, Sigma, A-7164) was added. After incubation for 45 minutes at 37° C. and washing, the TMB substrate (KPL #50-76-04) was added. After incubation for 10 minutes, the reaction was stopped by the addition of 1 M sulfuric acid and the optical density was read at 450 nm. A purified human IgG1/human Kappa immunoglobulin (Sigma, I-3889) of known concentration was used as a standard reference antibody.

### EXAMPLE 18

### Elisa Method for Determining The Recognition Activity of 7C10 Recombinant Antibodies of Human IGG1/Kappa Type on the Receptor For IGF-I (IGF-IR)

The cos7 culture supernatants were tested for their capacity to recognize IGF-I R by an ELISA method. 96-well ELISA plates (Dynex Immulon 2HB) were coated with 100 μl per well of a solution of PBS containing 0.31 ng/μl of IGF-I R (Human Insulin-Like Growth Factor I soluble Receptor, R & D Systems, #391-GR) by incubation for one night at 4° C. After washing with PBS containing 0.05% Tween 20, the plates were saturated by the addition of a solution of PBS containing 0.5% gelatin solution and incubation at 37° C. for 1 hour. After three washes with PBS, the samples of cos supernatants to be tested, previously diluted in series in PBS containing 0.1% gelatin and 0.05% Tween 20, were added to the plates. After incubation at 37° C. for 1 hour followed by three washes (PBS containing 0.05% Tween 20), an anti-human IgG antibody (specific for the Fc fragment) conjugated to peroxidase (HRP, Jackson Immuno-Research Laboratories Inc., #109-035-098) was added (dilution to 1/5000 in PBS containing 0.1% gelatin and 0.05% Tween 20). After incubation for 45 minutes at 37° C. and 3 washes (PBS containing 0.05% Tween 20), the TMB substrate (KPL #50-76-04) was added. After incubation for 10 minutes, the reaction was stopped by addition of 1 M sulfuric acid and the optical density was read at 450 nm.

### EXAMPLE 19

### Determination of the Recognition Activity of IGF1-R by Different Versions of the Humanized 7C10 Antibody by “CDR Grafting”

At first, we compared the recognition activity of humanized forms 1 of the heavy and light chains of 7C10 for the IGF-I receptor with respect to the chimeric form. FIG. 28 shows the results of an ELISA test of recognition of the IGF-IR (see Example 18) from supernatants of the cos7 cells whose concentration of IgG1/human Kappa had been previously determined by ELISA (see Example 17). The titration curves of the four recombinant antibodies tested overlap perfectly indicating that their relative affinities for IGF-IR are very similar. It is therefore concluded from this that the humanized form 1 of 7C10, composed of the humanized light chain 1 (1 single mouse residue present in the framework regions) in combination with the humanized heavy chain 1 (4 mouse residues present in the framework regions), specifically recognizes the IGF-I receptor and has an affinity very similar to that of the chimeric antibody (mouse variable regions).

Subsequently, we looked at the influence of the residue 2 (according to Kabat's nomenclature) of the humanized light chain of 7C10 (humanized version 1 versus humanized 2, see FIG. 19) on the recognition of the IGF-IR. FIG. 29 shows the results of the ELISA test for recognition of the IGF-IR (see Example 18) from supernatants of cos7 cells whose concentration of IgG1/human Kappa had been previously determined by ELISA (see Example 17). The two humanized versions 1 and 2 of the light chain had been combined successively with humanized 7C10 VH 1. The titration curves of the two combinations are superimposed indicating that the mutation of residue 2 of the light chain, which has been changed from one valine in the humanized version 1 to an isoleucine in the humanized form 2, apparently has no influence on the relative affinity of recognition of the IGF1 receptor. The humanized form 2 of the light chain of 7C10 thus forms one version where no mouse residue (apart from CDRs) has been conserved. This version, totally humanized, represents the preferred version of 7C10 VL.

The totally humanized version of the 7C10 light chain (humanized version 2, see above) was tested in combination with the three humanized versions of the heavy chain of 7C10. FIG. 30 shows the results of the ELISA test for recognition of the IGF-IR from supernatants of cos7 cells whose concentration of IgG1/human Kappa had been previously determined by ELISA (see Example 17). The titration curves are very similar and virtually overlap with the reference curve corresponding to the chimeric antibody, indicating that the three humanized versions 1, 2 and 3 of 7C10 VH give an identical relative affinity for IGF-IR when they are combined with humanized 7C10 VL 2. Other ELISA tests conducted in parallel (results not shown) have however revealed that a point mutation of the residue 71 (Kabat's nomenclature) from an arginine (mouse) to a valine (human) involved a small loss of affinity of the corresponding antibody for IGF-IR. It is thus reasonable to think that humanized 7C10 VH 2 has the same relative affinity for IGF-IR as humanized 7C10 VH1. This humanized form 2 will therefore be preferred with respect to the form 1 since it only has two mouse amino acids (residues 30 and 71, see FIG. 24). The humanized form 3 which does not have any mouse residue (apart from CDRs) will also be preferred since it only seems to involve a minimal loss of affinity.

In conclusion, it appears that two humanized forms of the antibody 7C10 according to the present invention are particularly preferred. A form constituted by the combination of humanized 7C10 VH 2 (2 conserved mouse residues) with humanized 7C10 VL 2 (no conserved mouse residue) and another form constituted by the combination of humanized 7C10 VH 3 (no conserved mouse residue) with humanized 7C10 VL 2 (no conserved mouse residue). This last form constitutes the ultimate humanized version since no mouse residue is present at the same time in the heavy and light chains.

### EXAMPLE 20

### Expression of EGFR and of IGF-IR on the Surface of A549 Cells

The synergy of action obtained by the coadministration of two MABs directed respectively against IGF-IR and EGFR was studied in nude mice carrying a non-small cell lung tumor established by subcutaneous injection (s.c.) of A549 cells (lung carcinoma cell line).

At first, and in order to ensure the presence of the two receptors IGF-IR and EGFR on the surface of the A549 cell before injecting this into the mouse, labeling for FACS reading of these cells was carried out with, respectively, the murine 7C10 anti-IGF-IR MAB (FIG. 37B) and the murine 225 anti-EGFR MAB (FIG. 37D). In order to do this, the cells were saturated for 30 min at 4° C. with a solution of PBS 10% FCS (fetal calf serum), washed and then incubated for 30 min at 4° C. with the MAB of interest. After 3 new washes, the secondary anti-species antibody coupled to FITC (fluorescein isothiocyanate) is added. After incubation for 30 min, reading on the FACS (Fluorescence Activated Cells Sorter) is carried out at 520 nm (excitation 488 nm).

The results presented in FIGS. 37A to 37D show that the A549 cells have on their surface a comparable number of receptors for EGF and IGF1. In the two cases, the population is homogeneous with respect to the distribution of each of the receptors. The specificity of the labeling is confirmed by the use of an isotype control (FIG. 37C). These results validate the use of the A549 cell as a model for the study of a synergy of action on two IGF-IR and EGFR receptors and for the study of a collaboration of these two receptors.

### EXAMPLE 21

Synergy of action of an anti-IGF-IR MAB and of an anti-EGFR MAB coadministered in vivo, in the nude mouse in the context of an antitumor treatment

For this study, nude mice are grafted s.c. with 5.10⁶ A549 cells. Five days after the cell graft, the tumors are measured and a homogeneous batch of mice in terms of tumor volume is formed. Starting from this batch, groups of 6 mice are generated at random. These mice will be treated intraperitoneally (i.p.), twice per week with each of the MAB 7C10 and 225 individually at the dose of 250 μg/mouse or with the two MAB in coadministration. The MAB 9G4 is administered as an experiment isotype control.

The results presented in FIG. 38 show that each of the antibodies 7C10 and 225 administered alone is capable of inducing a significant decrease in the tumor growth in vivo. It can be noted that the two MAB tested have a comparable activity on the growth of the tumor A549. In a surprising fashion with respect to the literature, a significant synergy is observed during simultaneous administration of the two MAB (p< or =0.01 at each of the times of the kinetics in a t-test) suggesting that a collaboration of the two receptors exists for the optimum growth of a tumor in vivo and that, contrary to the data in the literature, the blockage of one of the two axes does not suffice to totally inhibit the growth mediated by the second.

### EXAMPLE 22

Study of the antitumor activity of the murine antibodies 7C10 and 225 coadministered in mice orthotopically implanted with A549 cells

The use of orthotopic models for the evaluation of the antitumor activity presents a particular interest with respect to the process of metastatic dissemination of a tumor. In order to evaluate the antitumor activity of an antibody mixture directed respectively against IGF-IR and EGFR, 10⁶ A549 cells (non-small cell lung cancer) were implanted in the intrapleural cavity of nude mice. It is to be noted that the consequence of this type of tumor implantation is a metastatic dissemination similar to that observed in man and leads to the death of the animals. FIG. 39 shows that the administration of the antibodies 225 and 7C10 alone allows a comparable and a significant gain in survival to be observed. In a surprising fashion, the coadministration of these two antibodies increases in a considerable fashion the survival of the animals suggesting that this treatment could have an impact on the metastatic dissemination of the tumor cells.

### EXAMPLE 23

7C10 and 7H2HM inhibit the phosphorylation of the tyrosine of the β chain of IGF-IR and of IRS-I

MCF7 cells are cultured for 24 hours at 5.10⁴ cells/cm² (75 cm² plates, COSTAR) in 20 ml of RPMI without phenol red, mixed with 5 mM of glutamine, penicillin/streptomycin (respectively 100 U/100 μg/ml) and 10% of fetal calf serum. After three washes in PBS, the cells were incubated for 12 hours in medium (RPMI) without phenol red, devoid of fetal calf serum and mixed with 5 mM of glutamine, penicillin/streptomycin, bovine serum albumin at 0.5 μg/ml (Sigma A-8022) and transferrin at 5 μg/ml (Sigma T8158).

For activation, the cells were first incubated at 37° C. for 2 minutes with blocking antibodies (10 μg/ml) and then IGF-I (Sigma 13769, 50 ng/ml) was added for two additional minutes. The reaction was stopped by aspiration of the incubation medium and the plates were laid on ice. The cells were solubilized by addition of 0.5 ml of lysis buffer (50 mM tris-HCl pH 7.5, 150 mM NaCl, 1% Nonidet P40, 0.5% sodium deoxycholate), mixed with protease inhibitors (1 tablet per 50 ml, Boehringer Ref.: 1697 498), and phosphatase inhibitors (Calbiochem Ref.: 524625 (1/100)). The cells were scraped off and the suspension was recovered and placed on a shaker at 4° C. for 1.5 hours. The solutions were centrifuged at 12,000 rpm for ten minutes (4° C.) and the protein concentrations of the supernatants were quantified by BCA.

500 μg of proteins of the cell lysate were mixed with the anti-IGF-IR (Santa Cruz Ref.: sc-713) for immunoprecipitation and incubated on the shaker at 4° C. for 1.5 hours. The immunoprecipitates were recovered by addition of protein A-agarose (Boehringer Ref.: 1 134 515) and incubated all night on the shaker at 4° C. For the immunoprecipitation of IRS-1, anti-IRS-1 antibodies coupled to agarose beads (Santa Cruz Ref.: 559Ac) were used. The agarose beads were washed twice with 1 ml of lysis buffer, twice with a wash buffer 1 (50 mM tris-HCl pH 7.5; 500 mM NaCl; 0.1% Nonidet P40; 0.05% sodium deoxycholate (Boehringer 1 332 597), mixed with protease inhibitors and phosphatase inhibitors) and once with a wash buffer 2 (50 mM tris-HCl; 0.1% Nonidet P40; 0.05% sodium deoxycholate (Boehringer Ref.: 1 332 597), mixed with protease inhibitors and phosphatase inhibitors 1/100). The immunoprecipitates were resuspended in a Laemmli buffer, heated to 100° C. for 5 minutes. The supernatants were analyzed by electrophoresis on polyacrylamide SDS gel (8% Novex EC6015). The proteins were transferred to a nitrocellulose membrane followed by either an immunoblot with anti-phosphotyrosine antibodies conjugated to HRP (upstate Biotechnology 4G10) or beta anti-chain of IGF-IR or anti-IRS-1 (Santa Cruz Ref.: sc 8038) followed by an anti-rabbit antibody conjugated to HRP. The imprints were revealed by chemiluminescence (Amersham RPN 2209) followed by autoradiography on Kodak X-mat AR films.

FIG. 40A represents MCF7 cells nonstimulated (0) or stimulated either with IGF-I (50 ng/ml) alone (0+IGF-I) or combined with monoclonal or humanized anti-IGF-IR antibodies (10 μg/ml) 7C10, 1H7, 7H2HM. The antibodies 9G4 or hIgG1 are murine or human immunoglobulins of isotype IgG1 used as an experiment negative control. The beta chains of the IGF-IR were immunoprecipitated and blotted with phosphorylated anti-tyrosine antibodies. The results obtained show that the monoclonal or humanized anti-IGF-IR 7C10, 1H7 and 7H2HM antibodies inhibit the phosphorylation of the tyrosine of the beta chain of the IGF-IR.

FIG. 40B represents MCF7 cells nonstimulated (0) or stimulated either with IGF-I (50 ng/ml) alone (0+IGF-1) or combined with monoclonal or humanized anti-IGF-IR antibodies (10 μg/ml) 7C10, 1H7, 7H2HM. As described above, the antibodies 9G4 or hIgG1 are murine or human immunoglobulins of isotype IgG1 used as an experiment negative control. The IRS-1 was immunoprecipitated and blotted with phosphorylated anti-tyrosine antibodies. The results obtained show that the monoclonal antibodies 7C10, 7H2HM and 1H7 inhibit the phosphorylation of the tyrosine of the IRS-1.

### EXAMPLE 24

### 7C10 And 7H2HM Induces the Internalization of the IGF-IR

MCF7 and A549 cells were suspended to 1.10⁷ cells/ml in PBS with 10% of fetal calf serum (FACS buffer). 1.10⁶ cells were incubated for 30 minutes at 37° C. with the monoclonal antibodies at 10 μg/ml (7C10, 7G3, 9G4) or at 20 μg/ml for 7H2HM. After washing, the cells were labeled at 4° C. for 30 minutes with a biotinylated anti-IGF-IR (monoclonal antibody 12B1) and finally incubated at 4° C. for 30 minutes with a conjugate of streptavidin-488 alexa Fluor®. The cells were analyzed by FACScan (Becton-Dickinson, Enembogegem, Belgium) with the Cellquest software after elimination of debris.

FIG. 41 shows the A549 cells without coloration (1^{st} peak), the A549 cells incubated with 7C10 or 7H2HM (2^{nd} peak) and the A549 cells incubated with an irrelevant mouse or rat IgG1 (3d peak). A decrease by two of the surface expression of the IGF-IR by the cells is seen when the cells have been previously incubated with 7C10 or 7H2HM.

### EXAMPLE 25

### 7C10 And 7H2HM Induce the Degradation of the IGF-IR

MCF-7 cells were cultured for 24 hours at 10.10⁴ cells/cm² (75 cm², Costar) in 15 ml of complete medium. Next, the cultures were washed three times with PBS and incubated for 12 hours with medium devoid of serum. Next, the cells were incubated with cycloheximide at 25 μg/ml alone or with 10 μg/ml of monoclonal antibody 7C10, 9G4, 7G3 or of IGF-I (50 ng/ml). In certain experiments, before incubation with the monoclonal antibodies, the cells were treated for 1 hour at 37° C. with MG-1 32 (10 μM, Calbiochem 474791) in order to inhibit the proteasome activities. After incubation, the cells were washed and solubilized by addition of a lysis buffer. 20 μg of proteins were analyzed by electrophoresis on polyacrylamide gel at 8% of SDS and transferred to a nitrocellulose membrane followed by a beta anti-chain immunoblot of the IGF-IR such as described further above.

The analysis by Western-blot (FIG. 42A) of the integrity of the IGF-IR shows that 7C10 and 7H2HM induce the degradation of the receptor while the natural ligand does not cause any degradation of the latter. No degradation of the receptor is observed with the 9G4, an irrelevant antibody used as an isotype control. FIG. 42B demonstrates, and with respect thereto, that the degradation is inhibited by a proteasome inhibitor MG132 (incubation period of 2 hours).

Comparable results were obtained with the humanized antibody 7H2HM (FIG. 42C).

### EXAMPLE 26

### Evaluation of 7C10 and H7C10 Ability to Bind to IGF-IR and Insulin/IGF-I Hybrid

### EXAMPLE 26.1

### Evaluation of 7C10 and h7C10 ability to immunoprecipitate IGF-IR and IR/IGF-IR Receptors Purified from Transfected Cells Respectively with IGF-IR and IR-A or IGF-IR and IR-B (Thereafter Referred as R+/IR−A or R+/IR−B

The goal of this study is to evaluate the ability of 7C10 and h7C10 to immunoprecipitate IGF-IR, IR or Hybrid-R.

7C10 and h7C10 are compared to 17-69 (which recognizes both IGF-IR well and Hybrid-R).

Method:

The used cells for this study are listed thereafter:

R+: R− fibroblasts stably transfected with the IGF-I receptor (IGF-IR) cDNA

R−/IR−A: R− fibroblasts stably transfected with the insulin receptor isoform A (IR-A) cDNA

R−/IR−B: R− fibroblasts stably transfected with the insulin receptor isoform B (IR-B) cDNA

R+/IR−A: R− fibroblasts stably co-transfected with the IGF-I and the insulin receptor isoform A cDNA and, therefore, expressing hybrid receptors A (Hybrid-RA)

R+/IR−B: R− fibroblasts stably co-transfected with the IGF-I and the insulin receptor isoform B cDNA and, therefore, expressing hybrid receptors A (Hybrid-RB)

For the obtention of cellular lysat, cells were solubilized in RIPA buffer and 4 mg protein used for immunoprecipitation.

Cell lysates were immuprecipitated as follows:
R+ with either 7C10 or h7C10
R+/IR−A and R+/IR−B with either 7C10 or h7C10 or 17-69
R−/IR−A and R−/IR−B with either MA-20 (an anti-IR antibody) or 7C10 or h7C10

Following immunoprecipitation, the pellet was resuspended in 2× sample buffer and subjected to SDS-PAGE (7.5% polyacrylamide).

Filters were blotted as follows: Filters containing R+ lysates (and therefore only IGF-IR) with an anti-IGF-IR β-subunit (Santa Cruz). Filters containing lysates from all the remaining cells with an antibody anti-IR β-subunit (Santa Cruz).

Results:

Two independent experiments are shown (FIG. 43A and FIG. 43B)

Comments:
1) 7C10 and h7C10 are equally efficient in immunoprecipitating the IGF-IR (lanes 1 and 2)
2) Neither 7C10 nor h7C10 appreciably immunoprecipitate IR
3) Both 7C10 and h7C10 recognizes Hybrid-Rs.

### EXAMPLE 26-2

### Displacement analysis of IGf-I On IGF-IR by 7C10, H7C10 and 1H7

IGF-IR from R+ cell lysates were immunocaptured in Maxisorb plates coated with 17-69 antibody.

¹²⁵I-IGF-I (FIG. 44) was then allowed to bind to immunocaptured receptors in the absence or the presence of increasing concentrations of unlabeled ligand (IGF-I) or antibodies (7C10, h7C10, 1H7, 9G4). Results are plotted as percent of maximal binding.

Both 7C10 and h7C10 displace labeled IGF-I with a very similar efficiency. By comparison, 1H7 was much less effective (FIG. 44).

### EXAMPLE 26-3

### Displacement Analysis of IGF-1 On Hybrid-RA by 7C10, H7C11 and 1H7

Hybrid-RA from R−/IR-A cell lysates were immunocaptured in Maxisorb plates coated with anti IR antibody 83-7.

125I-IGF-I (FIG. 45) was then allowed to bind to immunocaptured receptors in the absence or the presence of increasing concentrations of unlabeled ligand (IGF-I) or antibodies (7C10, h7C10, 1H7, 9G4). Results are plotted as percent of maximal binding.

Both 7C10 and h7C10 displace labeled IGF-I with a very similar efficiency. By comparison, 1H7 was much less effective (FIG. 45).

### EXAMPLE 26-4

### Displacement Analysis of IGF-10N Hybrid-Rb by 7C10, H7C10 and 1H7

Hybrid-RB from R−/IR-B cell lysates were immunocaptured in Maxisorb plates coated with 83-7 antibody.

125I-IGF-I (FIG. 46) was then allowed to bind to immunocaptured receptors in the absence or the presence of increasing concentrations of IGF-I or antibodies (7C10, h7C10, 1H7, 9G4). Results are plotted as percent of maximal binding.

Both 7C10 and h7C10 displace labeled IGF-I with a very similar efficiency. By comparison, 1H7 was much less effective (FIG. 46).

### EXAMPLE 27

### Internalization and Degradation Studies of the IGF-IR

Internalization and degradation studies were analyzed by FACS and western-blot analysis. Internalization studies were performed by FACS analysis using a murine biotinylated anti-IGF-IR monoclonal antibody (Mab) thereafter described as 12B1 MAb and binding to an epitope different from the one recognized by 7C10 and h7C10 antibodies. The 7G3 MAb, a non neutralizing anti-IGF-IR was introduced as negative control. Both antibodies were generated in our laboratory. Confluent MCF-7 cells were trypsinized and 1×10⁶ cells from each cellular suspension was plated in 96-well plates in FACS buffer. Plates were incubated, either with or without 25 μg/ml of cycloheximide (Calbiochem), 30 min at 37° C. with either IGF1 (50 ng/ml) or with 10 μg/ml of 7C10, 7G3, h7C10, mIgG1, hIgG1. Cells incubated with FACS buffer alone were used to determine the basal level of expression of the IGF-IR. Then cells were washed twice and 12 μg/ml of biotinylated-12B1 MAb were added to the plate. After 30 min of incubation at 4° C. to avoid receptor internalization, cells were washed 3 times at 4° C. and stained by addition of a streptavidin Alexa Fluor® 488conjugate (Molecular Probes Europe BV, Leiden, Netherlands).

Both 7C10 and h7C10 cause a rapid down regulation of the IGF-IR with a maximum after 4 hours of incubation with the antibodies (Table 11). No down regulation was observed when cells were incubated either with IGF1, 7G3 non neutralizing Mab, murine (mIgG1) or human (hIgG1) isotype control. The absence of internalization when cells were incubated with IGF-I is probably due to the rapid recycling of IGF-IR; indeed this rapid recycling phenomenon is well known by the man skill in the art for this type of receptor. These results were observed either in presence or in absence of cyclohexemide. Observed results are shown in the following Table 11.

TABLE 11

| | | | Cells incubated without Cyclohexemide | | | Cells incubated with Cyclohexemide | | |
|---|---|---|---|---|---|---|---|---|
| | | | | mIgG1 | 12B1 | | mIgG1 | |
| | | | Buffer | Biotinylé | Biotinylé | Buffer | Biotinylé | 12B1 Biotinylé |
| 5 | min | Buffer | 8 | 8 | 135 | 8 | 8 | 90 |
| | | IGF1 | 8 | 9 | 137 | 8 | 9 | 93 |
| 1 | h | Buffer | 9 | 9 | 153 | 8 | 8 | 89 |
| | | hIG1 | 8 | 9 | 150 | 8 | 9 | 92 |
| | | h7C10 | 9 | 9 | 64 | 8 | 8 | 37 |
| | | mIgG1 | 8 | 9 | 144 | 8 | 8 | 88 |
| | | 7C10 | 9 | 9 | 61 | 8 | 9 | 36 |
| | | 7G3 | 8 | 9 | 137 | 8 | 8 | 85 |
| 4 | h | Buffer | 8 | 8 | 136 | 8 | 8 | 95 |
| | | hIgG1 | 8 | 8 | 139 | 7 | 8 | 94 |
| | | h7C10 | 8 | 8 | 39 | 8 | 8 | 29 |
| | | mIgG1 | 9 | 9 | 130 | 8 | 8 | 78 |
| | | 7C10 | 8 | 8 | 37 | 8 | 8 | 27 |
| | | 7G | 8 | 8 | 109 | 8 | 8 | 72 |
| 16 | h | Buffer | 8 | 9 | 135 | 8 | 9 | 85 |
| | | HIgG1 | 9 | 9 | 144 | 8 | 8 | 85 |
| | | H7C10 | 9 | 10 | 34 | 8 | 9 | 26 |
| | | MIgG1 | 9 | 10 | 100 | 10 | 10 | 56 |
| | | 7C10 | 9 | 9 | 31 | 9 | 9 | 25 |
| | | 7G3 | 9 | 9 | 90 | 9 | 9 | 57 |

### Table 11

### Study of Antibody Induced IGF-IR Internalization by FACS Analysis

For immunoblotting experiments 7.5×10⁶ cells were plated in 75 cm² flasks in 15 ml of complete medium (red phenol-free RPMI and Ham-F12K respectively for MCF-7 and A549 both supplemented with 10% FCS and 1% L-Glutamine). Twenty four hours after plating, cells were washed 3 times with PBS and incubated for 24 additional hours at 37° C. Then medium was removed and cells incubated either 1 h, 4 h or 16 h at 37° C. with 15 ml of serum-free medium with or without antibodies to be tested or with IGF-I. Cells were then harvested and lysed in Tris HCl buffer pH 7.5, 15% NaCl 1M (Sigma), 10% detergent mix (10 mM Tris-HCl, 10% Igepal) (Sigma), 5% sodium deoxycholate (Sigma), 1 protease inhibitor cocktail complete TM tablet (Roche) and 1% phosphatase inhibitor Cocktail Set II (Calbiochem). For Western blot analysis, equal amount of cell lysates were separated on 10% SDS-PAGE, transferred to nitrocellulose filters, probed with an anti-β IGF-IR rabbit polyclonal IgG (Santa Cruz Biotech), revelated with an anti rabbit IgG coupled to the HRP (Amersham Bioscience) and visualized by ECL (Amersham Bioscience).

FIGS. 47A and 47B represent the study of antibody induced degradation of the IGF-IR.

For immuno-blotting analysis (FIGS. 47A and 47B), experiments were done without cyclohexemide as the above experiment shows that no difference was observed in presence or in absence of this compound. 7C10 and h7C10 cause a comparable internalization of the IGF-IR in both A549 (A) and MCF-7 (B) cells. In MCF-7 cells the maximal internalization was observed after four hours incubation with 7C10 and h7C10, whereas, for A549 the maximal internalization is observed as earlier as 1 hour. No degradation was observed when cells were incubated either with IGF-I, 7G3 or murine (mIgG1) or human (hIgG1) isotype control.

### EXAMPLE 28

### Study of the Degradation Pathway of IGF-IR

7.5×10⁶ MCF-7 cells were plated in 75 cm² flasks in 15 ml of complete medium (red phenol-free RPMI supplemented with 10% FCS and 1% L-Glutamine). Twenty four hours after plating, cells were washed 3 times with PBS and incubated for 24 additional hours at 37° C. in 15 ml serum-free medium. Then medium was removed and cells incubated for two hours in 7.5 ml of serum-free medium either containing 30 μM MG115 or DMSO. Then, 7.5 ml of serum-free medium with or without h7C10, hIgG1 or IGF-I were added for 4 additional hours. Cells were then harvested and lysed in Tris HCl buffer pH 7.5, 15% NaCl 1M (Sigma), 10% detergent mix (10 mM Tris-HCl, 10% Igepal) (Sigma), 5% sodium deoxycholate (Sigma), 1 protease inhibitor cocktail complete TM tablet (Roche) and 1% phosphatase inhibitor Cocktail Set II (Calbiochem). For Western blot analysis, equal amount of cell lysates were separated on 10% SDS-PAGE, transferred to nitrocellulose filters, probed with an anti-P IGF-IR rabbit polyclonal IgG (Santa Cruz Biotech), revelated with an anti rabbit IgG coupled to the HRP (Amersham Bioscience) and visualized by ECL (Amersham Bioscience). FIG. 48 shows the obtained results.

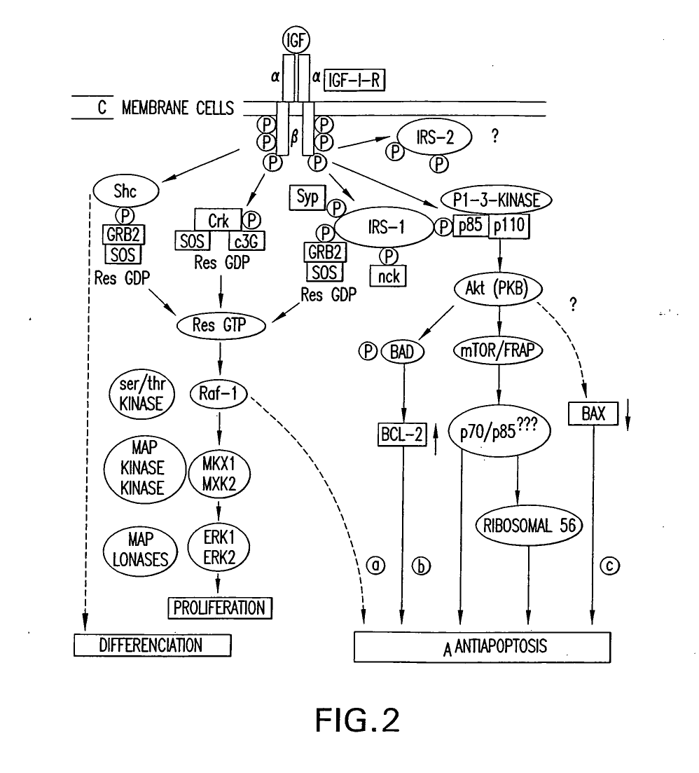

To further characterize the pathway of degration of the h7C10 antibody, cells were incubated 4 hours with either IGF-I or human isotype control (hIgG1) in presence or in absence of the proteasome inhibitor MG115. In the herein described experiment h7C10 induced, a dramatic degradation of the IGF-IR either in presence or in absence of DMSO. No degradation was observed when IGF-I or hIgG1 were added. When cells were incubated with 30 μM MG115, no down regulation of the IGF-IR was observed demonstrating that the down regulation of IGF-IR on MCF-7 observed in FIG. 2 occurs through the proteasome pathway. This property is surprising and of particular interest. Indeed none of the anti-IGF-IR antibody already described for inducing a degradation of the IGF-IR (Malauney E K and al, Cancer Research, 2003; Sachdev D and al, Cancer Research, 2003) involved the proteasome pathway for degradation.

Actually, it has been reported that IGF-IR is internalized and degraded via a lysosome-dependent pathway (Alessi et Al., B. Curr. Biol., 1997). In addition, both Mab391 (Hailey et Al., Molecular Cancer Therapeutics, 2002) and scFv-Fc (Sachdev et Al., Cancer Research, 2003) down regulate IGF-IR by the endocytic pathway.

As a consequence, regarding the present knowledge, it can not be exclude that h7C10 also down regulate, in addition to the proteasome pathway as previously described, via other known and described pathways for anti-IGF-IR antibodies, i.e. lisosome-dependent and/or endocytic pathways.

Such a property, if validated, is of particular interest as it would demonstrate the capacity of the h7C10 to interact with different signalization/degradation pathways, and thus its therapeutic efficacy. Supplementary studies are in progress in order to validate this hypothesis.

### EXAMPLE 29

### Anti-Tumoral Activity of the Murine Antibody 7C10 Co-Administrated With an Anti-VEGF Antibody on Mice Orthopically Implanted with A549 Cells

One million of A549 NSCLC were implanted through the chest wall into the left pleural cavity space of 6 weeks old Swiss nude mice following the protocol described by Klaus-Berthier et al. (Kraus-Berthier, L., Jan, M., Guilbaud, N., Naze, M., Pierre, A., and Atassi, G., Histology and sensitivity to anticancer drugs of two human non-small cell lung carcinomas implanted in the pleural cavity of nude mice. Clin. Cancer Res. 6 (1): 297-304, 2000). Seven days after the cell injection, mice were treated i.p. with a loading dose of 250 μg of antibodies, and them, twice a week with 125 μg of antibodies. For the combined therapy, antibodies were mixed prior to the injection.

The anti-VEGF antibody used was an IgG2b, clone 26503.11 commercialized by SIGMA. It was described as a neutralizing antibody (Ferrara N. et al., Biochem. Res. Com. 161:851. 1999; Ferrara et al., Endocrinol. Review 13:18.1992; Leung D. W. et al., Science 246:1306, 1989).

FIG. 48 shows that a combined therapy increase dramatically the time survival compared to untreated mice or to mice treated with single therapy.

The T/C % are calculated according the following formula, [MEDIAN OF TREATED MICE/MEDIAN OF CONTROL MICE×100]. The obtained T/C % are about 134% and 144% for the 7C10 and anti-VEF antibody respectively. For the combined treatment 7C10+ anti-VEGF antibodies, the T/C % is 188%.

As a conclusion, similarly to the co-administration of 7C10+225 (see example 22), the co-administration of 7C10+ anti-VEGF antibodies increase the mice survival.

### EXAMPLE 30

### Production of Deoxyvinblastine

4′-R deoxyvinblastine (structure see below Scheme 1) is obtained by ionic reduction of anhydrovinblastine according to a process known to those skilled in the art (Lafitte C et al., Tetrahedron Letters, 1998, Volume 39, pp. 8281-8282).

4′-S deoxyvinblastine, or 4′-S deoxyleurosidine, is obtained by catalytic hydrogenation of anhydrovinblastine according to the technique also known to those skilled in the art (De-Bruyn A. et al., Bulletin of the Belgian Chemical Society, 1983, Volume 92, number 5, pp 485-494).

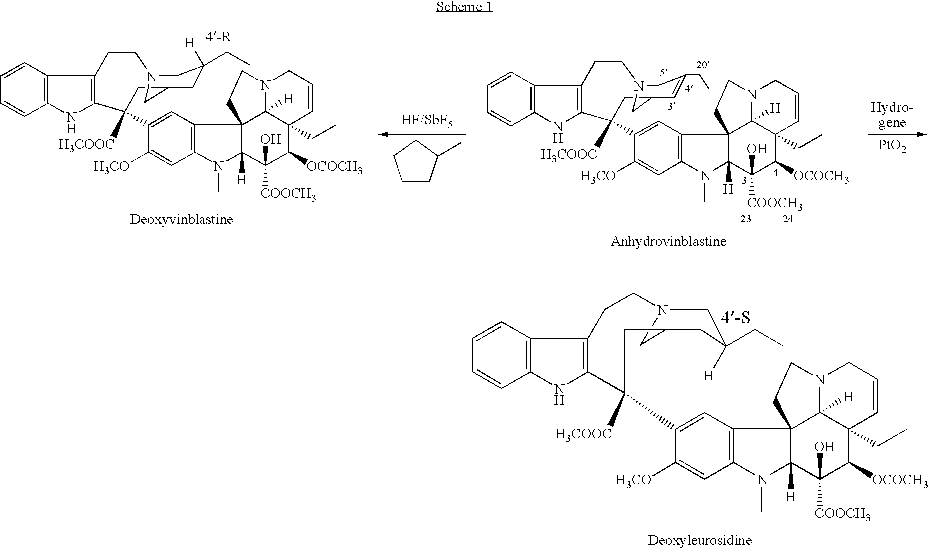

### EXAMPLE 31

### Deacetylation of Vinca Dimeric Alkaloids

Deoxyvinblastine or deoxyleurosidine is dissolved and stirred for 4 hours at 50° C. in 30 ml of methanol containing 1.2 equivalents of sodium methoxide. This solution is then poured into ice-cold water in order to precipitate the compound formed. After filtration, washing with water and drying under vacuum at 40° C., 4-deacetyldeoxyvinblastine or 4-deacetyldeoxyleurosidine is obtained, with a purity of greater than 95%.

### EXAMPLE 32

### Direct Coupling of 4′-Deoxyvinblastine (4′ R) or 4′-Deoxyleurosidine (4′ S) By Reaction of a 4-Carboxyhydrazide Function on the Pre-Oxidized ANTI-IGF-IR Antibodies

The 4′-deoxyvinblastine or the 4′-deoxyleurosidine is treated with anhydrous hydrazine in solution in methanol and at ambient temperature. The reaction is monitored by Analytical High Performance Liquid Chromatography (HPLC) and, when 95% of the starting alkaloid has reacted, the reaction medium is treated with water in order for the 4′-deoxyvinblastine-3-deacetyl-4-carbohydrazide or the 4′-deoxyleurosidine-3-deacetyl-4-carbohydrazide to be separated by filtration.

After silica gel chromatography and then crystallization, the 4′-deoxyvinblastine-3-deacetyl-4-carbohydrazide or the 4′-deoxyleurosidine-3-deacetyl-4-carbohydrazide is greater than 96% pure.

The anti-IGF-IR antibody is oxidized under cold conditions in a sodium acetate buffer by treatment with sodium meta-periodate. After exclusion chromatography, the oxidized anti-IGF-IR antibody, in solution in an acetate buffer, is treated under cold conditions with the 4′-deoxyvinblastine-3-deacetyl-4-carbohydrazide or the 4′-deoxyleurosidine-3-deacetyl-4-carbohydrazide.

The immunoconjugate thus obtained is separated from the unconjugated residual Vinca alkaloid and purified by exclusion chromatography with a phosphate buffer at pH 7.4, and then intensive dialysis. The absence of free Vinca alkaloid is verified by analytical HPLC.

The immunoconjugate is characterized on an SDS PAGE-type electrophoresis gel (Coomassie blue and/or silver nitrate), by exclusion chromatography (SEC, UV at 280 nm) and by MALDI-TOF mass spectrometry. The mapping of the coupling sites is carried out by means of analysis by liquid chromatography coupled to mass spectrometry (LC MS), subsequent to enzyme digestion (trypsin and PNGase F) (Laguzza et al., J. Med. Chem., 1989, 32:548).

### EXAMPLE 33

### Coupling of the 4′-Deoxyvinblastine (4′ R) or the 4′-Deoxyleurosidine (4′ S) To the ANTI-IGF-IR Antibodies by Virtue of Succinic Anhydride

The 3-deacetyl-4′-deoxyvinblastine or the 3-deacetyl-4′-deoxyleurosidine is treated with succinic anhydride in pyridine for 24 hours at 20° C. The reaction is monitored by analytical HPLC and, when 95% of the starting alkaloid has reacted, the reaction medium is treated with water in order to precipitate the 3-deacetyl-4′-deoxyvinblastine hemisuccinate or the 3-deacetyl-4′-deoxyleurosidine hemisuccinate. After filtration and drying, the compound is purified by reverse-phase preparative HPLC using C18 grafted silica and an eluent made up of acetonitrile, methanol and ammonium acetate buffer.

The 3-deacetyl-4′-deoxyvinblastine hemisuccinate or the 3-deacetyl-4′-deoxyleurosidine hemisuccinate is treated with hydroxybenzotriazole and dicyclohexylcarbodiimide in dimethylformamide at ambient temperature for 24 hours and in the presence of a catalytic amount of dimethylaminopyridine. After filtration, the solution is mixed with the anti-IGF-IR monoclonal antibody at pH 8.6 for 4 hours. The immunoconjugate is separated from the unconjugated Vinca alkaloid by exclusion chromatography with a phosphate buffer at pH 7.4. Intensive dialysis makes it possible to eliminate the unconjugated Vinca alkaloid. The immunoconjugate is characterized by SDS PAGE gel electrophoresis, by exclusion chromatography and by MALDI TOF mass spectrometry. The mapping of the coupling sites is carried out by means of liquid chromatography analysis coupled to mass spectrometry (LC MS), subsequent to enzyme (trypsin) digestion, compared to a reference tryptic map obtained for the non-derived monoclonal antibody (Schneck et al., Clin. Pharmacol. Ther., 1990, 47:36; Rowland et al., Cancer. Immunol. Immunother., 1985, 19:1).

### EXAMPLE 34

### Coupling of the 4′-Deoxyvinblastine (4′ R) or the 4′-Deoxyleurosidine (4′ S) On a Nitrogen-Containing Residue of the ANTI-IGF-IR Antibodies by Virtue of a Disulphide Bridge Included in the Linkage

The 3-deacetyl-4′-deoxyvinblastine or the 3-deacetyl-4′-deoxyleurosidine is treated, in methylene chloride, at ambient temperature for 24 hours, in the presence of a catalytic amount of dimethylaminopyridine, with a large excess of 3-methyldisulphanylpropanoic acid and a large excess of dicyclohexylcarbodiimide. The reaction medium is treated conventionally and the 3-deacetyl-4′-deoxyvinblastine 3-methyldisulphanylpropanoate or the 3-deacetyl-4′-deoxyleurosidine 3-methyldisulphanylpropanoate is then purified by reverse-phase preparative HPLC using C18 grafted silica and an eluent made up of acetonitrile, methanol and ammonium acetate buffer.

The 3-deacetyl-4′-deoxyvinblastine 3-methyldisulphanylpropanoate or the 3-deacetyl-4′-deoxyleurosidine 3-methyldisulphanylpropanoate is treated with dithiothreitol in a mixture of water and methanol so as to obtain 3-deacetyl-4′-deoxyvinblastine 3-sulphanylpropanoate or 3-deacetyl-4′-deoxyleurosidine 3-sulphanylpropanoate, which is purified by reverse-phase preparative HPLC using C18 grafted silica and an eluent made up of acetonitrile, methanol and ammonium acetate buffer.

The anti-IGF-IR antibody is derivatized with N-succinimidyl 4-(2-pyridyldithio)propanoate (the trade name of which is SPDP) in a 50 mM potassium phosphate buffer, pH 6.5, containing 50 mM NaCl and 2 mM EDTA, for 90 minutes. Added to this solution of antibody thus derivatized is the 3-deacetyl-4′-deoxyvinblastine 3-sulphanylpropanoate or the 3-deacetyl-4′-deoxyleurosidine 3-sulphanylpropanoate dissolved in a minimum of DMSO. After contact for 24 hours, the immunoconjugate is isolated by exclusion chromatography and is characterized on an SDS PAGE electrophoresis gel, by exclusion chromatography and by MALDI TOF mass spectrometry (Ojima et al., J. Med. Chem., 2002, 45:5320).

### EXAMPLE 35

### Coupling of the 4′-Deoxyvinblastine (4′ R) or the 4′-Deoxyleurosidine (4′ S) To the ANTI-IGF-IR Antibodies by Virtue of a Terminal Hydrazide Function Carried by a Linkage Connected to the Vinca Alkaloid

The 3-deacetyl-4′-deoxyvinblastine or the 3-deacetyl-4′-deoxyleurosidine is treated, in methylene chloride at ambient temperature for 24 hours, in the presence of a catalytic amount of dimethylaminopyridine, with an excess of methyl monoester of 1,6-hexanedicarboxylic acid and an excess of dicyclohexylcarbodiimide. The reaction medium is treated conventionally and the 3-deacetyl-4′-deoxyvinblastine 3-methoxycarbonyl pentanoate or the 3-deacetyl-4′-deoxyleurosidine 3-methoxycarbonyl pentanoate is then purified by reverse-phase preparative HPLC using C18 grafted silica and an eluent made up of acetonitrile, methanol and ammonium acetate buffer.

The 3-deacetyl-4′-deoxyvinblastine 3-methoxycarbonyl pentanoate or the 3-deacetyl-4′-deoxyleurosidine 3-methoxycarbonyl pentanoate is treated by default with anhydrous hydrazine in solution in methanol at ambient temperature. The reaction is monitored by analytical HPLC and, when 70% of the starting alkaloid has reacted, the reaction medium is evaporated and the 3-deacetyl-4′-deoxyvinblastine 3-hydrazinocarbonyl pentanoate or the 3-deacetyl-4′-deoxyleurosidine 3-hydrazinocarbonyl pentanoate is purified by reverse-phase preparative HPLC using C18 grafted silica and an eluent made up of acetonitrile, methanol and ammonium acetate buffer.

The oxidation of the anti-IGF-IR antibody, the coupling with 3-deacetyl-4′-deoxyvinblastine 3-hydrazinocarbonyl pentanoate or 3-deacetyl-4′-deoxyleurosidine 3-hydrazinocarbonyl pentanoate, the purification and the identification are carried out according to the same techniques as those described in Example 32.

### EXAMPLE 36

### Activity, Compared In Vivo, of the 7C10 and H7C10 Antibodies on the A549 And MCF-7 Models

In order to confirm the activity of the humanized antibody h7C10 in vivo, the latter was compared with 7C10 in the MCF-7 oestrogen-dependent breast tumour model and in the A549 non-small-cell lung tumour model.

To do this, 5.106 A549 cells were implanted subcutaneously in nude mice. Five days after this implantation, the tumours were measured and groups of 6 mice were formed. These groups were treated, respectively, with 1) the 7C10 antibody injected ip (intraperitoneally) at a rate of 125 μg/dose twice a week; 2) the h7C10 antibody injected under the same conditions as its murine form; 3) PBS (it has been shown previously that murine and human control isotypes do not modify the tumour growth profile compared to treatment of the animals with PBS). In the MCF-7 breast tumour model, a sustained-release estradiol granule (0.72 mg/tablet released over 60 days) is implanted subcutaneously 24 hours before implantation of the cells. This granule is essential to the establishment of any E2-dependent human tumour in this animal species.

FIGS. 50 and 51 show, as expected, that significant inhibition of tumour growth is observed with the 7C10 murine antibody. As regards the h7C10 humanized antibody, the activity observed is of exactly the same intensity as that observed with its murine counterpart, whatever the model used. This datum indicates that the humanization has not modified the properties of the antibody generated.

### EXAMPLE 37

### Demonstration of the Compared Activities of Vinblastine, of Vincristine, of 4′ S Deoxyvinblastine and of 4′ R Deoxyleurosidine

The greater activity of the (4′ R) deoxyvinblastine and of the (4′ S) deoxyleurosidine was demonstrated in vivo against intravenously-grafted P388 murine leukaemia and compared with the activity of vinblastine and of vincristine tested under the same conditions. The protocol for this test is described by Kruczynski A. et al., Cancer Chemotherapy and Pharmacology, 1998, volume 41, pages 437 to 447.

To do this, a total of 106 P388 murine leukaemia cells were implanted i.v. in CDF1 mice on day 0. After randomization of the animals in cages for treatment with each alkaloid and control cages, the compounds were administered i.p. on day 1.

Conventionally, the in vivo activity of compounds is expressed by the increase in survival time. The survival time is expressed by the T/C at a dose expressed in mg per kg (mg/kg). The T/C corresponds to the ratio, multiplied by 100, of the median of the survival time of the treated animals to the median of the survival time of the control animals. In agreement with the standard criteria of the NCI, a T/C of 120 corresponds to a minimum level for concluding that activity is present.

A T/C of between 120 and 175 makes it possible to conclude that there is significant activity and a T/C above 175 makes it possible to conclude that there is a high level of anti-leukaemia activity. A T/C below 75 expresses toxicity of the test compound at the dose administered.

Table 12 below gives the results obtained with a minimum of 7 and a maximum of 15 treated mice for each group of animals treated with a Vinca alkaloid or for the control group.

Table 12 gives the results of T/C values obtained for each Vinca alkaloid tested.

FIGS. 52 and 53 show the greater anti-leukaemia activity of the 4′R and 4′S deoxyvinblastines compared to vinblastine and vincristine.

TABLE 12

| | Dose in mg/kg | | | | | | |
|---|---|---|---|---|---|---|---|
| | 0.63 | 1.25 | 2.5 | 5 | 10 | 20 | 40 |
| T/C for vinblastine | 114 | 114 | 129 | 143 | 57 | | |
| T/C for vincristine | 114 | 143 | 143 | 100 | 57 | | |
| T/C for 4′-S- | 114 | 143 | 200 | 100 | 57 | | |
| deoxyvinblastine | | | | | | | |
| T/C for 4′-R- | 100 | 100 | 129 | 143 | 200 | 214 | 43 |
| deoxyvinblastine | | | | | | | |

### EXAMPLE 38

### Demonstration of the In Vivo Antitumour Activity of 4′ R- and 4′ S-Deoxyvinblastine Conjugated with IGR-IR Antibodies on Human Tumours of Various Origins

In order to demonstrate the benefit of addressing the chemotherapy compounds (4′ R) and (4′ S) deoxyvinblastine (respectively called RDV and SDV in FIG. 5) with a humanized antibody directed against IGF-IR, 5.106 A549 non-small-cell lung cancer cells were implanted in a subcutaneous position on the right flank of Swiss Nude mice. Seven days after implantation of the cells, the tumours can be measured and the animals are distributed randomly into 6 groups of 6 mice and treated according to the following protocol:
h7C10: twice a week at a rate of 250 μg/dose throughout the entire duration of the experiment;
RDV and SDV: 4 intraperitoneal injections 7 days apart at the dose of 0.35 mg/kg, which corresponds to the dose of each of the compounds present in the conjugates;
the groups of animals given the chemotherapy compounds coupled to the antibody receive respectively 0.35 mg/kg of each of the chemotherapy agents and 250 μg/dose of antibodies. These conjugates are administered according to the same modes as the groups given the chemotherapy compounds alone;
the animals of the control batch are given injections of PBS, administered according to the same frequency.

The weight of the mice and the tumour volume are evaluated twice a week. The tumour volumes are calculated according to the formula: ½ (length. width. height).

The results are shown in FIG. 53.

The animals given only RDV or SDV evolve in the same manner as the control group, which seems coherent with respect to the optimum doses usually injected for these two compounds, which are respectively 20 mg/kg and 2.5 mg/kg. Surprisingly, when each of the compounds is coupled to the h7C10 antibody, a very significant inhibition of the tumour growth is observed. This inhibition is significantly greater than that observed with the antibody alone, administered at the same concentration.

All these results appear to indicate that targeting of the cells with the h7C10 antibody promotes concentration of the drug in the cell to be targeted and makes it possible to observe, as a result, significant inhibitions of tumour proliferation at low doses of chemotherapy product, and in particular at doses which are completely non-toxic in mice, as is demonstrated by the lack of weight loss of the animals (data not communicated).

### EXAMPLE 39

### Anti-Tumor Activity of the Humanized Mk-0646 Antibody Alone or Combined with Avastin® (A5490905)

### Materials and Methods

A549 cells from ATCC were routinely cultured in F12K medium (Invitrogen Corporation, Scotland, UK), 10% FCS (Invitrogen Corporation). Cells were split two days before engraftment so that they were in exponential phase of growth. Before engrafrnent, animals were anesthetized with a 4/1 mixture of ketamine (Imalgène® 500; Rhône Mérieux, Lyon, France) and xylasine (Rompun® at 2%; Bayer, Puteaux, France) administered i.m. One million tumor cells were implanted through the chest wall into the left pleural space of 8 weeks old nude mice (i.pl.) in a volume of 100 μl using a 26 gauge needle. The primary tumor evolved on day 4 already spread locally to continuous structures, including mediastinum, lung and diaphragm. To better mimic a clinical sample, treatment only started when the disease had developed, 7 days after i.pl. injection of A549 tumor cells.

Four groups of 12 mice were generated at random and treated twice a week with either MK-0646 or Avastin® Abs alone at 500 μg/mice for the loading dose and then twice a week for 5 weeks at 250 μg/mice. A group of mice receiving both MK-0646 and Avastin® Abs was also included. The control group was injected with PBS. Mice were monitored for life span.

The anti-tumor activity was evaluated as follows: T/C %=median survival time of treated group/median survival time of control group X 100. Statistical analysis were performed using a log Rank test.

### Results:

As shown in FIG. 55, mice receiving the combination of MK-0646 and Avastin® antibodies exhibited increased survival in the orthotopic A549 model with a T/C value of 173% on day 146 post-cell injection compared to 116% and 146% for MK-0646 and Avastin® single modality treatments, respectively. Statistical analysis demonstrated that each antibody, when administered alone, did not significantly increase survival. On the other hand, the combination therapy comprising Mk-0646 and Avastin® significantly increased the survival of mice versus the control group (p=0.004) as well when compared to mice treated with either MK-0646 alone (p=0.0003) or Avastin® alone (p=0.0003).

### EXAMPLE 40

### Anti-Tumor Activity of the Humanized Mk-0646 Antibody Alone or Combined with Avastin® in the A549 Xenograft Model (Experiment 01A54901406)

### Materials and Methods

A549 cells were routinely cultured in F12K (Gibco/Invitrogen, Verviers Belgium) supplemented with 10% heat inactivated Foetal Calf Serum (Sigma Chemical Co. St Louis, Mo.). Cells were split two days before engraftment so that they were in exponential phase of growth. Ten million A549 cells (P96) were engrafted on 7 weeks old Swiss-Nude mice (strain Crl: NU(Ico)-Foxn1nu, Charles River Laboratories).

Four days after engraftment, tumors were measurable and animals were divided into homogeneous tumor size groups of 6 mice.

Five days after engraftment (D0) mice were treated i.p. with the following schemes:

Control group: PBS, twice/week

MK-0646 group: MK-0646, 1 mg/dose per mice, twice/week

Avastin group: Avastin, 0.1 mg/dose per mice, twice/week

Co-administration combination group: extemporaneous mix of MK-0646 (1 mg/dose) and Avastin (0.1 mg/dose), twice/week

Sequence combination group: D0 injection of a loading dose of Avastin (0.2 mg/dose per mice) followed on D3 by an injection with MK-0646 at the loading dose of 2 mg/dose per mice. Then, from D6 to end of experiment: same dosage as the one described for the co-administration combination group.

Tumor volume was determined twice a week using the formula: π/6.length.width.height.

### Results:

Data from this experiment is set forth in FIG. 56. As shown, when administered alone, each of MK-0646 and Avastin demonstrated an inhibitory effect on A549 tumor cell growth. For example, 44% tumor growth inhibition was observed with MK-0646 at D37, compared to the control group (p values <0.03 from D6 to D31). Mice in the control group were sacrificed at D37 for ethical reasons.

As regards the mice treated with Avastin, an 81% inhibiting effect was observed when compared to control mice at D37 (p values <0.04 from D10 to D37).

Compared to both single-agent therapies, the two combination groups designed in this study showed significantly higher tumor growth inhibition. For example, when Avastin and MK-0646 were strictly co-administered, tumor growth/tumor burden was dramatically reduced compared to:

control group: p value <0.03 from D3 to D37, 94% inhibition at D37,

MK-0646 group: p value ≦0.004 from D10 to D41, 89% inhibition at D41 (D41=euthanasia of MK-0646 group),

Avastin™ group: p value ≦0.04 from D6 to D52, 59% inhibition at D52 (end of the experiment).

As regards the mice in the sequence combination group, there appeared to be no difference in the tumor growth inhibition compared to the co-administration group, with strongly decreased tumor volumes compared to control and single-agent groups:

control group: p value=0.002 from D10 to D37, 95% inhibition at D37,

MK-0646 group: p value ≦0.015 from D6 to D41, 90% inhibition at D41 (D41=euthanasia of MK-0646 group), and Avastin™ group: p value ≦0.04 from D10 to D52, 58% inhibition at D52 (end of the experiment)

### EXAMPLE 41

### Anti-Tumor Activity of the Humanized MK-0646 Antibody Either Alone Or Combined with Herceptin® (A5491005)

### Materials and Methods

A549 cells from ATCC were routinely cultured in F12K medium (Invitrogen Corporation, Scotland, UK), 10% FCS (Invitrogen Corporation). Cells were split two days before engraftment so that they were in exponential phase of growth. Before engraftment, animals were anesthetized with a 4/1 mixture of ketamine (Imalgène® 500; Rhône Mérieux, Lyon, France) and xylasine (Rompun® at 2%; Bayer, Puteaux, France) administered i.m. One million tumor cells were implanted through the chest wall into the left pleural space of 9 weeks old nude mice (i.pl.) in a volume of 100 μl using a 26 gauge needle. The primary tumor evolved on day 4 already spread locally to continuous structures, including mediastinum, lung and diaphragm. To better mimic a clinical environment, treatment only started when the disease was developed, 8 days after i.pl. injection of A549 tumor cells.

Four groups of 10 mice were generated at random and treated twice a week with either MK-0646 or Herceptin® Abs alone at 500 μg/mice for the loading dose and then twice a week, for 5 weeks at 250 μg/mice. A group of mice receiving both MK-0646 and Herceptin® Abs was included. The control group was injected with PBS. Mice were monitored for life span.

The anti-tumor activity was evaluated as follows: T/C %=median survival time of treated group/median survival time of control group X 100. Statistical analysis was performed using a log Rank test.

### Results:

FIG. 57 demonstrates that the combination of both MK-0646 and Herceptin® antibodies increased the survival of mice in the orthotopic A549 model with a T/C value of 151% on day 180 post-cell injection compared to 104% and 93% for MK-0646 and Herceptin® single modality treatments, respectively. Statistically, each antibody, when administered alone, did not significantly increase survival. On the other hand, the combination of both antibodies significantly increased the survival of mice versus control group (p=0.00003) as well as mice treated with either MK-0646 alone (p=0.005) or Herceptin® alone (p=0.000003).

### EXAMPLE 42

### In Vivo Activity of Gemcitabine and Mk-0646 in Combination in BXPC3 Xenograft Nude Mice (BXPC31005)

### Materials and Methods

BxPC-3 cells from ATCC were routinely cultured in RPMI 1640 medium (Invitrogen Corporation, Scotland, UK), 10% FCS (Sigma Chemical Co. St Louis, Mo.), 2 mM L-Glutamine (Invitrogen Corporation), 10 mM hepes (Gibco Verviers Belgium), 1 mM sodium pyruvate (Biowhittaker/Cambrex Walkerville Md., USA), 2.5 g/l glucose (Sigma). Cells were split two days before engraftment so that they were in exponential phase of growth. Seven million BxPC-3 (P31) were engrafted in PBS to 7 weeks old Athymic nude mice (Harlan, France).

Four days after implantation, tumors were measurable and animals were divided into groups of 6 mice with comparable tumor size. Mice were treated i.p. with a loading dose of MK-0646 of 25 μg/mouse, then twice a week with 12.5 μg/dose/mouse of MK-0646 or once a week with gemcitabine, at 138.5 mg/kg or with both compounds. A PBS group was introduced as a control in this experiment.

Tumor volume was measured twice a week and calculated by the formula: 7c/6×length×width×height.

Statistical analysis was performed at each measure using a Mann-Whitney test.

### Results:

The results are depicted in FIG. 58. As shown therein, after 6 weeks of treatment, tumor volume/tumor burden on average shrank markedly, e.g., 28%, 79%, and 89% for MK-0646 12.5 μg/mouse, gemcitabine, MK-0646 12.5 μg/mouse+gemcitabine, respectively. When comparing the treated groups versus the control group, it is apparent that there was significant tumor growth inhibition between D8 and D16 (p<0.03) for the MK-0646 12.5 μg/dose described as a non-active dose for this model. For gemcitabine 138.5 mg/kg, significant tumor growth inhibition relative to the PBS group was observed between D22 and D37 (p<0.03). Indeed, tumor growth was significantly inhibited in the combination group with a calculated p value ≦0.04 between D5 and D43.

A significant difference between mice treated with gemcitabine and animals receiving gemcitabine in combination with MK-0646 12.5 μg/dose was observed on D41 and D43 (p≦0.04). Likewise a significant difference reduction/activity in tumor growth was observed between mice treated with MK-0646 and those receiving gemcitabine in combination with MK-0646 12.5 μg/dose between D33 and D43 (p≦0.03).

In this experiment, no mortality occurred during treatments. For gemcitabine injections, toxicity was observed neither in mice injected with gemcitabine alone nor when gemcitabine was administered in combination with MK-0646. See FIG. 59.

Taken together these results demonstrate that contrary to the published literature, gemcitabine was active in the BxPC3 in vivo xenograft model and that a statistical benefit of combining MK-0646 with gemcitabine was observed when MK-0646 was administered at low dose (12.5 μg/mouse).

### EXAMPLE 43

### Anti-Tumor Activity of Irinotecan and Mk-0646 (Humanized Anti-IGF-1R Antibody), Alone or in Combination, in the COLO 205 Xenograft Model (COL02050305)

### Materials and Methods

COLO 205 cells were routinely cultured in RPMI 1640 medium (Biowhittaker/Cambrex, Verviers, Belgium), supplemented with 10% FCS (Sigma Chemical Co. St Louis, Mo., USA), 2 mM L-Glutamine (Gibco/Invitrogen Corporation, Cergy-Pontoise, France), 10 mM HEPES (Gibco/Invitrogen), 1 mM sodium pyruvate (Biowhittaker/Cambrex), and adjusted to contain 2.5 g/l glucose (Sigma).

Cells were split two days before engraftment so that they were in exponential phase of growth. Five million COLO 205 cells (P+7) were engrafted on 7 weeks old Athymic-Nude mice (strain Hsd: Athymic-Nude Foxnlnu, Harlan, France).

Four days after engraftment (D0), tumors were measurable and animals were divided into homogeneous tumor size groups of 6 mice. Five days after engraftment (D1) mice were treated i.p. with a loading dose of MK-0646 (either alone or combined, 1 mg/dose per mice, injected 6 h post-Irinotecan injection when combined) and/or Irinotecan (diluted in DMSO, 100 mg/kg). From D3 to the end of experiment, MK-0646 was administered i.p. twice a week at 0.5 mg/dose per mice (6 h post-Irinotecan injection when combined). Irinotecan was administered at D1, D8, D15 and D22 (diluted in DMSO, 100 mg/kg). Control group was treated with PBS (vehicle for MK-0646) and DMSO (vehicle for Irinotecan), 6 h apart.

Tumor volume/browth was determined twice a week using the formula: π/6×length×width×height

Results:

Referring to FIG. 60, the data show that Irinotecan, when administered alone, inhibited in vivo tumor cell growth in the COLO 205 tumor cells, compared to the control group, with 87% inhibition at D25 (p≦0.04 from D15 to D25) when control group was sacrificed for ethical purposes.

In this xenograft experiment, it appears as if MK-0646 was not very effective in inhibiting tumor growth, when administered alone when compared to control group.

However, the rate of inhibition increased substantially when the MK-0646 was combined with Irinotecan. Specifically, in mice treated with the combination of MK-0646 and Irinotecan, the rate of inhibition was 94% compared to the control mice at D25. A significant effect was seen for the Irinotecan and MK-0646 combination compared to Irinotecan alone with up to 56% inhibition at D49 (p<0.04 from D21 to D49).

Referring to FIG. 61, toxicity for both Irinotecan alone or in combination was similar, transient and limited weight loss compared to control and MK-0646 groups.

### EXAMPLE 44

### In Vivo Efficacy of the Mk-0646 Humanized Antibody in Combination With Doxorubicin on In Vivo Growth of MCF-7 Oestrogen-Dependent Breast Cancer Cells (MCF70406)

### Materials and Methods

MCF-7 cells from ATCC (Rockville, Md., USA) were routinely cultured in phenol red free-RPMI medium (Invitrogen Corporation, Scotland, UK), 10% FCS (Invitrogen Corporation), 2 mM L-Glutamine (Invitrogen Corporation). Cells were split two days before engraftment so that they were in exponential phase of growth. Five million MCF-7 cells (P161) were engrafted in PBS to 8 weeks old Swiss nude mice. Mice received subcutaneous (s.c.) implants of slow release estrogen pellets (0.72 mg 17β-estradiol; Innovative Research of America, Toledo, Ohio, USA) one day before receiving tumor cell inoculation. Five days after implantation, tumors were measurable and animals were divided into 4 groups of 6 mice with comparable tumor size. Mice were treated either i.p. with a loading dose of 500 μg of MK-0646 followed by treatment doses of 250 μg/dose of MK-0646 alone or i.v. with 5 mg/kg of Doxorubicin (Sigma Chemical Co. St Louis, Mo., USA, Ref. D1515) alone or with a combination of both compounds.

Both Doxorubicin and MK-0646 were injected 4 times on D0, D7, D14 and D21. In groups receiving both the antibody and the chemotherapeutic compound, Doxorubicin was injected 6 hours before MK-0646 dosage. A control group receiving the same regimen of injections as the one of the combination group was introduced in this experiment. In this latter group mice received either PBS (i.p.) or water (i.v) injections depending on the compound administered to treated groups.

Tumor volume was measured twice a week and calculated by the formula: π/6×length×width×height. Statistical analysis was performed at each measure using a Mann-Whitney test.

### Results:

In this experiment, tumor volume/growth of single modality treated groups was reduced, on average, by 50% and 43% compared to the PBS group for MK-0646 and Doxorubicin 5 mg/kg respectively, 35 days after the first injection of the treatment. Indeed, on day 35 the groups of mice receiving a combination of MK-0646 and Doxorubicin exhibited a marked increase in tumor growth inhibition compared to the control group and to single modality treatments. Indeed, the rate of inhibition was 86% compared to the control group of mice and 73% and 76% compared to MK-0646 and Doxorubicin respectively. Refer to FIG. 62.

A statistical analysis of the data (Mann-Whitney test) demonstrated that, when mice were treated 4 times with the MK-0646 alone, a significant anti-tumor activity was observed in the MCF-7 model from day 7 to day 35 (p≦0.04). In the group of mice receiving Doxorubicin alone a significant inhibition of in vivo tumor growth was noticed on D7, D14, D17 and D35 (p≦0.04). Combined therapy of MK-0646+Doxorubicin 5 mg/kg improved tumor growth inhibition versus the PBS group (p≦0.002 from day 3 to day 35) and was statistically superior to Doxorubicin alone (p<0.03 from day 3 to day 35) or MK-0646 alone (p<0.02 from day 10 to day 35).

No lethal toxicity of Doxorubicin was noticed either alone or in combination with MK-0646 and curves plotting the % weight loss were comparable in both groups demonstrating that no additional toxicity was observed in the combined group compared to the single modality treatment group receiving Doxorubicin. Refer to FIG. 63.

No difference of the anti-tumoral activity and of the toxicity was observed when Doxorubicin was injected prior to the MK-0646 dosage (data not shown).

### EXAMPLE 45

### Anti-Tumor Activity of the Humanized MK-0646 Antibody Either Alone Or Combined with Docetaxel (TAXOTERE®)(MCF72004)

### Materials and Methods

MCF-7 cells from ATCC (Rockville, Md., USA) were routinely cultured in phenol red free-RPMI medium (Invitrogen Corporation, Scotland, UK), 10% FCS (Invitrogen Corporation), 2 mM L-Glutamine (Invitrogen Corporation). Cells were split two days before engraftment so that they were in exponential phase of growth. Five million MCF-7 cells (P152) were engrafted in PBS to 7 weeks old Swiss nude mice. Mice received subcutaneously (s.c.) implants of slow release estrogen pellets (0.72 mg 17β-estradiol; Innovative Research of America, Toledo, Ohio, USA) one day before receiving tumor cell inoculation. Six days after implantation, tumors were measurable and animals were divided into 7 groups of 6 mice with comparable tumor size. Mice were treated i.p. either once (D0) or 3 times (D0, D3, D6 or D1, D4, D7 depending on the schedule of antibody versus Docetaxel injections) with a dose of 1 mg/mouse of either MK-0646 or once with 40 mg/kg of Docetaxel (Fluka Ref. 01885 Sigma Chemical Co. St Louis, Mo., USA) or both. In groups receiving both the antibody and the chemotherapeutic compound, Docetaxel was injected either 6 hours after MK-0646 dosage or 18 hours prior to the first injection of MK-0646. A control group receiving the same regimen of injections as a combination group was introduced in this experiment. In this latter group, mice received either PBS or DMSO injection depending on the compound administered to treated groups.

Tumor volume was measured twice a week and calculated by the formula: π/6×length×width×height. Statistical analysis was performed at each measure using a Mann-Whitney test.

### Results:

In this experiment, the average tumor volume/growth of single modality treated groups was reduced by 25%, 16% and 71% compared to the control group of mice for MK-0646 injected once, MK-0646 injected 3 times and Docetaxel respectively, 50 days after the first injection of the treatment.

In the groups of mice receiving a combination of MK-0646 and Docetaxel, tumor growth inhibition was markedly improved compared to single modality treatment, independent of the tested schedule. The rate of inhibition reached 95, 93 and 91% for mice injected once with MK-0646 before Docetaxel treatment, 3 times with MK-0646 before Docetaxel treatment and with Docetaxel injected before MK-0646. See FIG. 64.

A statistical analysis of the data (Mann-Whitney test) demonstrates that in mice were treated once with the MK-0646 alone, significant anti-tumor activity was observed in the MCF-7 model from day 7 to day 32 (p<0.03). When MK-0646 was administered 3 times instead of once, significant inhibition of tumor growth was noticed from D7 to D24 (p<0.03). In the group of mice receiving Docetaxel alone, a significant inhibition of in vivo tumor growth was observed from day 7 to day 50 post first injection of Docetaxel (p<0.004). Combined therapy of MK-0646+Docetaxel improved tumor growth inhibition in all tested conditions when compared to the control group, p≦0.004 from day 7 to day 50 for MK-0646 D0+Docetaxel and p≦0.002 from day 7 to day 50 for both MK-0646 D0, D3, D6+Docetaxel and Docetaxel+MK-0646 D1, D4, D7. The results relating to the combination therapy were also statistically superior to Docetaxel alone (p≦0.03 from day 36 to day 50 for MK-0646 D0+Docetaxel; p<0.03 from day 39 to day 50 for MK-0646 D0, D3, D6+Docetaxel and p≦0.03 from day 39 to day 46 for Docetaxel+MK-0646 D1, D4, D7) or MK-0646 alone (p<0.004 from day 7 to day 50 versus MK-0646 D0+Docetaxel; MK-0646 D0, D3, D6+Docetaxel and Docetaxel+MK-0646 D0, D3, D6).

No additional toxicity was observed in groups receiving the combined therapies compared to the group treated with Docetaxel alone as shown in FIG. 65.

Humanized MK-0646 antibody enhanced the anti-tumor effect of Docetaxel in the MCF-7 xenograft model in all tested conditions (FIG. 64) without increasing the toxicity observed with the chemotherapeutic compound injected alone (FIG. 65).

### EXAMPLE 46

### Anti-Tumor Activity of the Humanized MK-0646 Antibody Either Alone Or Combined with Paclitaxel (TAXOL®)(MCF72505)

### Materials and Methods

MCF-7 cells from ATCC (Rockville, Md., USA) were routinely cultured in phenol red free-RPMI medium (Invitrogen Corporation, Scotland, UK), 10% FCS (Invitrogen Corporation), 2 mM L-Glutamine (Invitrogen Corporation). Cells were split two days before engraftment so that they were in exponential phase of growth. Five million MCF-7 cells (P159) were engrafted in PBS to 6 weeks old Swiss nude mice. Mice received subcutaneous (s.c.) implants of slow release estrogen pellets (0.72 mg 17β-estradiol; Innovative Research of America, Toledo, Ohio USA) one day before receiving tumor cell inoculation. Five days after implantation, tumors were measurable and animals were divided into 4 groups of 6 mice with comparable tumor size. Mice were treated i.p. three times a week with 5 μg/dose of MK-0646 alone or five times (D0, D1, D2, D3 and D4) i.p. with 6.25 mg/kg of Paclitaxel (Sigma Chemical Co. St Louis, Mo., USA, Ref. T1912) alone or with a combination of both compounds. In groups receiving both the antibody and the chemotherapeutic compound, Paclitaxel was injected 6 hours after MK-0646 dosage. A control group receiving the same regimen of injections as the one of the combination group was introduced in this experiment. In this latter group mice received either PBS or DMSO injection depending on the compound administered to treated groups.

Tumor volume was measured twice a week and calculated by the formula: π/6×length×width×height. Statistical analysis was performed at each measure using a Mann-Whitney test.

### Results:

In this experiment, mice of the control group were sacrificed on day 44 based on ethical criteria. As shown in FIG. 66, the average tumor volume of single modality treated groups was reduced by 46% and 67% compared to the PBS group for MK-0646 and Paclitaxel respectively, 44 days after the first injection of the treatment. In the groups of mice receiving a combination comprising MK-0646 and Paclitaxel, tumor growth inhibition was markedly improved compared to single modality treatment reaching 69% and 50% inhibition rates compared to MK-0646 and Paclitaxel respectively on day 44. See FIG. 66.

A statistical analysis of the data (Mann-Whitney test) shows that when mice were treated 3 times a week with the MK-0646 alone, a significant anti-tumor activity was observed in the MCF-7 model from day 3 to day 44 (p≦0.03). In the group of mice receiving Paclitaxel alone, significant inhibition of in vivo tumor growth was observed from day 3 to day 24 post first injection of Paclitaxel (p≦0.04). Combined therapy of MK-0646+Paclitaxel improved tumor growth inhibition (p≦0.004 from day 3 to day 44 versus Control) and was statistically superior to Paclitaxel alone (p≦0.009 from day 8 to day 48) or MK-0646 alone (p<0.02 from day 3 to day 27).

No additional toxicity was observed in groups receiving the combined therapies compared to the group treated with Paclitaxel alone.

According to the data presented in FIG. 66, humanized Mk-0646 antibody enhanced the anti-tumor effect of Paclitaxel in the MCF-7 xenograft model without increasing the toxicity observed with the chemotherapeutic compound injected alone.

### EXAMPLE 47

### Efficacy of MK-0646 and Herceptin Combination in Orthotopic SKOV3Ip (Ovarian) Mouse Xenograft Model

Seventy NCR-nude mice were injected intraperitoneally (i.p.) with 5×105 human ovarian adenocarcinoma cells SKOV31p. Five days following the injection all mice were divided into groups of 10 (see Table 13). A loading dose, which is a double dose for the first injection, was given to each group for all treatments to guarantee optimal dosing on the very first injection. See FIG. 67. Consequently, each group was treated twice a week with either a single antibody or a combination of both antibodies as described below. The MK-0646 antibody was used at one concentration of 500 μg per dose. The second antibody (Herceptin) was used at 10, 50 or 100 μg per dose.

TABLE 13

MK-0646 500 mg
Herceptin 10 mg
Herceptin 50 mg
Herceptin 100 mg
MK-0646 500 mg + Herecptin 10 mg
MK-0646 500 mg + Herceptin 50 mg
MK-0646 500 mg + Herceptin 100 mg

The three combinations were 500 μg MK-0646 10 μg plus Herceptin, 500 μg MK-0646 plus 50 μg Herceptin, 500 μg MK-0646 plus 100 μg Herceptin (see Table 13)

The animals were treated for four weeks and were monitored for survival for 60 days. See FIG. 67.

Results: Referring to FIG. 68, a significant increase of survival, as represented in the dose response, was observed when mice were treated with increasing doses of Herceptin as compared to the control buffer treated group. All control mice succumbed to disease by day thirty, whereas 50% and 100% of the mice were still alive in the 10, 50 and 100 μg treatment groups respectively at that time.

Referring to FIG. 69, an additive effect on survival was observed in the group treated with the combination of 500 μg of MK-0646 and 10 μg of Herceptin compared to the groups treated with 500 μg MK-0646 or 10 μg of Herceptin antibody alone (Spearman-Karber method of statistical analysis was used).

However, no additive effect was observed when 50 μg (FIG. 70) or 100 μg of Herceptin (data not shown) were used in combination with 500 μg of MK-0646. This may be due to the higher sensitivity of the cell line to Herceptin treatment

### EXAMPLE 48

### Preclinical Results Demonstrating Efficacy of MK-0646 in Colon Tumors

I. Efficacy of MK-0646 antibody in colon tumor xenografts.

To evaluate the efficacy of MK-0646 antibody in vivo, NCR nude mice were injected subcutaneously with various human colon tumor cell lines. Each tumor cell line was in vitro characterized for IGF1R, IR, EGFR, receptor level expression as well as the percentage MK-0646 mediated IGF1R internalization by flow cytometry. Five to 10 days after tumor-cell injection, the size of tumors were determined and the mice were randomized into groups with equivalent average tumor size (n=7-10). On the day of randomization, treatment with MK-0646 was started and it was continued for 4 weeks. Weekly administration of 500 μg of MK-0646 delayed the tumor growth of Colo 205 (FIG. 71A) and HT29 (FIG. 71B) tumor xenografts compared to control buffer treated mice. Efficacy was also observed in mice implanted with Geo tumors cells following biweekly treatments with 500 μg of MK-0646 (FIG. 71C). In addition to being susceptible to MK-0646 treatment in vivo, these cell lines showed a high percentage MK-0646 mediated receptor internalization in vitro (Table 14) 69, 49 and 51% for Colo205, HT29 and Geo cells respectively. Tumor cell lines LS123, LS411N and SW403, which had low or non-detectable levels of MK-0646 receptor internalization (Table 14) were resistant to MK-0646 treatment in vivo.

TABLE 14

| | at | Tumor | internal- | MK0646 | | | | | | |
|---|---|---|---|---|---|---|---|---|---|---|
| Cell Line | site | Type | ization | efficacy | IGF1R:IR | IGF1R | IR | EGFR | -erbB2 | PTEN |
| HT29 | wp | Colon | 49% | sus | 1.61 | 12214 | 7594 | 34202 | 15364 | (+) |
| Colo 205 | wp | Colon | 69% | sus | 2.32 | 16502 | 5532 | 17682 | 29789 | (+) |
| WiDr | wp | Colon | 55% | res | 2.67 | 12752 | 4777 | tbd | tbd | tbd |
| Geo | wp | Colon | 51% | sus | 1.39 | 4928 | 3539 | 43528 | 9580 | (+) |
| LoVo | wp | Colon | 25% | res | 0.64 | 10047 | 15764 | 40929 | 8424 | tbd |
| LS123 | wp | Colon | 0% | res | 0.47 | 2438 | 6300 | 8155 | 12950 | tbd |
| LS411N | wp | Colon | 0% | res | 0.39 | 5433 | 13821 | 19518 | 7002 | tbd |
| SW403 | wp | Colon | 0% | res | 0.27 | 1494 | 5583 | 14165 | 14572 | neg |

II. Effect of MK-0646 on the in vivo growth of human colon tumor segments implanted s.c into nude mice.

Colon tumor pieces from six individual patients were collected and subsequently expended in vivo in nude mice to generate enough tumor material for an efficacy study. Each mouse received two tumor fragments from each patient derived tumor, which were implanted into both flanks. Tumor-bearing mice were randomized and were stratified into treatment and vehicle control groups according to tumor volume, using “Lindner's Randomization Tables”. In vivo therapy study: IGF-1R at 500 μg/mouse and one vehicle were given i.p. on day 0, 7, 14, and 21. Tumor volumes were calculated according to the formula a*b*b/2 where “a” is the longest and “b” the perpendicular axis. Efficacy of MK-0646 was observed in 50% (3/6) of the tested tumors with optimal treated/control [%] lower than 60% (Table 15).

TABLE 15
Efficacy of MK-0646 in reducing tumor growth of 50% (3/6) human
colon tumors pieces implanted subcutaneously in nude mice

| colon carcinoma | Treated/Control[%] |
|---|---|
| CXF 94LX | 46.6* |
| CXF 158 | 66.7 |
| CXF 280 | 51.7* |
| CXF 975 | 95.4 |
| CXF 1103 | 78.8 |
| CXF 1729 | 57.2* |

III. Enhanced efficacy of MK-0646 antibody in combination with Erbitux in HT29 colon mouse xenograft model.

NCR-nude mice were injected intraperitoneally (i.p.) with 5×10⁶ human colon adenocarcinoma HT29 cells. Ten days following the injection, all mice bearing tumors were divided into 5 groups of 9 mice. Each group was treated twice a week with a single antibody or a combination of both antibodies. To evaluate if the order of antibody administration is important for the efficacy of the combination, mice were treated first with MK-0646 followed by Erbitux 6 hours later or vice versa (see FIG. 72 legend).

Synergy between MK-0646 and cetuximab (Erbitux) was observed in HT29 human colorectal subcutaneous mouse xenografts. Tumors in mice treated with the combination of antibodies grew significantly more slowly than tumors in mice treated with either antibody alone.

### EXAMPLE 49

### Orthotopic In Vivo Model of Pancreatic Cancer

MiaPaca2 cell line was maintained in DMEM (Gibco) supplemented with 10% fetal calf serum and 2.5% horse serum. When 70%-80% confluent, cells were trypsinized, washed twice with phosphate buffered saline (PBS) and finally resuspended in PBS at 2×107 cells/ml. Female athymic mice 5 weeks old were purchased from Harlan Nossan and housed under specific pathogen free conditions according to institutional guidelines. 60 mice received surgically intra-pancreas inoculation of 50 μl of MiaPaca2 cell suspension. Treatment started 4 hrs post-cell implantation. MK-0646 was diluted in histidine buffer (15 mM histidine, 150 mM NaCl) to 1 and 4 mg/ml (20 mice/group) and administered bi-weekly i.p. at the amount of 100 μl/mouse. Control group (20 mice) was treated i.p. with 100 μl of histidine buffer. Mice were monitored twice a week for adverse effects (body weight, body temperature, swollen abdomen, presence of sub-cutaneous tumoral mass).

10 mice/group were euthanized 35 and 63 days post-cell implantation. Mouse weight, primary tumor weight, distribution and number of the metastases, volume of ascites were recorded as set forth in FIGS. 73-75.

Referring to FIG. 73, Athymic mice received intra-pancreas 1×106 MiaPaca2 cells and were treated with either 100 or 400 μg of MK-0646. Reported data represent the average of the primary tumor weight recorded in the group (n=10). The error bars represent the standard error. For group comparison Student's t-test was applied. p-value <0.05 was considered statistically significant 74. Athymic mice received intra-pancreas 1×106 MiaPaca2 cells and were treated with either 100 or 400 μg of MK-0646. Reported data represent the average of the primary tumor weight recorded in the group (n=10). The error bars represent the standard error. For group comparison Student's t-test was applied. p-value <0.05 was considered statistically significant

FIG. 75 Athymic mice received intra-pancreas 1×106 MiaPaca2 cells and were treated with either 100 or 400 μg of MK-0646. Reported data represent the total volume of ascites (ml) and the total number of metastases counted in mice from the same group (n=10).

For statistical analysis, Student's t test for two group comparison was applied (N=10, p<0.05).

### EXAMPLE 50

### Effect of MK-0646 on Ovarian Carcinoma

| | |
|---|---|
| Mouse strain: | NMRI nu/nu |
| Animals supplied by: | Elevage Janvier, France |
| Body weight range at randomization | 20.7-35.5 g |
| Approximate age at implantation: | 4-6 weeks |
| Approximate age at randomization: | 5-12 weeks |

### 1.2 Study Design—Grouping and Randomization of Animals

One group of 7 mice received a vehicle control (Group 1), 20 mM-L Histidine, 150 mM NaCl, 0.5% PS-80 w/w, pH6.5 at 250 μL/mouse ip once weekly and a second group treated received MK-0646 IGF-1R mab at 500 μg/mouse ip once weekly (Group 2). Mice received bilateral tumor implants. At randomization tumor bearing animals were stratified into treatment and vehicle control groups according to tumor volume. Only animals carrying at least one tumor of appropriate size (mean tumor diameter: 6-8 mm, minimum acceptable tumor diameter: 5 mm) were considered for randomization. The day of randomization was designated as Day 0.

### 1.3 Animal Identification

Animals were arbitrarily numbered during tumor implantation using ear clips. At the beginning of the experiments, each cage was labeled with a record card, indicating the experiment number, date of tumor implantation, date of randomization, tumor type, tumor number, mouse strain, gender, and individual mouse number. After randomization group identity, test compound, dosage, schedule, and route of administration were added.

### 2. Housing Conditions

### 2.1 Husbandry

The animals were housed in Tecniplast™ individually ventilated cages. According to group size the animals were housed either in Macrolon™ type III cages (maximum 10 mice/cage). The cages were sterilized at 121° C. before use and changed twice a week. The temperature inside the cages was maintained at 25±1° C. and relative humidity at 60±10%. The animals were kept under a natural daylight cycle.

### 2.2 Diet and Water Supply

The animals were fed Altromin Extrudat 1439 Rat/Mouse diet. The diet was purchased from Altromin GmbH (Lage, Germany).

Water was sterilized at 121° C. for 30 minutes. After sterilization 0.9 g/l potassium sorbate was added, the pH was adjusted to 2 with 1N HCl. Water consumption was visually monitored daily, the bottles were changed twice a week. Food and water were provided ad libitum.

### 2.3 Bedding

The dustfree animal bedding Lignocel FS 14 produced by Rettenmaier & Söhne Faserstoffwerke (Ellwangen-Holzmühle, Germany) was purchased from ssniff Spezialdiäten GmbH (Soest, Germany). The bedding was renewed twice a week. The producer analyzes the dust-free bedding every 3 months with respect to biological/fungal contamination and content of phosphate esters, arsenic, cadmium, lead and mercury. These analyses are carried out at the Agriculture Analyses and Research Institute, Ministry of Agriculture, Kiel, Germany. The quality certificates are deposited at Rettenmaier & Söhne Faserstoffwerke (Ellwangen-Holzmühle, Germany).

### 3. Tumor Information

### 3.1 Characterization of Tumor Models

The OVXF 899 xenografts were derived from a surgical specimen of a moderately differentiated ovarian carcinoma from a 76 year old patient and directly implanted into nude mice. The tumor xenografts were passaged in nude mice until establishment of a stable growth pattern. The tumors used in this study had been passaged a total of 35 times in mice and have a doubling time of 4-8 days in mice.

### 3.2 Implantation of Human Tumor Xenografts

Tumor fragments were obtained from xenografts in serial passage in nude mice. After removal of tumors from donor mice, they were cut into fragments (1-2 mm diameter) and placed in RPMI 1640 culture medium until subcutaneous implantation. Recipient mice were anaesthetized by inhalation of isoflurane. For the implantation one small incision was made in the skin of the back. The tumor fragments (either 1 or 2 fragments per mouse) were transplanted with tweezers. The mice were monitored daily.

### 4. Supply and Formulation of Test Substances

For the final dosing concentration of 2 mg/mL the antibody solution (11.3 mg/ml) was diluted with 20 mM-L Histidine, 150 mM NaCl, 0.5% PS-80 w/w, pH6.5 at a ratio of 1:5.65. This diluted solution was administered at an application volume of 250 μL/mouse for the dose level of 500 μg/mouse. The control vehicle was 20 mM-L Histidine, 150 mM NaCl, 0.5% PS-80 w/w, pH6.5. In the preparation of this vehicle 3-Histidine Monohydrochloride Monohydrate (Sigma Prod No H8125 Lot No. 064K0380 Formula Wt. 209.6) was used. The vehicle was administered at 250 μL/mouse.

### 5. Treatment Procedure

### 5.1 Route of Administration

MK-0646 and the control vehicle were injected intraperitoneally.

### 5.2 Drug Dosage and Treatment Regimen

For efficacy testing MK-0646 was dosed once weekly at 500 μg/mouse ip for the duration of the experiments. The control vehicle 20 mM-L Histidine, 150 mM NaCl, 0.5% PS-80 w/w, pH6.5 was injected at 250 μL/mouse on the same days.

### 6. Observations

### 6.1 Mortality

Mortality checks were conducted daily.

### 6.2 Tumor Volume

The tumor volume was determined by two-dimensional measurement with a caliper on the day of randomization (Day 0) and then twice weekly (ie on the same days on which mice were weighed). Tumor volumes were calculated according to the formula: (a×b2)×0.5 where a represents the largest and b the perpendicular tumor diameter.

### Results:

As shown in FIG. 76, mice implanted with OVXF 899 ovarian cancer xenografts and subsequently treated with MK-0646 showed a significant tumor reduction compared to animals treated with vehicle.

### EXAMPLE 51

### BXPC3 Xenograft Model

For BxPC-3 xenografts 7 million cells in PBS were engrafted subcutaneously into 6-weeks old Ncr nude mice. Twenty eight days after implantation animals were divided into 4 groups of 10 mice with comparable tumor size. Mice were treated i.p. with 100, 150 and 500 μg MK-0646 twice a week. Referring to FIG. 77, the highest efficacy compared to PBS control group was observed in mice treated with 500 μg.

### EXAMPLE 52

Rationale: The insulin-like growth factor and mTOR pathways have been connected to sarcoma development and progression. Preliminary data suggests that mTOR pathway inhibition can slow growth of sarcoma xenografts, including rhabdomyosarcoma. To date, mTOR inhibition alone has failed to eliminate tumors and appears to secondarily upregulate phospho-AKT in sarcomas. As a consequence, the investigators hypothesized that the use of an IGF-1R antibody (7C10/MK-0646) to specifically block IGF-1R mediated cell signaling pathway alone or in combination with an agent that blocks the mTOR pathway (rapamycin) may be effective in reducing tumor burden or cell proliferation thereby leading to improved antitumor activity and preventing deleterious upregulation of phospho-AKT. Towards this end, experiments were designed using an IGF-1R specific antibody alone (7C10) or in combination with an mTOR inhibitor to evaluate the effect on tumor growth and/or cell proliferation. Refer to FIGS. 78-81.

Using rhabdomyosarcoma (RMS) cell lines and xenografts, the investigators observed that the IGF-IR antibody (h7C10) alone decreased alveolar RMS (Rh30) cell number by 50% at 48 hrs, but may have had inconsistent effect on an embryonal RMS (RD) cell line as measured by MTT assay (10 ug/mL at 48 hrs). According to the data, h7C10 decreased phospho-AKT in Rh30 cell lysates (10 ug/mL at 48 hrs). Surprisingly, in vivo growth (measured by mean chemiluminescent intensity units) of established Rh30 cells expressing luciferase (Rh30-Luc) in SCID/beige mice was significantly decreased with h7C10 monotherapy (52.2±25.3) compared to mice treated with vehicle control (19 mm+0.82) by day 36. See FIG. 81.

The combination of h7C10 and rapamycin yielded results similar to h7C10 (11.9+10.4, p<0.001), whereas single agent rapamycin had no significant effects (55+20.6, p=0.7) at this time point. Preliminary results support a role for the IGF-1R antibody (7C10/MK-0646) in treating sarcoma primary tumors as a single agent as well as in combination therapy with, for example, an mTOR inhibitor. Specifically, the data support a strategy that calls for blocking IGF-1R mediated signaling via use of an IGF-1R monoclonal antibody such as 7C10 as a means of treating pediatric primary soft tissue sarcomas either as a stand alone therapy (single agent) or in combination with an mTOR inhibitor.

### Methods:

h7C10 (10 ug/mL) decreases Rh30 and RH41 rhabdomyosarcoma cell number. MTT (3-(4,5-dimethylthiazol-2-yl)-2,5-diphenyl tetrazolium bromide) proliferation assay was used to assess the effect of h7C10 treatment on Rh30, RH1, RH41, RD4A and RD rhabdomyosarcoma cells in vitro. Briefly, cells were plated in a 96-well format at 1×10⁴ cells/well. Cells were allowed to adhere overnight treated with either complete RPMI medium alone or h7C10 (10 ng/mL) in complete RPMI medium. Media was suctioned and then MTT reagent was added at 100 uL/well to each plate. Cells were incubated at 37° C. for 4 hrs and then isopropyl alcohol was added at 100 ul/well for a final volume of 200 ul/well. Plates were allowed to sit for 10 min at room temperature after thoroughly mixing the isopropyl alcohol and MTT reagent. Optical density was analyzed with a 96-well plate VERSAmax reader. The cells were read at λ1=570, λ2=690. Refer to FIGS. 78 A & B, 79 A & B. See also FIGS. 80 (A) & (B) and 81, which detail the various endpoint measurements, e.g., chemiluminescent measurement (luciferaes) & caliper measurement of primary tumor.

h7C10 (10 ug/mL) decreases Rh30 and RH41 phosphorylation of downstream targets of IGF-1R. Rhabdomyosarcoma cells were treated with h7C10 (10 ng/mL) for 48 hrs or 96 hrs in complete RPMI and then lysed in lysis buffer for western blot analysis for phosphorylation and expression of p-AKT and p-p-MAPK p44/p42 (primary antibody concentrations=0.01 ug/mL; secondary antibody concentrations=0.2 ug/mL). Protein lysates (20-50 μg/lane) collected from h7C10 treated and untreated cells for Western analysis. Confluent cells were lysed in SDS lysis buffer (Cell Signaling Technology Inc., Beverly, Mass.) at room temp for phospho-AKT, AKT phospho-p44/42 MAPK, p44/42 MAPK, and β-actin. The data showed that there was a decrease in the phosphorylation of downstream targets of IGF-1R. See FIGS. 78 A & B.

IGF-IR (h7C10) alone and in combination with rapamyin decreases primary tumor growth in Rh30-Luc xenografts. Refer to FIGS. 80 A & B. Mice bearing Rh30 rhabdomyosarcoma xenografts were treated IP with h7C10 (12.5 mg/kg) q4d alone, rapamycin (5 mg/kg) q3d alone, the combination of h7C10 and rapamycin, or vehicle for 57 days. Two million Rh30-Luc tumor cells were injected to the left gastrocnemius muscle group by IM injection of 100 ul of tumor cell suspension using a 27 g needle, to Beige-SCID or athymic nude mice. All mice were imaged weekly by D-luciferin Xenogen imaging and using caliper measurements to assess primary tumor growth. Using both measurement methods, a significant reduction in primary tumor growth was seen in mice receiving h7C10, rapamycin and the combination in vivo. All animal studies were conducted following the approval of the NCI—Animal Care and Use Committee.

1-54. (canceled)

55. A method for the treatment or prevention of an oncogenic disorder characterized by cells expressing aberrant levels of IGF-1R relative to normal, said method comprising administering to the patient a therapeutically effective amount of a humanized anti-IGF-IR specific antibody or antigen-binding fragment thereof effective in treating said disorder, wherein
(a) said oncogenic disorder is selected from the group consisting of, ovarian cancer, colon cancer, pancreatic cancer, breast cancer, and prostate cancer, and
(b) wherein said IGF-1R antibody or antigen-binding fragment comprises a light chain complementarity determining region (CDR) selected from the group consisting of SEQ ID Nos. 2. 4 and 6 or at least one CDR having at least 95% sequence identity with one or more of SEQ ID Nos 2, 4 or 6 and a heavy chain complementarity determining region (CDR) selected from the group consisting of SEQ ID Nos. 8, 10 and 12 or at least one CDR having at least 95% sequence identity with one or more of SEQ ID Nos 8. 10 or 12, wherein said antibody, or binding fragment thereof, binds to human insulin-like growth factor I receptor (IGF-IR) and inhibits binding of IGF1 and/or IGF2 to said IGF-IR

56. The method according to claim 55, wherein the heavy chain CDR comprises an amino acid sequence selected from the group consisting of SEQ ID NOs. 8, 10 or 12 and the light chain CDR comprises an amino acid sequence selected from the group consisting of SEQ ID NOs. 2, 4 or 6.

57. The method according to claim 55, wherein said humanized antibody, or one of its functional fragments, comprises a light chain comprising the amino acid sequence selected from the group consisting of SEQ ID No. 61 or 65, or a heavy chain comprising the amino acid sequence selected from the group consisting of SEQ ID NOs.: 75, 79 or 83.

58. A method of assaying a subject for an IGF-1R mediated oncogenic disorder, comprising: a) obtaining a test sample of cells derived from said subject; b) assaying said test sample to determine the amount of expression of IGF-1R that is present on a surface of said cells; c) comparing the amount of IGF-1R expression determined in step b) with the amount of IGF-1R expression in a control sample of cells; and d) determining if said subject is at increased risk of having or developing said IGF-1R mediated disorder based upon the comparison of step c), wherein said oncogenic disorder is selected from the group consisting of, ovarian cancer, colon cancer, pancreatic cancer, breast cancer, and prostate cancer.

59. The method according to claim 55, further comprising administering to said subject said humanized antibody in combination with an additional cancer therapeutic agent.

60. The method according to claim 55, wherein said additional cancer therapeutic agent is selected from the group consisting of tumor irradiation or a chemotherapeutic agent.

61. The method according to claim 60, wherein said additional chemotherapeutic agent is an agent selected from the group consisting of a corticosteroid, anti-emetic, cancer vaccine, analgesic, anti-vascular agent, cytokines, non-specific immunostimulant and anti-proliferative agent.

62. The method according to claim 60, wherein said comprises a DNA damaging agent selected from the group consisting of verapamil, podophyllotoxin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide (VP 16), tamoxifen, taxol, transplatinum, 5-fluorouracil, vincristin, vinblastin, methotrexate, cisplatin, carboplatin, oxaliplatin, and combinations thereof.

63. The method of claim 60, wherein said irradiation is selected from the group consisting of X-ray radiation, UV-radiation, γ-radiation, microwave radiation, and combinations thereof, further comprising administering to said mammal said antibody in combination with an agent selected from the group consisting of a corticosteroid, anti-emetic, cancer vaccine, analgesic, anti-vascular agent, and anti-proliferative agent.

64. The method according to claim 55, further comprising administering to said subject said humanized antibody in combination with an anti-emetic agent, wherein said agent is selected from the group consisting of ondansetron hydrochloride, granisetron hydrochloride, metroclopramide, domperidone, haloperidol, cyclizine, lorazepam, prochlorperazine, dexamethasone, levomepromazine, or tropisetron.

65. The method according to claim 55, further comprising administering said antibody in combination with an analgesic agent, wherein said agent is selected from the group consisting of ibuprofen, naproxen, choline magnesium trisalicylate, or oxycodone hydrochloride.

66. The method according to claim 55, further comprising administering said humanized antibody in combination with an anti-proliferative agent, wherein said agent is selected from the group consisting of farnesyl protein transferase inhibitors, .alpha.v.beta.3 inhibitors, alpha.v.beta.5 inhibitors, p53 inhibitors, VEGF inhibitors, VEGFR inhibitors, EGFR inhibitors and Her2neu inhibitors or heterodimers thereof, and PDGFR inhibitors.

67. (canceled)

68. A method for the treatment or prevention of an oncogenic disorder characterized by cells expressing high levels of IGF-1R relative to normal, said method comprising administering to the patient a therapeutically effective amount of a humanized anti-IGF-1R specific antibody or antigen-binding fragment thereof effective in treating said disorder, wherein
(a) said oncogenic disorder is selected from the group consisting of neuroblastoma, rhabdomyosarcoma, osteosarcoma, pediatric cancer, Wilm's cancer and Ewing Sarcoma, and
(b) wherein said IGF-1R antibody or antigen-binding fragment comprises a light chain complementarity determining region (CDR) selected from the group consisting of SEQ ID Nos. 2, 4 and 6 or at least one CDR having at least 95% sequence identity with one or more of SEQ ID Nos 2, 4 or 6 and a heavy chain complementarity determining region (CDR) selected from the group consisting of SEQ ID Nos. 8, 10 and 12 or at least one CDR having at least 95% sequence identity with one or more of SEQ ID Nos 8, 10 or 12, wherein said antibody, or binding fragment thereof, binds to human insulin-like growth factor I receptor (IGF-IR) and inhibits binding of IGF1 and/or IGF2 to said IGF-IR.

69. The method according to claim 68, further comprising administering to said subject said humanized antibody in combination with an additional cancer therapeutic agent.

70. The method according to claim 69, wherein said additional cancer therapeutic agent is selected from the group consisting of tumor irradiation or a chemotherapeutic agent.

71. The method according to claim 70, wherein said additional chemotherapeutic agent is an agent selected from the group consisting of a corticosteroid, anti-emetic, cancer vaccine, analgesic, anti-vascular agent, cytokines, non-specific immunostimulant and anti-proliferative agent.
